(19) United States
(12) Patent Application Publication
(10) Pub. No.: US 2024/0309345 A1
(43) Pub. Date: Sep. 19, 2024

(54) COMPOSITIONS AND METHODS FOR GENE EDITING

(71) Applicant: The Regents of the University of California, Oakland, CA (US)

(72) Inventors: Luke Gilbert, San Francisco, CA (US); James Nunez, San Francisco, CA (US); Jonathan Weissman, San Francisco, CA (US); Jin Chen, San Francisco, CA (US)

(21) Appl. No.: 17/999,756

(22) PCT Filed: Jun. 1, 2021
(86) PCT No.: PCT/US2021/035244

§ 371 Date: Nov. 23, 2022

Related U.S. Application Data
(60) Provisional application No. 63/171,698, filed on Apr. 7, 2021.
(60) Provisional application No. 63/114,850, filed on Nov. 17, 2020.
(60) Provisional application No. 63/033,397, filed on Jun. 2, 2020.

(51) Int. Cl. C12N 9/22; A61K 48/00; A61P 31/14; C12N 9/10

(52) U.S. Cl. CPC C12N 9/22; A61K 48/005; A61P 31/14; C12N 9/1007; C07K 2319/09; C12N 2310/20

(57) ABSTRACT

Provided herein are, inter alia, fusion proteins, compositions and methods for manipulation of genomes of living organisms.

### CROSS-REFERENCE TO RELATED APPLICATIONS

This application is a national stage entry, filed under 35 U.S.C. § 371, of International Application No. PCT/US2021/035244, filed Jun. 1, 2021, which claims priority to U.S. Provisional Application No. 63/171,698 filed Apr. 7, 2021, U.S. Provisional Application No. 63/114,850 filed Nov. 17, 2020, and U.S. Provisional Application No. 63/033,397 filed Jun. 2, 2020, the disclosures of which are incorporated by reference herein in their entireties.

### STATEMENT AS TO RIGHTS TO INVENTIONS MADE UNDER FEDERALLY SPONSORED RESEARCH AND DEVELOPMENT

This invention was made with government support under grant no. HR0011-17-2-0043 awarded by The Defense Advanced Research Projects Agency. The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 048536-682001WO_SequenceListing.txt, created Jun. 3, 2021, 531 Kilobytes, machine format IBM-PC, MS Windows operating system, is hereby incorporated by reference.

### BACKGROUND

Although considered a promising therapeutic approach for treatment of disease, genome editing carries inherent risks due to the potential for genotoxicity from double strand breaks. Further, genome editing often is associated with an all-or-none effect on the target gene (i.e., it produces a full knockout). In contrast, targeted epigenome engineering does not carry the risk of DSB-induced genotoxicity; further, it affords the opportunity to create a more graded effect on gene expression and thus function from a complete silencing through a less pronounced effect. Provided herein are solutions to these and other needs in the art.

### BRIEF SUMMARY

The disclosure provides fusion proteins comprising a DNA methyltransferase domain, a first XTEN linker comprising from about 5 to about 864 amino acid residues, a nuclease-deficient RNA-guided endonuclease enzyme, a second XTEN linker comprising from about 5 to about 864 amino acid residues, and a Krüppel-associated box domain. In aspects, the first XTEN linker comprises from greater than 50 to about 864 amino acid residues, and the second XTEN linker comprises from about 5 to 50 amino acid residues. In embodiments, the nuclease-deficient RNA-guided endonuclease enzyme is a CRISPR-associated protein.

The disclosure provides methods of silencing a target nucleic acid sequence in a cell, including delivering a first polynucleotide encoding a fusion protein as described herein, including embodiments and aspects thereof, to a cell containing the target nucleic acid, and delivering to the cell a second polynucleotide comprising sgRNA or cr:tracrRNA. In aspects, the target nucleic acid comprises a CpG island. In aspects, the target nucleic acid does not comprise a CpG island. In embodiments, the nuclease-deficient RNA-guided endonuclease enzyme is a CRISPR-associated protein. Methods of silencing a target nucleic acid sequence can be used to treat numerous diseases, such as infectious diseases.

The disclosure provides fusion proteins comprising a DNA methyltransferase domain, a first XTEN linker comprising from about 5 to about 864 amino acid residues, a nuclease-deficient endonuclease enzyme (e.g., a zinc finger domain, a TALE), a second XTEN linker comprising from about 5 to about 864 amino acid residues, and a Krüppel-associated box domain. In aspects, the first XTEN linker comprises from greater than 50 to about 864 amino acid residues, and the second XTEN linker comprises from about 5 to 50 amino acid residues.

The disclosure provides methods of silencing a target nucleic acid sequence in a cell, including delivering a first polynucleotide encoding a fusion protein as described herein, including embodiments and aspects thereof, to a cell containing the target nucleic acid. Methods of silencing a target nucleic acid sequence can be used to treat numerous diseases, such as infectious diseases.

These and other embodiments and aspects of the disclosure are described in detail herein.

### BRIEF DESCRIPTION OF THE DRAWINGS

FIGS. 1A-1G show durable and multiplexed gene silencing by CRISPRoff. FIG. 1A: a schematic of dCas9 epigenetic editor fusion proteins that were tested for gene silencing activity. 3L denotes Dnmt3L. FIG. 1B: plasmids encoding dCas9 fusions and sgRNAs were co-transfected into HEK293T cells stably expressing a DNA methylation-sensitive Snrpn-GFP reporter. Transfected cells were sorted 2 days after transfection and GFP silencing was monitored over time. FIG. 1C: a time course comparing GFP silencing activities of CRISPRoff-V1, dCas9-3A-3L, and dCas9-KRAB. FIG. 1D: bisulfite PCR analysis of the Snrpn locus before or after CRISPRoff targeting. The white circles indicate unmethylated CpG dinucleotides and black circles represent methylated CpG dinucleotides. Each row represents one sequencing read. The red square denotes the sgRNA binding site. FIG. 1E: a comparison of CRISPRoff-V1 (black) and CRISPRoff-V2 (blue) editors in silencing the endogenously GFP-tagged H2B gene. The dotted lines represent protein expression of CRISPRoff-V1 and -V2. FIG. 1F: a representative flow cytometry plot of H2B-GFP expression of cells at 50 days post-transfection of CRISPRoff V2. FIG. 1G: bisulfite sequencing analysis of a 126 bp region of the H2B CpG island. The red square denotes the sgRNA binding site. FIG. 1H: quantification of cells with ITGB1, CD81, or CD151 silenced 3 weeks post-transfection (p.t.) of CRISPRoff-V1 or -V2 with individual sgRNAs (a-c) or a pool of three sgRNAs (a, b, c). FIG. 1I: quantification of cells with ITGB1, CD81, CD151 silenced 30 days p.t. from single or double gene targeting experiments. FIG. 1J: quantification of multiplexed triple gene silencing by either gating on ITGB1-off cells then gating for CD81- and CD151-off cells (left bar) or by first gating on ITGB1-off cells, then CD151-off cells, and finally CD81-off cells (right bar). The asterisks denote the population of cells with the marked gene turned off. FIG. 1K: a representative flow cytometry plot of cells targeted for ITGB1, CD81, and CD151 silencing. Cells were first gated on ITGB1 silencing and the represented population displays CD81 and CD151 silencing. FIG. 1L: a histogram plot of CLTA expression at 15 months p.t. showing 38 clones that retained CLTA repression and one clone that reactivated CLTA expression. The mean values in FIGS. 1C, 1E, 1H, 1I, 1J, and 1K were measured from three independent experiments. Error bars represent SD of the mean.

FIGS. 2A-2H show highly specific and robust transcriptional silencing by CRISPRoff. FIGS. 2A-2D: RNA-seq plots of HEK293T cells transfected with CRISPRoff and non-targeting (NT) sgRNAs compared to sgRNAs targeting (FIG. 2B) ITGB1, (FIG. 2C) CD81, or (FIG. 2D) CD151. A comparison of untransfected cells and CRISPRoff with NT sgRNA is shown in (FIG. 2A). The volcano plots (bottom) display the targeted genes as the most significantly repressed transcripts globally. The data are representative of the average of two independent replicates. FIG. 2E: a Manhattan plot displaying differentially methylated CpGs between cells treated with CRISPRoff and CLTA-targeting or NT sgRNAs (30 days post-transfection) analyzed by WGBS. Red dots represent CpGs that gained DNA methylation in targeting sgRNA cells and blue dots represent CpGs that gained DNA methylation in NT sgRNA cells. The arrow denotes the genomic position of CL TA. FIG. 2F: a comparison of CpG methylation along a 55 kb window that includes the CLTA locus. Tracks labelled ‘Untr.’ represent untransfected cells; the ‘NT’ tracks represent cells transfected with CRISPRoff and non-targeting sgRNA; the ‘T’ tracks represent cells transfected with CRISPRoff and targeting sgRNA. R1 and R2 represent two technical replicates. Red marks represent methylated (beta-value >0.5) and the blue marks represent unmethylated (<0.5) CpG dinucleotides. CpG islands are shown in green. FIG. 2G: a comparison of H3K9me3 ChIP-seq signal across the H2B gene in cells transfected with CRISPRoff and H2B-targeting (purple) or NT sgRNAs (blue) taken at 5 days and 30 days p.t. The sgRNA binding site is denoted along with the CpG islands and neighboring genes. The BOLA1 gene contains two annotated transcriptional start sites, labeled TSS1 and TSS2. FIG. 2H: volcano plot comparing H3K9me3 ChIP-seq data between CRISPRoff transfected with either H2B-targeting or NT sgRNAs. Red dots highlight the genes proximal to the H2B target.

FIGS. 3A-3D show that Flavivirus infections can be blocked by epigenome editing. FIG. 3A is a schematic showing epigenome editing of HEK293T cells for blocking viral infections. FIG. 3B YFV infection and detected a 50% reduction in YFV infection of CLTA-off cells (right panel) compared to WT HEK293T (left panel). FIG. 3C shows the infection of DENV-2 is reduced significantly in SPCS1 and STT3A targeted cells. FIG. 3D shows gene silencing of SPCS1 and STT3A mirrored the DENV-2 infection in edited cells.

FIGS. 4A-4G show genome-wide gene silencing by CRISPRoff. FIG. 4A: a schematic of the dual sgRNA lentiviral vector used in the CRISPRoff genome-wide screens that contains two unique sgRNAs targeting the same gene. FIG. 4B: a schematic of a pooled genome-wide screen to determine the targeting landscape of CRISPRoff. FIG. 4C: a time course of CLTA expression in HEK293T after transfection of dCas9-KRAB (gray), CRISPRoff-V2 (black), or mutant CRISPRoff-D3A^{E765A} (orange). FIG. 4D: a comparison of phenotype scores (γ) between CRISPRoff (y-axis) and CRISPRoff mutant (x-axis) screens. Three types of expected negative controls are highlighted as negative control pseudo-genes (blue), olfactory genes (orange), and Y chromosome genes (green). FIG. 4E: a violin plot of the phenotype scores (γ) for genes defined as essential or nonessential from DepMap. Each replicate screen is plotted for CRISPRoff (green) and CRISPRoff mutant (orange). FIG. 4F: a plot of true and false positive rates of genes defined as essential by DepMap. FIG. 4G: a plot illustrating the distance of an essential gene hit, defined as having a γ≤−0.2, from the nearest essential gene hit. Each dot corresponds to a gene hit's nearest neighboring essential gene, with the x-axis showing the distance between the two genes and the y-axis as the neighboring gene's phenotype score.

FIGS. 5A-5K show CRISPRoff-mediated silencing of genes without promoter CpG island annotations. FIG. 5A: a plot comparing the phenotype score of genes between the CRISPRoff and CRISPRoff mutant screens with genes that lack a CGI annotation highlighted in red. FIG. 5B: histograms of mNeonGreen fluorescence of five HEK293T cell lines, each with the indicated gene endogenously tagged with split mNeonGreen. FIG. 5C: quantification of cells with CALD1, DYNC2LI1, LAMP2, MYL6, or VPS25 silenced after CRISPRoff or CRISPRoff mutant treatment. The data were measured at 14 days p.t., except for VPS25 which was collected at 11 days p.t. due to a growth defect upon gene knockdown. FIG. 5D: quantification of percent of cells with DYNC2LI1 or LAMP2 reactivated after TETv4 treatment with targeting or non-targeting sgRNAs, obtained at 14 days p.t. FIG. 5E: CpG methylation profiling within the LAMP2, DYNC2LI1, and MYL6 promoters after CRISPRoff treatments. White circles represent the CpG methylation status of untransfected HEK293T cells. Each dot is an average of eight independent clones. FIGS. 5F-5H: time course plots of DYNC2LI1 (FIG. 5F), LAMP2 (FIG. 5G), and MYL6 (FIG. 5H) expression after transfection of either CRISPRoff or CRISPRoff mutant. Error bars represent the SD of three independent replicates. FIG. 5I: a histogram of DYNC2LI1 expression in 33 clonal lines, measured at 50 days p.t. A positive control of untransfected cells is labeled. FIG. 5J: a Manhattan plot displaying differentially methylated CpGs between cells treated with CRISPRoff and either DYNC2LI1-targeting or NT sgRNA, as analyzed by WGBS. The arrow points to the genomic location of DYNC2LI1. FIG. 5K: a view of a 10 kb genomic window containing the DYNC2LI1 locus, highlighting gain of CpG methylation (red) at the promoter in cells transfected with CRISPRoff and DYNC2LI1-targeting sgRNAs.

FIGS. 6A-6L show pooled sgRNA tiling screens that reveal a wide targetable window of CRISPRoff-mediated gene repression. FIG. 6A: a schematic of the sgRNA library that tiles PAM-containing sgRNAs within a +/−1 kb window from annotated transcription start sites (TSS). FIG. 6B: a summary of the number of genes per indicated category that comprise the tiling sgRNA library. FIG. 6C: a comparison of the phenotype score (γ) for genes with annotated CGI between CRISPRoff (y-axis) and CRISPRoff mutant (x-axis). Each dot is the average of the three most active sgRNAs for each gene. The red dots highlight genes that lack a promoter CGI annotation. FIG. 6D: an aggregate plot comparing the normalized phenotype score for each sgRNA targeting genes with one annotated CGI. The green line represents screen data from CRISPRoff in HEK293 Ts, orange from CRISPRoff mutant in HEK293 Ts, and purple from CRISPRi in K562s. FIGS. 6E-6G: representative sgRNA activity score profiles for DKC1, GPN2, and ZCCHC9 from the indicated screen (y-axis). The green bar depicts the annotated CGI obtained from UCSC Genome Browser. FIG. 6H: representative sgRNA activity score profile for ORC5 from the indicated screen (y-axis). FIG. 6I: an aggregate plot comparing the normalized phenotype score for each sgRNA for genes without annotated CGIs. FIG. 6J: an overlay of normalized sgRNA phenotype score from the CRISPRoff screen (green) with MNase signal that represents nucleosome occupancy (gray). The plot is an aggregate of genes with one annotated CGI. FIG. 6K: an overlay of normalized sgRNA phenotype score from the CRISPRoff screen (green) with MNase signal that represents nucleosome occupancy (gray). The plot is an aggregate of the 39 genes with no annotated CGI. FIG. 6L: a plot of sgRNA activity along with MNase signal for H2B, derived from the sgRNA tiling screen outlined in Figure S6F.

FIGS. 7A-7J: show CRISPRoff gene silencing in iPSCs, iPSC-derived neurons, and enhancers. FIG. 7A: an experimental workflow of CD81 knockdown by CRISPRoff in iPSCs, followed by NGN2-mediated differentiation of edited cells into neurons. FIG. 7B: quantification of cells with CD81 silenced by CRISPRi or CRISPRoff with CD81-targeting or NT sgRNAs, measured at 30 days p.t. The error bars represent SD from three independent experiments. FIG. 7C: quantification of cells with CD81 silenced at the indicated time points from (A). The gray bars indicate the percent of iPSC-edited cells with CD81 silenced that were not differentiated during the experiment. The red bars represent cells that were carried through the neuronal differentiation protocol. The error bars represent SD from three independent experiments. FIG. 7D: a representative histogram of CD81 expression at days 8 of neuronal differentiation of parental-unedited (gray) or CD81-edited iPSCs (red). FIG. 7E: bisulfite PCR of a 140 bp region of the CD81 promoter in cells transfected with CRISPRoff and NT or CD81-targeting sgRNA. FIG. 7F: representative bright field microscopy images of differentiated neurons derived from iPSCs transfected with CRISPRoff and MAPT-targeting or NT sgRNA. FIG. 7G: quantification of cells with Tau-off in cells transfected with CRISPRoff and NT or MAPT-targeting sgRNA, measured at 10 days post-differentiation. FIG. 7H: representative flow cytometry plots of Tau protein staining in iPSC-derived neurons after CRISPRoff transfection with NT or MAPT-targeting sgRNA. The gates are based on unperturbed iPSC-derived neurons. FIG. 7I: a schematic of the PVT1 locus with the promoter and four enhancer elements (ET-E4) labeled with the distance from the TSS. FIG. 7J: plots of normalized PVT1 transcript levels from quantitative RT-qPCR of cells treated with CRISPRoff (left) or CRISPRoff D3A mutant (right) and sgRNAs targeting either the promoter (Pr.) or the four enhancer elements (E1-E4), normalized to control sgRNAs. Asterisks denote statistical significance by t-test and each technical replicate is shown as red dots.

FIGS. 8A-8K show optimization of CRISPRoff design for durable gene silencing, related to FIG. 1. FIG. 8A: a schematic of the CRISPRoff-V1 construct and various linker sequences used to generate protein variants. FIG. 8B: a time course of CLTA-GFP silencing after transfection of CRISPRoff-V1 variants or controls dCas9-KRAB (gray) and dCas9-D3A-D3L (orange). FIG. 8C: a crystal structure of DNMT3A (orange) and DNMT3L (yellow) in complex with CpG-containing DNA (PDB 5YX2). The arrows point to the dCas9 attachment positions for CRISPRoff-V1 and CRISPRoff-V2. FIG. 8D: a schematic of four CRISPRoff-V2 constructs that varies BFP as a linker between dCas9 and KRAB or separated from CRISPRoff by a P2A sequence. The V2.3 and V2.4 constructs encode NLS sequences at the amino and carboxyl termini of dCas9. FIG. 8E: a western blot of dCas9, dCas9-KRAB, CRISPRoff construct protein expression. FIG. 8F: a time course of CLTA-GFP silencing after transfection of the V1 and V2.1, V2.3, V2.4 constructs, along with dCas9-KRAB and dCas9 only controls. FIG. 8G: a time course of Snrpn-GFP silencing after transfection of dCas9-D3A-3L (orange), dCas9-KRAB (gray), or CRISPRoff-V1 (black) and V2 (blue). FIG. 8H: a representative flow cytometry plot of HEK293T CLTA-GFP cells 6 days after transfection of mRNA encoding CRISPRoff. FIG. 8I: representative flow cytometry plots of multiplexing gene targeting of two genes simultaneously, measured at 30 days post-transfection. FIG. 8J: quantification of gene silencing measured at 31 days post-transfection of CRISPRoff with four simultaneous sgRNAs targeting ITGB1, CLTA, CD81, and CD151. FIG. 8K: quantification of cells with CLTA, CD81, and CD151 silenced in cells that have ITGB1 either silenced (blue) or unsilenced (gray) in the four gene knockdown experiment. The error bars in FIGS. 8B, F, G, J, and K represent SD from three independent experiments.

FIGS. 9A-9F: who that CRISPRoff is applicable in various cell lines and with orthogonal RNA-guided CRISPR proteins, related to FIG. 1. FIG. 9A: flow cytometry histograms of CRISPRoff expression (BFP) before and after doxycycline (dox) treatment. After 24 hours of dox administration, the media was replaced with media without doxycycline (1d and 2d post dox-wash) to turn off CRISPRoff expression. FIG. 9B: quantification of K562 cells with CD81 silenced 10 days after initial dox-induction of CRISPRoff expression. Doxycycline was included in the media for either 3 days (middle) or 4 days (right) prior to washing cells to remove doxycycline. FIG. 9C: quantification of cells with ITGB1, CD81, or CD151 silenced in HeLa and U2OS measured at 18 days post-CRISPRoff-V1 transfection with sgRNAs targeting the indicated genes. FIG. 9D: a representative flow cytometry plot of CD81 expression in iPS cells after transfection of CRISPRoff with either non-targeting sgRNAs or sgRNAs targeting CD81. FIGS. 9E-9F: a comparison of cells with CLTA (FIG. 9E) and H2B (FIG. 9F) silenced 10 days after transfection of CRISPRoff with dCas9 from S. pyogenes (dSpyCas9) or S. aureus (dSauCas9) or dCas12a from Lachnospiraceae bacterium (dLbCas12a). The error bars in FIGS. 9B, 9C, 9E, and 9F are SD from three independent experiments.

FIGS. 10A-10L show transcriptional specificity of CLTA, H2B, and RAB11A silencing, related to FIG. 2. FIG. 10A: an RNA-sequencing TPM (transcripts per kilobase million) plot for HEK293T cells transfected with CRISPRoff and NT (non-targeting) sgRNAs compared to untransfected HEK293T cells. FIGS. 10B-10D: RNA-sequencing TPM (transcripts per kilobase million) are plotted for HEK293T cells transfected with CRISPRoff and either NT sgRNAs compared to sgRNAs targeting CLTA (FIG. 10B), HIST2H2BE (FIG. 10C), or RAB11A (FIG. 10D). The data are representative of the average of two independent replicates. FIGS. 10E-10F: Representation of gene expression changes +/−1000 kb from the annotated targeted gene for CD81, CD151, ITGB1 (FIG. 10E) and RAB11, HIST2H2BE, CLTA (FIG. 10F). Each box represents a gene. FIG. 10G: a barplot of genome-wide average CpG coverage obtained from whole genome bisulfite sequencing. WT indicates untransfected cells; NT indicates CRISPRoff delivered with non-targeting sgRNAs; T indicates CRISPRoff delivered with sgRNAs targeting CLTA or DYNC2LI1. The experiments were performed in two replicates. CLTA experiments are presented in FIG. 2, DYNC2LI1 experiments are presented in FIG. 5. FIG. 10H: a barplot of genome-wide average CpG methylation (beta-values) in the samples described in FIG. 10G. FIG. 10I: heatmap plots comparing average CpG methylation (beta-values) of 20-CpG sliding windows between untransfected (WT) replicates (left), WT and NT (middle), and WT and targeting sgRNAs (right) for CLTA (top row) and DYNC2LI1 (bottom row) experiments. Red color indicates highest density and blue color indicates lowest density. White areas indicate the absence of windows with the respective average methylation levels. FIG. 10J: a heat map showing pairwise correlations between genome-wide CpG methylation profiles of the samples described in Figure S3G, including replicates. Samples are sorted by unsupervised hierarchical clustering. Dark brown color indicates highest correlation, light yellow color indicates lowest correlation. This analysis highlights the variations in global CpG methylation between samples for CLTA and DYNC2LI1 experiments. FIG. 10K: a close up of a 17 kb genomic region containing the ZSCAN16 gene. DNA methylation profiles for untransfected, NT, and targeting sgRNA cells from the CLTA experiment are shown. The box highlights a differentially methylated region at the gene promoter, indicating a gain of CpG methylation in the targeting sgRNA cells compared to the control cells. FIG. 10L: a close up of a 30 kb genomic region containing the RPS6KA6 gene. The box highlights a gain of CpG methylation at the promoter in the targeting sgRNA cells. A CpG island located in the promoter of the gene is indicated in green.

FIGS. 11A-11E show that genome-wide silencing by CRISPRoff is reproducible and specific (related to FIG. 4). FIG. 11A: a plot comparing the phenotype score (γ) of genes between technical replicates of the CRISPRoff (left) and CRISPRoff D3A methyltransferase mutant (right) genome-wide screens. The negative control sgRNAs are highlighted in blue. FIG. 11B: violin plots of the phenotype score (γ) of all genes from each screen. FIG. 11C: a histogram of the number of genes with the indicated phenotype score (γ) from the CRISPRoff and CRISPRoff mutant screens. The light green and light orange lines correspond to the phenotype scores of negative control sgRNAs. FIGS. 11D-11E: gene set enrichment analysis (GSEA) for genes associated with DNA replication and ribosome, confirming enrichment of expected essential genes. Genes are ranked from lowest (red) to highest (blue) phenotype scores.

FIGS. 12A-12I show design and validation of tiling sgRNA screens show flexible targeting genomic window of CRISPRoff activity (related to FIG. 6). FIG. 12A: the genes chosen for the sgRNA tiling screens are highlighted on a volcano plot depicting gene phenotype scores from previous genome-wide CRISPRi screens in K562 cells (Horlbeck et al. 2016). The colors represent genes with one (orange), multiple (purple), or no annotated CGI (green). FIG. 12B: an aggregate plot comparing the normalized phenotype score for each sgRNA for genes with multiple CGIs. The green line represents screen data from CRISPRoff transfection into HEK293T cells, orange from CRISPRoff mutant into HEK293T, and purple from K562 CRISPRi. FIG. 12C: representative sgRNA activity score profile for TBCD from the three screens. The green bar depicts the annotated CGIs obtained from UCSC Genome Browser. FIGS. 12D-12E: plots overlaying the sgRNA phenotype scores and MNase signals for GFER and IMMT. FIG. 12F: an experimental workflow of tiling sgRNA screens to determine optimal sgRNAs for four endogenously GFP-tagged genes: CLTA, H2B, RAB11A, and VIM. The indicated population in the histograms represent the population of cells that have maintained gene silencing 4 weeks after CRISPRoff transfection. FIGS. 12G-12I: overlay plots of sgRNA activity and MNase signal for CLTA, RAB11A, and VIM from the sgRNA tiling screen.

FIGS. 13A-13I: show confinement of H3K9me3 and CpG methylation despite distal sites of epigenetic establishment (related to FIG. 6). FIG. 13A: a schematic of the H2B promoter with two sgRNA sites annotated: sg-A at the TSS and sg-B located about 2 kb upstream of the TSS. The CpG island spans 1.4 kb. Sites 1, 2, and 3 represent regions probed for CpG methylation by bisulfite PCR as described in FIGS. 13E-13I. FIG. 13B: a time course of H2B silencing after transfection of CRISPRoff with sg-A or sg-B. FIG. 13C: a comparison of H3K9me3 profiles at the H2B (HIST2H2BE, colored red) promoter at day 5 (green tracks) and day 30 (purple tracks) post-transfection of CRISPRoff with sg-A, sg-B, or non-targeting (NT) sgRNA. The sgRNA binding sites are labeled, along with CpG island annotations (green), and the basal unmethylated CpG region of the H2B promoter prior to transfection obtained from WGBS of WT untransfected cells in FIG. 2. FIG. 13D: a comparison of H3K9me3 profiles at the H2B promoter, as described in C, except in experiments using CRISPRoff with a D3A methyltransferase mutation. FIGS. 13E-13F: quantification of CpG methylation at site 1 and site 3 of the H2B promoter (labeled in FIG. 13A) in cells transfected with CRISPRoff and either sg-A (blue), sg-B (orange), or non-targeting (gray) sgRNA. The cells were harvested for bisulfite PCR at day 5 post transfection. FIGS. 13G-13I: quantification of CpG methylation at sites 1, 2, and 3 of the H2B promoter, obtained at 30 days post transfection.

In the Figures and Examples, CRISPRoff-V1 refers to SEQ ID NO:1, and CRISPRoff-V2 refers to SEQ ID NO: 107 (V2.1), unless otherwise stated.

### DETAILED DESCRIPTION

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., Rieger et al. (eds.), Springer Verlag (1991); and Hale & Marham, The Harper Collins Dictionary of Biology (1991). As used herein, the following terms have the meanings ascribed to them unless specified otherwise.

The use of a singular indefinite or definite article (e.g., “a,” “an,” “the,” etc.) in this disclosure and in the following claims follows the traditional approach in patents of meaning “at least one” unless in a particular instance it is clear from context that the term is intended in that particular instance to mean specifically one and only one. Likewise, the term “comprising” is open ended, not excluding additional items, features, components, etc.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

For specific proteins described herein (e.g., KRAB, dCas9, Dnmt3A, Dnmt3L), the named protein includes any of the protein's naturally occurring forms, or variants or homologs that maintain the protein activity (e.g., within at least 50%, 80%, 90%, 95%, 96%, 97%, 98%, 99% or 100% activity compared to the native protein). In aspects, variants or homologs have at least 50%, 55%, 60%, 65%, 70%, 75%, 80%, 85%, 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In aspects, the protein is the protein as identified by its NCBI sequence reference. In aspects, the protein is the protein as identified by its NCBI sequence reference or functional fragment or homolog thereof.

The term “nuclease-deficient RNA-guided DNA endonuclease enzyme” and the like refer, in the usual and customary sense, to an RNA-guided DNA endonuclease (e.g. a mutated form of a naturally occurring RNA-guided DNA endonuclease) that targets a specific phosphodiester bond within a DNA polynucleotide, wherein the recognition of the phosphodiester bond is facilitated by a separate polynucleotide sequence (for example, a RNA sequence (e.g., single guide RNA (sgRNA)), but is incapable of cleaving the target phosphodiester bond to a significant degree (e.g. there is no measurable cleavage of the phosphodiester bond under physiological conditions). A nuclease-deficient RNA-guided DNA endonuclease thus retains DNA-binding ability (e.g. specific binding to a target sequence) when complexed with a polynucleotide (e.g., sgRNA), but lacks significant endonuclease activity (e.g. any amount of detectable endonuclease activity). In aspects, the nuclease-deficient RNA-guided DNA endonuclease enzyme is a CRISPR-associated protein. In aspects, the nuclease-deficient RNA-guided DNA endonuclease enzyme is dCas9, dCpf1, ddCpf1, Cas-phi, a nuclease-deficient Cas9 variant, a nuclease-deficient Class II CRISPR endonuclease, a zinc finger domain, a transcription activator-like effector (TALE), a leucine zipper domain, a winged helix domain, a helix-turn-helix motif, a helix-loop-helix domain, an HMB-box domain, a Wor3 domain, an OB-fold domain, an immunoglobulin domain, or a B3 domain. In aspects, the nuclease-deficient RNA-guided DNA endonuclease enzyme is a zinc finger domain, a leucine zipper domain, a winged helix domain, a helix-turn-helix motif, a helix-loop-helix domain, an HMB-box domain, a Wor3 domain, an OB-fold domain, an immunoglobulin domain, or a B3 domain. In aspects, the nuclease-deficient RNA-guided DNA endonuclease enzyme is a leucine zipper domain. In aspects, the nuclease-deficient RNA-guided DNA endonuclease enzyme is a winged helix domain. In aspects, the nuclease-deficient RNA-guided DNA endonuclease enzyme is a helix-turn-helix motif. In aspects, the nuclease-deficient RNA-guided DNA endonuclease enzyme is a helix-loop-helix domain. In aspects, the nuclease-deficient RNA-guided DNA endonuclease enzyme is an HMB-box domain. In aspects, the nuclease-deficient RNA-guided DNA endonuclease enzyme is a Wor3 domain. In aspects, the nuclease-deficient RNA-guided DNA endonuclease enzyme is an OB-fold domain. In aspects, the nuclease-deficient RNA-guided DNA endonuclease enzyme is an immunoglobulin domain. In aspects, the nuclease-deficient RNA-guided DNA endonuclease enzyme is a B3 domain. In aspects, the nuclease-deficient RNA-guided DNA endonuclease enzyme is dCas9, ddCpf1, Cas-phi, a nuclease-deficient Cas9 variant, or a nuclease-deficient Class II CRISPR endonuclease. In aspects, the nuclease-deficient RNA-guided DNA endonuclease enzyme is dCas9. In aspects, the nuclease-deficient RNA-guided DNA endonuclease enzyme is dCas9 from S. pyogenes. In aspects, the nuclease-deficient RNA-guided DNA endonuclease enzyme is dCas9 from S. aureus. In aspects, the nuclease-deficient RNA-guided DNA endonuclease enzyme is dCas12a from Lachnospiracea (dLbCas12a). In aspects, the nuclease-deficient RNA-guided DNA endonuclease enzyme is dCas12a from Lachnospiracea bacterium. In aspects, the nuclease-deficient RNA-guided DNA endonuclease enzyme is dCas12a. In aspects, the nuclease-deficient RNA-guided DNA endonuclease enzyme is dCas12. In aspects, the nuclease-deficient RNA-guided DNA endonuclease enzyme is ddCas12a. In aspects, the nuclease-deficient RNA-guided DNA endonuclease enzyme is Cas-phi.

The term “CRISPR-associated protein” or “CRISPR protein” refers to any CRISPR protein that functions as a nuclease-deficient RNA-guided DNA endonuclease enzyme, i.e., a CRISPR protein in which catalytic sites for endonuclease activity are defective or lack activity. Exemplary CRISPR-associated proteins include dCas9, dCpf1, dCas12, Cas-phi, a nuclease-deficient Cas9 variant, a nuclease-deficient Class II CRISPR endonuclease, and the like.

A “CRISPR associated protein 9,” “Cas9,” “Csn1” or “Cas9 protein” as referred to herein includes any of the recombinant or naturally-occurring forms of the Cas9 endonuclease or variants or homologs thereof that maintain Cas9 endonuclease enzyme activity (e.g. within at least 50%, 80%, 90%, 95%, 96%, 97%, 98%, 99% or 100% activity compared to Cas9). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas9 protein. In aspects, the Cas9 protein is substantially identical to the protein identified by the UniProt reference number Q99ZW2 or a variant or homolog having substantial identity thereto. In aspects, the Cas9 protein has at least 75% sequence identity to the amino acid sequence of the protein identified by the UniProt reference number Q99ZW2. In aspects, the Cas9 protein has at least 80% sequence identity to the amino acid sequence of the protein identified by the UniProt reference number Q99ZW2. In aspects, the Cas9 protein has at least 85% sequence identity to the amino acid sequence of the protein identified by the UniProt reference number Q99ZW2. In aspects, the Cas9 protein has at least 90% sequence identity to the amino acid sequence of the protein identified by the UniProt reference number Q99ZW2. In aspects, the Cas9 protein has at least 95% sequence identity to the amino acid sequence of the protein identified by the UniProt reference number Q99ZW2.

In embodiments, the nuclease-deficient RNA-guided DNA endonuclease enzyme is dCas9. The terms “dCas9” or “dCas9 protein” as referred to herein is a Cas9 protein in which both catalytic sites for endonuclease activity are defective or lack activity. In aspects, the dCas9 protein has mutations at positions corresponding to D10A and H840A of S. pyogenes Cas9. In aspects, the dCas9 protein lacks endonuclease activity due to point mutations at both endonuclease catalytic sites (RuvC and HNH) of wild type Cas9. The point mutations can be D10A and H840A. In aspects, the dCas9 has substantially no detectable endonuclease (e.g., endodeoxyribonuclease) activity. In aspects, dCas9 includes the amino acid sequence of SEQ ID NO:23. In aspects, dCas9 has the amino acid sequence of SEQ ID NO:23. In aspects, dCas9 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:23. In aspects, dCas9 has an amino acid sequence that has at least 75% sequence identity to SEQ ID NO:23. In aspects, dCas9 has an amino acid sequence that has at least 80% sequence identity to SEQ ID NO:23. In aspects, dCas9 has an amino acid sequence that has at least 85% sequence identity to SEQ ID NO:23. In aspects, dCas9 has an amino acid sequence that has at least 90% sequence identity to SEQ ID NO:23. In aspects, dCas9 has an amino acid sequence that has at least 95% sequence identity to SEQ ID NO:23. In embodiments, the nuclease-deficient RNA-guided DNA endonuclease enzyme is dCas9 from S. pyogenes. In embodiments, the nuclease-deficient RNA-guided DNA endonuclease enzyme is dCas9 from S. aureus.

In embodiments, the nuclease-deficient RNA-guided DNA endonuclease enzyme is “ddCpf1” or “ddCas12a”. The terms “DNAse-dead Cpf1” or “ddCpf1” refer to mutated Acidaminococcus sp. Cpf1 (AsCpf1) resulting in the inactivation of Cpf1 DNAse activity. In aspects, ddCpf1 includes an E993A mutation in the RuvC domain of AsCpf1. In aspects, the ddCpf1 has substantially no detectable endonuclease (e.g., endodeoxyribonuclease) activity. In aspects, ddCpf1 includes the amino acid sequence of SEQ ID NO:34. In aspects, ddCpf1 has the amino acid sequence of SEQ ID NO:34. In aspects, ddCpf1 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:34. In aspects, ddCpf1 has an amino acid sequence that has at least 75% sequence identity to SEQ ID NO:34. In aspects, ddCpf1 has an amino acid sequence that has at least 80% sequence identity to SEQ ID NO:34. In aspects, ddCpf1 has an amino acid sequence that has at least 85% sequence identity to SEQ ID NO:34. In aspects, ddCpf1 has an amino acid sequence that has at least 90% sequence identity to SEQ ID NO:34. In aspects, ddCpf1 has an amino acid sequence that has at least 95% sequence identity to SEQ ID NO:34. In aspects, the nuclease-deficient RNA-guided DNA endonuclease enzyme is dCas12a from Lachnospiracea bacterium.

In embodiments, the nuclease-deficient RNA-guided DNA endonuclease enzyme is dLbCpf1. The term “dLbCpf1: refers to mutated Cpf1 from Lachnospiraceae bacterium ND2006 (LbCpf1) that lacks DNAse activity. In aspects, dLbCpf1 includes a D832A mutation. In aspects, the dLbCpf1 has substantially no detectable endonuclease (e.g., endodeoxyribo-nuclease) activity. In aspects, dLbCpf1 includes the amino acid sequence of SEQ ID NO:35. In aspects, dLbCpf1 has the amino acid sequence of SEQ ID NO:35. In aspects, dLbCpf1 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:35. In aspects, dLbCpf1 has an amino acid sequence that has at least 75% sequence identity to SEQ ID NO:35. In aspects, dLbCpf1 has an amino acid sequence that has at least 80% sequence identity to SEQ ID NO:35. In aspects, dLbCpf1 has an amino acid sequence that has at least 85% sequence identity to SEQ ID NO:35. In aspects, dLbCpf1 has an amino acid sequence that has at least 90% sequence identity to SEQ ID NO:35. In aspects, dLbCpf1 has an amino acid sequence that has at least 95% sequence identity to SEQ ID NO:35.

In embodiments, the nuclease-deficient RNA-guided DNA endonuclease enzyme is dFnCpf1. The term “dFnCpf1” refers to mutated Cpf1 from Francisella novicida U112 (FnCpf1) that lacks DNAse activity. In aspects, dFnCpf1 includes a D917A mutation. In aspects, the dFnCpf1 has substantially no detectable endonuclease (e.g., endodeoxyribo-nuclease) activity. In aspects, dFnCpf1 includes the amino acid sequence of SEQ ID NO: 36. In aspects, dFnCpf1 has the amino acid sequence of SEQ ID NO: 36. In aspects, dFnCpf1 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:36. In aspects, dFnCpf1 has an amino acid sequence that has at least 75% sequence identity to SEQ ID NO:36. In aspects, dFnCpf1 has an amino acid sequence that has at least 80% sequence identity to SEQ ID NO:36. In aspects, dFnCpf1 has an amino acid sequence that has at least 85% sequence identity to SEQ ID NO:36. In aspects, dFnCpf1 has an amino acid sequence that has at least 90% sequence identity to SEQ ID NO:36. In aspects, dFnCpf1 has an amino acid sequence that has at least 95% sequence identity to SEQ ID NO:36.

A “Cpf1” or “Cpf1 protein” as referred to herein includes any of the recombinant or naturally-occurring forms of the Cpf1 (CRISPR from Prevotella and Francisella 1) endonuclease or variants or homologs thereof that maintain Cpf1 endonuclease enzyme activity (e.g. within at least 50%, 80%, 90%, 95%, 96%, 97%, 98%, 99% or 100% activity compared to Cpf1). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cpf1 protein. In aspects, the Cpf1 protein is substantially identical to the protein identified by the UniProt reference number U2UMQ6 or a variant or homolog having substantial identity thereto. In aspects, the Cpf1 protein is identical to the protein identified by the UniProt reference number U2UMQ6. In aspects, the Cpf1 protein has at least 75% sequence identity to the amino acid sequence of the protein identified by the UniProt reference number U2UMQ6. In aspects, the Cpf1 protein has at least 80% sequence identity to the amino acid sequence of the protein identified by the UniProt reference number U2UMQ6. In aspects, the Cpf1 protein is identical to the protein identified by the UniProt reference number U2UMQ6. In aspects, the Cpf1 protein has at least 85% sequence identity to the amino acid sequence of the protein identified by the UniProt reference number U2UMQ6. In aspects, the Cpf1 protein is identical to the protein identified by the UniProt reference number U2UMQ6. In aspects, the Cpf1 protein has at least 90% sequence identity to the amino acid sequence of the protein identified by the UniProt reference number U2UMQ6. In aspects, the Cpf1 protein is identical to the protein identified by the UniProt reference number U2UMQ6. In aspects, the Cpf1 protein has at least 95% sequence identity to the amino acid sequence of the protein identified by the UniProt reference number U2UMQ6.

In embodiments, the nuclease-deficient RNA-guided DNA endonuclease enzyme is a nuclease-deficient Cas9 variant. The term “nuclease-deficient Cas9 variant” refers to a Cas9 protein having one or more mutations that increase its binding specificity to PAM compared to wild type Cas9 and further include mutations that render the protein incapable of or having severely impaired endonuclease activity. Without wishing to be bound by theory, it is believed that the target sequence should be associated with a PAM (protospacer adjacent motif); that is, a short sequence recognized by the CRISPR complex. The precise sequence and length requirements for the PAM differ depending on the CRISPR enzyme used, but PAMs are typically 2-5 base pair sequences adjacent the protospacer (that is, the target sequence). The binding specificity of nuclease-deficient Cas9 variants to PAM can be determined by any method known in the art. Descriptions and uses of known Cas9 variants may be found, for example, in Shmakov et al., Diversity and evolution of class 2 CRISPR-Cas systems. Nat. Rev. Microbiol. 15, 2017 and Cebrian-Serrano et al, CRISPR-Cas orthologues and variants: optimizing the repertoire, specificity and delivery of genome engineering tools. Mamm. Genome 7-8, 2017. Exemplary Cas9 variants are listed in the Table 1 below.

TABLE 1

| Cas9 Variants | PAM domains | References |
|---|---|---|
| Strep pyogenes | NGG | Hsu et al. 2014 Cell |
| (Sp) Cas9 | | |
| Staph aureus | NNGRRT or NNGRR | Ran et al. 2015 Nature |
| (Sa) Cas9 | NNGGGT, NNGAAT, | |
| | NNGAGT (Zetsche) | |
| SpCas9 VQR mutant | NGAG > NGAT = | Kleinstiver et al. 2015 |
| (D1135V, R1335Q, | NGAA > NGAC | Nature |
| T1337R) | NGCG | |
| SpCas9 VRER mutant | NGCG | Kleinstiver et al. 2015 |
| (D1135V/G1218R/ | | Nature |
| R1335E/T1337R) | | |
| SpCas9 D1135E | NGG, greater fidelity, | Kleinstiver et al. 2015 |
| | less cutting at | Nature |
| | NAG and NGA sites | |
| eSpCas9 1.1 mutant | NGG | Slaymaker et al. |
| (K848A/K1003A/ | | Science 2015 |
| R1060A) | | |
| SpCas9 HF1 | NGG | Kleinstiver et al. 2016 |
| (Q695A, Q926A, | | Nature |
| N497A, R661A) | | |
| AsCpf1 | TTTN (5′ of sgRNA) | Zetsche et al. 2015 Cell |
| HypaCas9 (N692A, | | Chen et al., Nature |
| M694A, Q695A, | | volume 550, pages |
| H698A) | | 407-410 (19 Oct. 2017) |

In embodiments, the nuclease-deficient RNA-guided DNA endonuclease enzyme is a nuclease-deficient Class II CRISPR endonuclease. The term “nuclease-deficient Class II CRISPR endonuclease” as used herein refers to any Class II CRISPR endonuclease having mutations resulting in reduced, impaired, or inactive endonuclease activity.

The term “nuclease-deficient DNA endonuclease enzyme” refers to a DNA endonuclease (e.g. a mutated form of a naturally occurring DNA endonuclease) that targets a specific phosphodiester bond within a DNA polynucleotide, but that does not require an RNA guide. In embodiments, the “nuclease-deficient DNA endonuclease enzyme” is a zinc finger domain or a TALE.

In embodiments, the nuclease-deficient DNA endonuclease enzyme is a “zinc finger domain.” The term “zinc finger domain” or “zinc finger binding domain” or “zinc finger DNA binding domain” are used interchangeably and refer to a protein, or a domain within a larger protein, that binds DNA in a sequence-specific manner through one or more zinc fingers, which are regions of amino acid sequence within the binding domain whose structure is stabilized through coordination of a zinc ion. In embodiments, the zinc finger domain is non-naturally occurring in that it is engineered to bind to a target site of choice. In aspects, the zinc finger binding domain refers to a protein, a domain within a larger protein, or a nuclease-deficient RNA-guided DNA endonuclease enzyme that is capable of binding to any zinc finger known in the art, such as the C2H2 type, the CCHC type, the PHD type, or the RING type of zinc fingers.

As used herein, a “zinc finger” is a polypeptide structural motif folded around a bound zinc cation. In embodiments, the polypeptide of a zinc finger has a sequence of the form X₃-Cys-X₂₋₄-Cys-X₁₂-His-X₃₋₅-His-X₄, wherein X is any amino acid (e.g., X₂₋₄ indicates an oligopeptide 2-4 amino acids in length). There is generally a wide range of sequence variation in the 28-31 amino acids of the known zinc finger polypeptides. Only the two consensus histidine residues and two consensus cysteine residues bound to the central zinc atom are invariant. Of the remaining residues, three to five are highly conserved, while there may be significant variation among the other residues. Despite the wide range of sequence variation in the polypeptide, zinc fingers of this type have a similar three dimensional structure. However, there is a wide range of binding specificities among the different zinc fingers, i.e. different zinc fingers bind double stranded polynucleotides having a wide range of nucleotides sequences. In aspects, the zinc finger is the C2H2 type. In aspects, the zinc finger is the CCHC type. In aspects, the zinc finger is the PHD type. In aspects, the zinc finger is the RING type.

In embodiments, the nuclease-deficient DNA endonuclease enzyme is a TALE. “TALE” or “transcription activator-like effector” refer to artificial restriction enzymes generated by fusing the TAL effector DNA binding domain to a DNA cleavage domain. TALEs enable efficient, programmable, and specific DNA cleavage and represent powerful tools for genome editing in situ. Transcription activator-like effectors (TALEs) can be quickly engineered to bind practically any DNA sequence. The term TALE, as used herein, is broad and includes a monomeric TALE that can cleave double stranded DNA without assistance from another TALE. The term TALE is also used to refer to one or both members of a pair of TALEs that are engineered to work together to cleave DNA at the same site, TALEs that work together may be referred to as a left-TALE and a right-TALE, which references the handedness of DNA. TALE are proteins secreted by Xanthomonas bacteria. The DNA binding domain contains a highly conserved 33-34 amino acid sequence with the exception of the 12th and 13th amino acids. These two locations are highly variable (repeat variable diresidue (RVD)) and show a strong correlation with specific nucleotide recognition. This simple relationship between amino acid sequence and DNA recognition has allowed for the engineering of specific DNA binding domains by selecting a combination of repeat segments containing the appropriate RVDs.

The term “Krüppel associated box domain” or “KRAB domain” as provided herein refers to a category of transcriptional repression domains present in approximately 400 human zinc finger protein-based transcription factors. KRAB domains typically include about 45 to about 75 amino acid residues. A description of KRAB domains, including their function and use, may be found, for example, in Ecco, G., Imbeault, M., Trono, D., KRAB zinc finger proteins, Development 144, 2017; Lambert et al. The human transcription factors, Cell 172, 2018; Gilbert et al., Cell (2013); and Gilbert et al., Cell (2014). In aspects, the KRAB domain is a KRAB domain of Kox 1. In aspects, the KRAB domain includes the sequence set forth by SEQ ID NO:16. In aspects, the KRAB domain is the sequence of SEQ ID NO:16. In aspects, the KRAB domain includes an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:16. In aspects, the KRAB domain includes an amino acid sequence that has at least 75% sequence identity to SEQ ID NO:16. In aspects, the KRAB domain includes an amino acid sequence that has at least 80% sequence identity to SEQ ID NO:16. In aspects, the KRAB domain includes an amino acid sequence that has at least 85% sequence identity to SEQ ID NO:16. In aspects, the KRAB domain includes an amino acid sequence that has at least 90% sequence identity to SEQ ID NO:16. In aspects, the KRAB domain includes an amino acid sequence that has at least 95% sequence identity to SEQ ID NO:16. In embodiments, the KRAB domain is a ZIM3 KRAB domain or an amino acid sequence having 85%, 90%, or 95% sequence identity thereto. In embodiments, the KRAB domain is KOX1 or an amino acid sequence having 85%, 90%, or 95% sequence identity thereto. Gilbert et al, Cell, 159:647-661 (2014); Alerasool et al, Nature Methods, (2020).

The term “DNA methyltransferase” as provided herein refers to an enzyme that catalyzes the transfer of a methyl group to DNA. Non-limiting examples of DNA methyltransferases include Dnmt1, Dnmt3A, and Dnmt3B. In aspects, the DNA methyltransferase is mammalian DNA methyltransferase. In aspects, the DNA methyltransferase is human DNA methyltransferase. In aspects, the DNA methyltransferase is mouse DNA methyltransferase. In aspects, the DNA methyltransferase is a bacterial cytosine methyltransferase and/or a bacterial non-cytosine methyltransferase. Depending on the specific DNA methyltransferase, different regions of DNA are methylated. For example, Dnmt3A typically targets CpG dinucleotides for methylation. Through DNA methylation, DNA methyltransferases can modify the activity of a DNA segment (e.g., gene expression) without altering the DNA sequence. In aspects, DNA methylation results in repression of gene transcription and/or modulation of methylation sensitive transcription factors or CTCF. As described herein, fusion proteins may include one or more (e.g., two) DNA methyltransferases. When a DNA methyltransferase is included as part of a fusion protein, the DNA methyltransferase may be referred to as a “DNA methyltransferase domain.” In aspects, a DNA methyltransferase domain includes one or more DNA methyltransferases. In aspects, a DNA methyltransferase domain includes two DNA methyltransferases. In aspects, the DNA methyltransferase domain further comprises a catalytically inactive regulatory factor of DNA methyltransferase (e.g., Dnmt3L) that is essential for the functioning of Dnmt1, Dnmt3A, and Dnmt3B. In aspects, the DNA methyltransferase domain comprises Dnmt1. In aspects, the DNA methyltransferase domain comprises Dnmt3B. In aspects, the DNA methyltransferase domain comprises Dnmt3A. In aspects, the DNA methyltransferase domain further comprises Dnmt3L. In aspects, the DNA methyltransferase domain has the amino acid sequence of SEQ ID NO:26. In aspects, the DNA methyltransferase domain has an amino acid sequence that has at least 50%, 55%, 60%, 65%, 70%, 75%, 80%, 85%, 90%, 91%, 92%9, 93%, 94%, 95%, 96%, 97%, 98%, 99% or 100% sequence identity to SEQ ID NO:26. In aspects, the DNA methyltransferase domain has an amino acid sequence that has at least 75% sequence identity to SEQ ID NO:26. In aspects, the DNA methyltransferase domain has an amino acid sequence that has at least 80% sequence identity to SEQ ID NO:26. In aspects, the DNA methyltransferase domain has an amino acid sequence that has at least 85% sequence identity to SEQ ID NO:26. In aspects, the DNA methyltransferase domain has an amino acid sequence that has at least 90% sequence identity to SEQ ID NO:26. In aspects, the DNA methyltransferase domain has an amino acid sequence that has at least 95% sequence identity to SEQ ID NO:26. In aspects, the DNA methyltransferase domain is Dnmt3L. In aspects, the DNA methyltransferase domain has the amino acid sequence of SEQ ID NO:28. In aspects, the DNA methyltransferase domain has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:28. In aspects, the DNA methyltransferase domain has an amino acid sequence that has at least 75% sequence identity to SEQ ID NO:28. In aspects, the DNA methyltransferase domain has an amino acid sequence that has at least 80% sequence identity to SEQ ID NO:28. In aspects, the DNA methyltransferase domain has an amino acid sequence that has at least 85% sequence identity to SEQ ID NO:28. In aspects, the DNA methyltransferase domain has an amino acid sequence that has at least 90% sequence identity to SEQ ID NO:28. In aspects, the DNA methyltransferase domain has an amino acid sequence that has at least 95% sequence identity to SEQ ID NO:28. In aspects, the DNA methyltransferase domain includes Dnmt3A and Dnmt3L. In aspects, the DNA methyltransferase domain has the amino acid sequence of SEQ ID NO:33. In aspects, the DNA methyltransferase domain has an amino acid sequence that has at least 50%, 55%, 60%, 65%, 70%, 75%, 80%, 85%, 90%, 91%, 92%9, 93%, 94%, 95%, 96%, 97%, 98%, 99% or 100% sequence identity to SEQ ID NO:33. In aspects, the DNA methyltransferase domain has an amino acid sequence that has at least 75% sequence identity to SEQ ID NO:33. In aspects, the DNA methyltransferase domain has an amino acid sequence that has at least 80% sequence identity to SEQ ID NO:33. In aspects, the DNA methyltransferase domain has an amino acid sequence that has at least 85% sequence identity to SEQ ID NO:33. In aspects, the DNA methyltransferase domain has an amino acid sequence that has at least 90% sequence identity to SEQ ID NO:33. In aspects, the DNA methyltransferase domain has an amino acid sequence that has at least 95% sequence identity to SEQ ID NO:33. In aspects, the DNA methyltransferase domain further comprises the Dnmt3L regulatory factor, as described, for example, in Siddique et al, Targeted methylation and gene silencing of VEGF-A in human cells by using a designed Dnmt3a-Dnmt3L single-chain fusion protein with increased DNA methylation activity, J. Mol. Biol. 425, 2013 and Stepper et al, Efficient targeted DNA methylation with chimeric dCas9-Dnmt3a-Dnmt3L methyltransferase, Nucleic Acids Res. 45, 2017.

A “Dnmt3A”, “Dnmt3a,” “DNA (cytosine-5)-methyltransferase 3A” or “DNA methyltransferase 3a” protein as referred to herein includes any of the recombinant or naturally-occurring forms of the Dnmt3A enzyme or variants or homologs thereof that maintain Dnmt3A enzyme activity (e.g. within at least 50%, 55%, 60%, 65%, 70%, 75%, 80%, 85%, 90%, 91%, 92%, 93%, 94%, 95%, 96%, 97%, 98%, 99% or 100% activity compared to Dnmt3A). In aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring Dnmt3A protein. In aspects, the Dnmt3A protein is substantially identical to the protein identified by the UniProt reference number Q9Y6K1 or a variant or homolog having substantial identity thereto. In aspects, the Dnmt3A polypeptide is encoded by a nucleic acid sequence identified by the NCBI reference sequence Accession number NM_022552, homologs or functional fragments thereof. In aspects, Dnmt3A includes the sequence set forth by SEQ ID NO:26. In aspects, Dnmt3A is the sequence set forth by SEQ ID NO:26. In aspects, Dnmt3A has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:26. In aspects, the DNA methyltransferase domain has an amino acid sequence that has at least 75% sequence identity to SEQ ID NO:26. In aspects, the DNA methyltransferase domain has an amino acid sequence that has at least 80% sequence identity to SEQ ID NO:26. In aspects, the DNA methyltransferase domain has an amino acid sequence that has at least 85% sequence identity to SEQ ID NO:26. In aspects, Dnmt3A has an amino acid sequence that has at least 90% sequence identity to SEQ ID NO:26. In aspects, Dnmt3A has an amino acid sequence that has at least 95% sequence identity to SEQ ID NO:26. In aspects, Dnmt3A is s Dnmt3A transcript 1 (Dnmt3A1). In aspects, Dnmt3A is s Dnmt3A transcript 2 (Dnmt3A2). In aspects, Dnmt3A is s Dnmt3A transcript 3 (Dnmt3A3). In aspects, is s Dnmt3A transcript 4 (Dnmt3A4).

A “Dnmt3L”, “DNA (cytosine-5)-methyltransferase 3L” or “DNA methyltransferase 3L” protein as referred to herein includes any of the recombinant or naturally-occurring forms of the Dnmt3L regulatory factor or variants or homologs thereof that maintain Dnmt3L regulatory activity (e.g., within at least 50%, 55%, 60%, 65%, 70%, 75%, 80%, 85%, 90%, 91%, 92%, 93%, 94%, 95%, 96%, 97%, 98%, 99% or 100% activity compared to Dnmt3L). In aspects, the variants or homologs have at least 50%, 55%, 60%, 65%, 70%, 75%, 80%, 85%, 90%, 91%, 92%, 93%, 94%, 95%, 96%, 97%, 98%, 99% or 100% amino acid sequence identity across the whole sequence or a portion of the sequence compared to a naturally occurring Dnmt3L protein. In aspects, the Dnmt3L protein is substantially identical to the protein identified by the UniProt reference number Q9CWR8 or a variant or homolog having substantial identity thereto. In aspects, the Dnmt3L protein is identical to the protein identified by the UniProt reference number Q9CWR8. In aspects, the Dnmt3L protein has at least 75% sequence identity to the amino acid sequence of the protein identified by the UniProt reference number Q9CWR8. In aspects, the Dnmt3L protein has at least 80% sequence identity to the amino acid sequence of the protein identified by the UniProt reference number Q9CWR8. In aspects, the Dnmt3L protein has at least 85% sequence identity to the amino acid sequence of the protein identified by the UniProt reference number Q9CWR8. In aspects, the Dnmt3L protein has at least 95% sequence identity to the amino acid sequence of the protein identified by the UniProt reference number Q9CWR8.

In aspects, the Dnmt3L protein is substantially identical to the protein identified by the UniProt reference number Q9UJW or a variant or homolog having substantial identity thereto. In aspects, the Dnmt3L protein is identical to the protein identified by the UniProt reference number Q9UJW. In aspects, the Dnmt3L protein has at least 50% sequence identity to the amino acid sequence of the protein identified by the UniProt reference number Q9UJW. In aspects, the Dnmt3L protein has at least 55% sequence identity to the protein identified by the UniProt reference number Q9UJW. In aspects, the Dnmt3L protein has at least 60% sequence identity to the amino acid sequence of the protein identified by the UniProt reference number Q9UJW. In aspects, the Dnmt3L protein has at least 65% sequence identity to the amino acid sequence of the protein identified by the UniProt reference number Q9UJW. In aspects, the Dnmt3L protein has at least 70% sequence identity to the amino acid sequence of the protein identified by the UniProt reference number Q9UJW. In aspects, the Dnmt3L protein has at least 75% sequence identity to the amino acid sequence of the protein identified by the UniProt reference number Q9UJW. In aspects, the Dnmt3L protein has at least 80% sequence identity to the amino acid sequence of the protein identified by the UniProt reference number Q9UJW. In aspects, the Dnmt3L protein has at least 85% sequence identity to the amino acid sequence of the protein identified by the UniProt reference number Q9UJW. In aspects, the Dnmt3L protein has at least 90% sequence identity to the amino acid sequence of the protein identified by the UniProt reference number Q9UJW. In aspects, the Dnmt3L protein has at least 95% sequence identity to the amino acid sequence of the protein identified by the UniProt reference number Q9UJW. In aspects, the Dnmt3L polypeptide is encoded by a nucleic acid sequence identified by the NCBI reference sequence Accession number NM_001081695, or homologs or functional fragments thereof. In aspects, Dnmt3L includes the sequence set forth by SEQ ID NO:28. In aspects, Dnmt3L is the sequence set forth by SEQ ID NO:28. In aspects, Dnmt3L has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:28. In aspects, Dnmt3L has an amino acid sequence that has at least 50% sequence identity to SEQ ID NO:28. In aspects, Dnmt3L has an amino acid sequence that has at least 55% sequence identity to SEQ ID NO:28. In aspects, Dnmt3L has an amino acid sequence that has at least 60% sequence identity to SEQ ID NO:28. In aspects, Dnmt3L has an amino acid sequence that has at least 65% sequence identity to SEQ ID NO:28. In aspects, Dnmt3L has an amino acid sequence that has at least 97% sequence identity to SEQ ID NO:28. In aspects, Dnmt3L has an amino acid sequence that has at least 75% sequence identity to SEQ ID NO:28. In aspects, Dnmt3L has an amino acid sequence that has at least 80% sequence identity to SEQ ID NO:28. In aspects, Dnmt3L has an amino acid sequence that has at least 85% sequence identity to SEQ ID NO:28. In aspects, Dnmt3L has an amino acid sequence that has at least 90% sequence identity to SEQ ID NO:28. In aspects, Dnmt3L has an amino acid sequence that has at least 95% sequence identity to SEQ ID NO:28.

The term “RNA-guided DNA endonuclease” and the like refer, in the usual and customary sense, to an enzyme that cleave a phosphodiester bond within a DNA polynucleotide chain, wherein the recognition of the phosphodiester bond is facilitated by a separate RNA sequence (for example, a single guide RNA).

The term “Class II CRISPR endonuclease” refers to endonucleases that have similar endonuclease activity as Cas9 and participate in a Class II CRISPR system. An example Class II CRISPR system is the type II CRISPR locus from Streptococcus pyogenes SF370, which contains a cluster of four genes Cas9, Cas1, Cas2, and Csn1, as well as two non-coding RNA elements, tracrRNA and a characteristic array of repetitive sequences (direct repeats) interspaced by short stretches of non-repetitive sequences (spacers, about 30 bp each). The Cpf1 enzyme belongs to a putative type V CRISPR-Cas system. Both type II and type V systems are included in Class II of the CRISPR-Cas system.

A “nuclear localization sequence” or “nuclear localization signal” or “NLS” is a peptide that directs proteins to the nucleus. In aspects, the NLS includes five basic, positively charged amino acids. The NLS may be located anywhere on the peptide chain. In aspects, the NLS is an NLS derived from SV40. In aspects, the NLS includes the sequence set forth by SEQ ID NO:25. In aspects, the NLS is the sequence set forth by SEQ ID NO:25. In aspects, NLS has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO: 25. In aspects, NLS has an amino acid sequence that has at least 75% sequence identity to SEQ ID NO:25. In aspects, NLS has an amino acid sequence that has at least 80% sequence identity to SEQ ID NO:25. In aspects, NLS has an amino acid sequence that has at least 85% sequence identity to SEQ ID NO: 25. In aspects, NLS has an amino acid sequence that has at least 90% sequence identity to SEQ ID NO:25. In aspects, NLS has an amino acid sequence that has at least 95% sequence identity to SEQ ID NO:25. In aspects, NLS has an amino acid sequence of SEQ ID NO:25.

In embodiments, the DNA methyltransferase domain is a Dnmt3A-3L domain. A “Dnmt3A-3L domain” as provided herein refers to a protein including both Dnmt3A (i.e., the DNA methyltransferase) and Dnmt3L (i.e., the catalytically inactive regulatory factor of DNA methyltransferase). In aspects, Dnmt3A and Dnmt3L are covalently linked. In aspects, Dnmt3A is covalently linked to Dnmt3L through a peptide linker. In aspects, the peptide linker includes the sequence set forth by SEQ ID NO:27. In aspects, the peptide linker is the sequence set forth by SEQ ID NO:27. In aspects, the peptide linker has an amino acid sequence that has at least 50%, 55%, 60%, 65%, 70%, 75%, 80%, 85%, 90%, 91%9, 92%, 93%, 94%, 95%, 96%, 97%, 98%, 99% or 100% sequence identity to SEQ ID NO:27. In aspects, the peptide linker has an amino acid sequence that has at least 90% sequence identity to SEQ ID NO:27. In aspects, the peptide linker has an amino acid sequence that has at least 95% sequence identity to SEQ ID NO:27. In aspects, the Dnmt3A-3L domain includes the sequence set forth by SEQ ID NO:33. In aspects, the Dnmt3A-3L domain is the sequence set forth by SEQ ID NO:33. In aspects, the Dnmt3A-3L domain has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:33. In aspects, the Dnmt3A-3L domain has an amino acid sequence that has at least 75% sequence identity to SEQ ID NO:33. In aspects, the Dnmt3A-3L domain has an amino acid sequence that has at least 80% sequence identity to SEQ ID NO:33. In aspects, the Dnmt3A-3L domain has an amino acid sequence that has at least 85% sequence identity to SEQ ID NO:33. In aspects, the Dnmt3A-3L domain has an amino acid sequence that has at least 90% sequence identity to SEQ ID NO:33. In aspects, the Dnmt3A-3L domain has an amino acid sequence that has at least 95% sequence identity to SEQ ID NO:33.

The term “Krüppel associated box domain” or “KRAB domain” as provided herein refers to a category of transcriptional repression domains present in approximately 400 human zinc finger protein-based transcription factors. KRAB domains typically include about 45 to about 75 amino acid residues. A description of KRAB domains, including their function and use, may be found, for example, in Ecco, G., Imbeault, M., Trono, D., KRAB zinc finger proteins, Development 144, 2017; Lambert et al. The human transcription factors, Cell 172, 2018; Gilbert et al., Cell (2013); and Gilbert et al., Cell (2014). In aspects, the KRAB domain is a KRAB domain of Kox 1. In aspects, the KRAB domain includes the sequence set forth by SEQ ID NO:16. In aspects, the KRAB domain is the sequence of SEQ ID NO:16. In aspects, the KRAB domain includes an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:16. In aspects, the KRAB domain includes an amino acid sequence that has at least 75% sequence identity to SEQ ID NO:16. In aspects, the KRAB domain includes an amino acid sequence that has at least 80% sequence identity to SEQ ID NO:16. In aspects, the KRAB domain includes an amino acid sequence that has at least 85% sequence identity to SEQ ID NO:16. In aspects, the KRAB domain includes an amino acid sequence that has at least 90% sequence identity to SEQ ID NO:16. In aspects, the KRAB domain includes an amino acid sequence that has at least 95% sequence identity to SEQ ID NO:16.

The term “DNA methyltransferase” as provided herein refers to an enzyme that catalyzes the transfer of a methyl group to DNA. Non-limiting examples of DNA methyltransferases include Dnmt1, Dnmt3A, and Dnmt3B. In aspects, the DNA methyltransferase is mammalian DNA methyltransferase. In aspects, the DNA methyltransferase is human DNA methyltransferase. In aspects, the DNA methyltransferase is mouse DNA methyltransferase. In aspects, the DNA methyltransferase is a bacterial cytosine methyltransferase and/or a bacterial non-cytosine methyltransferase. Depending on the specific DNA methyltransferase, different regions of DNA are methylated. For example, Dnmt3A typically targets CpG dinucleotides for methylation. Through DNA methylation, DNA methyltransferases can modify the activity of a DNA segment (e.g., gene expression) without altering the DNA sequence. In aspects, DNA methylation results in repression of gene transcription and/or modulation of methylation sensitive transcription factors or CTCF. As described herein, fusion proteins may include one or more (e.g., two) DNA methyltransferases. When a DNA methyltransferase is included as part of a fusion protein, the DNA methyltransferase may be referred to as a “DNA methyltransferase domain.” In aspects, a DNA methyltransferase domain includes one or more DNA methyltransferases. In aspects, a DNA methyltransferase domain includes two DNA methyltransferases. In aspects, the DNA methyltransferase domain further comprises a catalytically inactive regulatory factor of DNA methyltransferase (e.g., Dnmt3L) that is essential for the functioning of Dnmt1, Dnmt3A, and Dnmt3B. In aspects, the DNA methyltransferase domain comprises Dnmt1. In aspects, the DNA methyltransferase domain comprises Dnmt3B. In aspects, the DNA methyltransferase domain comprises Dnmt3A. In aspects, the DNA methyltransferase domain has the amino acid sequence of SEQ ID NO:26. In aspects, the DNA methyltransferase domain has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:26. In aspects, the DNA methyltransferase domain has an amino acid sequence that has at least 75% sequence identity to SEQ ID NO:26. In aspects, the DNA methyltransferase domain has an amino acid sequence that has at least 80% sequence identity to SEQ ID NO:26. In aspects, the DNA methyltransferase domain has an amino acid sequence that has at least 85% sequence identity to SEQ ID NO:26. In aspects, the DNA methyltransferase domain has an amino acid sequence that has at least 90% sequence identity to SEQ ID NO:26. In aspects, the DNA methyltransferase domain has an amino acid sequence that has at least 95% sequence identity to SEQ ID NO:26. In aspects, the DNA methyltransferase domain is Dnmt3L. In aspects, the DNA methyltransferase domain has the amino acid sequence of SEQ ID NO:28. In aspects, the DNA methyltransferase domain has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:28. In aspects, the DNA methyltransferase domain has an amino acid sequence that has at least 75% sequence identity to SEQ ID NO:28. In aspects, the DNA methyltransferase domain has an amino acid sequence that has at least 80% sequence identity to SEQ ID NO:28. In aspects, the DNA methyltransferase domain has an amino acid sequence that has at least 85% sequence identity to SEQ ID NO:28. In aspects, the DNA methyltransferase domain has an amino acid sequence that has at least 90% sequence identity to SEQ ID NO:28. In aspects, the DNA methyltransferase domain has an amino acid sequence that has at least 95% sequence identity to SEQ ID NO:28. In aspects, the DNA methyltransferase domain includes Dnmt3A and Dnmt3L. In aspects, the DNA methyltransferase domain has the amino acid sequence of SEQ ID NO:33. In aspects, the DNA methyltransferase domain has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:33. In aspects, the DNA methyltransferase domain has an amino acid sequence that has at least 75% sequence identity to SEQ ID NO:33. In aspects, the DNA methyltransferase domain has an amino acid sequence that has at least 80% sequence identity to SEQ ID NO:33. In aspects, the DNA methyltransferase domain has an amino acid sequence that has at least 85% sequence identity to SEQ ID NO:33. In aspects, the DNA methyltransferase domain has an amino acid sequence that has at least 90% sequence identity to SEQ ID NO:33. In aspects, the DNA methyltransferase domain has an amino acid sequence that has at least 95% sequence identity to SEQ ID NO:33. In aspects, the DNA methyltransferase domain further comprises the Dnmt3L regulatory factor, as described, for example, in Siddique et al, Targeted methylation and gene silencing of VEGF-A in human cells by using a designed Dnmt3a-Dnmt3L single-chain fusion protein with increased DNA methylation activity, J. Mol. Biol. 425, 2013 and Stepper et al, Efficient targeted DNA methylation with chimeric dCas9-Dnmt3a-Dnmt3L methyltransferase, Nucleic Acids Res. 45, 2017.

A “Dnmt3A”, “Dnmt3a,” “DNA (cytosine-5)-methyltransferase 3A” or “DNA methyltransferase 3a” protein as referred to herein includes any of the recombinant or naturally-occurring forms of the Dnmt3A enzyme or variants or homologs thereof that maintain Dnmt3A enzyme activity (e.g. within at least 50%, 55%, 60%, 65%, 70%, 75%, 80%, 85%, 90%, 91%, 92%, 93%, 94%, 95%, 96%, 97%, 98%, 99% or 100% activity compared to Dnmt3A). In aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring Dnmt3A protein. In aspects, the Dnmt3A protein is substantially identical to the protein identified by the UniProt reference number Q9Y6K1 or a variant or homolog having substantial identity thereto. In aspects, the Dnmt3A polypeptide is encoded by a nucleic acid sequence identified by the NCBI reference sequence Accession number NM_022552, homologs or functional fragments thereof. In aspects, Dnmt3A includes the sequence set forth by SEQ ID NO:26. In aspects, Dnmt3A is the sequence set forth by SEQ ID NO:26. In aspects, Dnmt3A has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:26. In aspects, the DNA methyltransferase domain has an amino acid sequence that has at least 75% sequence identity to SEQ ID NO:26. In aspects, the DNA methyltransferase domain has an amino acid sequence that has at least 80% sequence identity to SEQ ID NO:26. In aspects, the DNA methyltransferase domain has an amino acid sequence that has at least 85% sequence identity to SEQ ID NO:26. In aspects, Dnmt3A has an amino acid sequence that has at least 90% sequence identity to SEQ ID NO:26. In aspects, Dnmt3A has an amino acid sequence that has at least 95% sequence identity to SEQ ID NO:26.

A “Dnmt3L”, “DNA (cytosine-5)-methyltransferase 3L” or “DNA methyltransferase 3L” protein as referred to herein includes any of the recombinant or naturally-occurring forms of the Dnmt3L regulatory factor or variants or homologs thereof that maintain Dnmt3L regulatory activity (e.g., within at least 50%, 55%, 60%, 65%, 70%, 75%, 80%, 85%, 90%, 91%, 92%, 93%, 94%, 95%, 96%, 97%, 98%, 99% or 100% activity compared to Dnmt3L). In aspects, the variants or homologs have at least 50%, 55%, 60%, 65%, 70%, 75%, 80%, 85%, 90%, 91%, 92%, 93%, 94%, 95%, 96%, 97%, 98%, 99% or 100% amino acid sequence identity across the whole sequence or a portion of the sequence compared to a naturally occurring Dnmt3L protein. In aspects, the Dnmt3L protein is substantially identical to the protein identified by the UniProt reference number Q9CWR8 or a variant or homolog having substantial identity thereto. In aspects, the Dnmt3L protein is identical to the protein identified by the UniProt reference number Q9CWR8. In aspects, the Dnmt3L protein has at least 75% sequence identity to the amino acid sequence of the protein identified by the UniProt reference number Q9CWR8. In aspects, the Dnmt3L protein has at least 80% sequence identity to the amino acid sequence of the protein identified by the UniProt reference number Q9CWR8. In aspects, the Dnmt3L protein has at least 85% sequence identity to the amino acid sequence of the protein identified by the UniProt reference number Q9CWR8. In aspects, the Dnmt3L protein has at least 95% sequence identity to the amino acid sequence of the protein identified by the UniProt reference number Q9CWR8.

In aspects, the Dnmt3L protein is substantially identical to the protein identified by the UniProt reference number Q9UJW or a variant or homolog having substantial identity thereto. In aspects, the Dnmt3L protein is identical to the protein identified by the UniProt reference number Q9UJW. In aspects, the Dnmt3L protein has at least 50% sequence identity to the amino acid sequence of the protein identified by the UniProt reference number Q9UJW. In aspects, the Dnmt3L protein has at least 55% sequence identity to the protein identified by the UniProt reference number Q9UJW. In aspects, the Dnmt3L protein has at least 60% sequence identity to the amino acid sequence of the protein identified by the UniProt reference number Q9UJW. In aspects, the Dnmt3L protein has at least 65% sequence identity to the amino acid sequence of the protein identified by the UniProt reference number Q9UJW. In aspects, the Dnmt3L protein has at least 70% sequence identity to the amino acid sequence of the protein identified by the UniProt reference number Q9UJW. In aspects, the Dnmt3L protein has at least 75% sequence identity to the amino acid sequence of the protein identified by the UniProt reference number Q9UJW. In aspects, the Dnmt3L protein has at least 80% sequence identity to the amino acid sequence of the protein identified by the UniProt reference number Q9UJW. In aspects, the Dnmt3L protein has at least 85% sequence identity to the amino acid sequence of the protein identified by the UniProt reference number Q9UJW. In aspects, the Dnmt3L protein has at least 90% sequence identity to the amino acid sequence of the protein identified by the UniProt reference number Q9UJW. In aspects, the Dnmt3L protein has at least 95% sequence identity to the amino acid sequence of the protein identified by the UniProt reference number Q9UJW. In aspects, the Dnmt3L polypeptide is encoded by a nucleic acid sequence identified by the NCBI reference sequence Accession number NM_001081695, or homologs or functional fragments thereof. In aspects, Dnmt3L includes the sequence set forth by SEQ ID NO:28. In aspects, Dnmt3L is the sequence set forth by SEQ ID NO:28. In aspects, Dnmt3L has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:28. In aspects, Dnmt3L has an amino acid sequence that has at least 50% sequence identity to SEQ ID NO:28. In aspects, Dnmt3L has an amino acid sequence that has at least 55% sequence identity to SEQ ID NO:28. In aspects, Dnmt3L has an amino acid sequence that has at least 60% sequence identity to SEQ ID NO:28. In aspects, Dnmt3L has an amino acid sequence that has at least 65% sequence identity to SEQ ID NO:28. In aspects, Dnmt3L has an amino acid sequence that has at least 97% sequence identity to SEQ ID NO:28. In aspects, Dnmt3L has an amino acid sequence that has at least 75% sequence identity to SEQ ID NO:28. In aspects, Dnmt3L has an amino acid sequence that has at least 80% sequence identity to SEQ ID NO:28. In aspects, Dnmt3L has an amino acid sequence that has at least 85% sequence identity to SEQ ID NO:28. In aspects, Dnmt3L has an amino acid sequence that has at least 90% sequence identity to SEQ ID NO:28. In aspects, Dnmt3L has an amino acid sequence that has at least 95% sequence identity to SEQ ID NO:28.

The term “RNA-guided DNA endonuclease” and the like refer, in the usual and customary sense, to an enzyme that cleave a phosphodiester bond within a DNA polynucleotide chain, wherein the recognition of the phosphodiester bond is facilitated by a separate RNA sequence (for example, a single guide RNA).

A “nuclear localization sequence” or “nuclear localization signal” or “NLS” is a peptide that directs proteins to the nucleus. In aspects, the NLS includes five basic, positively charged amino acids. The NLS may be located anywhere on the peptide chain. In aspects, the NLS is an NLS derived from SV40. In aspects, the NLS includes the sequence set forth by SEQ ID NO:25. In aspects, the NLS is the sequence set forth by SEQ ID NO:25. In aspects, NLS has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO: 25. In aspects, NLS has an amino acid sequence that has at least 75% sequence identity to SEQ ID NO:25. In aspects, NLS has an amino acid sequence that has at least 80% sequence identity to SEQ ID NO:25. In aspects, NLS has an amino acid sequence that has at least 85% sequence identity to SEQ ID NO:25. In aspects, NLS has an amino acid sequence that has at least 90% sequence identity to SEQ ID NO:25. In aspects, NLS has an amino acid sequence that has at least 95% sequence identity to SEQ ID NO:25. In aspects, NLS has an amino acid sequence of SEQ ID NO:25.

A “guide RNA” or “gRNA” as provided herein refers to any polynucleotide sequence having sufficient complementarity with a target polynucleotide sequence to hybridize with the target sequence and direct sequence-specific binding of a CRISPR complex to the target sequence. In aspects, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

In embodiments, the polynucleotide (e.g., gRNA) is a single-stranded ribonucleic acid. In aspects, the polynucleotide (e.g., gRNA) is from about 10 to about 200 nucleic acid residues in length. In aspects, the polynucleotide (e.g., gRNA) is from about 50 to about 150 nucleic acid residues in length. In aspects, the polynucleotide (e.g., gRNA) is from about 80 to about 140 nucleic acid residues in length. In aspects, the polynucleotide (e.g., gRNA) is from about 90 to about 130 nucleic acid residues in length. In aspects, the polynucleotide (e.g., gRNA) is from about 100 to about 120 nucleic acid residues in length. In aspects, the length of the polynucleotide (e.g., gRNA) is about 113 nucleic acid residues in length.

In general, a guide sequence (i.e., a DNA-targeting sequence) is any polynucleotide sequence having sufficient complementarity with a target polynucleotide sequence to hybridize with the target sequence (e.g., a genomic or mitochondrial DNA target sequence) and direct sequence-specific binding of a complex (e.g., CRISPR complex) to the target sequence. In aspects, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. In aspects, the degree of complementarity between a guide sequence and its corresponding target sequence, when optimally aligned using a suitable alignment algorithm, is at least about 80%, 85%, 90%, 95%, or 100%. In aspects, the degree of complementarity is at least 90%. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif), SOAP (available at soap.genomics.org.cn), and Maq (available at maq.sourceforge.net). In aspects, a guide sequence is about or more than about 10, 20, 30, 35, 40, 45, 50, 75, or more nucleotides in length. In aspects, a guide sequence is about 10 to about 50, about 15 to about 30, or about 20 to about 25 nucleotides in length. In aspects, a guide sequence is less than about 75, 50, 45, 40, 35, 30, 25, 20, 15, 12, or fewer nucleotides in length. In aspects, the guide sequence is about or more than about 20 nucleotides in length. The ability of a guide sequence to direct sequence-specific binding of a complex (e.g., CRISPR complex) to a target sequence may be assessed by any suitable assay. For example, the components of a CRISPR system sufficient to form a complex (e.g., CRISPR complex), including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay known in the art. Similarly, cleavage of a target polynucleotide sequence may be evaluated in a test tube by providing the target sequence, components of a complex (e.g., CRISPR complex), including the guide sequence to be tested and a control guide sequence different from the test guide sequence, and comparing binding or rate of cleavage at the target sequence between the test and control guide sequence reactions. Other assays are possible, and will occur to those skilled in the art.

The terms “sgRNA,” “single guide RNA,” and “single guide RNA sequence” are used interchangeably and refer to the polynucleotide sequence including the crRNA sequence and the tracrRNA sequence. The crRNA sequence includes a guide sequence (i.e., “guide” or “spacer”) and a tracr mate sequence (i.e., direct repeat(s)”). The term “guide sequence” refers to the sequence that specifies the target site. In aspects, the two RNA can be encoded separately by a crRNA and tracrRNA as 2 RNA molecules which then form an RNA/RNA complex due to complementary base pairing between the crRNA and tracrRNA (i.e., before being competent to bind to nuclease-deficient RNA-guided DNA endonuclease enzyme). In aspects, a first nucleic acid includes a tracrRNA sequence, and a separate second nucleic acid includes a gRNA sequence lacking a tracrRNA sequence. In aspects, the first nucleic acid including the tracrRNA sequence and the second nucleic acid including the gRNA sequence interact with one another, and optionally are included in a complex (e.g., CRISPR complex). Exemplary sgRNA, and their targeted sequences, are shown in Tables 2, 3, and 4. Exemplary sgRNA described in the examples are shown in Table 5.

TABLE 2

| | Targeted sequence | sgRNA sequence |
|---|---|---|
| Name | (5′ to 3′) | (5′ to 3′) |
| A (JKNg156) | ACTGCGGAAATTTGAGCGT | ACGCUCAAAUUUCCGCAGU |
| | (SEQ ID NO: 37) | (SEQ ID NO: 38) |
| B (JKNg158) | AGGCAATGGCTGCACATGC | GCAUGUGCAGCCAUUGCCU |
| | (SEQ ID NO: 39) | (SEQ ID NO: 40) |
| C (JKNg160) | GACGCTTGGTTCTGAGGAG | CUCCUCAGAACCAAGCGUC |
| | (SEQ ID NO: 41) | (SEQ ID NO: 42) |

TABLE 3

| | Targeted sequence | sgRNA sequence |
|---|---|---|
| Name | (5′ to 3′) | (5′ to 3′) |
| CD29, sgRNA-A | TCCGGAAACGCATTCCTCT | AGAGGAAUGCGUUUCCGGA |
| | (SEQ ID NO: 43) | (SEQ ID NO: 44) |
| CD29, sgRNA-B | CCGCGTCAGCCCGGCCCGG | CCGGGCCGGGCUGACGCGG |
| | (SEQ ID NO: 45) | (SEQ ID NO: 46) |
| CD29, sgRNA-C | CGACTCCCGCTGGGCCTCT | AGAGGCCCAGCGGGAGUCG |
| | (SEQ ID NO: 47) | (SEQ ID NO: 48) |
| CD81, sgRNA-A | CCGTTGCGCGCTCGCTCTC | GAGAGCGAGCGCGCAACGG |
| | (SEQ ID NO: 49) | (SEQ ID NO: 50) |
| CD81, sgRNA-B | CCGCGCATCCTGCCAGGCC | GGCCUGGCAGGAUGCGCGG |
| | (SEQ ID NO: 51) | (SEQ ID NO: 52) |
| CD81, sgRNA-C | CCAACTTGGCGCGTTTCGG | CCGAAACGCGCCAAGUUGG |
| | (SEQ ID NO: 53) | (SEQ ID NO: 54) |
| CD151, sgRNA-A | ACCACGCGTCCGAGTCCGG | CCGGACUCGGACGCGUGGU |
| | (SEQ ID NO: 55) | (SEQ ID NO: 56) |
| CD151, sgRNA-B | TGCTCATTGTCCCTGGACA | UGUCCAGGGACAAUGAGCA |
| | (SEQ ID NO: 57) | (SEQ ID NO: 58) |
| CD151, sgRNA-C | GGACACCCTGCTCATTGTC | GACAAUGAGCAGGGUGUCC |
| | (SEQ ID NO: 59) | (SEQ ID NO: 60) |

TABLE 4

| | Targeted sequence | sgRNA sequence |
|---|---|---|
| Name | (5′ to 3′) | (5′ to 3′) |
| Pcsk9 sgRNA-1 | TCCGGAAACGCATTCCTCT | AGAGGAAUGCGUUUCCGGA |
| | (SEQ ID NO: 43) | (SEQ ID NO: 44) |
| Pcsk9 sgRNA-2 | ACCGGCAGCCTGCGCGTCC | GGACGCGCAGGCUGCCGGU |
| | (SEQ ID NO: 61) | (SEQ ID NO: 62) |
| Pcsk9 sgRNA-3 | CGATGGGCACCCACTGCTC | GAGCAGUGGGUGCCCAUCG |
| | (SEQ ID NO: 63) | (SEQ ID NO: 64) |
| Pcsk9 sgRNA-4 | CCTTCACGTGGACGCGCAG | CUGCGCGUCCACGUGAAGG |
| | (SEQ ID NO: 65) | (SEQ ID NO: 66) |
| Pcsk9 sgRNA-5 | CGTGAAGGTGGAAGCCTTC | GAAGGCUUCCACCUUCACG |
| | (SEQ ID NO: 67) | (SEQ ID NO: 68) |
| Npc1 sgRNA-1 | CTCCTTGGTCAGGCGCCGG | CCGGCGCCUGACCAAGGAG |
| | (SEQ ID NO: 69) | (SEQ ID NO: 70) |
| Npc1 sgRNA-2 | TGGTCAGGCGCCGGTTCCG | CGGAACCGGCGCCUGACCA |
| | (SEQ ID NO: 71) | (SEQ ID NO: 72) |
| Npc1 sgRNA-3 | TAGAGGTCGCCTTCTCCTC | GAGGAGAAGGCGACCUCUA |
| | (SEQ ID NO: 73) | (SEQ ID NO: 74) |
| Npc1 sgRNA-4 | CGACGCTCGGGTCGCGGTG | CACCGCGACCCGAGCGUCG |
| | (SEQ ID NO: 75) | (SEQ ID NO: 76) |
| Npc1 sgRNA-5 | ATGCTGTCGCCGCGCGGGG | CCCCGCGCGGCGACAGCAU |
| | (SEQ ID NO: 77) | (SEQ ID NO: 78) |
| Spcs1 sgRNA-1 | CTCACCCTCACCGGAGCCA | UGGCUCCGGUGAGGGUGAG |
| | (SEQ ID NO: 79) | (SEQ ID NO: 80) |
| Spcs1 sgRNA-2 | CCGCAAACTTTACTCCTTA | UAAGGAGUAAAGUUUGCGG |
| | (SEQ ID NO: 81) | (SEQ ID NO: 82) |
| Spcs1 sgRNA-3 | CTCGGAGACATCCGCTTCC | GGAAGCGGAUGUCUCCGAG |
| | (SEQ ID NO: 60) | (SEQ ID NO: 60) |
| Spcs1 sgRNA-4 | CTCCTAAGATTGGCTTCAC | GUGAAGCCAAUCUUAGGAG |
| | (SEQ ID NO: 83) | (SEQ ID NO: 84) |
| Spcs1 sgRNA-5 | CCGGAGCCACTCCTAAGAT | AUCUUAGGAGUGGCUCCGG |
| | (SEQ ID NO: 85) | (SEQ ID NO: 86) |
| Cd81 sgRNA-1 | TTCTCTACCCTACGTCTCA | UGAGACGUAGGGUAGAGAA |
| | (SEQ ID NO: 87) | (SEQ ID NO: 88) |
| Cd81 sgRNA-2 | TACGTCTCATTCTCCGCAA | UUGCGGAGAAUGAGACGUA |
| | (SEQ ID NO: 89) | (SEQ ID NO: 90) |
| Cd81 sgRNA-3 | GCTAGGCCTCCAGCCCTTC | GAAGGGCUGGAGGCCUAGC |
| | (SEQ ID NO: 91) | (SEQ ID NO: 92) |
| Cd81 sgRNA-4 | ACAGGTGGCGCCGCAACTT | AAGUUGCGGCGCCACCUGU |
| | (SEQ ID NO: 93) | (SEQ ID NO: 94) |
| Cd81 sgRNA-5 | AGCCGGAGGCGCGAGAGTC | GACUCUCGCGCCUCCGGCU |
| | (SEQ ID NO: 95) | (SEQ ID NO: 96) |

TABLE 5

| Name | Protospacer (5′ to 3′) | SEQ ID NO: |
|---|---|---|
| Snrpn promoter | CTCCTCAGAACCAAGCGTC | SEQ ID NO: 127 |
| H2B sgRNA-a | GTAAGACACAGTACAAACG | SEQ ID NO: 128 |
| H2B sgRNA-b | GAACCGGCAAAATCCGCTC | SEQ ID NO: 129 |
| H2B sgRNA-c | GGGCCGGAGCGGATTTTGC | SEQ ID NO: 130 |
| CLTA sgRNA-a | GAAGCCCTACCCGTGTATC | SEQ ID NO: 131 |
| CLTA sgRNA-b | CTTGTCCTCCTCTCCCAGT | SEQ ID NO: 132 |
| CLTA sgRNA-c | CTCCCAGTCGGCACCACAG | SEQ ID NO: 133 |
| ITGB1 sgRNA-a | AGAGGAATGCGTTTCCGGA | SEQ ID NO: 134 |
| ITGB1 sgRNA-b | CCGGGCCGGGCTGACGCGG | SEQ ID NO: 135 |
| ITGB1 sgRNA-c | AGAGGCCCAGCGGGAGTCG | SEQ ID NO: 136 |
| CD81 sgRNA-a | GAGAGCGAGCGCGCAACGG | SEQ ID NO: 137 |
| CD81 sgRNA-b | GGCCTGGCAGGATGCGCGG | SEQ ID NO: 138 |
| CD81 sgRNA-c | CCGAAACGCGCCAAGTTGG | SEQ ID NO: 139 |
| CD151 sgRNA-a | CCGGACTCGGACGCGTGGT | SEQ ID NO: 140 |
| CD151 sgRNA-b | TGTCCAGGGACAATGAGCA | SEQ ID NO: 141 |
| CD151 sgRNA-c | GACAATGAGCAGGGTGTCC | SEQ ID NO: 142 |
| CALD1 sgRNA-a | TTGGCTGGGGTGTCGTCAT | SEQ ID NO: 143 |
| CALD1 sgRNA-b | TCTGGGAGGGGCCAAGGAA | SEQ ID NO: 144 |
| CALD1 sgRNA-c | GAGATGCCTGTCATTCCCT | SEQ ID NO: 145 |
| CALD1 sgRNA-d | CCTCCCGACTGTAAACATA | SEQ ID NO: 146 |
| CALD1 sgRNA-e | AGGCAGGCTGCATCCACCT | SEQ ID NO: 147 |
| DYNC2LI1 sgRNA-a | GCCGAGAATGAGATGTAAA | SEQ ID NO: 148 |
| DYNC2LI1 sgRNA-b | CGCGGTTGCGTGGGGAGAC | SEQ ID NO: 149 |
| DYNC2LI1 sgRNA-c | GTGGGGAGACGGGCATCAT | SEQ ID NO: 150 |
| DYNC2LI1 sgRNA-d | GTCGCGGCCGCGGTTGCGT | SEQ ID NO: 151 |
| DYNC2LI1 sgRNA-e | GGGAGCGACGTCGCGGCCG | SEQ ID NO: 152 |
| LAMP2 sgRNA-a | TCTCTCAGGAGCATAGGAA | SEQ ID NO: 153 |
| LAMP2 sgRNA-b | GAGAGGGGTGGTGATGTAG | SEQ ID NO: 154 |
| LAMP2 sgRNA-c | CCGTCCGCGCATATCTCTC | SEQ ID NO: 155 |
| LAMP2 sgRNA-d | CGACCACGCCCTGGCTTTT | SEQ ID NO: 156 |
| LAMP2 sgRNA-e | CGCTTGAAAGCGGCGAGAG | SEQ ID NO: 157 |
| MYL6 sgRNA-a | CGGAGGTGGGGGGGGTCGT | SEQ ID NO: 158 |
| MYL6 sgRNA-b | GGGCACAGAACCCGCTCGG | SEQ ID NO: 159 |
| MYL6 sgRNA-c | ACCCCCCCCACCTCCGAGC | SEQ ID NO: 160 |
| MYL6 sgRNA-d | AACCCGCTCGGAGGTGGGG | SEQ ID NO: 161 |
| MYL6 sgRNA-e | CACAGAACCCGCTCGGAGG | SEQ ID NO: 162 |
| VPS25 sgRNA-a | TAGGATAACAGCGAGGGAC | SEQ ID NO: 163 |
| VPS25 sgRNA-b | TGGAAAGAGAGAGACCGGC | SEQ ID NO: 164 |
| VPS25 sgRNA-c | AAGGCCGGAAGTCCTTAGC | SEQ ID NO: 165 |
| VPS25 sgRNA-d | CCAAAAGATAGGGCTTTTT | SEQ ID NO: 166 |
| VPS25 sgRNA-e | AGGCAATGCAGAAAGGTTC | SEQ ID NO: 167 |
| PVT1 E1-a | GAGGTCATGATTCATCCCAC | SEQ ID NO: 168 |
| PVT1 E1-b | GGCTACTGGTCTCCACCGGC | SEQ ID NO: 169 |
| PVT1 E1-c | GAAGCTCGCTTACAGGAGGG | SEQ ID NO: 170 |
| PVT1 E2-a | GCTGCTGAGCGATGACTCAG | SEQ ID NO: 171 |
| PVT1 E2-b | GTCCCTGCGCCAGGGTAAAC | SEQ ID NO: 172 |
| PVT1 E2-c | GCAATCTCGGTCCGCCTTAC | SEQ ID NO: 173 |
| PVT1 E3-a | GCCGGCTGTTGAGTCAGCTC | SEQ ID NO: 174 |
| PVT1 E3-b | GCTGGATGACACCACTCTGT | SEQ ID NO: 175 |
| PVT1 E3-c | GCGGTGGAGACCTCATGATC | SEQ ID NO: 176 |
| PVT1 E4-a | GCAGCCTCTTGTTCGCTGCT | SEQ ID NO: 177 |
| PVT1 E4-b | GTTAAGATAGAGAATGCCAT | SEQ ID NO: 178 |
| PVT1 E4-c | GTTCCGTACACAGTCCTTTC | SEQ ID NO: 179 |
| PVTI promoter | GCCTCCGGGCAGAGCGCGTG | SEQ ID NO: 180 |
| Lambda negative | GATCGTCGGTCGGGTCATACG | SEQ ID NO: 181 |
| control-1 | | |
| Lambda negative | GTTCGTCGCGATAGATGATCG | SEQ ID NO: 182 |
| control-2 | | |
| Lambda negative | GCGCGTATCGTTTCATCGGCG | SEQ ID NO: 183 |
| control-3 | | |

The sequences in the Tables are the targeting crRNA sequences. As an example, the full single guide RNA (sgRNA) for SEQ ID NO: 38 is: GACGCUCAAAUUUCCGCAGUGUUUAAGAGCUAAGCUGGAAACAGCAUAGCAAGU UUAAAUAAGGCUAGUCCGUUAUCAACUUGAAAAAGUGGCACCGAGUCGGUGCUU UUUUU (SEQ ID NO:100). A common tracr sequence of each single guide for Sp Cas9 is GUUUAAGAGCUAAGCUGGAAACAGCAUAGCAAGUUUAAAUAAGGCUAGUCCGUU AUCAACUUGAAAAAGUGGCACCGAGUCGGUGCUUUUUUU (SEQ ID NO:101). The skilled artisan will appreciate that the sgRNA sequences in the Tables are 19 base pairs and do not reflect that each sgRNA starts with a G which is required if expressed from a pol-III promoter for initiation of transcription. Thus, for SEQ ID NO:38, the sequence would be GACGCUCAAAUUUCCGCAGU (SEQ ID NO:102) rather than ACGCUCAAAUUUCCGCAGU (SEQ ID NO:38). In embodiments, SEQ ID NOS:38, 40, 42, 44, 46, 48, 50, 52, 54, 56, 58, 60, 62, 64, 66, 68, 70, 72, 74, 76, 78, 80, 82, 84, 86, 88, 90, 92, 94, and 96 each contain a G as the first nucleotide. In embodiments, SEQ ID NOS:127-183 each contain a G as the first nucleotide.

In general, a tracr mate sequence includes any sequence that has sufficient complementarity with a tracrRNA sequence to promote one or more of: (1) excision of a guide sequence flanked by tracr mate sequences in a cell containing the corresponding tracr sequence; and (2) formation of a complex (e.g., CRISPR complex) at a target sequence, wherein the complex (e.g., CRISPR complex) comprises the tracr mate sequence hybridized to the tracr sequence. In general, degree of complementarity is with reference to the optimal alignment of the tracr mate sequence and tracrRNA sequence, along the length of the shorter of the two sequences. Optimal alignment may be determined by any suitable alignment algorithm, and may further account for secondary structures, such as self-complementarity within either the tracrRNA sequence or tracr mate sequence. In aspects, the degree of complementarity between the tracrRNA sequence and tracr mate sequence along the length of the shorter of the two when optimally aligned is about or more than about 25%, 30%, 40%, 50%, 60%, 70%, 80%, 90%, 95%, 97.5%, 99%, or higher. In aspects, the degree of complementarity is about or at least about 80%, 90%, 95%, or 100%. In aspects, the tracrRNA sequence is about or more than about 5, 10, 15, 20, 30, 40, 50, or more nucleotides in length. In aspects, the tracrRNA sequence and tracr mate sequence are contained within a single transcript, such that hybridization between the two produces a transcript having a secondary structure, such as a hairpin.

“Nucleic acid” refers to nucleotides (e.g., deoxyribonucleotides or ribonucleotides) and polymers thereof in either single-, double- or multiple-stranded form, or complements thereof. The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acids, e.g. polynucleotides, contemplated herein include, but are not limited to, any type of RNA, e.g., mRNA, siRNA, miRNA, sgRNA, and guide RNA and any type of DNA, genomic DNA, plasmid DNA, and minicircle DNA, and any fragments thereof. In aspects, the nucleic acid is messenger RNA. In aspects, the messenger RNA is messenger ribonucleoprotein (RNP). The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, sgRNA, guide RNA, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g., phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In aspects, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

The term “complementary” or “complementarity” refers to the ability of a nucleic acid to form hydrogen bond(s) with another nucleic acid sequence by either traditional Watson-Crick or other non-traditional types. For example, the sequence A-G-T is complementary to the sequence T-C-A. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%. 97%, 98%, 99%, or 100% over a region of 10, 15, 20, 25, 30, 35, 40, 45, 50, or more nucleotides, or refers to two nucleic acids that hybridize under stringent conditions (i.e., stringent hybridization conditions).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. One of ordinary skill will readily recognize that alternative hybridization and wash conditions can be utilized to provide conditions of similar stringency. Additional guidelines for determining hybridization parameters are provided in numerous references, e.g., Current Protocols in Molecular Biology, ed. Ausubel, et al., supra.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., sgRNA) may be detected by standard PCR or Northern blot methods well known in the art. See, Sambrook et al., 1989 Molecular Cloning: A Laboratory Manual, 18.1-18.88.

The term “transcriptional regulatory sequence” as provided herein refers to a segment of DNA that is capable of increasing or decreasing transcription (e.g., expression) of a specific gene within an organism. Non-limiting examples of transcriptional regulatory sequences include promoters, enhancers, and silencers.

The terms “transcription start site” and transcription initiation site” may be used interchangeably to refer herein to the 5′ end of a gene sequence (e.g., DNA sequence) where RNA polymerase (e.g., DNA-directed RNA polymerase) begins synthesizing the RNA transcript. The transcription start site may be the first nucleotide of a transcribed DNA sequence where RNA polymerase begins synthesizing the RNA transcript. A skilled artisan can determine a transcription start site via routine experimentation and analysis, for example, by performing a run-off transcription assay or by definitions according to FANTOM5 database.

The term “promoter” as used herein refers to a region of DNA that initiates transcription of a particular gene. Promoters are typically located near the transcription start site of a gene, upstream of the gene and on the same strand (i.e., 5′ on the sense strand) on the DNA. Promoters may be about 100 to about 1000 base pairs in length.

The term “enhancer” as used herein refers to a region of DNA that may be bound by proteins (e.g., transcription factors) to increase the likelihood that transcription of a gene will occur. Enhancers may be about 50 to about 1500 base pairs in length. Enhancers may be located downstream or upstream of the transcription initiation site that it regulates and may be several hundreds of base pairs, several thousands of base pairs, or several millions of base pairs away from the transcription initiation site.

The term “silencer” as used herein refers to a DNA sequence capable of binding transcription regulation factors known as repressors, thereby negatively effecting transcription of a gene. Silencer DNA sequences may be found at many different positions throughout the DNA, including, but not limited to, upstream of a target gene for which it acts to repress transcription of the gene (e.g., silence gene expression).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides may also be referred to by their commonly accepted single-letter codes.

The terms “polypeptide,” “peptide” and “protein” are used interchangeably herein to refer to a polymer of amino acid residues, wherein the polymer may, in aspects, be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M) (see, e.g., Creighton, Proteins (1984)).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

As used herein, the term “vector” refers to a nucleic acid molecule capable of transporting another nucleic acid to which it has been linked. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. Additionally, some viral vectors are capable of targeting a particular cells type either specifically or non-specifically. Replication-incompetent viral vectors or replication-defective viral vectors refer to viral vectors that are capable of infecting their target cells and delivering their viral payload, but then fail to continue the typical lytic pathway that leads to cell lysis and death.

The terms “transfection”, “transduction”, “transfecting” or “transducing” can be used interchangeably and are defined as a process of introducing a nucleic acid molecule (e.g., mRNA, DNA, RNP) and/or a protein to a cell. Nucleic acids may be introduced to a cell using non-viral or viral-based methods. The nucleic acid molecule can be a sequence encoding complete proteins or functional portions thereof. Typically, a nucleic acid vector, comprising the elements necessary for protein expression (e.g., a promoter, transcription start site, etc.). Non-viral methods of transfection include any appropriate method that does not use viral DNA or viral particles as a delivery system to introduce the nucleic acid molecule into the cell. Exemplary non-viral transfection methods include nanoparticle encapsulation of the nucleic acids that encode the fusion protein (e.g., lipid nanoparticles, gold nanoparticles, and the like), calcium phosphate transfection, liposomal transfection, nucleofection, sonoporation, transfection through heat shock, magnetifection and electroporation. For viral-based methods, any useful viral vector can be used in the methods described herein. Examples of viral vectors include, but are not limited to retroviral, adenoviral, lentiviral and adeno-associated viral vectors. In aspects, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

A “peptide linker” as provided herein is a linker including a peptide moiety. In embodiments, the peptide linker is a divalent peptide, such as an amino acid sequence attached at the N-terminus and the C-terminus to the remainder of the compound (e.g., fusion protein provided herein. The peptide linker may be a peptide moiety (a divalent peptide moiety) capable of being cleaved (e.g., a P2A cleavable polypeptide). A peptide linker as provided herein may also be referred to interchangeably as an amino acid linker. In aspects, the peptide linker includes 1 to about 80 amino acid residues. In aspects, the peptide linker includes 1 to about 70 amino acid residues. In aspects, the peptide linker includes 1 to about 60 amino acid residues. In aspects, the peptide linker includes 1 to about 50 amino acid residues. In aspects, the peptide linker includes 1 to about 40 amino acid residues. In aspects, the peptide linker includes 1 to about 30 amino acid residues. In aspects, the peptide linker includes 1 to about 25 amino acid residues. In aspects, the peptide linker includes 1 to about 20 amino acid residues. In aspects, the peptide linker includes about 2 to about 20 amino acid residues. In aspects, the peptide linker includes about 2 to about 19 amino acid residues. In aspects, the peptide linker includes about 2 to about 18 amino acid residues. In aspects, the peptide linker includes about 2 to about 17 amino acid residues. In aspects, the peptide linker includes about 2 to about 16 amino acid residues. In aspects, the peptide linker includes about 2 to about 15 amino acid residues. In aspects, the peptide linker includes about 2 to about 14 amino acid residues. In aspects, the peptide linker includes about 2 to about 13 amino acid residues. In aspects, the peptide linker includes about 2 to about 12 amino acid residues. In aspects, the peptide linker includes about 2 to about 11 amino acid residues. In aspects, the peptide linker includes about 2 to about 10 amino acid residues. In aspects, the peptide linker includes about 2 to about 9 amino acid residues. In aspects, the peptide linker includes about 2 to about 8 amino acid residues. In aspects, the peptide linker includes about 2 to about 7 amino acid residues. In aspects, the peptide linker includes about 2 to about 6 amino acid residues. In aspects, the peptide linker includes about 2 to about 5 amino acid residues. In aspects, the peptide linker includes about 2 to about 4 amino acid residues. In aspects, the peptide linker includes about 2 to about 3 amino acid residues. In aspects, the peptide linker includes about 3 to about 19 amino acid residues. In aspects, the peptide linker includes about 3 to about 18 amino acid residues. In aspects, the peptide linker includes about 3 to about 17 amino acid residues. In aspects, the peptide linker includes about 3 to about 16 amino acid residues. In aspects, the peptide linker includes about 3 to about 15 amino acid residues. In aspects, the peptide linker includes about 3 to about 14 amino acid residues. In aspects, the peptide linker includes about 3 to about 13 amino acid residues. In aspects, the peptide linker includes about 3 to about 12 amino acid residues. In aspects, the peptide linker includes about 3 to about 11 amino acid residues. In aspects, the peptide linker includes about 3 to about 10 amino acid residues. In aspects, the peptide linker includes about 3 to about 9 amino acid residues. In aspects, the peptide linker includes about 3 to about 8 amino acid residues. In aspects, the peptide linker includes about 3 to about 7 amino acid residues. In aspects, the peptide linker includes about 3 to about 6 amino acid residues. In aspects, the peptide linker includes about 3 to about 5 amino acid residues. In aspects, the peptide linker includes about 3 to about 4 amino acid residues. In aspects, the peptide linker includes about 10 to about 20 amino acid residues. In aspects, the peptide linker includes about 15 to about 20 amino acid residues. In aspects, the peptide linker includes about 2 amino acid residues. In aspects, the peptide linker includes about 3 amino acid residues. In aspects, the peptide linker includes about 4 amino acid residues. In aspects, the peptide linker includes about 5 amino acid residues. In aspects, the peptide linker includes about 6 amino acid residues. In aspects, the peptide linker includes about 7 amino acid residues. In aspects, the peptide linker includes about 8 amino acid residues. In aspects, the peptide linker includes about 9 amino acid residues. In aspects, the peptide linker includes about 10 amino acid residues. In aspects, the peptide linker includes about 11 amino acid residues. In aspects, the peptide linker includes about 12 amino acid residues. In aspects, the peptide linker includes about 13 amino acid residues. In aspects, the peptide linker includes about 14 amino acid residues. In aspects, the peptide linker includes about 15 amino acid residues. In aspects, the peptide linker includes about 16 amino acid residues. In aspects, the peptide linker includes about 17 amino acid residues. In aspects, the peptide linker includes about 18 amino acid residues. In aspects, the peptide linker includes about 19 amino acid residues. In aspects, the peptide linker includes about 20 amino acid residues. In aspects, the peptide linker includes about 21 amino acid residues. In aspects, the peptide linker includes about 22 amino acid residues. In aspects, the peptide linker includes about 23 amino acid residues. In aspects, the peptide linker includes about 24 amino acid residues. In aspects, the peptide linker includes about 25 amino acid residues.

In aspects, the peptide linker includes the sequence set forth by SEQ ID NO:17. In aspects, the peptide linker is the sequence set forth by SEQ ID NO:17. In aspects, the peptide linker includes the sequence set forth by SEQ ID NO:18. In aspects, the peptide linker is the sequence set forth by SEQ ID NO:18. In aspects, the peptide linker includes the sequence set forth by SEQ ID NO: 19. In aspects, the peptide linker is the sequence set forth by SEQ ID NO:19. In aspects, the peptide linker includes the sequence set forth by SEQ ID NO:20. In aspects, the peptide linker is the sequence set forth by SEQ ID NO:20. In aspects, the peptide linker includes the sequence set forth by SEQ ID NO:21. In aspects, the peptide linker is the sequence set forth by SEQ ID NO:21. In aspects, the peptide linker includes the sequence set forth by SEQ ID NO:22. In aspects, the peptide linker is the sequence set forth by SEQ ID NO:22. In aspects, the peptide linker includes the sequence set forth by SEQ ID NO:27. In aspects, the peptide linker is the sequence set forth by SEQ ID NO:27. In aspects, the peptide linker includes the sequence set forth by SEQ ID NO:24. In aspects, the peptide linker is the sequence set forth by SEQ ID NO:24. In aspects, the peptide linker includes the sequence set forth by SEQ ID NO:29. In aspects, the peptide linker is the sequence set forth by SEQ ID NO:29. In aspects, the peptide linker is an XTEN polypeptide. In aspects, the peptide linker has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:17, 18, 19, 20, 21, 22, 24, 27, or 29. In aspects, the peptide linker has an amino acid sequence that has at least 90% sequence identity to SEQ ID NO:17, 18, 19, 20, 21, 22, 24, 27, or 29.

In aspects, the peptide linker has an amino acid sequence that has at least 90% sequence identity to SEQ ID NO:17. In aspects, the peptide linker has an amino acid sequence that has at least 90% sequence identity to SEQ ID NO:18. In aspects, the peptide linker has an amino acid sequence that has at least 90% sequence identity to SEQ ID NO:19. In aspects, the peptide linker has an amino acid sequence that has at least 90% sequence identity to SEQ ID NO:20. In aspects, the peptide linker has an amino acid sequence that has at least 90% sequence identity to SEQ ID NO:21. In aspects, the peptide linker has an amino acid sequence that has at least 90% sequence identity to SEQ ID NO:22. In aspects, the peptide linker has an amino acid sequence that has at least 90% sequence identity to SEQ ID NO:24. In aspects, the peptide linker has an amino acid sequence that has at least 90% sequence identity to SEQ ID NO:27. In aspects, the peptide linker has an amino acid sequence that has at least 90% sequence identity to SEQ ID NO:29. In aspects, the peptide linker has an amino acid sequence that has at least 95% sequence identity to SEQ ID NO:17, 18, 19, 20, 21, 22, 24, 27, or 29. In aspects, the peptide linker has an amino acid sequence that has at least 95% sequence identity to SEQ ID NO:17. In aspects, the peptide linker has an amino acid sequence that has at least 95% sequence identity to SEQ ID NO:18. In aspects, the peptide linker has an amino acid sequence that has at least 95% sequence identity to SEQ ID NO:19. In aspects, the peptide linker has an amino acid sequence that has at least 95% sequence identity to SEQ ID NO:20. In aspects, the peptide linker has an amino acid sequence that has at least 95% sequence identity to SEQ ID NO:21. In aspects, the peptide linker has an amino acid sequence that has at least 95% sequence identity to SEQ ID NO:22. In aspects, the peptide linker has an amino acid sequence that has at least 95% sequence identity to SEQ ID NO:24. In aspects, the peptide linker has an amino acid sequence that has at least 95% sequence identity to SEQ ID NO:27. In aspects, the peptide linker has an amino acid sequence that has at least 95% sequence identity to SEQ ID NO:29.

The terms “XTEN,” “XTEN linker,” or “XTEN polypeptide” as used herein refer to an recombinant polypeptide (e.g. unstructured recombinant peptide) lacking hydrophobic amino acid residues. The development and use of XTEN can be found in, for example, Schellenberger et al., Nature Biotechnology 27, 1186-1190 (2009). In aspects, the XTEN linker includes the sequence set forth by SEQ ID NO:31. In aspects, the XTEN linker is the sequence set forth by SEQ ID NO:31. In aspects, the XTEN linker includes the sequence set forth by SEQ ID NO:32. In aspects, the XTEN linker is the sequence set forth by SEQ ID NO:32. In aspects, the XTEN linker has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:31. In aspects, the XTEN linker has an amino acid sequence that has at least 90% sequence identity to SEQ ID NO:31. In aspects, the XTEN linker has an amino acid sequence that has at least 95% sequence identity to SEQ ID NO:31. In aspects, the XTEN linker has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:32. In aspects, the XTEN linker has an amino acid sequence that has at least 90% sequence identity to SEQ ID NO:32. In aspects, the XTEN linker has an amino acid sequence that has at least 95% sequence identity to SEQ ID NO:32.

“Epitope tag” refers to a biological moiety, such as a peptide, that is genetically engineered into a recombinant protein and that functions as a universal epitope that is easily detected by commercially available assays or antibodies and that generally does not compromise the native structure or function of the protein.

A “detectable agent” or “detectable moiety” is a composition detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monocrystalline iron oxide nanoparticles, monocrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gases, perfluorcarbon, nitrogen, octafluoropropane, perflexane lipid microsphere, perflutren), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

A detectable moiety is a monovalent detectable agent or a detectable agent capable of forming a bond with another composition. In aspects, the detectable agent is an epitope tag. In aspects, the epitope tag is an HA tag. In aspects, the HA tag includes the sequence set forth by SEQ ID NO:24. In aspects, the HA tag is the sequence set forth by SEQ ID NO:24. In aspects, the HA tag has an amino acid sequence that has at least 80% sequence identity to SEQ ID NO:24. In aspects, the HA tag has an amino acid sequence that has at least 85% sequence identity to SEQ ID NO:24. In aspects, the HA tag has an amino acid sequence that has at least 90% sequence identity to SEQ ID NO:24. In aspects, the HA tag has an amino acid sequence that has at least 95% sequence identity to SEQ ID NO:24.

In aspects, the detectable agent is a fluorescent protein. In aspects, the fluorescent protein is blue fluorescent protein (BFP). In aspects, the BFP includes the sequence set forth by SEQ ID NO:30. In aspects, the BFP is the sequence set forth by SEQ ID NO:30. In aspects, the BFP has an amino acid sequence that has at least 80% sequence identity to SEQ ID NO:30. In aspects, the BFP has an amino acid sequence that has at least 85% sequence identity to SEQ ID NO:30. In aspects, the BFP has an amino acid sequence that has at least 90% sequence identity to SEQ ID NO:30. In aspects, the BFP has an amino acid sequence that has at least 95% sequence identity to SEQ ID NO:30.

Radioactive substances (e.g., radioisotopes) that may be used as imaging and/or labeling agents in accordance with the aspects of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga ⁶⁸Ga, ⁷⁷As, ⁸⁶Y, ⁹⁰Y. ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the aspects of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

“Contacting” is used in accordance with its plain ordinary meaning and refers to the process of allowing at least two distinct species to become sufficiently proximal to react, interact or physically touch. It should be appreciated, however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, a fusion protein as provided herein and a nucleic acid sequence (e.g., target DNA sequence).

As defined herein, the term “inhibition”, “inhibit”, “inhibiting,” “repression,” repressing,” “silencing,” “silence” and the like when used in reference to a composition as provided herein (e.g., fusion protein, complex, nucleic acid, vector) refer to negatively affecting (e.g., decreasing) the activity (e.g., transcription) of a nucleic acid sequence (e.g., decreasing transcription of a gene) relative to the activity of the nuclei acid sequence (e.g., transcription of a gene) in the absence of the composition (e.g., fusion protein, complex, nucleic acid, vector). In aspects, inhibition refers to reduction of a disease or symptoms of disease (e.g., cancer). Thus, inhibition includes, at least in part, partially or totally blocking activation (e.g., transcription), or decreasing, preventing, or delaying activation (e.g., transcription) of the nucleic acid sequence. The inhibited activity (e.g., transcription) may be 90%, 80%, 70%, 60%, 50%, 40%, 30%, 20%, 10%, or less than that in a control. In aspects, the inhibition is 1.5-fold, 2-fold, 3-fold, 4-fold, 5-fold, 10-fold, or more in comparison to a control.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

### Fusion Proteins

Provided herein are, inter alia, fusion proteins that can turn off genes permanently (e.g., irreversibly) and reversibly in mammalian cells using CRISPR-based epigenome editing. In embodiments, the fusion protein includes a single polypeptide fusion of proteins (e.g., catalytically inactive Cas9 (e.g., dCas9), a KRAB domain, Dnmt3A and Dnmt3L) which can be transiently delivered as mRNA, DNA or RNP and expressed transiently in cells. The fusion protein can be directed to a specific site in a mammalian genome using a sgRNA or cr.tracrRNA. Once properly positioned and without intending to be bound by a theory, the fusion protein adds DNA methylation and/or repressive chromatin marks to the target nucleic acid, resulting in gene silencing that is inheritable across subsequent cell divisions. In this way, the fusion protein can perform epigenome editing that bypasses the need to generate DNA double-strand breaks in the host genome, making it a safe and reversible way of manipulating the genome of a living organism.

In embodiments, the fusion protein comprises a nuclease-deficient RNA-guided DNA endonuclease enzyme; a KRAB domain, and a DNA methyltransferase domain. In aspects, the fusion protein comprises, from N-terminus to C-terminus, a DNA methyltransferase domain, a nuclease-deficient RNA-guided DNA endonuclease enzyme, and KRAB domain. In aspects, the fusion protein comprises, from N-terminus to C-terminus, a KRAB domain a nuclease-deficient RNA-guided DNA endonuclease enzyme, and a DNA methyltransferase domain. In embodiments, the nuclease-deficient RNA-guided endonuclease enzyme is a CRISPR-associated protein. In embodiments, the fusion protein further comprises one or more peptide linkers. In aspects, the fusion protein further comprises one or more detectable tags. In aspects, the fusion protein further comprises one or more nuclear localization sequences. In aspects, the fusion protein further comprises one or more peptide linkers, one or more detectable tags, one or more nuclear localization sequences, or a combination of two or more of the foregoing. When the fusion protein comprises one or more peptide linkers, each peptide liner can be the same or different. When the fusion protein comprises one or more detectable tags, each detectable tag can be the same or different. In aspects, the fusion protein comprises from 1 to 10 detectable tags. In aspects, the fusion protein comprises from 1 to 9 detectable tags. In aspects, the fusion protein comprises from 1 to 8 detectable tags. In aspects, the fusion protein comprises from 1 to 7 detectable tags. In aspects, the fusion protein comprises from 1 to 6 detectable tags. In aspects, the fusion protein comprises from 1 to 5 detectable tags. In aspects, the fusion protein comprises from 1 to 4 detectable tags. In aspects, the fusion protein comprises from 1 to 3 detectable tags. In aspects, the fusion protein comprises from 1 to 2 detectable tags. In aspects, the fusion protein comprises 1 detectable tag. In aspects, the fusion protein comprises 2 detectable tags. In aspects, the fusion protein comprises 3 detectable tags. In aspects, the fusion protein comprises 4 detectable tags. In aspects, the fusion protein comprises 5 detectable tags.

In embodiments, the fusion protein comprises a nuclease-deficient DNA endonuclease enzyme; a KRAB domain, and a DNA methyltransferase domain. In aspects, the fusion protein comprises, from N-terminus to C-terminus, a DNA methyltransferase domain, a nuclease-deficient DNA endonuclease enzyme, and KRAB domain. In aspects, the fusion protein comprises, from N-terminus to C-terminus, a KRAB domain a nuclease-deficient DNA endonuclease enzyme, and a DNA methyltransferase domain. In embodiments, the fusion protein further comprises one or more peptide linkers. In aspects, the fusion protein further comprises one or more detectable tags. In aspects, the fusion protein further comprises one or more nuclear localization sequences. In aspects, the fusion protein further comprises one or more peptide linkers, one or more detectable tags, one or more nuclear localization sequences, or a combination of two or more of the foregoing. When the fusion protein comprises one or more peptide linkers, each peptide liner can be the same or different. In embodiments, the nuclease-deficient DNA endonuclease enzyme is a zinc finger domain or TALE. In embodiments, the nuclease-deficient DNA endonuclease enzyme is a zinc finger domain. In embodiments, the nuclease-deficient DNA endonuclease enzyme is a TALE.

In embodiments, the disclosure provides a fusion protein comprising, from N-terminus to C-terminus, a DNA methyltransferase domain, a first XTEN linker, a nuclease-deficient RNA-guided endonuclease enzyme, a second XTEN linker, and a Krüppel-associated box domain. In aspects, the first XTEN linker comprises one or more amino acid residues than the second XTEN linker. In embodiments, the fusion protein comprises, from N-terminus to C-terminus, a DNA methyltransferase domain, a first XTEN linker comprising from about 5 to about 864 amino acid residues, a nuclease-deficient RNA-guided endonuclease enzyme, a second XTEN linker comprising from about 5 to about 864 amino acid residues, and a Krüppel-associated box domain. In aspects, the first and second XTEN linkers comprise from about 20 to about 100 amino acid residues. In embodiments, the nuclease-deficient RNA-guided endonuclease enzyme is a CRISPR-associated protein. In aspects, the nuclease-deficient RNA-guided DNA endonuclease enzyme is dCas9, dCpf1, ddCpf1, a nuclease-deficient Cas9 variant, a nuclease-deficient Class II CRISPR endonuclease, a zinc finger domain, a leucine zipper domain, a winged helix domain, a TALE, a helix-turn-helix motif, a helix-loop-helix domain, an HMB-box domain, a Wor3 domain, an OB-fold domain, an immunoglobulin domain, or a B3 domain. In aspects, the nuclease-deficient RNA-guided DNA endonuclease enzyme is dCas9. In aspects, the nuclease-deficient RNA-guided DNA endonuclease enzyme is Cas-phi. In aspects, the DNA methyltransferase domain comprises a Dnmt3A. In aspects, the DNA methyltransferase domain (Dnmt3A) further comprises a Dnmt3L regulatory factor (referred to herein as a Dnmt3A-3L domain or a Dnmt3B-3L domain). In aspects, the fusion protein further comprises an epitope tag, a 2A peptide, a fluorescent protein tag, a nuclear localization signal peptide, or a combination of two or more thereof.

In embodiments, the disclosure provides a fusion protein comprising, from N-terminus to C-terminus, a DNA methyltransferase domain, a first XTEN linker, a nuclease-deficient endonuclease enzyme, a second XTEN linker, and a Krüppel-associated box domain. In aspects, the first XTEN linker comprises one or more amino acid residues than the second XTEN linker. In embodiments, the fusion protein comprises, from N-terminus to C-terminus, a DNA methyltransferase domain, a first XTEN linker comprising from about 5 to about 864 amino acid residues, a nuclease-deficient endonuclease enzyme, a second XTEN linker comprising from about 5 to about 864 amino acid residues, and a Krüppel-associated box domain. In aspects, the first and second XTEN linkers comprise from about 20 to about 100 amino acid residues. In aspects, the nuclease-deficient DNA endonuclease enzyme is a zinc finger domain or a TALE. In aspects, the nuclease-deficient DNA endonuclease enzyme is a zinc finger domain. In aspects, the nuclease-deficient DNA endonuclease enzyme is a TALE. In aspects, the DNA methyltransferase domain comprises a Dnmt3A. In aspects, the DNA methyltransferase domain (Dnmt3A) further comprises a Dnmt3L regulatory factor (referred to herein as a Dnmt3A-3L domain or a Dnmt3B-3L domain). In aspects, the fusion protein further comprises an epitope tag, a 2A peptide, a fluorescent protein tag, a nuclear localization signal peptide, or a combination of two or more thereof.

In embodiments, the disclosure provides a fusion protein comprising, from N-terminus to C-terminus, a DNA methyltransferase domain, a first XTEN linker, a nuclease-deficient RNA-guided endonuclease enzyme, a second XTEN linker, and a Krüppel-associated box domain. In aspects, the first XTEN linker comprises one or more amino acid residues than the second XTEN linker. In embodiments, the fusion protein comprises, from N-terminus to C-terminus, a DNA methyltransferase domain, a first XTEN linker comprising from greater than 50 to about 864 amino acid residues, a nuclease-deficient RNA-guided endonuclease enzyme, a second XTEN linker comprising from about 5 to 50 amino acid residues, and a Krüppel-associated box domain. In aspects, the first XTEN linker comprises from about 60 to about 864 amino acid residues, and the second XTEN linker comprises from about 10 to about 40 amino acid residues. In aspects, the first XTEN linker comprises from about 70 to about 864 amino acid residues, and the second XTEN linker comprises from about 10 to about 30 amino acid residues. In aspects, the nuclease-deficient RNA-guided DNA endonuclease enzyme is a CRISPR-associated protein. In aspects, the nuclease-deficient RNA-guided DNA endonuclease enzyme is dCas9, dCpf1, ddCpf1, a nuclease-deficient Cas9 variant, a nuclease-deficient Class II CRISPR endonuclease, a leucine zipper domain, a winged helix domain, a helix-turn-helix motif, a helix-loop-helix domain, an HMB-box domain, a Wor3 domain, an OB-fold domain, an immunoglobulin domain, or a B3 domain. In aspects, the nuclease-deficient RNA-guided DNA endonuclease enzyme is dCas9. In aspects, the nuclease-deficient RNA-guided DNA endonuclease enzyme is Cas-phi. In aspects, the DNA methyltransferase domain comprises a Dnmt3A. In aspects, the DNA methyltransferase domain (Dnmt3A) further comprises a Dnmt3L regulatory factor (referred to herein as a Dnmt3A-3L domain or a Dnmt3B-3L domain). In aspects, the fusion protein further comprises an epitope tag, a 2A peptide, a fluorescent protein tag, a nuclear localization signal peptide, or a combination of two or more thereof.

In embodiments, the disclosure provides a fusion protein comprising, from N-terminus to C-terminus, a DNA methyltransferase domain, a first XTEN linker, a nuclease-deficient endonuclease enzyme, a second XTEN linker, and a Krüppel-associated box domain. In aspects, the first XTEN linker comprises one or more amino acid residues than the second XTEN linker. In embodiments, the fusion protein comprises, from N-terminus to C-terminus, a DNA methyltransferase domain, a first XTEN linker comprising from greater than 50 to about 864 amino acid residues, a nuclease-deficient endonuclease enzyme, a second XTEN linker comprising from about 5 to 50 amino acid residues, and a Krüppel-associated box domain. In aspects, the first XTEN linker comprises from about 60 to about 864 amino acid residues, and the second XTEN linker comprises from about 10 to about 40 amino acid residues. In aspects, the first XTEN linker comprises from about 70 to about 864 amino acid residues, and the second XTEN linker comprises from about 10 to about 30 amino acid residues. In aspects, the nuclease-deficient DNA endonuclease enzyme is a zinc finger domain or a TALE. In aspects, the nuclease-deficient DNA endonuclease enzyme is a zinc finger domain. In aspects, the nuclease-deficient DNA endonuclease enzyme is TALE. In aspects, the DNA methyltransferase domain comprises a Dnmt3A. In aspects, the DNA methyltransferase domain (Dnmt3A) further comprises a Dnmt3L regulatory factor (referred to herein as a Dnmt3A-3L domain or a Dnmt3B-3L domain). In aspects, the fusion protein further comprises an epitope tag, a 2A peptide, a fluorescent protein tag, a nuclear localization signal peptide, or a combination of two or more thereof.

In embodiments, the fusion protein comprises, from N-terminus to C-terminus, a DNA methyltransferase domain, a first XTEN linker, a nuclease-deficient RNA-guided endonuclease enzyme, an epitope tag, a nuclear localization signal peptide, a second XTEN linker, a Krüppel-associated box domain, a 2A cleavable peptide, and a fluorescent protein tag. In aspects, the first XTEN linker comprises one or more amino acid residues than the second XTEN linker. In aspects, the first XTEN linker comprises from greater than 50 to about 864 amino acid residues, and the second XTEN linker comprises from about 5 to 50 amino acid residues. In aspects, the first XTEN linker comprises from about 60 to about 864 amino acid residues, and the second XTEN linker comprises from about 10 to about 40 amino acid residues. In aspects, the first XTEN linker comprises from about 70 to about 864 amino acid residues, and the second XTEN linker comprises from about 10 to about 30 amino acid residues. In aspects, the nuclease-deficient RNA-guided DNA endonuclease enzyme is dCas9, dCpf1, ddCpf1, a nuclease-deficient Cas9 variant, a nuclease-deficient Class II CRISPR endonuclease, a leucine zipper domain, a winged helix domain, a helix-turn-helix motif, a helix-loop-helix domain, an HMB-box domain, a Wor3 domain, an OB-fold domain, an immunoglobulin domain, or a B3 domain. In aspects, the nuclease-deficient RNA-guided DNA endonuclease enzyme is a CRISPR-associated protein. In aspects, the nuclease-deficient RNA-guided DNA endonuclease enzyme is dCas9. In aspects, the nuclease-deficient RNA-guided DNA endonuclease enzyme is Cas-phi. In aspects, the DNA methyltransferase domain comprises a Dnmt3A domain. In aspects, the Dnmt3A domain is linked to a Dnmt3L regulatory factor (referred to herein as a Dnmt3A-3L domain). In aspects, the DNA methyltransferase domain comprises a Dnmt3B domain. In aspects, the Dnmt3B domain is linked to a Dnmt3L regulatory factor (referred to herein as a Dnmt3B-3L domain).

In embodiments, the fusion protein comprises, from N-terminus to C-terminus, a DNA methyltransferase domain, a first XTEN linker, a nuclease-deficient endonuclease enzyme, an epitope tag, a nuclear localization signal peptide, a second XTEN linker, a Krüppel-associated box domain, a 2A cleavable peptide, and a fluorescent protein tag. In aspects, the first XTEN linker comprises one or more amino acid residues than the second XTEN linker. In aspects, the first XTEN linker comprises from greater than 50 to about 864 amino acid residues, and the second XTEN linker comprises from about 5 to 50 amino acid residues. In aspects, the first XTEN linker comprises from about 60 to about 864 amino acid residues, and the second XTEN linker comprises from about 10 to about 40 amino acid residues. In aspects, the first XTEN linker comprises from about 70 to about 864 amino acid residues, and the second XTEN linker comprises from about 10 to about 30 amino acid residues. In aspects, the nuclease-deficient DNA endonuclease enzyme is a zinc finger domain or a TALE. In aspects, the nuclease-deficient DNA endonuclease enzyme is zinc finger domain. In aspects, the nuclease-deficient DNA endonuclease enzyme is TALE. In aspects, the DNA methyltransferase domain comprises a Dnmt3A domain. In aspects, the Dnmt3A domain is linked to a Dnmt3L regulatory factor (referred to herein as a Dnmt3A-3L domain). In aspects, the DNA methyltransferase domain comprises a Dnmt3B domain. In aspects, the Dnmt3B domain is linked to a Dnmt3L regulatory factor (referred to herein as a Dnmt3B-3L domain).

In embodiments, the fusion protein includes an amino acid sequence having at least 75% sequence identity to SEQ ID NO:97, 98, 99, 107, 108, 109, or 110. In aspects, the fusion protein includes an amino acid sequence having at least 75% sequence identity to SEQ ID NO:97. In aspects, the fusion protein includes an amino acid sequence having at least 75% sequence identity to SEQ ID NO:98. In aspects, the fusion protein includes an amino acid sequence having at least 75% sequence identity to SEQ ID NO:99. In aspects, the fusion protein includes an amino acid sequence having at least 75% sequence identity to SEQ ID NO:107. In aspects, the fusion protein includes an amino acid sequence having at least 75% sequence identity to SEQ ID NO: 108. In aspects, the fusion protein includes an amino acid sequence having at least 75% sequence identity to SEQ ID NO:109. In aspects, the fusion protein includes an amino acid sequence having at least 75% sequence identity to SEQ ID NO: 110.

In embodiments, the fusion protein includes an amino acid sequence having at least 80% sequence identity to SEQ ID NO:97, 98, 99, 107, 108, 109, or 110. In aspects, the fusion protein includes an amino acid sequence having at least 80% sequence identity to SEQ ID NO:97. In aspects, the fusion protein includes an amino acid sequence having at least 80% sequence identity to SEQ ID NO:98. In aspects, the fusion protein includes an amino acid sequence having at least 80% sequence identity to SEQ ID NO:99. In aspects, the fusion protein includes an amino acid sequence having at least 80% sequence identity to SEQ ID NO:107. In aspects, the fusion protein includes an amino acid sequence having at least 80% sequence identity to SEQ ID NO: 108. In aspects, the fusion protein includes an amino acid sequence having at least 80% sequence identity to SEQ ID NO:109. In aspects, the fusion protein includes an amino acid sequence having at least 80% sequence identity to SEQ ID NO: 110.

In embodiments, the fusion protein includes an amino acid sequence having at least 85% sequence identity to SEQ ID NO:97, 98, 99, 107, 108, 109, or 110. In aspects, the fusion protein includes an amino acid sequence having at least 85% sequence identity to SEQ ID NO:97. In aspects, the fusion protein includes an amino acid sequence having at least 85% sequence identity to SEQ ID NO:98. In aspects, the fusion protein includes an amino acid sequence having at least 85% sequence identity to SEQ ID NO:99. In aspects, the fusion protein includes an amino acid sequence having at least 85% sequence identity to SEQ ID NO:107. In aspects, the fusion protein includes an amino acid sequence having at least 85% sequence identity to SEQ ID NO: 108. In aspects, the fusion protein includes an amino acid sequence having at least 85% sequence identity to SEQ ID NO:109. In aspects, the fusion protein includes an amino acid sequence having at least 85% sequence identity to SEQ ID NO: 110.

In embodiments, the fusion protein includes an amino acid sequence having at least 88% sequence identity to SEQ ID NO:97, 98, 99, 107, 108, 109, or 110. In aspects, the fusion protein includes an amino acid sequence having at least 88% sequence identity to SEQ ID NO:97. In aspects, the fusion protein includes an amino acid sequence having at least 88% sequence identity to SEQ ID NO:98. In aspects, the fusion protein includes an amino acid sequence having at least 88% sequence identity to SEQ ID NO:99. In aspects, the fusion protein includes an amino acid sequence having at least 88% sequence identity to SEQ ID NO:107. In aspects, the fusion protein includes an amino acid sequence having at least 88% sequence identity to SEQ ID NO: 108. In aspects, the fusion protein includes an amino acid sequence having at least 88% sequence identity to SEQ ID NO:109. In aspects, the fusion protein includes an amino acid sequence having at least 88% sequence identity to SEQ ID NO:110.

In embodiments, the fusion protein includes an amino acid sequence having at least 90% sequence identity to SEQ ID NO:97, 98, 99, 107, 108, 109, or 110. In aspects, the fusion protein includes an amino acid sequence having at least 90% sequence identity to SEQ ID NO:97. In aspects, the fusion protein includes an amino acid sequence having at least 90% sequence identity to SEQ ID NO:98. In aspects, the fusion protein includes an amino acid sequence having at least 90% sequence identity to SEQ ID NO:99. In aspects, the fusion protein includes an amino acid sequence having at least 90% sequence identity to SEQ ID NO:107. In aspects, the fusion protein includes an amino acid sequence having at least 90% sequence identity to SEQ ID NO: 108. In aspects, the fusion protein includes an amino acid sequence having at least 90% sequence identity to SEQ ID NO:109. In aspects, the fusion protein includes an amino acid sequence having at least 90% sequence identity to SEQ ID NO: 110.

In embodiments, the fusion protein includes an amino acid sequence having at least 95% sequence identity to SEQ ID NO:97, 98, 99, 107, 108, 109, or 110. In aspects, the fusion protein includes an amino acid sequence having at least 92% sequence identity to SEQ ID NO:97. In aspects, the fusion protein includes an amino acid sequence having at least 92% sequence identity to SEQ ID NO:98. In aspects, the fusion protein includes an amino acid sequence having at least 92% sequence identity to SEQ ID NO:99. In aspects, the fusion protein includes an amino acid sequence having at least 92% sequence identity to SEQ ID NO:107. In aspects, the fusion protein includes an amino acid sequence having at least 92% sequence identity to SEQ ID NO: 108. In aspects, the fusion protein includes an amino acid sequence having at least 92% sequence identity to SEQ ID NO:109. In aspects, the fusion protein includes an amino acid sequence having at least 92% sequence identity to SEQ ID NO: 110.

In embodiments, the fusion protein includes an amino acid sequence having at least 95% sequence identity to SEQ ID NO:97, 98, 99, 107, 108, 109, or 110. In aspects, the fusion protein includes an amino acid sequence having at least 94% sequence identity to SEQ ID NO:97. In aspects, the fusion protein includes an amino acid sequence having at least 94% sequence identity to SEQ ID NO:98. In aspects, the fusion protein includes an amino acid sequence having at least 94% sequence identity to SEQ ID NO:99. In aspects, the fusion protein includes an amino acid sequence having at least 94% sequence identity to SEQ ID NO:107. In aspects, the fusion protein includes an amino acid sequence having at least 94% sequence identity to SEQ ID NO: 108. In aspects, the fusion protein includes an amino acid sequence having at least 94% sequence identity to SEQ ID NO:109. In aspects, the fusion protein includes an amino acid sequence having at least 94% sequence identity to SEQ ID NO: 110.

In embodiments, the fusion protein includes an amino acid sequence having at least 95% sequence identity to SEQ ID NO:97, 98, 99, 107, 108, 109, or 110. In aspects, the fusion protein includes an amino acid sequence having at least 95% sequence identity to SEQ ID NO:97. In aspects, the fusion protein includes an amino acid sequence having at least 95% sequence identity to SEQ ID NO:98. In aspects, the fusion protein includes an amino acid sequence having at least 95% sequence identity to SEQ ID NO:99. In aspects, the fusion protein includes an amino acid sequence having at least 95% sequence identity to SEQ ID NO:107. In aspects, the fusion protein includes an amino acid sequence having at least 95% sequence identity to SEQ ID NO: 108. In aspects, the fusion protein includes an amino acid sequence having at least 95% sequence identity to SEQ ID NO:109. In aspects, the fusion protein includes an amino acid sequence having at least 95% sequence identity to SEQ ID NO: 110.

In embodiments, the fusion protein includes an amino acid sequence having at least 96% sequence identity to SEQ ID NO:97, 98, 99, 107, 108, 109, or 110. In aspects, the fusion protein includes an amino acid sequence having at least 96% sequence identity to SEQ ID NO:97. In aspects, the fusion protein includes an amino acid sequence having at least 96% sequence identity to SEQ ID NO:98. In aspects, the fusion protein includes an amino acid sequence having at least 96% sequence identity to SEQ ID NO:99. In aspects, the fusion protein includes an amino acid sequence having at least 96% sequence identity to SEQ ID NO:107. In aspects, the fusion protein includes an amino acid sequence having at least 96% sequence identity to SEQ ID NO: 108. In aspects, the fusion protein includes an amino acid sequence having at least 96% sequence identity to SEQ ID NO:109. In aspects, the fusion protein includes an amino acid sequence having at least 96% sequence identity to SEQ ID NO: 110.

In embodiments, the fusion protein includes an amino acid sequence having at least 95% sequence identity to SEQ ID NO:97, 98, 99, 107, 108, 109, or 110. In aspects, the fusion protein includes an amino acid sequence having at least 98% sequence identity to SEQ ID NO:97. In aspects, the fusion protein includes an amino acid sequence having at least 98% sequence identity to SEQ ID NO:98. In aspects, the fusion protein includes an amino acid sequence having at least 98% sequence identity to SEQ ID NO:99. In aspects, the fusion protein includes an amino acid sequence having at least 98% sequence identity to SEQ ID NO:107. In aspects, the fusion protein includes an amino acid sequence having at least 98% sequence identity to SEQ ID NO: 108. In aspects, the fusion protein includes an amino acid sequence having at least 98% sequence identity to SEQ ID NO:109. In aspects, the fusion protein includes an amino acid sequence having at least 98% sequence identity to SEQ ID NO: 110.

In embodiments, the fusion protein comprises the structure: A-B-C, or B-A-C or C-A-B, or C-B-A, or B-C-A, or A-C-B; where A comprises a nuclease-deficient RNA-guided DNA endonuclease enzyme or a nuclease-deficient DNA endonuclease enzyme; B comprises a KRAB domain, C comprises a DNA methyltransferase domain; and wherein the component on the left is the N-terminus and the component on the right is the C-terminus. In aspects, the fusion protein further comprises one or more peptide linkers and one or more detectable tags. In aspects, A-B, B-A, B-C, C-B, A-C, and C-A are each independently linked together via a covalent bond, a peptide linker, a detectable tag, a nuclear localization sequence, or a combination of two or more thereof. The peptide linker can be any known in the art (e.g., P2A cleavable peptide, XTEN linker, and the like). In aspects, the fusion protein comprises other components, such as detectable tags (e.g., HA tag, blue fluorescent protein, and the like). In embodiments, “A” is a nuclease-deficient RNA-guided DNA endonuclease enzyme. In embodiments, “A” is a CRISPR-associated protein. In embodiments, “A” is a nuclease-deficient DNA endonuclease enzyme. In embodiments, “A” is a zinc finger domain. In embodiments, “A” is TALE.

In embodiments, the fusion protein comprises the structure: A-L₁-B-L₂-C or C-L₂-B-L₁-A or C-L₂-A-L₁-B, where A comprises a nuclease-deficient RNA-guided DNA endonuclease enzyme or a nuclease-deficient DNA endonuclease enzyme; B comprises a KRAB domain, C comprises a DNA methyltransferase domain, L₁ is absent, a covalent bond, or a peptide linker, and L₂ is absent, a covalent bond, or a peptide linker; and where the component at the left is at the N-terminus and the component on the right is at the C-terminus. In aspects, A is covalently linked to B via a peptide linker. In aspects, A is covalently linked to B via a covalent bond. In aspects, B is covalently linked to C via a peptide linker. In aspects, B is covalently linked to C via a covalent bond. The peptide linker can be any known in the art (e.g., P2A cleavable peptide, XTEN linker, and the like). In aspects, the fusion protein comprises other components, such as detectable tags, nuclear localization sequences, and the like. In aspects, L₁ is a covalent bond, a peptide linker, a detectable tag, a nuclear localization sequence, or a combination thereof. In aspects, L₂ is a covalent bond, a peptide linker, a detectable tag, a nuclear localization sequence, or a combination thereof. In embodiments, “A” is a nuclease-deficient RNA-guided DNA endonuclease enzyme. In embodiments, “A” is a CRISPR-associated protein. In embodiments, “A” is a nuclease-deficient DNA endonuclease enzyme. In embodiments, “A” is a zinc finger domain. In embodiments, “A” is TALE.

In embodiments, the fusion protein has at least 80% sequence identity to the fusion protein having the amino acid sequence of Formula (A); where the amino acid sequence of Formula (A) is, from N-terminus to C-terminus:

C₁—R³-C₂-R²-A-R¹-R⁴-B (A),

wherein C₁ comprises SEQ ID NO:26 or SEQ ID NO:106; R³ is absent or R³ comprises SEQ ID NO:27; C₂ comprises SEQ ID NO:28; R² is absent or R² comprises SEQ ID NO:32; A comprises SEQ ID NO:23; R¹ is absent or R¹ comprises SEQ ID NO:25; R⁴ is absent or R⁴ comprises SEQ ID NO:31; and B comprises SEQ ID NO:16, SEQ ID NO:103, SEQ ID NO:104, or SEQ ID NO:105. In embodiments, R² comprises SEQ ID NO:32, R³ comprises SEQ ID NO:27, R¹ comprises SEQ ID NO:25, and R⁴ is absent. In embodiments, R² comprises SEQ ID NO:32, R³ comprises SEQ ID NO:27, R¹ is absent, and R⁴ is absent. In embodiments, R² comprises SEQ ID NO:32, R³ comprises SEQ ID NO:27, R¹ is absent, and R⁴ comprises SEQ ID NO:31. In embodiments, R2 comprises SEQ ID NO:32, R³ comprises SEQ ID NO:27, R¹ comprises SEQ ID NO:25, and R⁴ comprises SEQ ID NO:31. In embodiments, C₁ comprises SEQ ID NO:26, B comprises SEQ ID NO: 16, R² comprises SEQ ID NO:32, R³ comprises SEQ ID NO:27, R¹ comprises SEQ ID NO:25, and R⁴ is absent. In embodiments, C1 comprises SEQ ID NO:26, B comprises SEQ ID NO: 16, R² comprises SEQ ID NO:32, R³ comprises SEQ ID NO:27, R¹ is absent, and R⁴ is absent. In embodiments, C1 comprises SEQ ID NO:26, B comprises SEQ ID NO:16, R² comprises SEQ ID NO:32, R³ comprises SEQ ID NO:27, R¹ is absent, and R⁴ comprises SEQ ID NO:31. In embodiments, C1 comprises SEQ ID NO:26, B comprises SEQ ID NO:16, R² comprises SEQ ID NO:32, R³ comprises SEQ ID NO:27, R¹ comprises SEQ ID NO:25, and R⁴ comprises SEQ ID NO:31. In embodiments, C1 comprises SEQ ID NO:26, B comprises SEQ ID NO: 103, R² comprises SEQ ID NO:32, R³ comprises SEQ ID NO:27, R¹ comprises SEQ ID NO:25, and R⁴ is absent. In embodiments, C1 comprises SEQ ID NO:26, B comprises SEQ ID NO: 103, R² comprises SEQ ID NO:32, R³ comprises SEQ ID NO:27, R¹ is absent, and R⁴ is absent. In embodiments, C1 comprises SEQ ID NO:26, B comprises SEQ ID NO:103, R² comprises SEQ ID NO:32, R³ comprises SEQ ID NO:27, R¹ is absent, and R⁴ comprises SEQ ID NO:31. In embodiments, C₁ comprises SEQ ID NO:26, B comprises SEQ ID NO:103, R² comprises SEQ ID NO:32, R³ comprises SEQ ID NO:27, R¹ comprises SEQ ID NO:25, and R⁴ comprises SEQ ID NO:31. In embodiments, C₁ comprises SEQ ID NO:26, B comprises SEQ ID NO: 104, R² comprises SEQ ID NO:32, R³ comprises SEQ ID NO:27, R¹ comprises SEQ ID NO:25, and R⁴ is absent. In embodiments, C1 comprises SEQ ID NO:26, B comprises SEQ ID NO: 104, R² comprises SEQ ID NO:32, R³ comprises SEQ ID NO:27, R¹ is absent, and R⁴ is absent. In embodiments, C₁ comprises SEQ ID NO:26, B comprises SEQ ID NO:104, R² comprises SEQ ID NO:32, R³ comprises SEQ ID NO:27, R¹ is absent, and R⁴ comprises SEQ ID NO:31. In embodiments, C1 comprises SEQ ID NO:26, B comprises SEQ ID NO:104, R² comprises SEQ ID NO:32, R³ comprises SEQ ID NO:27, R¹ comprises SEQ ID NO:25, and R⁴ comprises SEQ ID NO:31. In embodiments, C1 comprises SEQ ID NO:26, B comprises SEQ ID NO: 105, R² comprises SEQ ID NO:32, R³ comprises SEQ ID NO:27, R¹ comprises SEQ ID NO:25, and R⁴ is absent. In embodiments, C1 comprises SEQ ID NO:26, B comprises SEQ ID NO: 105, R² comprises SEQ ID NO:32, R³ comprises SEQ ID NO:27, R¹ is absent, and R⁴ is absent. In embodiments, C1 comprises SEQ ID NO:26, B comprises SEQ ID NO:105, R² comprises SEQ ID NO:32, R³ comprises SEQ ID NO:27, R¹ is absent, and R⁴ comprises SEQ ID NO:31. In embodiments, C1 comprises SEQ ID NO:26, B comprises SEQ ID NO:105, R² comprises SEQ ID NO:32, R³ comprises SEQ ID NO:27, R¹ comprises SEQ ID NO:25, and R⁴ comprises SEQ ID NO:31. In embodiments, C1 comprises SEQ ID NO:106, B comprises SEQ ID NO: 16, R² comprises SEQ ID NO:32, R³ comprises SEQ ID NO:27, R¹ comprises SEQ ID NO:25, and R⁴ is absent. In embodiments, C1 comprises SEQ ID NO: 106, B comprises SEQ ID NO: 16, R² comprises SEQ ID NO:32, R³ comprises SEQ ID NO:27, R¹ is absent, and R⁴ is absent. In embodiments, C1 comprises SEQ ID NO: 106, B comprises SEQ ID NO:16, R² comprises SEQ ID NO:32, R³ comprises SEQ ID NO:27, R¹ is absent, and R⁴ comprises SEQ ID NO:31. In embodiments, C1 comprises SEQ ID NO:106, B comprises SEQ ID NO:16, R² comprises SEQ ID NO:32, R³ comprises SEQ ID NO:27, R¹ comprises SEQ ID NO:25, and R⁴ comprises SEQ ID NO:31. In embodiments, C1 comprises SEQ ID NO:106, B comprises SEQ ID NO: 103, R² comprises SEQ ID NO:32, R³ comprises SEQ ID NO:27, R¹ comprises SEQ ID NO:25, and R⁴ is absent. In embodiments, C1 comprises SEQ ID NO: 106, B comprises SEQ ID NO: 103, R² comprises SEQ ID NO:32, R³ comprises SEQ ID NO:27, R¹ is absent, and R⁴ is absent. In embodiments, C1 comprises SEQ ID NO: 106, B comprises SEQ ID NO:103, R² comprises SEQ ID NO:32, R³ comprises SEQ ID NO:27, R¹ is absent, and R⁴ comprises SEQ ID NO:31. In embodiments, C1 comprises SEQ ID NO:106, B comprises SEQ ID NO:103, R² comprises SEQ ID NO:32, R³ comprises SEQ ID NO:27, R¹ comprises SEQ ID NO:25, and R⁴ comprises SEQ ID NO:31. In embodiments, C1 comprises SEQ ID NO:106, B comprises SEQ ID NO: 104, R^{z} comprises SEQ ID NO:32, R³ comprises SEQ ID NO:27, R¹ comprises SEQ ID NO:25, and R⁴ is absent. In embodiments, C₁ comprises SEQ ID NO: 106, B comprises SEQ ID NO: 104, R² comprises SEQ ID NO:32, R³ comprises SEQ ID NO:27, R¹ is absent, and R⁴ is absent. In embodiments, C₁ comprises SEQ ID NO: 106, B comprises SEQ ID NO:104, R² comprises SEQ ID NO:32, R³ comprises SEQ ID NO:27, R¹ is absent, and R⁴ comprises SEQ ID NO:31. In embodiments, C₁ comprises SEQ ID NO:106, B comprises SEQ ID NO:104, R² comprises SEQ ID NO:32, R³ comprises SEQ ID NO:27, R¹ comprises SEQ ID NO:25, and R⁴ comprises SEQ ID NO:31. In embodiments, C1 comprises SEQ ID NO:106, B comprises SEQ ID NO: 105, R² comprises SEQ ID NO:32, R³ comprises SEQ ID NO:27, R¹ comprises SEQ ID NO:25, and R⁴ is absent. In embodiments, C₁ comprises SEQ ID NO: 106, B comprises SEQ ID NO: 105, R² comprises SEQ ID NO:32, R³ comprises SEQ ID NO:27, R¹ is absent, and R⁴ is absent. In embodiments, C₁ comprises SEQ ID NO: 106, B comprises SEQ ID NO:105, R² comprises SEQ ID NO:32, R³ comprises SEQ ID NO:27, R¹ is absent, and R⁴ comprises SEQ ID NO:31. In embodiments, C₁ comprises SEQ ID NO:106, B comprises SEQ ID NO:105, R2 comprises SEQ ID NO:32, R³ comprises SEQ ID NO:27, R¹ comprises SEQ ID NO:25, and R⁴ comprises SEQ ID NO:31. In embodiments, R² comprises SEQ ID NO:32. In embodiments, R² is absent. In embodiments, R¹ and R⁴ are absent. In embodiments, R¹ is absent and R⁴ comprises SEQ ID NO:31. In embodiments, R¹ comprises SEQ ID NO:25 and R⁴ is absent. In embodiments, R¹ comprises SEQ ID NO:25 and R⁴ comprises SEQ ID NO:31. In embodiments, R³ is absent. In embodiments, R³ comprises SEQ ID NO:27. In embodiments, C₁ comprises SEQ ID NO: 26. In embodiments, C₁ comprises SEQ ID NO: 105. In embodiments, B comprises SEQ ID NO: 16. In embodiments, B comprises SEQ ID NO:103. In embodiments, B comprises SEQ ID NO: 104. In embodiments, the fusion protein has at least 85% sequence identity to the fusion protein having the structure of Formula (A). In embodiments, the fusion protein has at least 88% sequence identity to the amino acid sequence of Formula (A). In embodiments, the fusion protein has at least 90% sequence identity to the amino acid sequence of Formula (A). In embodiments, the fusion protein has at least 91% sequence identity to the amino acid sequence of Formula (A). In embodiments, the fusion protein has at least 92% sequence identity to the amino acid sequence of Formula (A). In embodiments, the fusion protein has at least 93% sequence identity to the amino acid sequence of Formula (A). In embodiments, the fusion protein has at least 94% sequence identity to the amino acid sequence of Formula (A). In embodiments, the fusion protein has at least 95% sequence identity to the amino acid sequence of Formula (A). In embodiments, the fusion protein has at least 96% sequence identity to the amino acid sequence of Formula (A). In embodiments, the fusion protein has at least 97% sequence identity to the amino acid sequence of Formula (A). In embodiments, the fusion protein has at least 98% sequence identity to the amino acid sequence of Formula (A). In embodiments, the fusion protein has at least 99% sequence identity to the amino acid sequence of Formula (A).

In embodiments, the fusion protein comprises the structure: B-L₁-A-L₂-C or C-L₁-A-L₂-B where A comprise a nuclease-deficient RNA-guided DNA endonuclease enzyme or a nuclease-deficient DNA endonuclease enzyme; B comprises a KRAB domain, C comprises a DNA methyltransferase domain, L₁ is a covalent bond or a peptide linker, and L₂ is a covalent bond or a peptide linker. In embodiments the fusion protein comprises the structure: B-L₁-A-L₂-C. In embodiments the fusion protein comprises the structure: C-L₁-A-L₂-B. In aspects, L₁ is a peptide linker. In aspects, L₁ is a covalent bond. In aspects, L₂ is a peptide linker. In aspects, L₂ is a covalent bond. The peptide linker can be any known in the art or described herein (e.g., P2A cleavable peptide, XTEN linker, and the like). In aspects, the fusion protein comprises other components, such as detectable tags. In aspects, L₁ is a covalent bond, a peptide linker, a detectable tag, or a combination thereof. In aspects, L₂ is a covalent bond, a peptide linker, a detectable tag, or a combination thereof. In aspects, the fusion protein further comprises a nuclear localization sequence. In embodiments, “A” is a nuclease-deficient RNA-guided DNA endonuclease enzyme. In embodiments, “A” is a CRISPR-associated protein. In embodiments, “A” is a nuclease-deficient DNA endonuclease enzyme. In embodiments, “A” is a zinc finger domain. In embodiments, “A” is TALE.

In embodiments, the fusion protein comprises the structure: B-L₃-A-L₄-C-L₅-D or C-L₃-A-L₄-B-L₅-D, where A comprises a nuclease-deficient RNA-guided DNA endonuclease enzyme or a nuclease-deficient DNA endonuclease enzyme; B comprises a KRAB domain, C comprises a DNA methyltransferase domain, D is absent or D comprises one or more detectable tags, L₃ comprises a covalent bond, a peptide linker, a detectable tag, or a combination of two or more thereof, L₄ comprises a covalent bond, a peptide linker, a detectable tag, or a combination of two or more thereof, L₅ is absent or L₅ comprises a covalent bond or a peptide linker. In embodiments, the fusion protein comprises the structure: B-L₃-A-L₄-C-L₅-D. In embodiments, the fusion protein comprises the structure: C-L₃-A-L₄-B-L₅-D. In aspects, L₃ is a peptide linker. In aspects, L₃ is a covalent bond. In aspects, L₃ comprises a peptide linker and a detectable tag. In aspects, L₃ comprises a detectable tag. In aspects, L₄ is a peptide linker. In aspects, L₄ comprises a peptide linker and a detectable tag. In aspects, L₄ is a covalent bond. In aspects, L₄ comprises a detectable tag. In aspects, L₅ is a peptide linker. In aspects, L₅ is a covalent bond. In aspects, D comprises one or a plurality of detectable tags. In aspects, D comprises one detectable tag. In aspects, D comprises two detectable tags. In aspects, D comprises three detectable tags. In aspects, D comprises a plurality of detectable tags. D can be any detectable tag known in the art and/or described herein (e.g., HA tag, blue fluorescent protein, and the like). In aspects L₅ and D are absent. When L₃, L₄, L₅, and D comprise two or more detectable tags, each detectable tag is the same or different. The peptide linker can be any known in the art and/or described herein (e.g., P2A cleavable peptide, XTEN linker, and the like). In aspects, the fusion protein further comprises a nuclear localization sequence. In embodiments, “A” is a nuclease-deficient RNA-guided DNA endonuclease enzyme. In embodiments, “A” is a CRISPR-associated protein. In embodiments, “A” is a nuclease-deficient DNA endonuclease enzyme. In embodiments, “A” is a zinc finger domain. In embodiments, “A” is TALE.

In embodiments, the fusion protein comprises the structure: C-L₃-A-L₄-B-L₅-D, where A comprises a nuclease-deficient RNA-guided DNA endonuclease enzyme or a nuclease-deficient DNA endonuclease enzyme; B comprises a KRAB domain, C comprises a DNA methyltransferase domain, D is absent or D comprises one or more detectable tags, L₃ comprises a covalent bond, a peptide linker, a detectable tag, or a combination of two or more thereof, L₄ comprises a covalent bond, a peptide linker, a detectable tag, or a combination of two or more thereof, L₅ is absent or L₅ comprises a covalent bond or a peptide linker; and where C is at the N-terminus and D is at the C-terminus. In aspects, L₃ is a peptide linker. In aspects, L₃ is a covalent bond. In aspects, L₃ comprises a detectable tag. In aspects, L₃ comprises a peptide linker and a detectable tag. In aspects, L₄ a peptide linker. In aspects, L₄ is a covalent bond. In aspects, L₄ comprises a detectable tag. In aspects, L₄ comprises a peptide linker and a detectable tag. In aspects, L₅ a peptide linker. In aspects, L₅ is a covalent bond. In aspects, D comprises one or a plurality of detectable tags. In aspects, D comprises one detectable tag. In aspects, D comprises two detectable tags. In aspects, D comprises three detectable tags. In aspects, D comprises a plurality of detectable tags. D can be any detectable tag known in the art and/or described herein (e.g., HA tag, blue fluorescent protein, and the like). In aspects L₅ and D are absent. When L₃, L₄, L₅, and D comprise two or more detectable tags, each detectable tag is the same or different. The peptide linker can be any known in the art and/or described herein (e.g., P2A cleavable peptide, XTEN linker, and the like). In aspects, the fusion protein further comprises a nuclear localization sequence. In embodiments, “A” is a nuclease-deficient RNA-guided DNA endonuclease enzyme. In embodiments, “A” is a CRISPR-associated protein. In embodiments, “A” is a nuclease-deficient DNA endonuclease enzyme. In embodiments, “A” is a zinc finger domain. In embodiments, “A” is TALE.

In embodiments, the fusion protein comprises, from N-terminus to C-terminus, a DNA methyltransferase domain, a first XTEN linker comprising from about 60 to about 150 amino acid residues, a nuclease-deficient RNA-guided endonuclease enzyme, a second XTEN linker comprising from about 5 to about 50 amino acid residues, and a Krüppel-associated box domain. In aspects, the first XTEN linker comprises from about 70 to about 90 amino acid residues, and the second XTEN linker comprises from about 10 to about 20 amino acid residues. In aspects, the first XTEN linker comprises about 80 amino acid residues, and the second XTEN linker comprises from about 16 amino acid residues. In aspects, the fusion protein further comprises a detectable tag (e.g., an epitope tag, a fluorescent protein tag), a 2A peptide (e.g., a P2A peptide), a nuclear localization signal peptide, or a combination of two or more thereof. In aspects, the fusion protein comprises from N-terminus to C-terminus, a DNA methyltransferase domain, a first XTEN linker comprising from about 60 to about 150 amino acid residues, a nuclease-deficient RNA-guided endonuclease enzyme, an epitope tag, a nuclear localization signal peptide, a second XTEN linker comprising from about 5 to about 50 amino acid residues, a Krüppel-associated box domain, a 2A cleavable peptide, and a fluorescent protein tag. In embodiments, the nuclease-deficient RNA-guided DNA endonuclease enzyme is a CRISPR-associated protein.

In embodiments, the fusion protein comprises, from N-terminus to C-terminus, a DNA methyltransferase domain, a first XTEN linker comprising from about 60 to about 150 amino acid residues, a nuclease-deficient endonuclease enzyme, a second XTEN linker comprising from about 5 to about 50 amino acid residues, and a Krüppel-associated box domain. In aspects, the first XTEN linker comprises from about 70 to about 90 amino acid residues, and the second XTEN linker comprises from about 10 to about 20 amino acid residues. In aspects, the first XTEN linker comprises about 80 amino acid residues, and the second XTEN linker comprises from about 16 amino acid residues. In aspects, the fusion protein further comprises a detectable tag (e.g., an epitope tag, a fluorescent protein tag), a 2A peptide (e.g., a P2A peptide), a nuclear localization signal peptide, or a combination of two or more thereof. In aspects, the fusion protein comprises from N-terminus to C-terminus, a DNA methyltransferase domain, a first XTEN linker comprising from about 60 to about 150 amino acid residues, a nuclease-deficient endonuclease enzyme, an epitope tag, a nuclear localization signal peptide, a second XTEN linker comprising from about 5 to about 50 amino acid residues, a Krüppel-associated box domain, a 2A cleavable peptide, and a fluorescent protein tag. In embodiments, the nuclease-deficient endonuclease enzyme is a zinc finger domain or a TALE.

In embodiments, the peptide linker is a XTEN linker. In aspects, the XTEN linker includes about 16 to about 80 amino acid residues. In aspects, the XTEN linker includes about 17 to about 80 amino acid residues. In aspects, the XTEN linker includes about 18 to about 80 amino acid residues. In aspects, the XTEN linker includes about 19 to about 80 amino acid residues. In aspects, the XTEN linker includes about 20 to about 80 amino acid residues. In aspects, the XTEN linker includes about 30 to about 80 amino acid residues. In aspects, the XTEN linker includes about 40 to about 80 amino acid residues. In aspects, the XTEN linker includes about 50 to about 80 amino acid residues. In aspects, the XTEN linker includes about 60 to about 80 amino acid residues. In aspects, the XTEN linker includes about 70 to about 80 amino acid residues. In aspects, the XTEN linker includes about 16 to about 70 amino acid residues. In aspects, the XTEN linker includes about 16 to about 60 amino acid residues. In aspects, the XTEN linker includes about 16 to about 50 amino acid residues. In aspects, the XTEN linker includes about 16 to about 40 amino acid residues. In aspects, the XTEN linker includes about 16 to about 35 amino acid residues. In aspects, the XTEN linker includes about 16 to about 30 amino acid residues. In aspects, the XTEN linker includes about 16 to about 25 amino acid residues. In aspects, the XTEN linker includes about 16 to about 20 amino acid residues. In aspects, the XTEN linker includes about 16 amino acid residues. In aspects, the XTEN linker includes about 17 amino acid residues. In aspects, the XTEN linker includes about 18 amino acid residues. In aspects, the XTEN linker includes about 19 amino acid residues. In aspects, the XTEN linker includes about 20 amino acid residues.

In aspects, the fusion protein comprises at least two XTEN linkers that are the same or different. In aspects, the fusion protein comprises a first XTEN linker having more amino acid residues than a second XTEN linker. In aspects, the fusion protein comprises a first XTEN linker having 10 to 150 amino acid residues than a second XTEN linker. In aspects, the fusion protein comprises a first XTEN linker having 20 to 120 amino acid residues than a second XTEN linker. In aspects, the fusion protein comprises a first XTEN linker having 30 to 110 amino acid residues than a second XTEN linker. In aspects, the fusion protein comprises a first XTEN linker having 40 to 110 amino acid residues than a second XTEN linker. In aspects, the fusion protein comprises a first XTEN linker having 50 to 100 amino acid residues than a second XTEN linker. In aspects, the fusion protein comprises a first XTEN linker having 60 to 100 amino acid residues than a second XTEN linker.

In embodiments, the XTEN linker comprises from about 50 to about 864 amino acid residues. In aspects, the XTEN linker comprises from about 50 to about 200 amino acid residues. In aspects, the XTEN linker comprises from about 55 to about 180 amino acid residues. In aspects, the XTEN linker comprises from about 60 to about 150 amino acid residues. In aspects, the XTEN linker comprises from about 60 to about 120 amino acid residues. In aspects, the XTEN linker comprises from about 60 to about 110 amino acid residues. In aspects, the XTEN linker comprises from about 60 to about 100 amino acid residues. In aspects, the XTEN linker comprises from about 70 to about 90 amino acid residues. In aspects, the XTEN linker comprises from about 75 to about 85 amino acid residues. In aspects, the XTEN linker comprises about 80 amino acid residues. In aspects, when a fusion protein comprises at least two XTEN peptide linkers, then the XTEN linker that comprise from about 50 to about 200 amino acid residues is referred to as a first XTEN peptide linker.

In embodiments, the XTEN linker comprises from about 5 to about 55 amino acid residues. In aspects, the XTEN linker comprises from about 5 to about 50 amino acid residues. In aspects, the XTEN linker comprises from about 5 to about 40 amino acid residues. In aspects, the XTEN linker comprises from about 10 to about 30 amino acid residues. In aspects, the XTEN linker comprises from about 10 to about 25 amino acid residues. In aspects, the XTEN linker comprises from about 10 to about 20 amino acid residues. In aspects, the XTEN linker comprises from about 14 to about 18 amino acid residues. In aspects, the XTEN linker comprises about 16 amino acid residues. In aspects, when a fusion protein comprises at least two XTEN peptide linkers, then the XTEN linker that comprise from about 5 to about 55 amino acid residues is referred to as a second XTEN peptide linker.

In embodiments, the XTEN linker includes the sequence set forth by SEQ ID NO:31. In aspects, the XTEN linker is the sequence set forth by SEQ ID NO:31. In aspects, the XTEN linker includes the sequence set forth by SEQ ID NO:32. In aspects, the XTEN linker is the sequence set forth by SEQ ID NO:32. In aspects, the XTEN linker has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:31. In aspects, the XTEN linker has an amino acid sequence that has at least 85% sequence identity to SEQ ID NO:31. In aspects, the XTEN linker has an amino acid sequence that has at least 90% sequence identity to SEQ ID NO:31. In aspects, the XTEN linker has an amino acid sequence that has at least 95% sequence identity to SEQ ID NO:31. In aspects, the XTEN linker has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:32. In aspects, the XTEN linker has an amino acid sequence that has at least 75% sequence identity to SEQ ID NO:32. In aspects, the XTEN linker has an amino acid sequence that has at least 80% sequence identity to SEQ ID NO:32. In aspects, the XTEN linker has an amino acid sequence that has at least 85% sequence identity to SEQ ID NO:32. In aspects, the XTEN linker has an amino acid sequence that has at least 90% sequence identity to SEQ ID NO:32. In aspects, the XTEN linker has an amino acid sequence that has at least 95% sequence identity to SEQ ID NO:32.

The fusion protein may include amino acid sequences useful for targeting the fusion protein to specific regions of a cell (e.g., cytoplasm, nucleus). Thus, in aspects, the fusion protein further includes a nuclear localization signal (NLS) peptide. In aspects, the NLS includes the sequence set forth by SEQ ID NO:25. In aspects, the NLS is the sequence set forth by SEQ ID NO:25. In aspects, the NLS has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO: 25. In aspects, the NLS has an amino acid sequence that has at least 75% sequence identity to SEQ ID NO:25. In aspects, the NLS has an amino acid sequence that has at least 80% sequence identity to SEQ ID NO:25. In aspects, the NLS has an amino acid sequence that has at least 85% sequence identity to SEQ ID NO:25. In aspects, the NLS has an amino acid sequence that has at least 90% sequence identity to SEQ ID NO:25. In aspects, the NLS has an amino acid sequence that has at least 95% sequence identity to SEQ ID NO:25.

In embodiments, the fusion protein includes, from N-terminus to C-terminus, a KRAB domain, a nuclease-deficient RNA-guided DNA endonuclease enzyme, and a DNA methyltransferase domain. In embodiments, the nuclease-deficient RNA-guided DNA endonuclease enzyme is CRISPR-associated protein and the DNA methyltransferase domain is a Dnmt3A-3L domain. In embodiments, the nuclease-deficient RNA-guided DNA endonuclease enzyme is dCas9 and the DNA methyltransferase domain is a Dnmt3A-3L domain. In embodiments, the dCas9 is covalently linked to the KRAB domain via a peptide linker and wherein the dCas9 is covalently linked to the Dnmt3A-3L domain via a peptide linker.

In embodiments, the fusion protein includes, from N-terminus to C-terminus, a KRAB domain, a nuclease-deficient DNA endonuclease enzyme, and a DNA methyltransferase domain. In embodiments, the nuclease-deficient DNA endonuclease enzyme is a zinc finger domain and the DNA methyltransferase domain is a Dnmt3A-3L domain. In embodiments, the nuclease-deficient DNA endonuclease enzyme is a TALE and the DNA methyltransferase domain is a Dnmt3A-3L domain. In embodiments, the nuclease-deficient DNA endonuclease enzyme is covalently linked to the KRAB domain via a peptide linker and wherein the nuclease-deficient DNA endonuclease enzyme is covalently linked to the Dnmt3A-3L domain via a peptide linker.

In embodiments, peptide linker is an XTEN linker. In aspects, the XTEN linker includes the sequence set forth by SEQ ID NO:31. In aspects, the XTEN linker is the sequence set forth by SEQ ID NO:31. In aspects, the XTEN linker has an amino acid sequence that has at least 75% sequence identity to SEQ ID NO:31. In aspects, the XTEN linker has an amino acid sequence that has at least 80% sequence identity to SEQ ID NO:31. In aspects, the XTEN linker has an amino acid sequence that has at least 85% sequence identity to SEQ ID NO:31. In aspects, the XTEN linker has an amino acid sequence that has at least 90% sequence identity to SEQ ID NO:31. In aspects, the XTEN linker has an amino acid sequence that has at least 95% sequence identity to SEQ ID NO:31. In aspects, the XTEN linker includes the sequence set forth by SEQ ID NO:32. In aspects, the XTEN linker is the sequence set forth by SEQ ID NO:32. In aspects, the XTEN linker has an amino acid sequence that has at least 75% sequence identity to SEQ ID NO:32. In aspects, the XTEN linker has an amino acid sequence that has at least 80% sequence identity to SEQ ID NO:32. In aspects, the XTEN linker has an amino acid sequence that has at least 85% sequence identity to SEQ ID NO:32. In aspects, the XTEN linker has an amino acid sequence that has at least 90% sequence identity to SEQ ID NO:32. In aspects, the XTEN linker has an amino acid sequence that has at least 95% sequence identity to SEQ ID NO:32.

In embodiments, the fusion protein includes the amino acid sequence of SEQ ID NO: 1, 2, 3, 4, 5, 6, 7, 8, 9, 10, 11 12, 13, 14 or 15. In aspects, the fusion protein includes the amino acid sequence of SEQ ID NO: 1. In aspects, the fusion protein is the amino acid sequence of SEQ ID NO:1. In aspects, the fusion protein includes the amino acid sequence of SEQ ID NO:2. In aspects, the fusion protein is the amino acid sequence of SEQ ID NO:2 In aspects, the fusion protein includes the amino acid sequence of SEQ ID NO:3. In aspects, the fusion protein is the amino acid sequence of SEQ ID NO:3. In aspects, the fusion protein includes the amino acid sequence of SEQ ID NO:4. In aspects, the fusion protein is the amino acid sequence of SEQ ID NO:4. In aspects, the fusion protein includes the amino acid sequence of SEQ ID NO:5. In aspects, the fusion protein is the amino acid sequence of SEQ ID NO:5. In aspects, the fusion protein includes the amino acid sequence of SEQ ID NO:6. In aspects, the fusion protein is the amino acid sequence of SEQ ID NO:6. In aspects, the fusion protein includes the amino acid sequence of SEQ ID NO:7. In aspects, the fusion protein is the amino acid sequence of SEQ ID NO:7. In aspects, the fusion protein includes the amino acid sequence of SEQ ID NO:8. In aspects, the fusion protein is the amino acid sequence of SEQ ID NO:8. In aspects, the fusion protein includes the amino acid sequence of SEQ ID NO:9. In aspects, the fusion protein is the amino acid sequence of SEQ ID NO:9. In aspects, the fusion protein includes the amino acid sequence of SEQ ID NO:10. In aspects, the fusion protein is the amino acid sequence of SEQ ID NO:10. In aspects, the fusion protein includes the amino acid sequence of SEQ ID NO: 11. In aspects, the fusion protein is the amino acid sequence of SEQ ID NO:11. In aspects, the fusion protein includes the amino acid sequence of SEQ ID NO:12. In aspects, the fusion protein is the amino acid sequence of SEQ ID NO: 12. In aspects, the fusion protein includes the amino acid sequence of SEQ ID NO:13. In aspects, the fusion protein is the amino acid sequence of SEQ ID NO:13. In aspects, the fusion protein includes the amino acid sequence of SEQ ID NO: 14. In aspects, the fusion protein is the amino acid sequence of SEQ ID NO:14. In aspects, the fusion protein includes the amino acid sequence of SEQ ID NO:15. In aspects, the fusion protein is the amino acid sequence of SEQ ID NO: 15.

In embodiments, the fusion protein includes an amino acid sequence having at least 75%, 80% 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:1, 2, 3, 4, 5, 6, 7, 8, 9, 10, 11 12, 13, 14 or 15. In aspects, the fusion protein includes an amino acid sequence having at least 75%, 80% 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:1. In aspects, the fusion protein includes an amino acid sequence having at least 75%, 80% 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:2. In aspects, the fusion protein includes an amino acid sequence having at least 75%, 80% 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:3. In aspects, the fusion protein includes an amino acid sequence having at least 75%, 80% 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:4. In aspects, the fusion protein includes an amino acid sequence having at least 75%, 80% 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:5. In aspects, the fusion protein includes an amino acid sequence having at least 75%, 80% 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:6. In aspects, the fusion protein includes an amino acid sequence having at least 75%, 80% 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:7. In aspects, the fusion protein includes an amino acid sequence having at least 75%, 80% 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:8. In aspects, the fusion protein includes an amino acid sequence having at least 75%, 80% 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:9. In aspects, the fusion protein includes an amino acid sequence having at least 75%, 80% 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:10. In aspects, the fusion protein includes an amino acid sequence having at least 75%, 80% 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:11. In aspects, the fusion protein includes an amino acid sequence having at least 75%, 80% 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 12. In aspects, the fusion protein includes an amino acid sequence having at least 75%, 80% 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:13. In aspects, the fusion protein includes an amino acid sequence having at least 75%, 80% 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:14. In aspects, the fusion protein includes an amino acid sequence having at least 75%, 80% 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:15.

### Complexes

In order for the fusion protein to carry out epigenome editing, the fusion protein interacts with (e.g. is non-covalently bound to) a polynucleotide (e.g., sgRNA) that is complementary to a target polynucleotide sequence (e.g., a target DNA sequence to be edited) and further includes a sequence (i.e., a binding sequence) to which the nuclease-deficient RNA-guided DNA endonuclease enzyme of the fusion protein as described herein can bind. In aspects, the polynucleotide that is complementary to a target polynucleotide sequence (e.g., a target DNA sequence to be edited) and further includes a binding sequence to which the nuclease-deficient RNA-guided DNA endonuclease enzyme of the fusion protein as described herein can bind is sgRNA. In aspects, the polynucleotide that is complementary to a target polynucleotide sequence (e.g., a target DNA sequence to be edited) and further includes a binding sequence to which the nuclease-deficient RNA-guided DNA endonuclease enzyme of the fusion protein as described herein can bind is cr:tracrRNA. By forming this complex, the fusion protein is appropriately positioned to perform epigenome editing. The term “complex” refers to a composition that includes two or more components, where the components bind together to make a functional unit. In aspects, a complex described herein includes a fusion protein described herein and a polynucleotide described herein. Thus, in an aspect is provided a fusion protein as described herein, including embodiments and aspects thereof, and sgRNA or cr:tracrRNA (i.e., a polynucleotide including: (1) a DNA-targeting sequence that is complementary to a target polynucleotide sequence; and (2) a binding sequence for the nuclease-deficient RNA-guided DNA endonuclease enzyme, wherein the nuclease-deficient RNA-guided DNA endonuclease enzyme is bound to the polynucleotide via the binding sequence (e.g., an amino acid sequence capable of binding to the DNA-targeting sequence)).

A DNA-targeting sequence refers to a polynucleotide that includes a nucleotide sequence complementary to the target polynucleotide sequence (DNA or RNA). In aspects, a DNA-targeting sequence can be a single RNA molecule (single RNA polynucleotide), which may include a “single-guide RNA,” or “sgRNA.” In aspects, a DNA-targeting sequence can comprise two RNA molecules (two RNA polynucleotides), referred to as a guide RNA (gRNA). In aspects, the DNA-targeting sequence includes two RNA molecules (e.g., joined together via hybridization at the binding sequence (e.g., dCas9-binding sequence). In aspects, the DNA-targeting sequence (e.g., sgRNA) is at least 50%, 55%, 60%, 65%, 70%, 75%, 80%, 85%, 90%, 95%, 96%, 97%, 98% or 99% complementary to the target polynucleotide sequence. In aspects, the DNA-targeting sequence (e.g., sgRNA) is at least 50%, 55%, 60%, 65%, 70%, 75%, 80%, 85%, 90%, 95%, 96%, 97%, 98% or 99% complementary to the sequence of a cellular gene. In aspects, the DNA-targeting sequence (e.g., sgRNA) binds a cellular gene sequence. In aspects, the DNA-targeting sequence (e.g., sgRNA) is at least 75% complementary to the sequence of a cellular gene. In aspects, the DNA-targeting sequence (e.g., sgRNA) is at least 80% complementary to the sequence of a cellular gene. In aspects, the DNA-targeting sequence (e.g., sgRNA) binds a cellular gene sequence. In aspects, the DNA-targeting sequence (e.g., sgRNA) is at least 85% complementary to the sequence of a cellular gene. In aspects, the DNA-targeting sequence (e.g., sgRNA) binds a cellular gene sequence. In aspects, the DNA-targeting sequence (e.g., sgRNA) is at least 90% complementary to the sequence of a cellular gene. In aspects, the DNA-targeting sequence (e.g., sgRNA) binds a cellular gene sequence. In aspects, the DNA-targeting sequence (e.g., sgRNA) is at least 95% complementary to the sequence of a cellular gene. In aspects, the DNA-targeting sequence (e.g., sgRNA) binds a cellular gene sequence.

A “target nucleic acid” or “target nucleic acid sequence” as provided herein is a nucleic acid sequence present in, or expressed by, a cell, to which a guide sequence (or a DNA-targeting sequence) is designed to have complementarity, where hybridization between a target sequence and a guide sequence (or a DNA-targeting sequence) promotes the formation of a complex (e.g., CRISPR complex). Full complementarity is not necessarily required, provided there is sufficient complementarity to cause hybridization and promote formation of a complex (e.g., CRISPR complex). In aspects, the target polynucleotide sequence is an exogenous nucleic acid sequence. In aspects, the target polynucleotide sequence is an endogenous nucleic acid sequence.

The target polynucleotide sequence may be any region of the polynucleotide (e.g., DNA sequence) suitable for epigenome editing. In aspects, the target polynucleotide sequence is part of a gene. In aspects, the target polynucleotide sequence is part of a transcriptional regulatory sequence. In aspects, the target polynucleotide sequence is part of a promoter, enhancer or silencer. In aspects, the target polynucleotide sequence is part of a promoter. In aspects, the target polynucleotide sequence is part of an enhancer. In aspects, the target polynucleotide sequence is part of a silencer.

In embodiments, the target polynucleotide sequence is a hypomethylated nucleic acid sequence. A “hypomethylated nucleic acid sequence” is used herein according to the standard meaning in the art and refers to a loss or lack of methyl groups on the 5-methylcytosine nucleotide (e.g., in CpG). The loss or lack of methyl groups may be relative to a standard control. Hypomethylation may occur, for example, in aging cells or in cancer (e.g., early stages of neoplasia) relative to the younger cell or non-cancer cell, respectively. Thus, the complex may be useful for reestablishing normal (e.g. non-aged of non-diseased) methylation levels.

In embodiments, the target polynucleotide sequence is within or adjacent to a transcription start site. In aspects, the target polynucleotide sequence is within about 3000, 2500, 2000, 1500, 500, 100, 80, 70, 60, 50, 40, 30, 20, 10, or fewer base pairs (bp) flanking a transcription start site.

In embodiments, the target polynucleotide sequence is at, near, or within a promoter sequence. In aspects, the target polynucleotide sequence is within a CpG island. In aspects, the target polynucleotide sequence is known to be associated with a disease or condition characterized by DNA hypomethylation or hypermethylation.

In embodiments, the target polynucleotide sequence include the sequence of SEQ ID NO:37, 39, 41, 43, 45, 47, 49, 51, 53, 55, 57, 59, 61, 63, 65, 67, 69, 71, 73, 75, 77, 79, 81, 83, 85, 87, 89, 91, 93, or 95. In aspects, the target polynucleotide sequence include an amino acid sequence having at least 80%, 85%, 90%, 91%, 92%, 93%, 94%, 95%, 96%, 97%, 98%, or 99% sequence identity to SEQ ID NO:37, 39, 41, 43, 45, 47, 49, 51, 53, 55, 57, 59, 61, 63, 65, 67, 69, 71, 73, 75, 77, 79, 81, 83, 85, 87, 89, 91, 93, or 95. In aspects, the target polynucleotide sequence is SEQ ID NO:37. In aspects, the target polynucleotide sequence is SEQ ID NO:39. In aspects, the target polynucleotide sequence is SEQ ID NO:41. In aspects, the target polynucleotide sequence is SEQ ID NO:43. In aspects, the target polynucleotide sequence is SEQ ID NO:45. In aspects, the target polynucleotide sequence is SEQ ID NO:47. In aspects, the target polynucleotide sequence is SEQ ID NO:49. In aspects, the target polynucleotide sequence is SEQ ID NO:51. In aspects, the target polynucleotide sequence is SEQ ID NO:53. In aspects, the target polynucleotide sequence is SEQ ID NO:55. In aspects, the target polynucleotide sequence is SEQ ID NO:57. In aspects, the target polynucleotide sequence is SEQ ID NO:59. In aspects, the target polynucleotide sequence is SEQ ID NO:61. In aspects, the target polynucleotide sequence is SEQ ID NO:63. In aspects, the target polynucleotide sequence is SEQ ID NO:65. In aspects, the target polynucleotide sequence is SEQ ID NO:67. In aspects, the target polynucleotide sequence is SEQ ID NO:69. In aspects, the target polynucleotide sequence is SEQ ID NO:71. In aspects, the target polynucleotide sequence is SEQ ID NO:73. In aspects, the target polynucleotide sequence is SEQ ID NO:75. In aspects, the target polynucleotide sequence is SEQ ID NO:77. In aspects, the target polynucleotide sequence is SEQ ID NO:79. In aspects, the target polynucleotide sequence is SEQ ID NO:81. In aspects, the target polynucleotide sequence is SEQ ID NO:83. In aspects, the target polynucleotide sequence is SEQ ID NO:85. In aspects, the target polynucleotide sequence is SEQ ID NO:87. In aspects, the target polynucleotide sequence is SEQ ID NO:89. In aspects, the target polynucleotide sequence is SEQ ID NO:91. In aspects, the target polynucleotide sequence is SEQ ID NO:93. In aspects, the target polynucleotide sequence is SEQ ID NO:95.

In aspects, the target polynucleotide sequence has at least 75%, 80%, 85%, 90%, 91%, 92%, 93%, 94%, 95%, 96%, 97%, 98%, or 99% sequence identity to SEQ ID NO:37. In aspects, the target polynucleotide sequence has at least 75%, 80%, 85%, 90%, 91%, 92%, 93%, 94%, 95%, 96%, 97%, 98%, or 99% sequence identity to SEQ ID NO:39. In aspects, the target polynucleotide sequence has at least 75%, 80%, 85%, 90%, 91%, 92%, 93%, 94%, 95%, 96%, 97%, 98%, or 99% sequence identity to SEQ ID NO:41. In aspects, the target polynucleotide sequence has at least 75%, 80%, 85%, 90%, 91%, 92%, 93%, 94%, 95%, 96%, 97%, 98%, or 99% sequence identity to SEQ ID NO:43. In aspects, the target polynucleotide sequence is SEQ ID NO:45. In aspects, the target polynucleotide sequence has at least 75%, 80%, 85%, 90%, 91%, 92%, 93%, 94%, 95%, 96%, 97%, 98%, or 99% sequence identity to SEQ ID NO:47. In aspects, the target polynucleotide sequence has at least 75%, 80%, 85%, 90%, 91%, 92%, 93%, 94%, 95%, 96%, 97%, 98%, or 99% sequence identity to SEQ ID NO:49. In aspects, the target polynucleotide sequence has at least 75%, 80%, 85%, 90%, 91%, 92%, 93%, 94%, 95%, 96%, 97%, 98%, or 99% sequence identity to SEQ ID NO:51. In aspects, the target polynucleotide sequence has at least 75%, 80%, 85%, 90%, 91%, 92%, 93%, 94%, 95%, 96%, 97%, 98%, or 99% sequence identity to SEQ ID NO:53. In aspects, the target polynucleotide sequence has at least 75%, 80%, 85%, 90%, 91%, 92%, 93%, 94%, 95%, 96%, 97%, 98%, or 99% sequence identity to SEQ ID NO:55. In aspects, the target polynucleotide sequence has at least 75%, 80%, 85%, 90%, 91%, 92%, 93%, 94%, 95%, 96%, 97%, 98%, or 99% sequence identity to SEQ ID NO:57. In aspects, the target polynucleotide sequence has at least 75%, 80%, 85%, 90%, 91%, 92%, 93%, 94%, 95%, 96%, 97%, 98%, or 99% sequence identity to SEQ ID NO:59. In aspects, the target polynucleotide sequence has at least 75%, 80%, 85%, 90%, 91%, 92%, 93%, 94%, 95%, 96%, 97%, 98%, or 99% sequence identity to SEQ ID NO:61. In aspects, the target polynucleotide sequence has at least 75%, 80%, 85%, 90%, 91%, 92%, 93%, 94%, 95%, 96%, 97%, 98%, or 99% sequence identity to SEQ ID NO:63. In aspects, the target polynucleotide sequence has at least 75%, 80%, 85%, 90%, 91%, 92%, 93%, 94%, 95%, 96%, 97%, 98%, or 99% sequence identity to SEQ ID NO:65. In aspects, the target polynucleotide sequence has at least 75%, 80%, 85%, 90%, 91%, 92%, 93%, 94%, 95%, 96%, 97%, 98%, or 99% sequence identity to SEQ ID NO:67. In aspects, the target polynucleotide sequence has at least 75%, 80%, 85%, 90%, 91%, 92%, 93%, 94%, 95%, 96%, 97%, 98%, or 99% sequence identity to SEQ ID NO:69. In aspects, the target polynucleotide sequence has at least 75%, 80%, 85%, 90%, 91%, 92%, 93%, 94%, 95%, 96%, 97%, 98%, or 99% sequence identity to SEQ ID NO:71. In aspects, the target polynucleotide sequence has at least 75%, 80%, 85%, 90%, 91%, 92%, 93%, 94%, 95%, 96%, 97%, 98%, or 99% sequence identity to SEQ ID NO:73. In aspects, the target polynucleotide sequence has at least 75%, 80%, 85%, 90%, 91%, 92%, 93%, 94%, 95%, 96%, 97%, 98%, or 99% sequence identity to SEQ ID NO:75. In aspects, the target polynucleotide sequence has at least 75%, 80%, 85%, 90%, 91%, 92%, 93%, 94%, 95%, 96%, 97%, 98%, or 99% sequence identity to SEQ ID NO:77. In aspects, the target polynucleotide sequence has at least 75%, 80%, 85%, 90%, 91%, 92%, 93%, 94%, 95%, 96%, 97%, 98%, or 99% sequence identity to SEQ ID NO:79. In aspects, the target polynucleotide sequence has at least 75%, 80%, 85%, 90%, 91%, 92%, 93%, 94%, 95%, 96%, 97%, 98%, or 99% sequence identity to SEQ ID NO:81. In aspects, the target polynucleotide sequence has at least 75%, 80%, 85%, 90%, 91%, 92%, 93%, 94%, 95%, 96%, 97%, 98%, or 99% sequence identity to SEQ ID NO:83. In aspects, the target polynucleotide sequence has at least 75%, 80%, 85%, 90%, 91%, 92%, 93%, 94%, 95%, 96%, 97%, 98%, or 99% sequence identity to SEQ ID NO:85. In aspects, the target polynucleotide sequence has at least 75%, 80%, 85%, 90%, 91%, 92%, 93%, 94%, 95%, 96%, 97%, 98%, or 99% sequence identity to SEQ ID NO:87. In aspects, the target polynucleotide sequence has at least 75%, 80%, 85%, 90%, 91%, 92%, 93%, 94%, 95%, 96%, 97%, 98%, or 99% sequence identity to SEQ ID NO:89. In aspects, the target polynucleotide sequence has at least 75%, 80%, 85%, 90%, 91%, 92%, 93%, 94%, 95%, 96%, 97%, 98%, or 99% sequence identity to SEQ ID NO:91. In aspects, the target polynucleotide sequence has at least 75%, 80%, 85%, 90%, 91%, 92%, 93%, 94%, 95%, 96%, 97%, 98%, or 99% sequence identity to SEQ ID NO:93. In aspects, the target polynucleotide sequence has at least 75%, 80%, 85%, 90%, 91%, 92%, 93%, 94%, 95%, 96%, 97%, 98%, or 99% sequence identity to SEQ ID NO:95.

In embodiments, the complex includes dCas9 bound to the polynucleotide through binding a binding sequence of the polynucleotide and thereby forming a ribonucleoprotein complex. In aspects, the binding sequence forms a hairpin structure. In aspects, the binding sequence is 10-200 nt, 15-150 nt, 20-140 nt, 30-100 nt, 35-50 nt, 37-47 nt, or 42 nt in length.

In embodiments, the binding sequence (e.g., Cas9-binding sequence) interacts with or binds to a Cas9 protein (e.g., dCas9 protein), and together they bind to the target polynucleotide sequence recognized by the DNA-targeting sequence. The binding sequence (e.g., Cas9-binding sequence) includes two complementary stretches of nucleotides that hybridize to one another to form a double stranded RNA duplex (a dsRNA duplex). These two complementary stretches of nucleotides may be covalently linked by intervening nucleotides known as linkers or linker nucleotides (e.g., in the case of a single-molecule polynucleotide), and hybridize to form the double stranded RNA duplex (dsRNA duplex, or “Cas9-binding hairpin”) of the binding sequence (e.g., Cas9-binding sequence), thus resulting in a stem-loop structure. Alternatively, in some aspects, the two complementary stretches of nucleotides may not be covalently linked, but instead are held together by hybridization between complementary sequences (e.g., a two-molecule polynucleotide).

The binding sequence (e.g., Cas9-binding sequence) can have a length of from 10 nucleotides to 200 nucleotides, e.g., from 20 nucleotides (nt) to 150 nt. In aspects, the binding sequence has a length of from 80 nucleotides (nt) to 100 nt. The dsRNA duplex of the binding sequence (e.g., Cas9-binding sequence) can have a length from 6 base pairs (bp) to 200 bp. For example, the dsRNA duplex of the binding sequence (e.g., Cas9-binding sequence) can have a length from 6 bp to 200 bp, from 10 bp to 180 bp, from 10 bp to 150 bp, from 80 bp to 100 bp, and the like.

In embodiments, the polynucleotide that forms a complex with a fusion protein described herein includes the sequence of SEQ ID NO: 38, 40, 42, 44, 46, 48, 50, 52, 54, 56, 58, 60, 62, 64, 66, 68, 70, 72, 74, 76, 78, 80, 82, 84, 86, 88, 90, 92, or 94 or their corresponding RNA sequence. In aspects, the polynucleotide that forms a complex with a fusion protein described herein includes the sequence having at least 80%, 85%, 90%, 91%, 92%, 93%, 94%, 95%, 96%, 97%, 98%, or 99% sequence identity to SEQ ID NO:38, 40, 42, 44, 46, 48, 50, 52, 54, 56, 58, 60, 62, 64, 66, 68, 70, 72, 74, 76, 78, 80, 82, 84, 86, 88, 90, 92, or 94 or their corresponding RNA sequence. In aspects, the polynucleotide that forms a complex with a fusion protein described herein includes the sequence of SEQ ID NO:38. In aspects, the polynucleotide that forms a complex with a fusion protein described herein includes the sequence of SEQ ID NO:40. In aspects, the polynucleotide that forms a complex with a fusion protein described herein includes the sequence of SEQ ID NO:42. In aspects, the polynucleotide that forms a complex with a fusion protein described herein includes the sequence of SEQ ID NO:44. In aspects, the polynucleotide that forms a complex with a fusion protein described herein includes the sequence of SEQ ID NO:46. In aspects, the polynucleotide that forms a complex with a fusion protein described herein includes the sequence of SEQ ID NO:48. In aspects, the polynucleotide that forms a complex with a fusion protein described herein includes the sequence of SEQ ID NO:50. In aspects, the polynucleotide that forms a complex with a fusion protein described herein includes the sequence of SEQ ID NO:52. In aspects, the polynucleotide that forms a complex with a fusion protein described herein includes the sequence of SEQ ID NO:54. In aspects, the polynucleotide that forms a complex with a fusion protein described herein includes the sequence of SEQ ID NO:56. In aspects, the polynucleotide that forms a complex with a fusion protein described herein includes the sequence of SEQ ID NO:58. In aspects, the polynucleotide that forms a complex with a fusion protein described herein includes the sequence of SEQ ID NO:60. In aspects, the polynucleotide that forms a complex with a fusion protein described herein includes the sequence of SEQ ID NO:62. In aspects, the polynucleotide that forms a complex with a fusion protein described herein includes the sequence of SEQ ID NO:64. In aspects, the polynucleotide that forms a complex with a fusion protein described herein includes the sequence of SEQ ID NO:66. In aspects, the polynucleotide that forms a complex with a fusion protein described herein includes the sequence of SEQ ID NO:68. In aspects, the polynucleotide that forms a complex with a fusion protein described herein includes the sequence of SEQ ID NO:70. In aspects, the polynucleotide that forms a complex with a fusion protein described herein includes the sequence of SEQ ID NO:72. In aspects, the polynucleotide that forms a complex with a fusion protein described herein includes the sequence of SEQ ID NO:74. In aspects, the polynucleotide that forms a complex with a fusion protein described herein includes the sequence of SEQ ID NO:76. In aspects, the polynucleotide that forms a complex with a fusion protein described herein includes the sequence of SEQ ID NO:78. In aspects, the polynucleotide that forms a complex with a fusion protein described herein includes the sequence of SEQ ID NO:80. In aspects, the polynucleotide that forms a complex with a fusion protein described herein includes the sequence of SEQ ID NO:82. In aspects, the polynucleotide that forms a complex with a fusion protein described herein includes the sequence of SEQ ID NO:84. In aspects, the polynucleotide that forms a complex with a fusion protein described herein includes the sequence of SEQ ID NO:86. In aspects, the polynucleotide that forms a complex with a fusion protein described herein includes the sequence of SEQ ID NO:88. In aspects, the polynucleotide that forms a complex with a fusion protein described herein includes the sequence of SEQ ID NO:90. In aspects, the polynucleotide that forms a complex with a fusion protein described herein includes the sequence of SEQ ID NO:92. In aspects, the polynucleotide that forms a complex with a fusion protein described herein includes the sequence of SEQ ID NO:94.

### Nucleic Acids and Vectors

The fusion protein described herein, including embodiments and aspects thereof, may be provided as a nucleic acid sequence that encodes for the fusion protein. Thus, in an aspect is provided a nucleic acid sequence encoding the fusion protein described herein, including embodiments and aspects thereof. In an aspect is provided a nucleic acid sequence encoding the fusion protein described herein (including the DNA-targeting sequence), including embodiments and aspects thereof. In aspects, the nucleic acid sequence encodes for a fusion protein described herein, including fusion proteins having amino acid sequences with certain % sequence identities described herein. In aspects, the nucleic acid is RNA. In aspects, the nucleic acid is messenger RNA. In aspects, fusion protein is delivered as DNA, mRNA, protein or an RNP. For RNP the protein would be dCas9 and the RNA would encode an sgRNA. Similarly the sgRNA could be delivered as DNA encoding a promoter and an sgRNA, RNA encoding a promoter and an sgRNA. In aspects, the nucleic acid sequence encodes for the fusion proteins described herein, including embodiments and aspects thereof. In aspects, the nucleic acid sequence encodes for the fusion protein of any one of SEQ ID NOS:1-15. In aspects, the nucleic acid sequence encodes for the fusion protein of SEQ ID NO: 97. In aspects, the nucleic acid sequence encodes for the fusion protein of SEQ ID NO: 98. In aspects, the nucleic acid sequence encodes for the fusion protein of SEQ ID NO:99.

It is further contemplated that the nucleic acid sequence encoding the fusion protein as described herein, including embodiments and aspects thereof, may be included in a vector. Therefore, in an aspect is provided a vector including a nucleic acid sequence as described herein, including embodiments and aspects thereof. In aspects, the vector comprises a nucleic acid sequence that encodes for a fusion protein described herein, including fusion proteins having amino acid sequences with certain % sequence identities described herein. In aspects, the nucleic acid is messenger RNA. In aspects, the messenger RNA is messenger RNP. In aspects, the vector comprises a nucleic acid sequence that encodes for the fusion protein of any one of SEQ ID NOS:1-15. In aspects, the vector comprises a nucleic acid sequence that encodes for the fusion protein of SEQ ID NO:97. In aspects, the vector comprises a nucleic acid sequence that encodes for the fusion protein of SEQ ID NO:98. In aspects, the vector comprises a nucleic acid sequence that encodes for the fusion protein of SEQ ID NO:99.

In embodiments, the vector further includes a polynucleotide, wherein the polynucleotide includes: (1) a DNA-targeting sequence that is complementary to a target polynucleotide sequence; and (2) a binding sequence for the nuclease-deficient RNA-guided DNA endonuclease enzyme. In aspects, the vector further includes a polynucleotide, wherein the polynucleotide includes sgRNA. In aspects, the vector further includes a polynucleotide, wherein the polynucleotide includes cr:tracrRNA. Thus, one or more vectors may include all necessary components for preforming epigenome editing.

### Cells

The compositions described herein may be incorporated into a cell. Inside the cell, the compositions as described herein, including embodiments and aspects thereof, may perform epigenome editing. Accordingly, in an aspect is provided a cell including a fusion protein as described herein, including embodiments and aspects thereof, a nucleic acid as described herein, including embodiments and aspects thereof, a complex as described herein, including embodiments and aspects thereof, or a vector as described herein, including embodiments and aspects thereof. In aspects is provided a cell including a fusion protein as described herein, including embodiments and aspects thereof. In aspects is provided a cell including a nucleic acid as described herein, including embodiments and aspects thereof. In aspects is provided a cell including a complex as described herein, including embodiments and aspects thereof. In aspects is provided a cell including a vector as described herein, including embodiments and aspects thereof. In aspects, the cell is a eukaryotic cell. In aspects, the cell is a mammalian cell.

### Methods

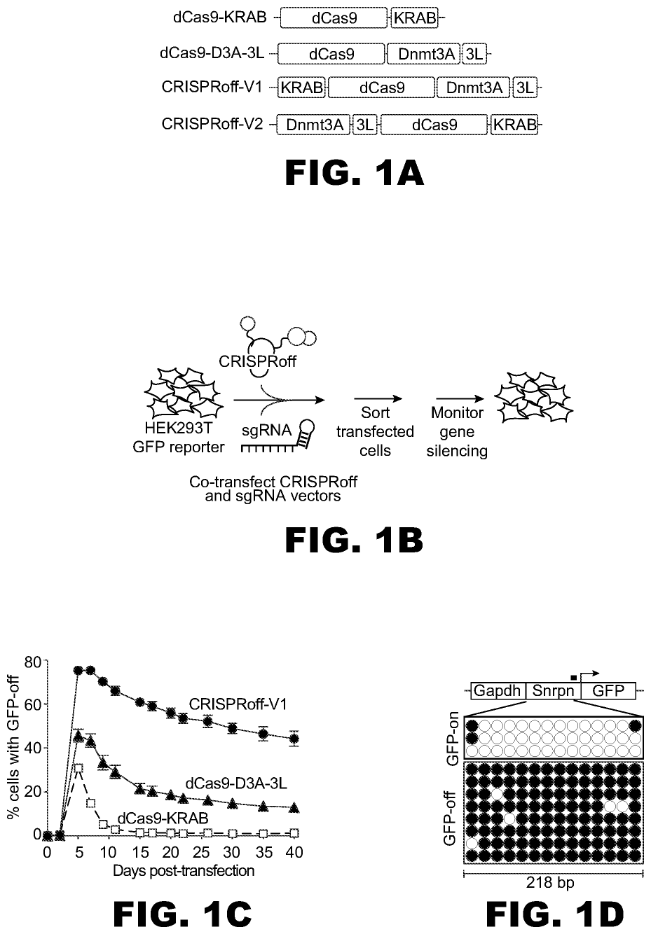

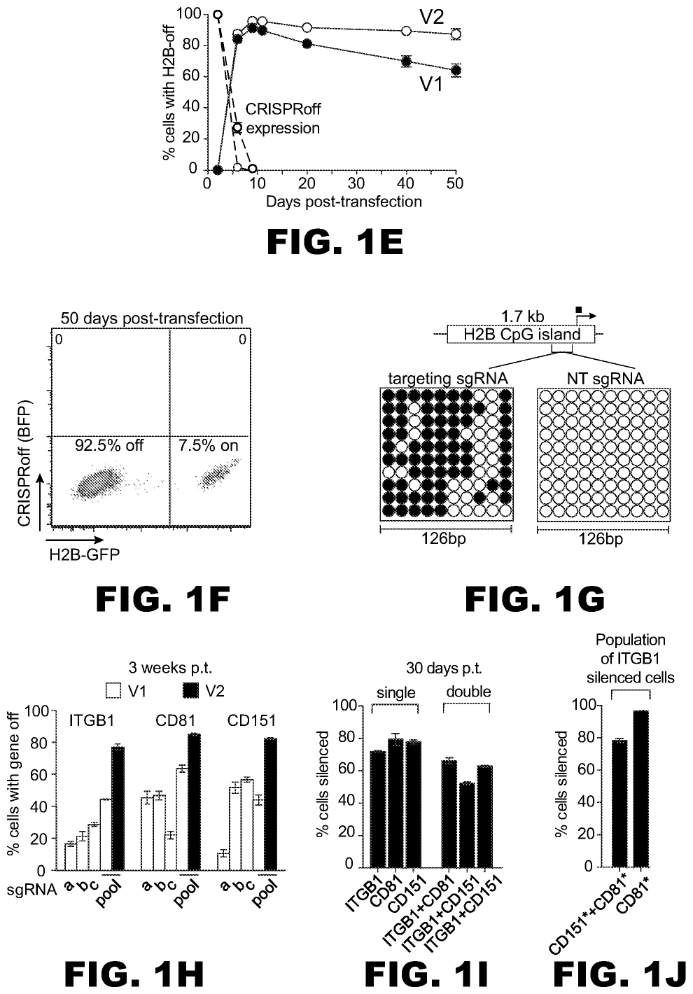

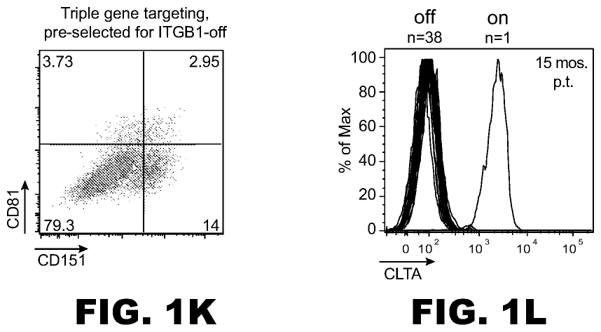

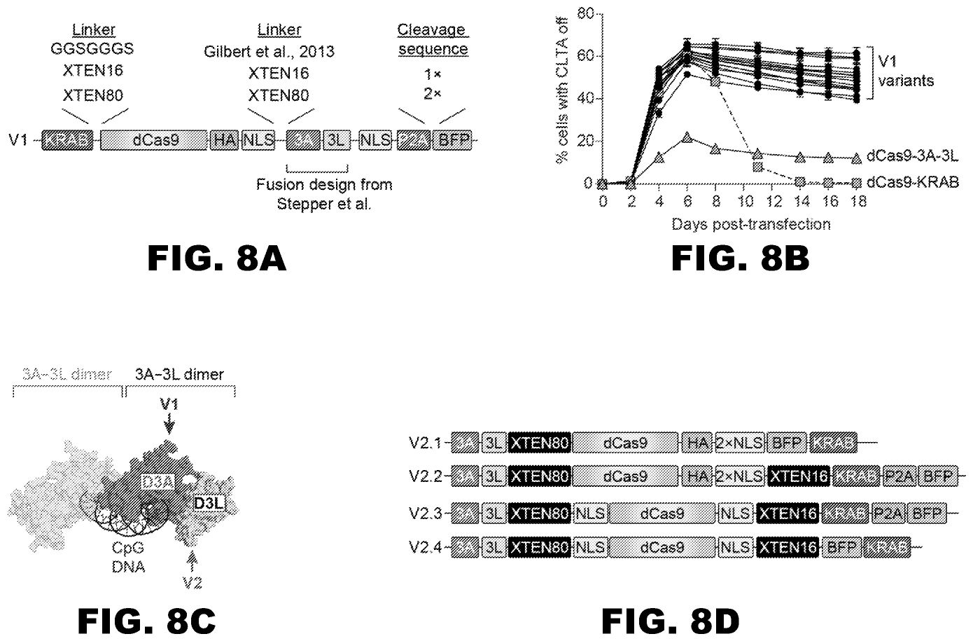

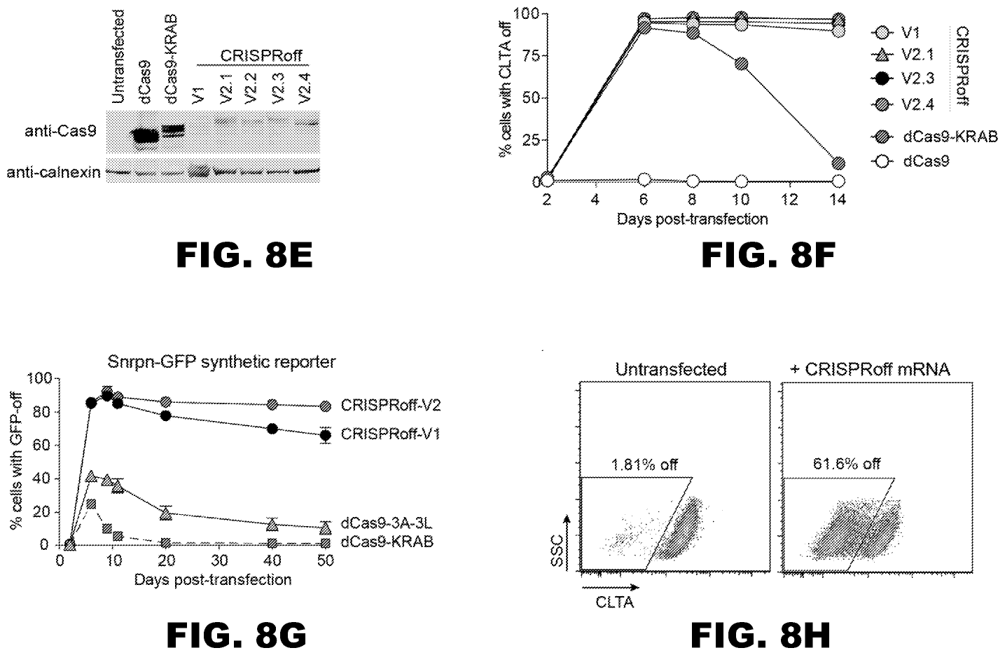

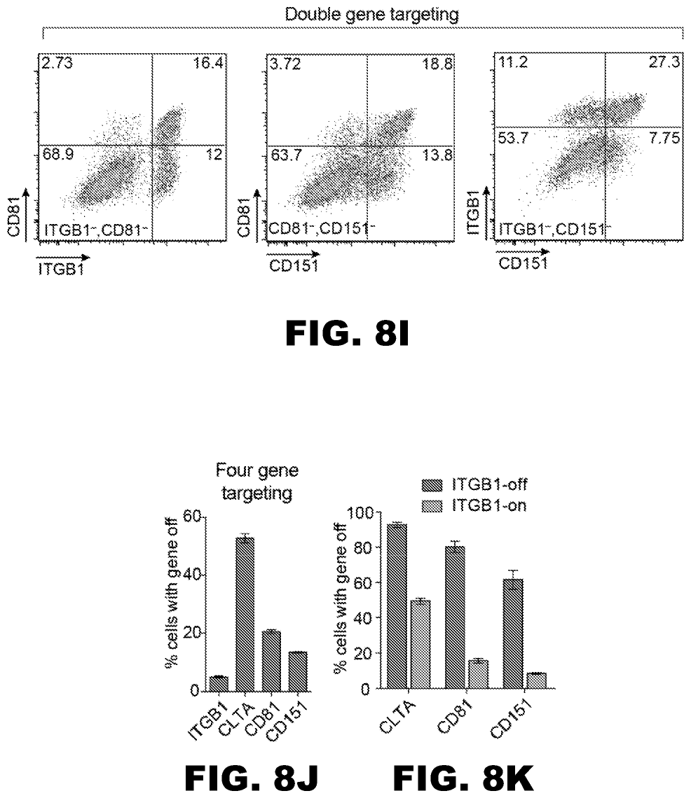

The fusion proteins described herein program a durable memory of gene silencing over time. As shown in the examples, the fusion proteins (e.g., SEQ ID NOS:15, 97, 98, 99, 107, 108) program a durable memory of gene silencing with over 80% of transfected cells silencing the Snrpn-GFP reporter and over 90% the endogenously GFP-tagged gene HIST2H2BE (H2B) at 50 days post-transfection (FIGS. 1E, 1F, 8F). Notably, starting at 10 days post-transfection, no fusion protein (i.e., CRISPR-off protein) was detected, indicating that the observed gene silencing was independent of constitutive expression of the fusion protein (FIG. 1E). The data in FIGS. 1E and 1F is transfection of DNA encoding CRISPRoff. This skilled artisan could alternatively transfect a CRISPRoff RNP consisting of CRISPRoff protein and an sgRNA that form a complex in vitro. FIG. 1L also demonstrates that transient expression of CRISPRoff for less than 10 days results in stable gene silencing of the endogenous CLTA gene at 15 months post transfection. 15 months of time is more than 450 cell divisions which is more than most cells in the adult human body divide over the course of a lifetime. Gene silencing is achieved by transfection of mRNA encoding the fusion proteins described herein. Thus, transient expression of the fusion protein leads to effective gene silencing (FIG. 8G). CRISPRoff epigenetic memory using the fusion proteins described herein is propagated by the cell rather than by sustained transgene expression.

In embodiments, the disclosure provides methods of silencing a target nucleic acid sequence in a cell, the method comprising: (i) delivering a first polynucleotide encoding a fusion protein, as described herein, including all embodiments and aspects thereof (e.g., comprising a nuclease-deficient RNA-guided DNA endonuclease enzyme), to a cell containing the target nucleic acid; and (ii) delivering to the cell a second polynucleotide comprising sgRNA or cr:tracrRNA; thereby silencing the target nucleic acid sequence. In embodiments, the second polynucleotide comprises sgRNA. In embodiments, the second polynucleotide comprises two different sgRNA (e.g., gRNA). In embodiments, the disclosure provides methods of silencing a target nucleic acid sequence in a cell, the method comprising delivering a first polynucleotide encoding a fusion protein, as described herein, including all embodiments and aspects thereof (e.g., comprising a nuclease-deficient DNA endonuclease enzyme), to a cell containing the target nucleic acid; thereby silencing the target nucleic acid sequence. In aspects, the target nucleic acid comprises a CpG island. In aspects, the target nucleic acid includes a non-CpG island. In aspects, the target nucleic acid comprises a CpG island and a non-CpG island. “Comprises a CpG island” or “comprises a non-CpG island” refers to one or more CpG islands or non-CpG islands, respectively. In aspects, the target nucleic acid sequence comprises a plurality of CpG islands (e.g., 2, 3, 4, 5, or more CpG islands). In aspects, the target nucleic acid sequence comprises a plurality of non-CpG islands (e.g., 2, 3, 4, 5, or more non-CpG islands). In aspects, the target nucleic acid sequence does not comprise a CpG island and does not comprises a non-CpG island. In aspects, the method of silencing the target nucleic acid sequence is a method of treating Angelman syndrome in a patient in need thereof. In aspects, the method of silencing the target nucleic acid sequence is a method of treating a viral infection in a patient in need thereof. In aspects, the method of silencing the target nucleic acid sequence is a method of treating an infectious disease in a patient in need thereof.

In embodiments, the disclosure provides methods of silencing a target nucleic acid sequence in a cell, the method comprising: delivering a polynucleotide: (a) encoding a fusion protein, as described herein, including all embodiments and aspects thereof (e.g., comprising a nuclease-deficient RNA-guided DNA endonuclease enzyme), and (b) comprising sgRNA or cr:tracrRNA; thereby silencing the target nucleic acid sequence. In embodiments, the polynucleotide comprises sgRNA. In embodiments, the polynucleotide comprises two different sgNRA (e.g., gRNA). In embodiments, the disclosure provides methods of silencing a target nucleic acid sequence in a cell, the method comprising delivering a first polynucleotide encoding a fusion protein, as described herein, including all embodiments and aspects thereof (e.g., comprising a nuclease-deficient DNA endonuclease enzyme), to a cell containing the target nucleic acid; thereby silencing the target nucleic acid sequence. In aspects, the target nucleic acid comprises a CpG island. In aspects, the target nucleic acid comprises a non-CpG island. In aspects, the target nucleic acid comprises a CpG island and a non-CpG island. “Comprises a CpG island” or “comprises a non-CpG island” refers to one or more CpG islands or non-CpG islands, respectively. In aspects, the target nucleic acid sequence comprises a plurality of CpG islands (e.g., 2, 3, 4, 5, or more CpG islands). In aspects, the target nucleic acid sequence comprises a plurality of non-CpG islands (e.g., 2, 3, 4, 5, or more non-CpG islands). In aspects, the target nucleic acid sequence does not comprise a CpG island and does not comprises a non-CpG island. In aspects, the method of silencing the target nucleic acid sequence is a method of treating Angelman syndrome in a patient in need thereof. In aspects, the method of silencing the target nucleic acid sequence is a method of treating a viral infection in a patient in need thereof. In aspects, the method of silencing the target nucleic acid sequence is a method of treating an infectious disease in a patient in need thereof. In aspects, the method of silencing the target nucleic acid sequence is a method of treating a neurodegenerative disease in a patient in need thereof.

In embodiments, the disclosure provides methods of treating an infectious disease in a subject in need thereof, the method comprising: (i) delivering to the subject an effective amount of a first polynucleotide encoding a fusion protein, as described herein, including all embodiments and aspects thereof (e.g., comprising a nuclease-deficient RNA-guided DNA endonuclease enzyme); and (ii) delivering to the subject an effective amount of a second polynucleotide comprising sgRNA or cr:tracrRNA; thereby treating the infectious disease in the subject. In embodiments, the second polynucleotide comprises sgRNA. In embodiments, the second polynucleotide comprises two different sgNRA (e.g., gRNA). In embodiments, the disclosure provides methods of treating an infectious disease in a subject in need thereof, the method comprising delivering to the subject an effective amount of a first polynucleotide encoding a fusion protein, as described herein, including all embodiments and aspects thereof (e.g., comprising a nuclease-deficient DNA endonuclease enzyme); thereby treating the infectious disease.

The term “infection” or “infectious disease” refers to a disease caused by organisms such as a bacterium, virus, fungi, or any other pathogenic microbial agents. In embodiments, the infectious disease is caused by bacteria. In embodiments, the infectious disease is a bacteria associated disease (e.g., tuberculosis, which is caused by Mycobacterium tuberculosis). Non-limiting infectious diseases caused by bacteria include pneumonia (e.g., Streptococcus, Pseudomonas); or foodborne illnesses (e.g., Shigella, Campylobacter, Salmonella). Infectious diseases caused by bacteria also includes tetanus, typhoid fever, diphtheria, syphilis, and leprosy. In embodiments, the infectious disease is bacterial vaginosis (i.e. bacteria that change the vaginal microbiota caused by an overgrowth of bacteria that crowd out the Lactobacilli species that maintain healthy vaginal microbial populations) (e.g., yeast infection, or Trichomonas vaginalis); bacterial meningitis (i.e., a bacterial inflammation of the meninges); bacterial pneumonia (i.e., a bacterial infection of the lungs); a urinary tract infection; bacterial gastroenteritis; or bacterial skin infections (e.g. impetigo, or cellulitis). In embodiments, the infectious disease is a Campylobacter jejuni, Enterococcus faecalis, Haemophilus influenzae, Helicobacter pylori, Klebsiella pneumoniae, Legionella pneumophila, Neisseria gonorrhoeae, Neisseria meningitides, Staphylococcus aureus, Streptococcus pneumonia, or Vibrio cholera infection. In embodiments, the infectious disease is caused by fungi. In embodiments, the infectious disease is caused by a virus.

In embodiments, the disclosure provides methods of treating a bacterial infection or a fungal infection in a subject in need thereof, the method comprising: (i) delivering to the subject an effective amount of a first polynucleotide encoding a fusion protein, as described herein, including all embodiments and aspects thereof (e.g., comprising a nuclease-deficient RNA-guided DNA endonuclease enzyme); and (ii) delivering to the subject an effective amount of a second polynucleotide comprising sgRNA or cr:tracrRNA; thereby treating the bacterial infection or the fungal infection in the subject. In embodiments, the second polynucleotide comprises sgRNA. In embodiments, the second polynucleotide comprises two different sgNRA (e.g., gRNA). In embodiments, the disclosure provides methods of treating a bacterial infection or a fungal infection in a subject in need thereof, the method comprising delivering to the subject an effective amount of a first polynucleotide encoding a fusion protein, as described herein, including all embodiments and aspects thereof (e.g., comprising a nuclease-deficient DNA endonuclease enzyme); thereby treating the bacterial infection or the fungal infection. In embodiments, the methods comprise treating a bacterial infection. In embodiments, the methods comprise treating a fungal infection.

In embodiments, the disclosure provides methods of treating a viral infection in a subject in need thereof, the method comprising: (i) delivering to the subject an effective amount of a first polynucleotide encoding a fusion protein, as described herein, including all embodiments and aspects thereof (e.g., comprising a nuclease-deficient RNA-guided DNA endonuclease enzyme); and (ii) delivering to the subject an effective amount of a second polynucleotide comprising sgRNA or cr:tracrRNA; thereby treating the viral infection in the subject. In embodiments, the second polynucleotide comprises sgRNA. In embodiments, the second polynucleotide comprises two different sgNRA (e.g., gRNA). In embodiments, the disclosure provides methods of treating a viral infection in a subject in need thereof, the method comprising delivering to the subject an effective amount of a first polynucleotide encoding a fusion protein, as described herein, including all embodiments and aspects thereof (e.g., comprising a nuclease-deficient DNA endonuclease enzyme); thereby treating the viral infection. In embodiments, the viral infection is a Flavivirus infection. In embodiments, the Flavivirus infection is caused by West Nile virus, dengue virus, tick-borne encephalitis virus, yellow fever virus, or Zika virus. In embodiments, the disclosure provides methods of treating a Flavivirus infection in a subject in need thereof by the methods described herein. In embodiments, the disclosure provides methods of treating a West Nile virus infection in a subject in need thereof by the methods described herein. In embodiments, the disclosure provides methods of treating a dengue virus infection in a subject in need thereof by the methods described herein. In embodiments, the disclosure provides methods of treating a tick-borne encephalitis virus infection in a subject in need thereof by the methods described herein. In embodiments, the disclosure provides methods of treating a yellow fever virus infection in a subject in need thereof by the methods described herein. In embodiments, the disclosure provides methods of treating a Zika virus infection in a subject in need thereof by the methods described herein.

The term “viral infection” or “viral disease” refers to a disease or condition that is caused by a virus. Non-limiting examples of viral infections include hepatic viral diseases (e.g., hepatitis A, B, C, D, E), herpes virus infection (e.g., HSV-1, HSV-2, herpes zoster), flavivirus infection (e.g., Zika virus infection, dengue virus infection, yellow fever virus infection, West Nile virus infection tick-borne encephalitis virus infection), cytomegalovirus infection, a respiratory viral infection (e.g., adenovirus infection, influenza, severe acute respiratory syndrome, coronavirus infection (e.g., SARS-CoV-1, SARS-CoV-2, MERS-CoV, COVID-19, MERS)), a gastrointestinal viral infection (e.g., norovirus infection, rotavirus infection, astrovirus infection), an exanthematous viral infection (e.g., measles, shingles, smallpox, rubella), viral hemorrhagic disease (e.g., Ebola, Lassa fever, dengue fever, yellow fever), a neurologic viral infection (e.g., West Nile viral infection, polio, viral meningitis, viral encephalitis, Japanese encephalitis, rabies), and human papilloma viral infection.

In embodiments, the disclosure provides methods of treating a tau pathology in a subject in need thereof, the method comprising: (i) delivering to the subject an effective amount of a first polynucleotide encoding a fusion protein, as described herein, including all embodiments and aspects thereof (e.g., comprising a nuclease-deficient RNA-guided DNA endonuclease enzyme); and (ii) delivering to the subject an effective amount of a second polynucleotide comprising sgRNA or cr:tracrRNA; thereby treating the tau pathology in the subject. In embodiments, the second polynucleotide comprises sgRNA. In embodiments, the second polynucleotide comprises two different sgNRA (e.g., gRNA). In embodiments, the disclosure provides methods of treating a tau pathology in a subject in need thereof, the method comprising delivering to the subject an effective amount of a first polynucleotide encoding a fusion protein, as described herein, including all embodiments and aspects thereof (e.g., comprising a nuclease-deficient DNA endonuclease enzyme); thereby treating the tau pathology. Tau pathology refers to neurodegenerative diseases characterized by pathological tau aggregation in neurofibrillary tangles (NFTs). Diseases with this typical pathological feature are called tauopathies and include, for example Alzheimer's disease, Parksinson's disease, progressive supranuclear palsy, Huntington's disease, amyotrophic lateral sclerosis, Pick's disease, dementia pugilistica, and frontotemporal dementia. In embodiments, the tau pathology is Alzheimer's disease. In embodiments, the tau pathology is Parksinson's disease. In embodiments, the tau pathology is Parksinson's disease linked to chromosome 17. In embodiments, the tau pathology is progressive supranuclear palsy. In embodiments, the tau pathology is Huntington's disease. In embodiments, the tau pathology is amyotrophic lateral sclerosis. In embodiments, the tau pathology is Pick's disease. In embodiments, the tau pathology is dementia pugilistica. In embodiments, the tau pathology is frontotemporal dementia. In embodiments, the tau pathology is frontotemporal dementia linked to chromosome 17.

In embodiments, the disclosure provides methods of treating a neurodegenerative disease in a subject in need thereof, the method comprising: (i) delivering to the subject an effective amount of a first polynucleotide encoding a fusion protein, as described herein, including all embodiments and aspects thereof (e.g., comprising a nuclease-deficient RNA-guided DNA endonuclease enzyme); and (ii) delivering to the subject an effective amount of a second polynucleotide comprising sgRNA or cr:tracrRNA; thereby treating the neurodegenerative disease in the subject. In embodiments, the second polynucleotide comprises sgRNA. In embodiments, the second polynucleotide comprises two different sgNRA (e.g., gRNA). In embodiments, the disclosure provides methods of treating a neurodegenerative disease in a subject in need thereof, the method comprising delivering to the subject an effective amount of a first polynucleotide encoding a fusion protein, as described herein, including all embodiments and aspects thereof (e.g., comprising a nuclease-deficient DNA endonuclease enzyme); thereby treating the neurodegenerative disease.

As used herein, the term “neurodegenerative disorder” or “neurodegenerative disease” refers to a disease or condition in which the function of a subject's nervous system becomes impaired. Examples of neurodegenerative diseases that may be treated with the fusion proteins and methods described herein include Alexander's disease, Alper's disease, Alzheimer's disease, amyotrophic lateral sclerosis, ataxia telangiectasia, Batten disease (also known as Spielmeyer-Vogt-Sjogren-Batten disease), bovine spongiform encephalopathy (BSE), canavan disease, chronic fatigue syndrome, cockayne syndrome, corticobasal degeneration, Creutzfeldt-Jakob disease, frontotemporal dementia, Gerstmann-Straussler-Scheinker syndrome, Huntington's disease, HIV-associated dementia, Kennedy's disease, Krabbe's disease, kuru, lewy body dementia, Machado-Joseph disease (Spinocerebellar ataxia type 3), multiple sclerosis, multiple system atrophy, myalgic encephalomyelitis, narcolepsy, neuroborreliosis, Parkinson's disease, Pelizaeus-Merzbacher Disease, Pick's disease, primary lateral sclerosis, prion diseases, Refsum's disease, Sandhoffs disease, Schilder's disease, subacute combined degeneration of spinal cord secondary to pernicious anaemia, schizophrenia, spinocerebellar ataxia (multiple types with varying characteristics), spinal muscular atrophy, Steele-Richardson-Olszewski disease, progressive supranuclear palsy, and tabes dorsalis.

Angelman syndrome (AS) is a neurological genetic disorder caused by loss of expression of the maternal copy of UBE3A in the brain. Due to brain-specific genetic imprinting at this locus, the paternal UBE3A is also silenced by an antisense transcript. The methods described herein can be used to inhibit the antisense transcript, thereby unsilencing the paternal UBE3A, which results in the treatment of Angelman syndrome. See, e.g., Bailus et al, Mol Ther, 24(3):548-555 (2016). In other words, the methods described herein silence the negative regulator of the silenced paternal UBE3A, which equates to unsilencing the paternal UBE3A.

In embodiments, the disclosure provides methods of treating Angelman syndrome in a subject in need thereof, the method comprising: (i) delivering to the subject an effective amount of a first polynucleotide encoding a fusion protein, as described herein, including all embodiments and aspects thereof (e.g., comprising a nuclease-deficient RNA-guided DNA endonuclease enzyme); and (ii) delivering to the subject an effective amount of a second polynucleotide comprising sgRNA or cr:tracrRNA; thereby treating Angelman syndrome in the subject. In embodiments, the second polynucleotide comprises sgRNA. In embodiments, the second polynucleotide comprises two different sgNRA. In embodiments, the disclosure provides methods of treating Angelman syndrome in a subject in need thereof, the method comprising delivering to the subject an effective amount of a first polynucleotide encoding a fusion protein, as described herein, including all embodiments and aspects thereof (e.g., comprising a nuclease-deficient DNA endonuclease enzyme); thereby treating Angelman syndrome in the subject

In aspects, the sequence is within the target nucleic acid sequence is methylated. In aspects, the sequence that is within about 3000, 2900, 2800, 2700, 2600, 2500, 2400, 2300, 2200, 2100, 2000, 1900, 1800, 1700, 1600, 1500, 1400, 1300, 1200, 1100, 1000, 900, 800, 700, 600, 500, 400, 300, 200, 100, 50, 20, or 10 base pairs of the target nucleic acid sequence is methylated. Without intending to be bound by any theory, methylating a chromatin means that DNA is methylated at the C nucleotide of CG sequences found in CpG islands or non-CpG islands (i.e., adding methyl marks at the C nucleotide of CG DNA sites found in CpG islands).

The term “repressive chromatin markers” as used herein refers to modifications made to the chromatin that result in silencing (e.g., decreasing or inhibiting of transcription) of the target nucleic acid sequence (e.g., a gene). Examples of repressive chromatin markers include, but are not limited to, mono-, di-, and/or tri-methylation, acetylation/deacetylation, phosphorylation, and ubiquitination of histones (e.g., H3K9, H3K27, H3K79, H2BK5).

The term “CpG island” is used in its customary sense to refer to regions in an nucleic acid that have a high frequency of the nucleotides G and C next to one another (i.e., CpG dinucleotides). In aspects, a CpG island refers to a region of a nucleic acid sequence having a region with a GC content greater than 50%, with an observed-to-expected CpG ratio greater than 60%. In aspects, a CpG island refers to a region of a nucleic acid sequence having at least 50 base pair, and a GC content greater than 50%, with an observed-to-expected CpG ratio greater than 60%. In aspects, a CpG island refers to a region of a nucleic acid sequence having at least 100 base pair, and a GC content greater than 50%, with an observed-to-expected CpG ratio greater than 60%. In aspects, a CpG island refers to a region of a nucleic acid sequence having at least 150 base pair, and a GC content greater than 50%, with an observed-to-expected CpG ratio greater than 60%. In aspects, a CpG island refers to a region of a nucleic acid sequence having at least 200 base pair, and a GC content greater than 50%, with an observed-to-expected CpG ratio greater than 60%. The percentage CpG is the ratio of CpG nucleotide bases (twice the CpG count) to the length. The ratio of observed to expected CpG is calculated according to the formula:

$$Obs / Exp CpG = Number of CpG * N / ( Number of C * Number of G ) ,$$

where N=length of sequence. See Gardiner-Garden et al, Journal of Molecular Biology, 196(2):261-282 (1987)).

The phrase “target nucleic acid does not comprise a CpG island” or “target nucleic acid that does not comprise a CpG island” or “non-CpG island” refers to a target nucleic acid that does not contain a “CpG island” as that term is defined herein. This region can be any region encoded by a mammalian (e.g., human) genome. In aspects, the phrase “target nucleic does not comprise a CpG island” refers to regions in a target nucleic acid that have do not have the nucleotides G and C next to one another (i.e., CpG dinucleotides) or that have a low frequency of the nucleotides G and C next to one another. In aspects, a non-CpG island refers to regions of a target nucleic acid having a region with a GC dinucleotide content less than 50%, with an observed-to-expected CpG ratio less than 60%. In aspects, a non-CpG island refers to regions of a target nucleic acid having a GC dinucleotide content less than 50%, with an observed-to-expected CpG ratio less than 60%. In aspects, a non-CpG island refers to regions of a target nucleic acid having a GC dinucleotide content less than 50%, with an observed-to-expected CpG ratio less than 60%. In aspects, a non-CpG island refers to regions of a target nucleic acid having a GC dinucleotide content less than 50%, with an observed-to-expected CpG ratio less than 60%. In aspects, a non-CpG island refers to regions of a target nucleic acid having a GC dinucleotide content less than 50%, with an observed-to-expected CpG ratio less than 60%. In aspects, a non-CpG island refers to regions of a target nucleic acid having a GC dinucleotide content less than 45%, with an observed-to-expected CpG ratio less than 55%. In aspects, a non-CpG island refers to regions of a target nucleic acid having a GC dinucleotide content less than 40%, with an observed-to-expected CpG ratio less than 50%. In aspects, a non-CpG island refers to regions of a target nucleic acid having a GC dinucleotide content of 1% to 45%, with an observed-to-expected CpG ratio of less than 60%. In aspects, a non-CpG island refers to regions of a target nucleic acid having a GC dinucleotide content of 1% to 45%, with an observed-to-expected CpG ratio less than 55%. In aspects, a non-CpG island refers to regions of a target nucleic acid a GC dinucleotide content of 1% to 45%, with an observed-to-expected CpG ratio less than 50%. In aspects, a non-CpG island refers to regions of a target nucleic acid having a GC dinucleotide content of 5% to 40%, with an observed-to-expected CpG ratio less than 60%. In aspects, a non-CpG island refers to regions of a target nucleic acid having a GC dinucleotide content of 5% to 40%, with an observed-to-expected CpG ratio less than 55%. In aspects, a non-CpG island refers to regions of a target nucleic acid having a GC dinucleotide content of 5% to 40%, with an observed-to-expected CpG ratio less than 50%. In aspects, a non-CpG island refers to regions of a target nucleic acid having a GC dinucleotide content of 10% to 40%, with an observed-to-expected CpG ratio less than 60%. In aspects, a non-CpG island refers to regions of a target nucleic acid having a GC dinucleotide content of 10% to 40%, with an observed-to-expected CpG ratio less than 55%. In aspects, a non-CpG island refers to regions of a target nucleic acid having a GC dinucleotide content of 10% to 40%, with an observed-to-expected CpG ratio less than 50%. In aspects, the target nucleic acid that does not comprise a CpG island has at least 10 base pairs. In aspects, the target nucleic acid that does not comprise a CpG island has at least 50 base pairs. In aspects, the target nucleic acid that does not comprise a CpG island has at least 100 base pairs. In aspects, the target nucleic acid that does not comprise a CpG island has at least 150 base pairs. In aspects, the target nucleic acid that does not comprise a CpG island has at least 200 base pairs.

In embodiments, silencing refers to a complete suppression of transcription. In aspects, silencing refers to a significant decrease in transcription compared to control levels of transcription.

In embodiments, the first polynucleotide is contained within a first vector. In aspects, the first polynucleotide is contained within a second vector. In aspects, the first vector and the second vector are the same. In aspects, the first vector is different from the second vector.

In embodiments, the polynucleotide described herein is delivered into the cell by any method known in the art, for example, by transfection, electroporation or transduction.

Alternatively, in an aspect is provided a method of silencing a target nucleic acid sequence in a cell, including delivering a complex as described herein, including embodiments and aspects thereof, to a cell containing the target nucleic acid. Without intending to be bound by any theory, the complex silences the target nucleic acid sequence in the cell by methylating a chromatin containing the target nucleic acid sequence and/or by introducing repressive chromatin marks to a chromatin containing the target nucleic acid sequence.

In embodiments, the method has a specificity that is 2-fold higher than a specificity to a non-target nucleic acid sequence. In aspects, the method has a specificity that is at least 2-fold (e.g., 2-, 3-, 4-, 5-, 6-, 7-, 8-, 9-, 10-, 15-, 20-, 25-fold) higher than a specificity to a non-target nucleic acid sequence. Methods for determining specificity are well known in the art and include, but are not limited to, RNA-seq, bisulfite sequencing, chromatin immunoprecipitation, flow cytometry, and qPCR. Thus, in aspects, specificity is determined by RNA-seq. In aspects, specificity is determined by bisulfite sequencing. In aspects, specificity is determined by chromatin immunoprecipitation. In aspects, specificity is determined by flow cytometry. In aspects, specificity is determined by qPCR.

In aspects, the complex is delivered into the cell via any method known in the art. In aspects, the complex is delivered to the cell via RNA, DNA, or ribonucleoprotein (RNP) delivery. In aspects, the complex is delivered into the cell via RNA. In aspects, the complex is delivered to the cell via DNA. In aspects, the complex is delivered to the cell via transfection, virus, lipid nanoparticle (LNP) or viral-like particles. In aspects, the complex is delivered to the cell via transfection. In aspects, the complex is delivered to the cell via virus. In aspects, the complex is delivered to the cell via lipid nanoparticle. Methods for delivery complexes into a cell are well known in the art.

### Embodiments 1 to 76

Embodiment 1. A fusion protein comprising, from N-terminus to C-terminus, a DNA methyltransferase domain, a first XTEN linker, a nuclease-deficient RNA-guided endonuclease enzyme, a second XTEN linker, and a Krüppel-associated box domain.

Embodiment 2. The fusion protein of Embodiment 1, wherein the first XTEN linker comprises from about 5 to about 864 amino acid residues, and the second XTEN linker comprises from about 5 to about 864 amino acid residues.

Embodiment 3. The fusion protein of Embodiment 2, wherein the first XTEN linker comprises from greater than 50 to about 864 amino acid residues, and the second XTEN linker comprises from about 5 to 50 amino acid residues, and a Krüppel-associated box domain.

Embodiment 4. The fusion protein of Embodiment 3, wherein the first XTEN linker comprises from about 60 to about 864 amino acid residues, and the second XTEN linker comprises from about 10 to about 40 amino acid residues.

Embodiment 5. The fusion protein of Embodiment 4, wherein the first XTEN linker comprises from about 70 to about 864 amino acid residues, and the second XTEN linker comprises from about 10 to about 30 amino acid residues.

Embodiment 6. The fusion protein of any one of Embodiments 1 to 5, wherein the nuclease-deficient RNA-guided DNA endonuclease enzyme is a CRISPR-associated protein, a leucine zipper domain, a winged helix domain, a helix-turn-helix motif, a helix-loop-helix domain, an HMB-box domain, a Wor3 domain, an OB-fold domain, an immunoglobulin domain, or a B3 domain.

Embodiment 7. The fusion protein of Embodiment 6, wherein the nuclease-deficient RNA-guided DNA endonuclease enzyme is dCas9.

Embodiment 8. The fusion protein of Embodiment 6, wherein the nuclease-deficient RNA-guided DNA endonuclease enzyme is dCpf1 or ddCpf1.

Embodiment 9. The fusion protein of any one of Embodiments 1 to 8, wherein the DNA methyltransferase domain comprises a Dnmt3A domain.

Embodiment 10. The fusion protein of Embodiment 9, wherein the Dnmt3A domain is linked to a Dnmt3L domain (Dnmt3A-3L domain).

Embodiment 11. The fusion protein of any one of Embodiments 1 to 10, further comprising an epitope tag, a 2A peptide, a fluorescent protein tag, a nuclear localization signal peptide, or a combination of two or more thereof.

Embodiment 12. The fusion protein of Embodiment 11, comprising, from N-terminus to C-terminus, the DNA methyltransferase domain, the first XTEN linker, the nuclease-deficient RNA-guided endonuclease enzyme, a nuclear localization signal peptide, and the Krüppel-associated box domain.

Embodiment 13. The fusion protein of Embodiment 11, comprising, from N-terminus to C-terminus, the DNA methyltransferase domain, the first XTEN linker, the nuclease-deficient RNA-guided endonuclease enzyme, a nuclear localization signal peptide, the second XTEN linker, and the Krüppel-associated box domain.

Embodiment 14. The fusion protein of Embodiment 11, comprising, from N-terminus to C-terminus, the DNA methyltransferase domain, the first XTEN linker, the nuclease-deficient RNA-guided endonuclease enzyme, an epitope tag, a nuclear localization signal peptide, the second XTEN linker, the Krüppel-associated box domain, a 2A cleavable peptide, and a fluorescent protein tag.

Embodiment 15. A fusion protein having at least 85% sequence identity to the amino acid sequence of Formula (A); where the amino acid sequence of Formula (A) is, from N-terminus to C-terminus: C₁-R³-C₂-R²-A-R¹-R⁴-B (A), wherein: C₁ comprises SEQ ID NO:26 or SEQ ID NO:106; R³ is absent or R³ comprises SEQ ID NO:27; C₂ comprises SEQ ID NO:28; R² is absent or R² comprises SEQ ID NO:32; A comprises SEQ ID NO:23; R¹ is absent or R¹ comprises SEQ ID NO:25; R⁴ is absent or R⁴ comprises SEQ ID NO:31; and B comprises SEQ ID NO:16, SEQ ID NO:103, SEQ ID NO:104, or SEQ ID NO: 105.

Embodiment 16. The fusion protein of Embodiment 15 having at least 90% sequence identity to the amino acid sequence of Formula (A).

Embodiment 17. The fusion protein of Embodiment 16 having at least 95% sequence identity to the amino acid sequence of Formula (A).

Embodiment 18. A fusion protein having at least 85% sequence identity to SEQ ID NO:97, 98, 99, 107, 108, 109, or 110.

Embodiment 19. The fusion protein of Embodiment 18 having at least 90% sequence identity to SEQ ID NO:97, 98, 99, 107, 108, 109, or 110.

Embodiment 20. A cell comprising the fusion protein of any one of Embodiments 1 to 19.

Embodiment 21. The cell of Embodiment 20, wherein the cell is a eukaryotic cell, a mammalian cell, or a stem cell.

Embodiment 22. A method of silencing a target nucleic acid sequence in a cell, the method comprising: (i) delivering a first polynucleotide encoding a fusion protein of any one of Embodiments 1 to 19 to a cell containing the target nucleic acid; and (ii) delivering to the cell a second polynucleotide comprising sgRNA or cr:tracrRNA; thereby silencing the target nucleic acid sequence.

Embodiment 23. The method of Embodiment 22, wherein the target nucleic acid comprises a CpG island

Embodiment 24. The method of Embodiment 22, wherein the target nucleic acid does not comprise a CpG island.

Embodiment 25. The method of any one of Embodiments 22 to 24, wherein the second polynucleotide comprises sgRNA.

Embodiment 26. A method of silencing a target nucleic acid sequence in a cell, the method comprising: (i) delivering a first polynucleotide encoding a fusion protein to a cell containing the target nucleic acid, wherein the target nucleic acid does not comprise a CpG island; wherein the fusion protein comprises a nuclease-deficient RNA-guided DNA endonuclease enzyme, a Krüppel associated box domain, and a DNA methyltransferase domain; and (ii) delivering to the cell a second polynucleotide comprising sgRNA or cr:tracrRNA; thereby silencing the target nucleic acid sequence in the cell.

Embodiment 27. A method of treating Angelman syndrome, an infectious disease, a tau pathology, or a neurodegenerative disease in a subject in need thereof, the method comprising: (i) delivering to the subject an effective amount of a first polynucleotide encoding a fusion protein comprising a nuclease-deficient RNA-guided DNA endonuclease enzyme, a Krüppel associated box domain, and a DNA methyltransferase domain; and (ii) delivering to the subject an effective amount of a second polynucleotide comprising sgRNA or cr:tracrRNA; thereby treating Angelman syndrome, the infectious disease, the tau pathology, or the neurodegenerative disease.

Embodiment 28. The method of Embodiment 26 or 27, wherein the second polynucleotide comprises sgRNA.

Embodiment 29. The method of any one of Embodiments 26 to 28, wherein the fusion protein comprises, from N-terminus to C-terminus, the DNA methyltransferase domain, the nuclease-deficient RNA-guided DNA endonuclease enzyme, and the Krüppel associated box domain.

Embodiment 30. The method of any one of Embodiments 26 to 29, wherein the nuclease-deficient RNA-guided DNA endonuclease enzyme is a CRISPR-associated protein, a leucine zipper domain, a winged helix domain, a helix-turn-helix motif, a helix-loop-helix domain, an HMB-box domain, a Wor3 domain, an OB-fold domain, an immunoglobulin domain, or a B3 domain.

Embodiment 31. The method of Embodiment 30, wherein the nuclease-deficient RNA-guided DNA endonuclease enzyme is dCas9.

Embodiment 32. The method of Embodiment 30, wherein the nuclease-deficient RNA-guided DNA endonuclease enzyme is dCpf1 or ddCpf1.

Embodiment 33. The method of any one of Embodiments 26 to 32, wherein the DNA methyltransferase domain comprises a Dnmt3A domain.

Embodiment 34. The method of Embodiment 33, wherein the Dnmt3A domain is linked to a Dnmt3L domain (Dnmt3A-3L domain).

Embodiment 35. The method of any one of Embodiments 26 to 34, wherein the dCas9 is covalently linked to the Dnmt3A domain via a peptide linker and wherein the Dnmt3A domain is covalently linked to the Krüppel associated box domain via a peptide linker.

Embodiment 36. The method of Embodiment 35, wherein the peptide linker is a XTEN linker.

Embodiment 37. The method of any one of Embodiments 26 to 28, wherein the fusion protein comprises, from N-terminus to C-terminus, the Krüppel associated box, the nuclease-deficient RNA-guided DNA endonuclease enzyme, and the DNA methyltransferase domain.

Embodiment 38. The method of Embodiment 37, wherein the nuclease-deficient RNA-guided DNA endonuclease enzyme is a CRISPR-associated protein, a leucine zipper domain, a winged helix domain, a helix-turn-helix motif, a helix-loop-helix domain, an HMB-box domain, a Wor3 domain, an OB-fold domain, an immunoglobulin domain, or a B3 domain.

Embodiment 39. The method of Embodiment 38, wherein the nuclease-deficient RNA-guided DNA endonuclease enzyme is dCas9.

Embodiment 40. The method of Embodiment 38, wherein the nuclease-deficient RNA-guided DNA endonuclease enzyme is dCpf1 or ddCpf1.

Embodiment 41. The method of any one of Embodiments 37 to 39, wherein the DNA methyltransferase domain comprises a Dnmt3A domain.

Embodiment 42. The method of Embodiment 41, wherein the Dnmt3A domain is linked to a Dnmt3L domain (Dnmt3A-3L domain).

Embodiment 43. The method of any one of Embodiments 39 to 42, wherein the dCas9 is covalently linked to the Dnmt3A domain via a peptide linker and wherein the Krüppel associated box domain is covalently linked to the dCas9 via a peptide linker.

Embodiment 44. The method of Embodiment 43, wherein the peptide linker is a XTEN linker.

Embodiment 45. The method of any one of Embodiments 37 to 44, wherein the nuclease-deficient RNA-guided DNA endonuclease enzyme is covalently linked to the Krüppel associated box domain via a peptide linker.

Embodiment 46. The method of any one of Embodiments 37 to 45, wherein the nuclease-deficient RNA-guided DNA endonuclease enzyme is covalently linked to the DNA methyltransferase domain via a peptide linker.

Embodiment 47. The method of any one of Embodiments 37 to 46, wherein the Krüppel associated box domain is covalently linked to the DNA methyltransferase domain via a peptide linker.

Embodiment 48. The method of any one of Embodiments 26 to 47, wherein the fusion protein further comprises an epitope tag, a 2A peptide, a fluorescent protein tag, a nuclear localization signal peptide, or a combination of two or more thereof.

Embodiment 49. The method of any one of Embodiments 26 to 47, wherein the fusion protein further comprises a nuclear localization signal peptide.

Embodiment 50. The method of any one of Embodiments 26 to 28, wherein the fusion protein having at least 85% sequence identity to the amino acid sequence of Formula (A); where the amino acid sequence of Formula (A) is, from N-terminus to C-terminus: C₁—R³-C₂-R²-A-R¹-R⁴-B (A), wherein: C₁ comprises SEQ ID NO:26 or SEQ ID NO:106; R³ is absent or R³ comprises SEQ ID NO:27; C₂ comprises SEQ ID NO:28; R² is absent or R² comprises SEQ ID NO:32; A comprises SEQ ID NO:23; R¹ is absent or R¹ comprises SEQ ID NO:25; R⁴ is absent or R⁴ comprises SEQ ID NO:31; and B comprises SEQ ID NO:16, SEQ ID NO:103, SEQ ID NO:104, or SEQ ID NO:105.

Embodiment 51. The method of Embodiment 50, wherein the fusion protein has at least 90% sequence identity to the amino acid sequence of Formula (A).

Embodiment 52. The method of Embodiment 51, wherein the fusion protein has at least 95% sequence identity to the amino acid sequence of Formula (A).

Embodiment 53. A method of treating Angelman syndrome, an infectious disease, a tau pathology, or a neurodegenerative disease in a subject in need thereof, the method comprising: (i) delivering to the subject an effective amount of a first polynucleotide encoding a fusion protein of any one of Embodiments 1 to 14; and (ii) delivering to the subject an effective amount of second polynucleotide comprising sgRNA or cr:tracrRNA; thereby treating Angelman syndrome, the infectious disease, the tau pathology, or the neurodegenerative disease in the subject.

Embodiment 54. The method of any one of Embodiments 26 to 53, wherein the infectious disease is a viral infections disease.

Embodiment 55. The method of Embodiment 54, wherein the infectious disease is a Flavivirus infectious disease.

Embodiment 56. A fusion protein comprising, from N-terminus to C-terminus, a DNA methyltransferase domain, a first XTEN linker, a nuclease-deficient endonuclease enzyme, a second XTEN linker, and a Krüppel-associated box domain.

Embodiment 57. The fusion protein of Embodiment 56, wherein the first XTEN linker comprises from about 5 to about 864 amino acid residues, and the second XTEN linker comprises from about 5 to about 864 amino acid residues.

Embodiment 58. The fusion protein of Embodiment 57, wherein the first XTEN linker comprises from greater than 50 to about 864 amino acid residues, and the second XTEN linker comprises from about 5 to 50 amino acid residues.

Embodiment 59. The fusion protein of Embodiment 58, wherein the first XTEN linker comprises from about 60 to about 864 amino acid residues, and the second XTEN linker comprises from about 10 to about 40 amino acid residues.

Embodiment 60. The fusion protein of Embodiment 59, wherein the first XTEN linker comprises from about 70 to about 864 amino acid residues, and the second XTEN linker comprises from about 10 to about 30 amino acid residues.

Embodiment 61. The fusion protein of any one of Embodiments 56 to 60, wherein the nuclease-deficient DNA endonuclease enzyme is a zinc finger domain or a transcription activator-like effector.

Embodiment 62. The fusion protein of Embodiment 62, wherein the nuclease-deficient DNA endonuclease enzyme is a zinc finger domain.

Embodiment 63. The fusion protein of Embodiment 62, wherein the nuclease-deficient DNA endonuclease enzyme is transcription activator-like effector.

Embodiment 64. The fusion protein of any one of Embodiments 56 to 63, wherein the DNA methyltransferase domain comprises a Dnmt3A domain.

Embodiment 65. The fusion protein of Embodiment 64, wherein the Dnmt3A domain is linked to a Dnmt3L domain (Dnmt3A-3L domain).

Embodiment 66. The fusion protein of any one of Embodiments 56 to 65, further comprising an epitope tag, a 2A peptide, a fluorescent protein tag, a nuclear localization signal peptide, or a combination of two or more thereof.

Embodiment 67. The fusion protein of any one of Embodiments 56 to 65, further comprising a nuclear localization signal peptide.

Embodiment 68. The fusion protein of Embodiment 56, comprising, from N-terminus to C-terminus, the DNA methyltransferase domain, the first XTEN linker, the nuclease-deficient endonuclease enzyme, an epitope tag, a nuclear localization signal peptide, the second XTEN linker, the Krüppel-associated box domain, a 2A cleavable peptide, and a fluorescent protein tag.

Embodiment 69. A cell comprising the fusion protein of any one of Embodiments 56 to 68.

Embodiment 70. The cell of Embodiment 69, wherein the cell is a eukaryotic cell, a mammalian cell, or a stem cell.

Embodiment 71. A method of silencing a target nucleic acid sequence in a cell, the method comprising delivering a first polynucleotide encoding a fusion protein of any one of Embodiments 56 to 68 to a cell containing the target nucleic acid; thereby silencing the target nucleic acid sequence.

Embodiment 72. The method of Embodiment 71, wherein the target nucleic acid comprises a CpG island.

Embodiment 73. The method of Embodiment 71, wherein the target nucleic acid does not comprise a CpG island.

Embodiment 74. A method of treating Angelman syndrome, an infectious disease, a tau pathology, or a neurodegenerative disease in a subject in need thereof, the method comprising delivering to the subject an effective amount of a first polynucleotide encoding a fusion protein of any one of Embodiments 56 to 68; thereby treating Angelman syndrome, the infectious disease, the tau pathology, or the neurodegenerative disease.

Embodiment 75. The method of Embodiment 74, wherein the infectious disease is a viral infectious disease.

Embodiment 76. The method of Embodiment 75, wherein the viral infectious disease is a Flavivirus infectious disease.

### EXAMPLES

Embodiments and aspects herein are further illustrated by the following examples. The examples are merely intended to illustrate embodiments and aspects, and are not to be construed to limit the scope herein.

The technology described herein allows for, inter alia, permanent silencing of genes in mammalian cells without generating double stranded DNA breaks in the host genome. In embodiments, the central component is a single polypeptide chain composed of catalytically inactive Cas9 (dCas9) fused to Dnmt3A, Dnmt3L, and a KRAB domain (herein referred to as an “all-in-one protein”). In embodiments, the central component is a single polypeptide chain composed of a zinc finger domain fused to Dnmt3A, Dnmt3L, and a KRAB domain (herein referred to as an “all-in-one protein”). This fusion proteins provided herein can be directed to a specific site in a mammalian genome using a single guide RNA (sgRNA) and may add DNA methylation and/or repressive chromatin marks to the site. The result is gene silencing that is inheritable across subsequent cell divisions. The fusion protein provided herein (and sgRNA) are only expressed transiently, bypassing the use of viral delivery methods to induce permanent silencing.

The fusion proteins provided herein provide a robust long-term or permanent silencing of endogenous gene expression by epigenome editing rather than genome editing. Both alleles of a gene may be targeted or a single pathogenic allele may be selectively targeted. An advantage of the fusion protein provided herein is that epigenetic editing is reversible and therefore inherently safer than genome editing. Thus, the fusion proteins provided herein are useful in prophylactic applications. For example, gene silencing can enable acute protection from an infection/biologic toxin and then be reversed after the risk of infection or intoxication is absent. Thus, the fusion proteins provided herein is useful for viral or toxin that enters a cell through interaction with a protein that is required for long term organ function or homeostasis. The fusion proteins provided herein are useful in genome editing based therapeutics.

Permanent gene silencing in mammalian cells can be accomplished with two components: a single polypeptide chain composed of dCas9 fused to three epigenetic modulators and a single guide RNA that directs the protein to a specific site in the host genome. In embodiments, permanent gene silencing in mammalian cells can be accomplished with two components: a single polypeptide chain composed of a zinc finger domain fused to three epigenetic modulators and a single guide RNA that directs the protein to a specific site in the host genome. In embodiments, the components are only expressed transiently in the host cell, thus reducing toxicity and off-target events. In embodiments, the fusion protein provided herein does not induce DNA breaks in the host cell for permanent gene silencing. In embodiments, the epigenetic marks that are added to the genomic site of interest are reversible, thus allowing for removal of any off-target events that may occur.

### Example 1

Advances in gene editing have transformed our ability to modify the human genome. In particular, CRISPR (clustered regularly interspaced short palindromic repeats)-Cas9 (CRISPR-associated protein 9) and other CRISPR systems can be programmed with a single guide (sg)RNA to introduce DNA breaks at a specified site to inactivate gene function or to stimulate precise DNA editing by homology-directed repair (Knott and Doudna, 2018). Additionally, base and prime editing strategies allow for precise DNA sequence modifications but generally rely on one or more DNA single strand nicks (Anzalone et al., 2020). These technologies have been optimized for targeted changes in the underlying DNA sequence and are therefore ideally suited for repairing or introducing pathogenic mutations. However, the reliance on endogenous DNA repair machinery presents challenges as the complexity of these pathways can make it difficult to limit the outcome to a single desired change (Yeh et al., 2019).

An alternative modality for modulating gene function is to rewrite the epigenetic landscape to control gene expression without changing the underlying DNA sequence. Fusing protein scaffold or enzyme domains to catalytically inactive dCas9 can enhance (CRISPRa) or repress (CRISPRi) transcription in mammalian cells (Holtzman and Gersbach, 2018; Xu and Qi, 2019). Programmable epigenome editing is tunable, reversible, and does not require DNA breaks, effectively bypassing the cellular toxicity associated with gene editing (Jost et al., 2020). However, current programmable epigenome editing technologies typically rely on constitutive expression of dCas9-fusion proteins to maintain transcriptional control. As such, these modalities remain less suitable for therapeutic cell and organismal engineering.

Recent work has demonstrated that it is possible for epigenome editing to write a stable transcriptional program that is remembered and propagated by human cells without constitutive expression of the programmable epigenetic modulators (Amabile et al., 2016; Bintu et al., 2016; Park et al., 2019; Van et al., 2021). In particular, Amabile et al. showed that it was possible to heritably silence human genes by recruitment of a cocktail of DNA methyltransferase and KRAB domains. However, to date only a small number of endogenous human loci have been tested for silencing by epigenetic memory writers (Amabile et al., 2016; O'Geen et al., 2019; Tarjan et al., 2019). Moreover, previous designs of programmable epigenetic silencers utilize either two or three fusion proteins for each target gene, which is experimentally cumbersome-especially for multiplexed gene targeting- and complicates an optimal gene targeting strategy. Furthermore, a TALE-based fusion of KRAB and the DNMT3A and DNMT3L domains resulted in low efficacy long-term gene silencing (Mlambo et al., 2018). Thus, it is unclear how generalizable these approaches are for establishing heritable gene silencing and whether there are genomic features that are required for writing and maintaining heritable epigenetic silencing. We hypothesized that an epigenetic editor composed of a single dead Cas9 fusion would enable us to broadly explore the biology and utility of heritable epigenetic gene silencing.

Here, we present the design, development, and characterization of CRISPRoff, a programmable epigenetic memory writer protein that can durably silence gene expression. Transient expression of CRISPRoff writes an epigenetic program that human cells maintain for more than 450 cell divisions, highlighting that this form of gene silencing is stable and heritable. Using genome-wide CRISPRoff screens, we show that this approach can durably and specifically silence the large majority of protein-coding genes and has a wide targeting window across gene promoters. Surprisingly, canonical CpG island annotations are not necessary for stable gene silencing by CRISPRoff. Lastly, we demonstrate that CRISPRoff can be used for silencing enhancers and engineering gene silencing programs in human stem cells that persist through differentiation to neurons. More generally, this system allows us to broadly explore the biological rules underlying epigenetic silencing and provides a robust tool for controlling gene expression, targeting enhancers, and exploring the principles of epigenetic inheritance.

### Results

### Rational Design of a Single Fusion Epigenome Memory Editor

We designed a CRISPR-based programmable epigenome editor protein, termed CRISPRoff-V1, composed of ZNF10 KRAB, Dnmt3A (D3A), and DNMT3L (D3L) protein domains fused to catalytically inactive S. pyogenes dCas9 (FIG. 1A). To test whether a transient pulse of CRISPRoff epigenetic editing could silence gene expression durably, we transiently co-transfected HEK293T cells stably expressing a DNA methylation-sensitive GAPDH-Snrpn GFP reporter with either CRISPRoff-V1, dCas9-KRAB (CRISPRi), or dCas9-D3A-3L, along with sgRNAs targeting the GAPDH-Snrpn synthetic promoter (Liu et al., 2016; Stelzer et al., 2015) (FIG. 1B). All three epigenetic editor proteins transiently silenced the GFP reporter (FIG. 1C). As expected for a transient transfection, expression of each epigenetic editor protein was lost over time, which for dCas9-KRAB and dCas9-D3A-3L resulted in restored expression of GFP. By contrast, for CRISPRoff-V1, gene silencing memory and CpG island (CGI) methylation was maintained long after CRISPRoff expression was lost (FIG. 1C).

Silencing by CRISPRoff-V1 appeared to be meta-stable as gene expression of the reporter gradually increased with time (FIG. 1C). To stabilize gene silencing memory, we encoded CRISPRoff with proteolysis-resistant linkers to minimize proteolysis that could result in untethered D3A-D3L and off-target DNA methylation as previously reported (Galonska et al., 2018; Hofacker et al., 2020). CRISPRoff variants programmed variably durable gene silencing (FIGS. 8A-8B). Second, we hypothesized that positioning D3A-3L at the N-terminus of dCas9 would allow Dnmt3A optimal access to CpG sites for DNA methylation (Zhang et al., 2018) (FIG. 8C). The CRISPRoff-V2 epigenetic editors we engineered each had similar gene silencing stability so we used CRISPRoff-V2.1 in all subsequent experiments (FIGS. 8D-8F).

Transient expression of CRISPRoff-V2 programmed a durable memory of gene silencing for at least 50 days post-transfection, with over 80% of transfected cells silencing the Snrpn-GFP reporter and over 90% silencing the endogenously GFP-tagged gene HIST2H2BE (H2B) (FIGS. 1E-1F, and 8G). H2B silencing was accompanied by CGI methylation (FIG. 1G). Notably, starting at 10 days post-transfection, no CRISPR-off protein was detected (FIG. 1E). Transfection of CRISPRoff-V2 mRNA also silenced expression of the endogenously GFP-tagged gene CLTA, supporting that transient expression of CRISPRoff leads to effective gene silencing (FIG. 8H). These results demonstrate that CRISPRoff epigenetic memory does not depend on sustained transgene expression.

To further compare CRISPRoff-V1 and V2, we silenced three cell surface-localized proteins (ITGB1, CD81, CD151) that are not required for cell proliferation or survival. Transfection of CRISPRoff-V1 with one sgRNA silenced each target gene in a fraction of cells and a pool of three sgRNAs improved silencing of ITGB1 and CD81 (FIG. 1H). CRISPRoff-V2 improves silencing of each gene, with at least 80% silencing at 3 weeks post-transfection (FIG. 1H).

### Durable and Multiplexed Silencing of Endogenous Genes

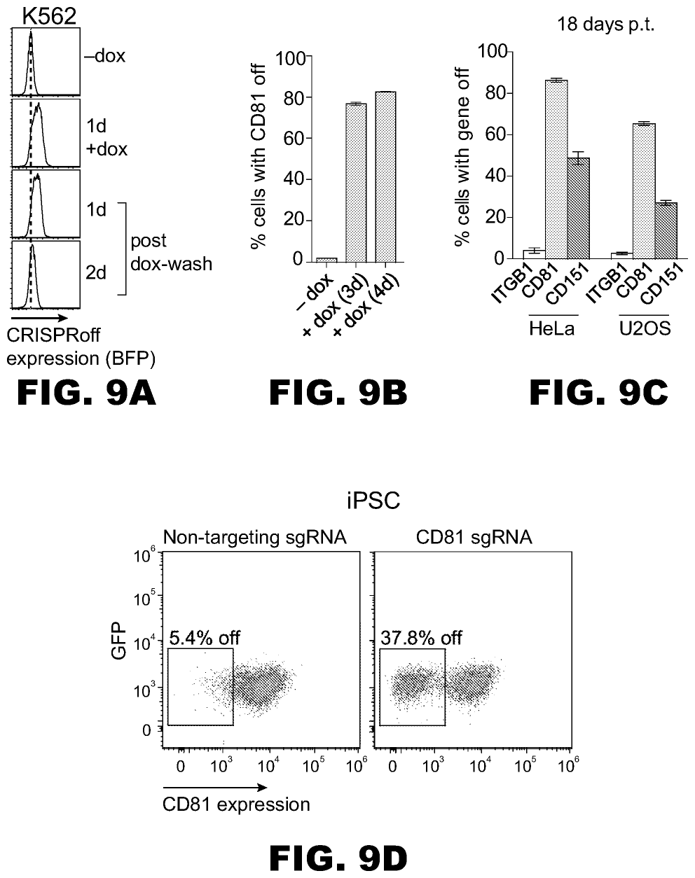

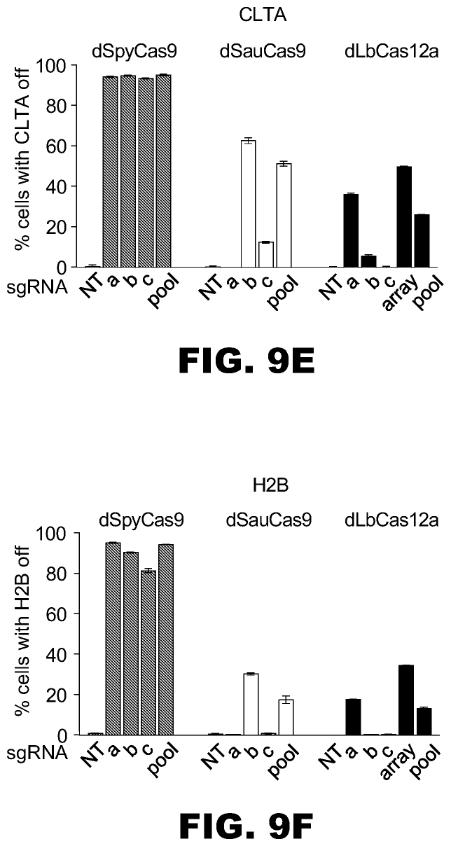

We demonstrated the efficacy of CRISPRoff-V2 in a variety of cell types, namely induced pluripotent stem cells (iPSCs), HeLa, U2OS, and K562 (as a doxycycline-inducible system) (FIGS. 9A-9D). We further show that CRISPRoff can be programmed by orthogonal DNA binding proteins: dCas9 from S. aureus (dSauCas9) and dCas12a from Lachnospiracea (dLbCas12a) (FIGS. 9E-9F). Silencing with dLbCas12a was improved when three crRNAs (CRISPR RNAs) were encoded as a single transcript that can be processed by dLbCas12a into individual crRNAs, indicating a route to multiplexed gene silencing.

To explore multiplexed silencing of endogenous human genes with S. pyogenes-based CRISPRoff, we targeted ITGB1, CD81, and CD151 in two, three, and four gene combinations (FIGS. 1I-1K and 8I-8K). At 30 days post-transfection, we observed robust multiplexed gene silencing of each gene combination (FIG. 1I). We observed that cells that silenced one gene have a higher likelihood of silencing the other targeted genes (FIGS. 11-1K, 8I, and 8K). For example, when co-targeting ITGB1 and CD81, cells that successfully silenced ITGB1 had a 25-fold higher percentage of cells that also silenced CD81 compared to cells that failed to silence ITGB1 (FIG. 1I).

To measure long term maintenance of epigenetic memory, we targeted the endogenous CLTA gene and followed CLTA expression in single cell clones (Leonetti et al., 2016a). Remarkably, at 15 months post-transfection or after ˜450 cell divisions, 38 out of 39 clones maintained silencing of CLTA (FIG. 1L).

### Epigenome Editing is Highly Specific

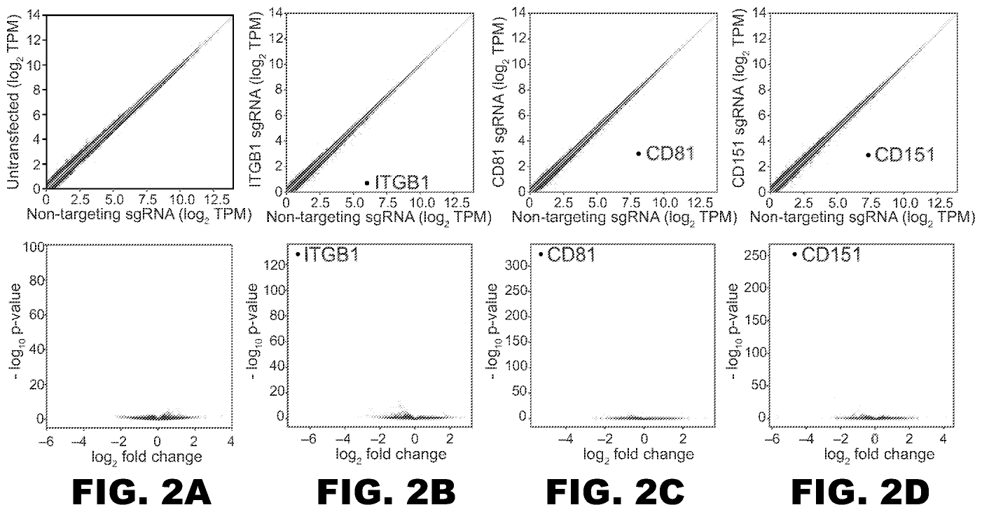

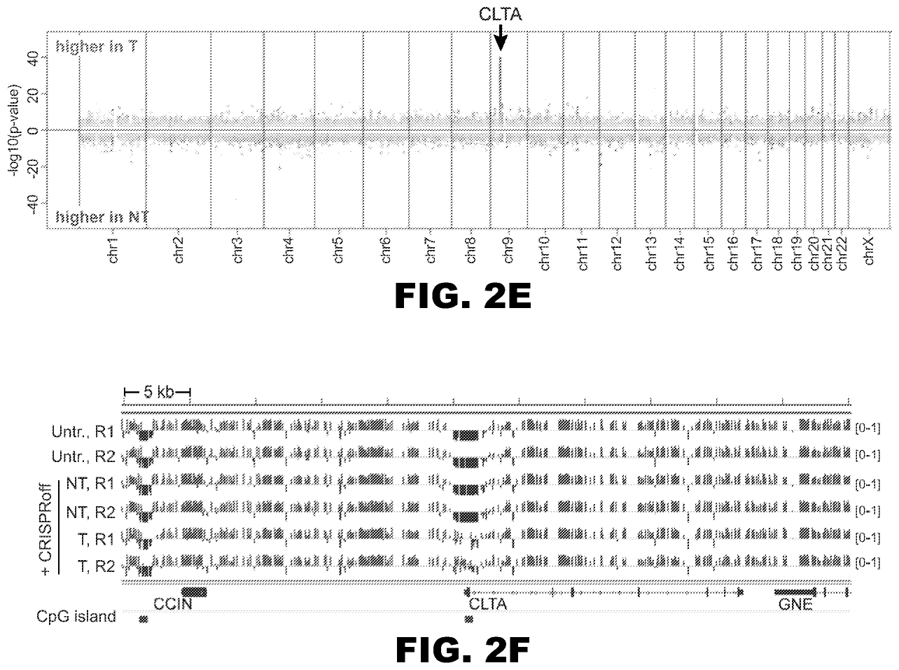

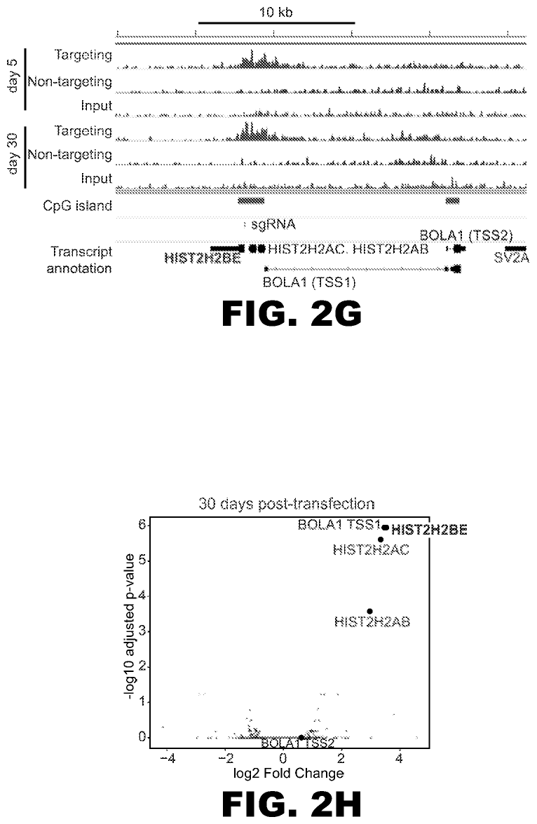

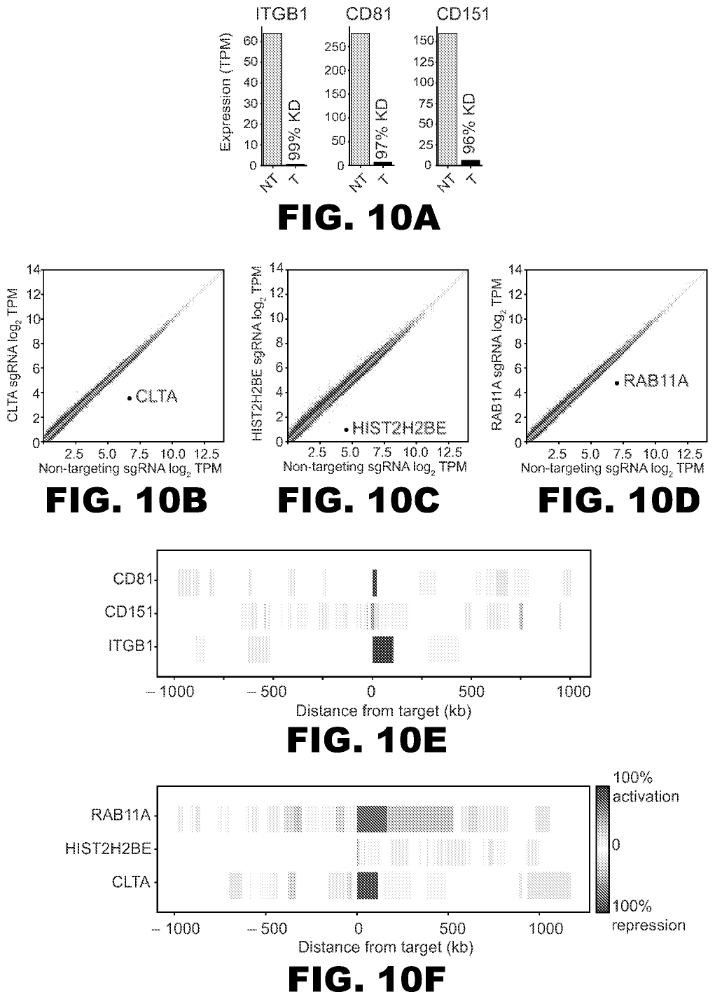

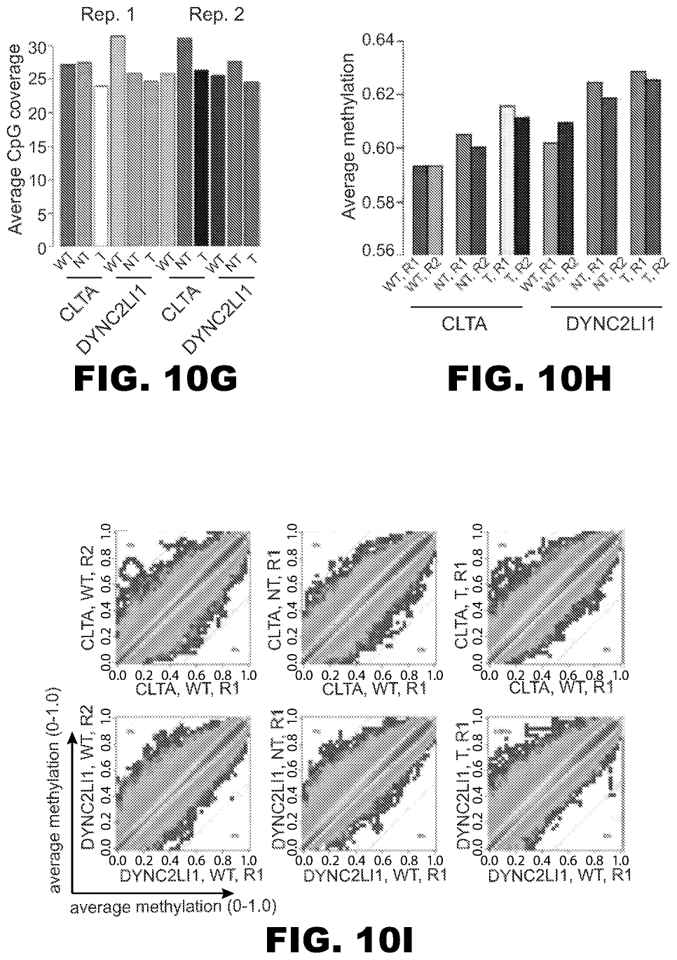

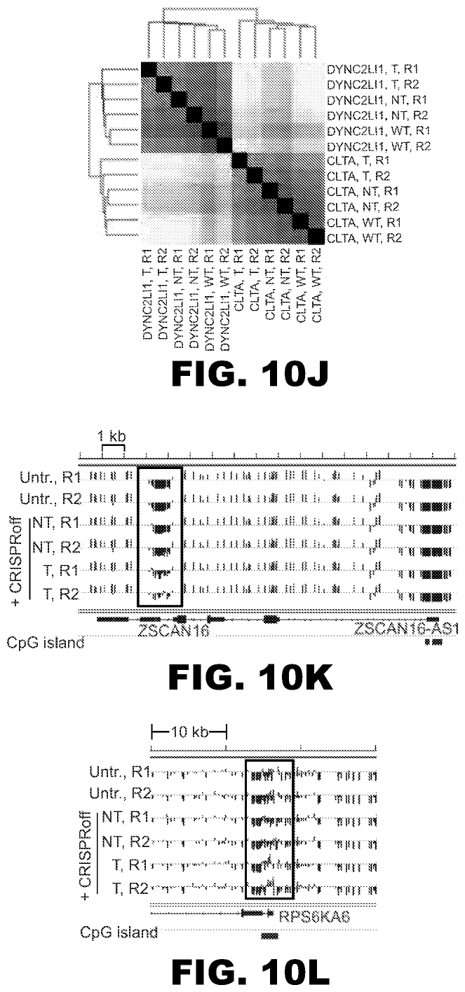

To assess the specificity of CRISPRoff, we performed RNA-seq of cells 33 days post-transfection of CRISPRoff and sgRNAs targeting ITGB1, CD81, and CD151 or a negative control sgRNA. Comparison of untransfected cells with cells transfected with CRISPRoff show minimal off-target gene knockdown (FIG. 2A). CRISPRoff targeting of ITGB1, CD81, and CD151 were highly specific and showed near complete repression of the targeted gene (FIGS. 2B-2D and 10A). RNA-seq analyses of three other cell lines with an endogenously GFP-tagged gene repressed by CRISPRoff (RAB11A, CLTA, and H2B) also showed robust and highly specific transcript knockdown (FIGS. 10B-10D). Analysis of neighboring genes within a 1 megabase window from the target gene showed no significant changes in gene expression (FIGS. 10E-10F). When analyzing the datasets from CRISPRoff targeting of ITGB1, CD81, and CD151, we observed 1-3 non-target transcripts with a log 2 fold-change >2 and adjusted p-value <0.5 in each gene knockdown experiment, albeit at much lower magnitude compared to the targeted gene. Differential expression of non-targeted transcripts may be due to indirect effects associated with target gene knockdown or off-target CRISPRoff activity.

We assessed CRISPRoff DNA methylation specificity by whole genome bisulfite sequencing (WGBS) 30 days post-silencing of CLTA (FIG. 10G). We detected a single dominant gain in DNA methylation at the CLTA promoter, in a 1.5 kb window across the CLTA promoter, highlighting the high specificity of CRISPRoff (FIG. 2E). We did not detect spreading of DNA methylation into the closest neighboring genes (FIG. 2F).

Consistent with previous analyses of DNA methylation in cells treated with DNMT-based epigenetic editors, we observed modestly higher global DNA methylation in CRISPRoff-transfected cells (<2%; FIGS. 10H-10I) (Galonska et al., 2018; O'Geen et al., 2019). However, global DNA methylation also varied between cell clones not exposed to CRISPRoff to a similar degree and the differences in local DNA methylation patterns at non-target DNA sites between cell clones was greater than any of the modest, non-specific changes in methylation seen following expression of CRISPRoff (FIGS. S3I-S3J). To examine whether possible CRISPRoff sgRNA dependent or independent off-target DNA methylation could alter gene expression, we inspected the top 10 most differentially methylated DNA regions. We did not detect any transcriptional differences for genes at or near the top 10 non-target differentially methylated regions (FIGS. 10K-10L). Thus, our WGBS data indicate that any off-target methylation differences are infrequent and unlikely to modulate gene expression or cellular phenotypes.

Lastly, we used ChIP-seq to profile changes in repressive H3K9me3 modifications after CRISPRoff targeting of the HIST2H2BE (H2B) gene. We detected a strong increase in H3K9me3 within a ˜5 kb region across the H2B promoter at 5 days post CRISPRoff transfection that was maintained at 30 days, demonstrating the stable propagation of H3K9me3 and DNA methylation as discussed further below (FIG. 2G). Comparing H2B-targeting and non-targeting sgRNA conditions showed that the most significant gain of H3K9me3 occurred at the H2B locus and three neighboring genes: HIST2H2AC, HIST2H2AB, and BOLA1 (FIG. 2H). We detected knockdown of HIST2H2AC expression whereas sequencing reads mapping to HIST2H2AB were not detected in our RNA-seq data. We did not detect transcriptional knockdown of BOLA1. These data, coupled with whole genome bisulfite sequencing data that showed confinement of CpG methylation, highlight the transcriptional and epigenomic specificity of CRISPRoff, while also documenting local epigenetic spreading and maintenance from the target site of establishment.

### Genome-Wide Targeting of CRISPRoff

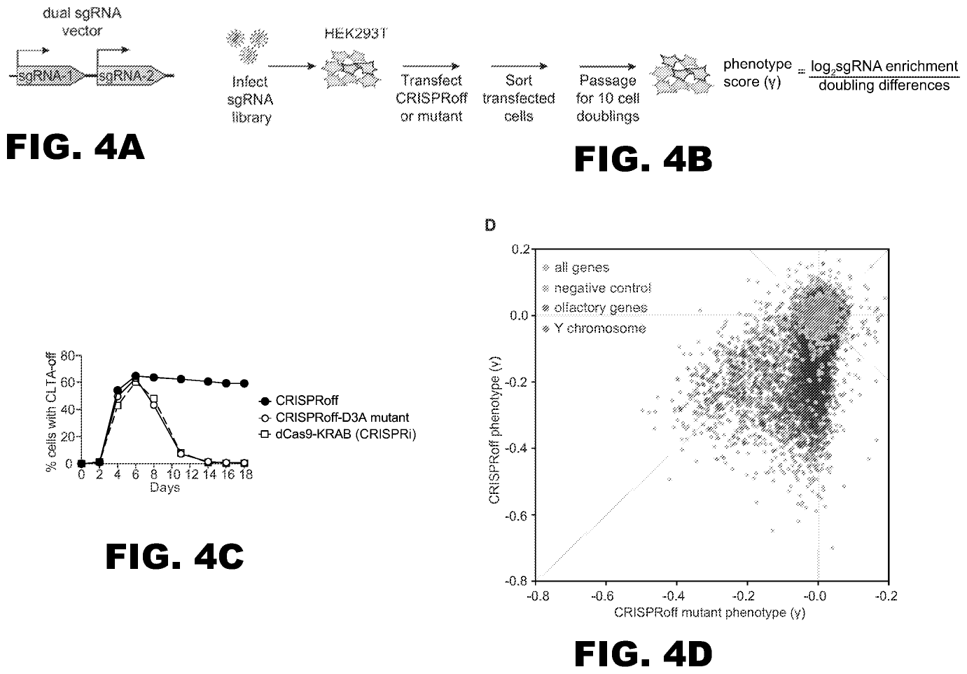

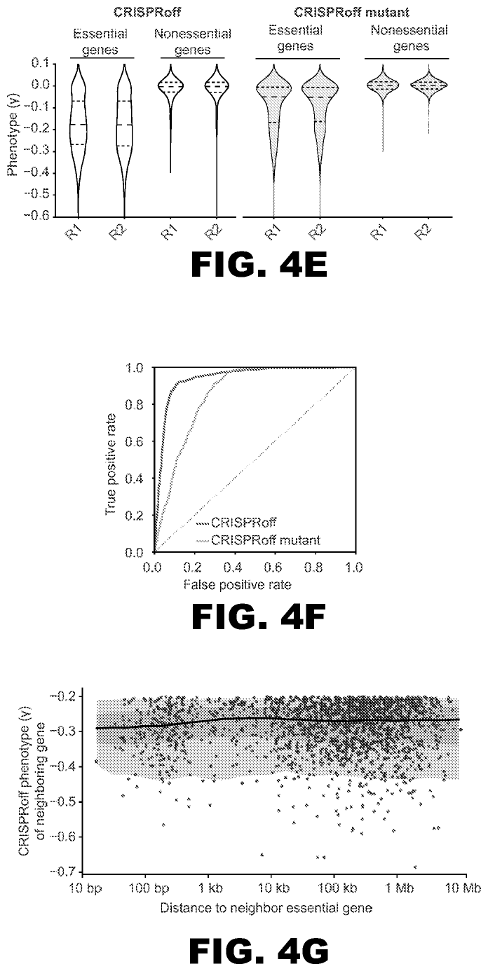

The simple design of CRISPRoff motivated us to perform pooled, genome-wide screens to determine its generalizability for silencing genes in the human genome. We designed a sgRNA library that targets over 20,000 protein-coding genes and includes about 1,000 non-targeting sgRNAs (Horlbeck et al., 2016a; Replogle et al., 2020). We constructed the sgRNA library to encode two unique protospacers targeting the same gene per lentiviral vector, as our experiments show improvement in CRISPRoff activity when using multiple sgRNAs targeting the same gene (Replogle et al., 2020) (FIG. 4A).

We performed growth-based pooled screens since gene essentiality datasets are available from previous functional genomics efforts, allowing us to compare the performance of CRISPRoff to other genome-wide dropout screens. To perform a CRISPRoff pooled screen, we packaged the sgRNA library into lentiviral particles then transduced and selected HEK293T cells such that on average each cell expresses one sgRNA vector. We then transiently transfected this pool of cells with plasmids encoding CRISPRoff and sorted cells that expressed the CRISPRoff protein. We harvested a population of CRISPRoff-transfected cells as a time zero (TO) sample and continued to passage a population of CRISPRoff-transfected cells for at least 10 cell doublings (T10), followed by deep sequencing of genomic DNA at both time points to read out and quantify the sgRNA sequences as a proxy for cell count. We inferred that sgRNAs that were depleted in the T10 population relative to T0 are active, as these sgRNAs effectively silenced the expression of essential genes and drop out of the population (FIG. 4B). As a control, we performed in parallel an identical screen with a CRISPRoff variant encoding the Dnmt3A^{E765A} mutation, which is catalytically inactive and thus unable to maintain durable gene silencing and instead mirrors the transient silencing effect of CRISPRi (FIG. 4C). By comparing these two screens, we identified sgRNAs in the CRISPRoff screen that silenced gene expression in a manner that is DNA methylation dependent.

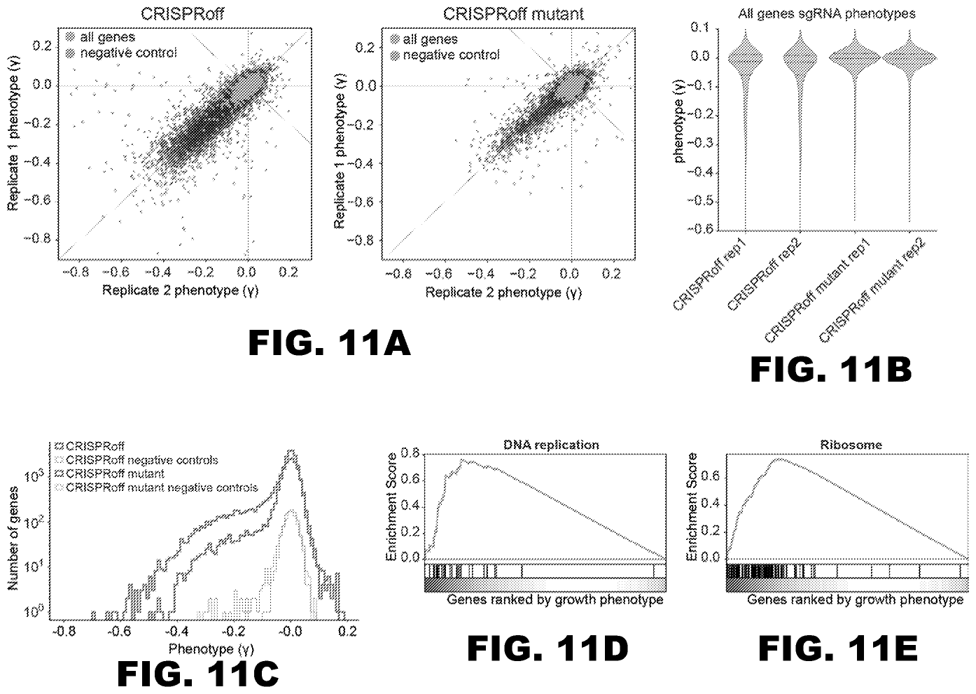

Analysis of the phenotype score (γ, with a more negative score indicating a stronger growth defect) for each gene showed that CRISPRoff expression reproducibly led to a more pronounced growth defect phenotypes compared to the CRISPRoff mutant (FIGS. 4D-4E, 11A-11C). A large set of genes showed drastic growth defects that were specific to CRISPRoff-mediated knockdown, highlighting the durable gene silencing effect of CRISPRoff. We also detected a subset of genes with comparable phenotypes between the two screens, likely due to their essentiality upon transient knockdown by the CRISPRoff mutant (FIG. 4D). We evaluated the specificity of silencing across the screens by analyzing the phenotype scores of control sgRNAs. Almost all negative control sgRNAs or sgRNAs targeting unexpressed genes (olfactory or Y chromosome genes) had little to no measured phenotype (1% with γ<−0.2) (FIG. 4D).

To evaluate the generality of CRISPRoff for gene silencing, we assessed the phenotype scores of genes that we expect to have growth phenotypes upon knockdown. Analysis of genes associated with DNA replication and the ribosome, which are predicted to be highly essential for cell proliferation, were among the most severe growth phenotypes (FIGS. 11D-11E). We then analyzed the phenotypes for common essential genes that are required for cell proliferation or survival in most cancer cell lines (Meyers et al., 2017). The growth defects of these genes were far more pronounced in CRISPRoff (median γ=−0.2) compared to the CRISPRoff mutant (median γ=−0.05), whereas the majority of nonessential genes did not produce growth phenotypes (FIG. 4E). The CRISPRoff mutant resulted in weak phenotype scores due to the lack of DNA methylation-dependent durable gene silencing. Collectively, the CRISPRoff screen resulted in high positive rate of calling essential genes with low false positive gene hits, suggesting that CRISPRoff has the ability to silence the majority of genes in the human genome (FIG. 4F).

Programmable epigenome editors can initiate epigenetic marks that spread from the site of establishment (Hathaway et al., 2012; Stepper et al., 2017). We wondered whether some CRISPRoff gene hits were due to a “neighboring gene effect” caused by DNA methylation spreading from a nearby essential gene. We catalogued gene “hits” (defined by genes with phenotype scores of −0.2 or lower) and determined their linear distance on the genome from the nearest gene hit. Although a subset of gene hits were within a 1 kb window distance, the majority of CRISPRoff hits were over 10 kb from the nearest gene hit (FIG. 4G). Since CpG islands are largely restricted within a 1-2 kb window (Deaton and Bird, 2011), we postulate that the majority of our observed gene hits are specific to the targeted gene promoter, consistent with the specificity demonstrated in our CRISPRoff RNA-seq, WGBS, and ChIP-seq experiments.

CRISPRoff Silencing of Genes that Lack CGI Annotations

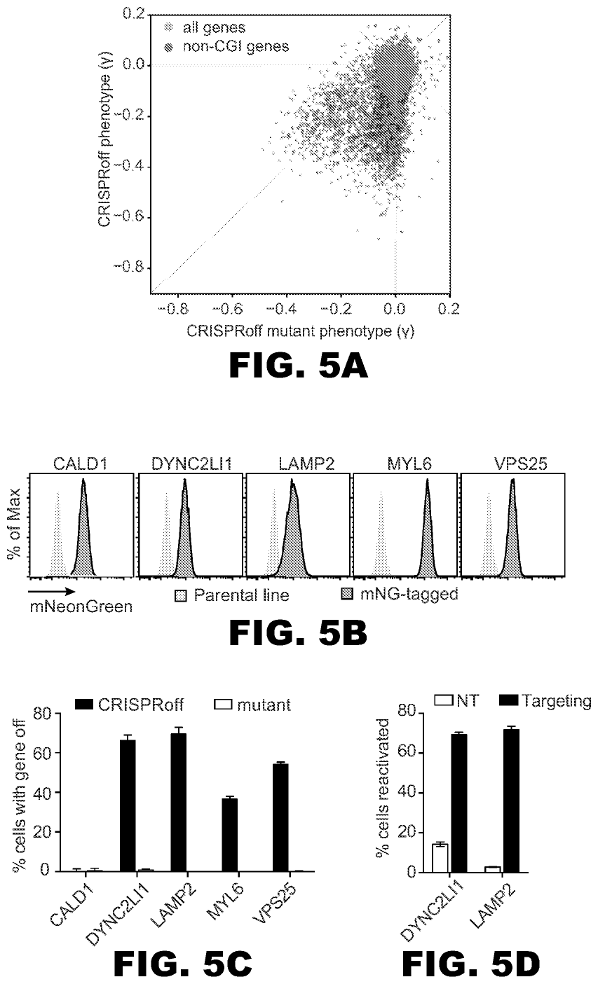

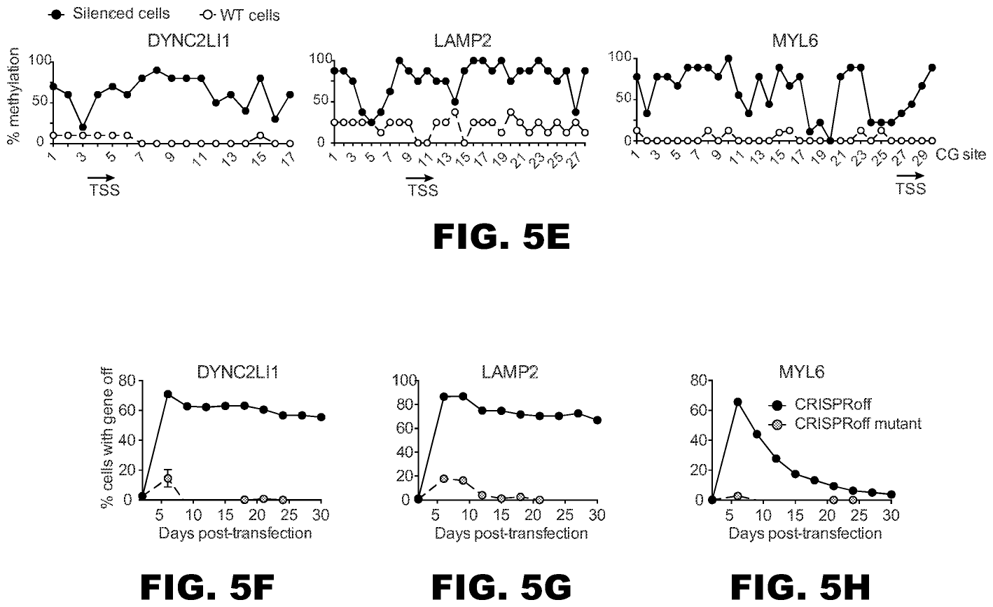

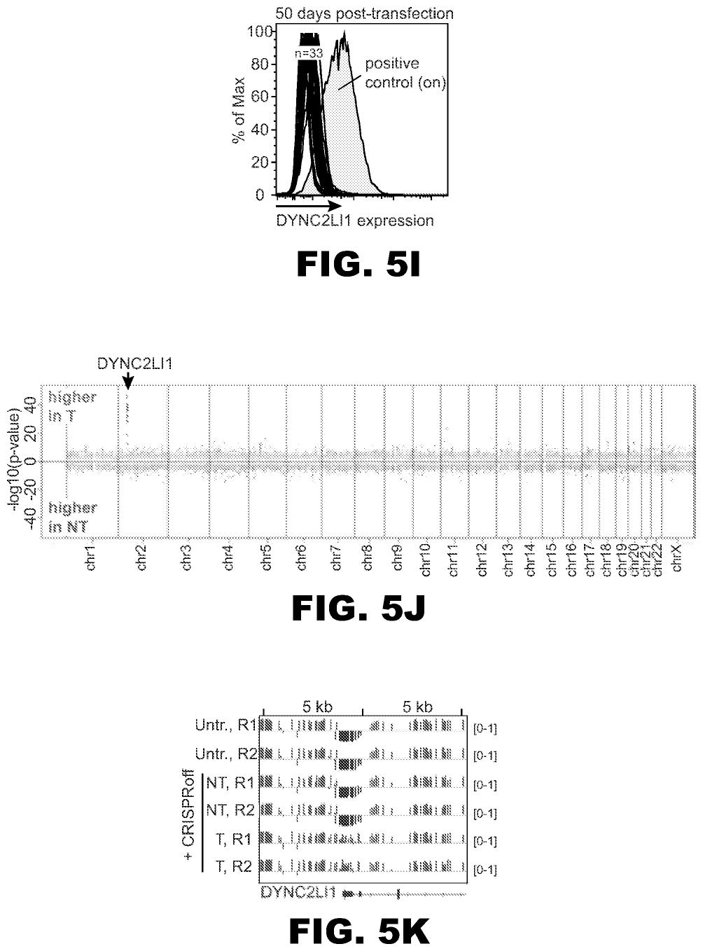

It is estimated that about 30% of human genes are not associated with a promoter CpG island (CGI) (Deaton and Bird, 2011). Given the observed generality of CRISPRoff for gene silencing, we wondered whether genes that lack CGI annotations can be silenced durably by CRISPRoff. Surprisingly, we found over 300 genes without CGI annotations with growth defects upon knockdown (γ<−0.1) by CRISPRoff, with 160 producing growth phenotypes γ<−0.2 (FIG. 5A). The majority of these genes had weak to no phenotype in the CRISPRoff mutant screen, indicating that their knockdown is DNA methylation dependent despite the absence of an annotated CGI.

To validate our observation that CRISPRoff can silence genes without annotated CGIs, we endogenously tagged five genes with no annotated CGI-CALD1, DYNC2LI1, LAMP2, MYL6, and VPS25—in HEK293T with mNeonGreen (mNG) and assessed durable silencing by CRISPRoff (FIG. 5B). At 14 days post-transfection of CRISPRoff, we detected a large percentage of cells that turned off DYNC2LI1, LAMP2, MYL6, and VPS25 (FIG. 5C). We did not detect stable silencing of CALD1 at 14 days, potentially due to its promoter being almost completely devoid of CpG dinucleotides or non-optimal sgRNAs used in the experiment (FIG. 5C). Transfection of the CRISPRoff mutant did not silence gene expression durably. Treatment of DYNC2LI1 and LAMP2-off cells with TETv4 led to reactivation of gene expression in about 70% of cells (FIG. 5D).

We isolated LAMP2, DYNC2LI1, and MYL6 silenced cells and profiled the DNA methylation status of the gene promoter by bisulfite sequencing. Cytosines within a CG context were highly methylated in silenced cells (FIG. 5E). We passaged DYNC2LI1, LAMP2, and MYL6-silenced cells for 30 days and observed stable silencing of DYNC2LI1 and LAMP2 (FIGS. 5F-5I). We also followed 33 single cell clones with DYNC2LI1 silenced and all clones repressed the gene by 50 days post-transfection with CRISPRoff (FIG. 5I). Although MYL6 underwent silencing associated with DNA methylation at an early time point, gene expression reactivated to near pre-CRISPRoff level by day 30.

Lastly, to probe the extent of DNA methylation across a non-CGI annotated gene, we performed WGBS of cells with DYNC2LI1 silenced after 30 days. We detected a single dominant gain in DNA methylation at the DYNC2LI1 promoter (FIG. 5J) consisting of a about 1.2 kb region of the promoter (FIG. 5K). Together, these data establish that epigenetic editing using CRISPRoff is not limited to genes with canonical CGI annotations and can be targeted to most genes encoded in the human genome. Moreover, based on these findings, we propose that the theoretical framework of CGI gene annotation does not always predict the presence of functional CpG sites, bolstering the power of CRISPRoff and CRISPRon for functional testing of CpG methylation in modulating gene expression.

### Targeting Rules for CRISPRoff Platform

We next explored the targeting landscape of CRISPRoff within gene promoters. Previously, we and others used sgRNA tiling screens and machine learning approaches to show that active sgRNAs for CRISPRi are localized in a narrow window at gene promoters, particularly at a nucleosome-depleted region immediately downstream of the transcription start site (TSS) (Gilbert et al., 2014). Despite successfully using these CRISPRi rules to design the genome-wide CRISPRoff essentiality screens, it remained untested whether the location of effective guides for CRISPRoff was similarly limited to this narrow window.

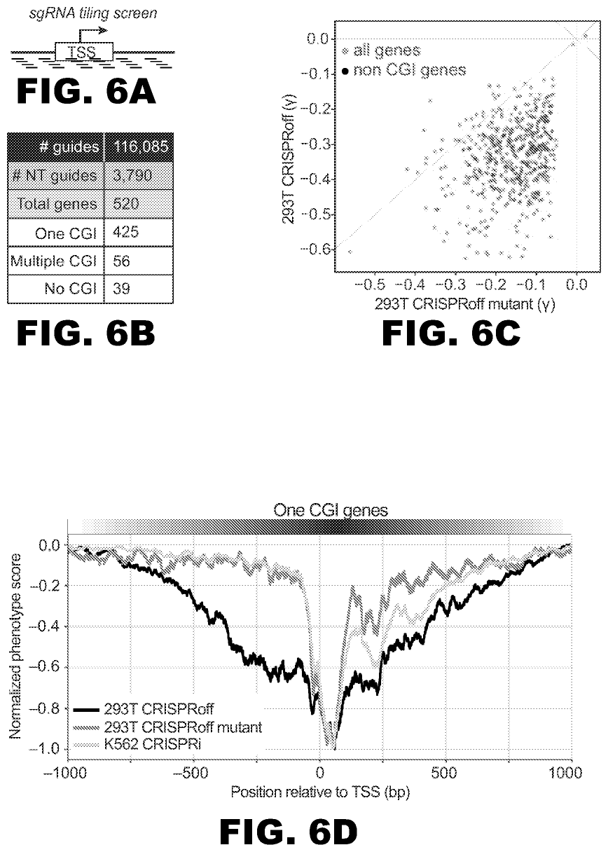

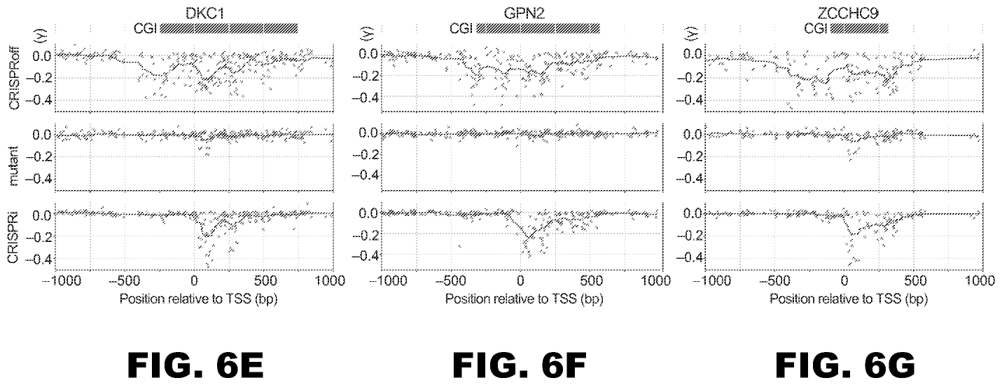

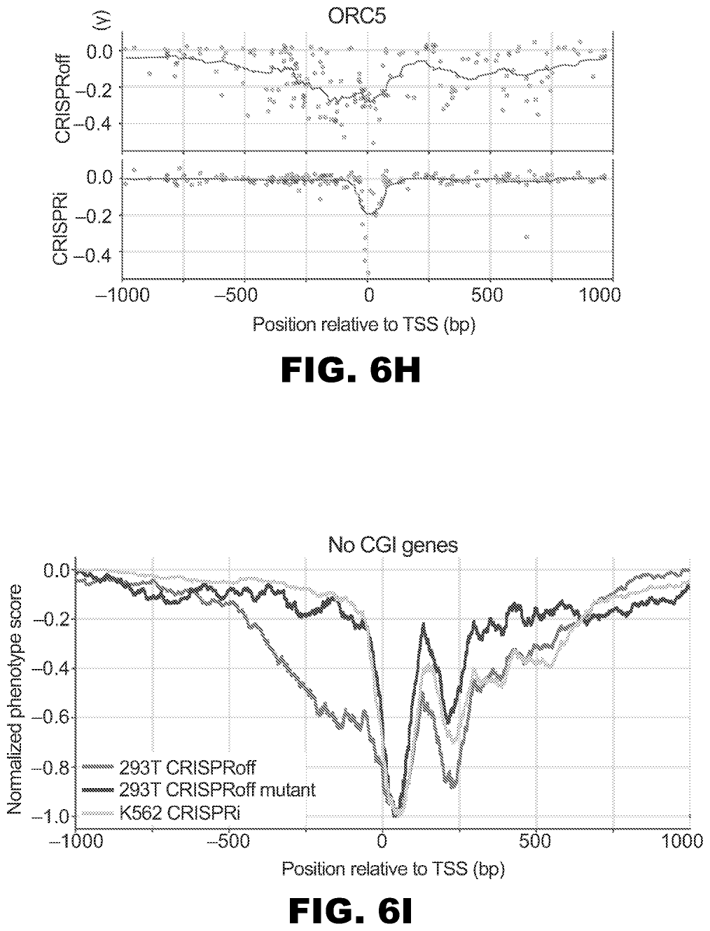

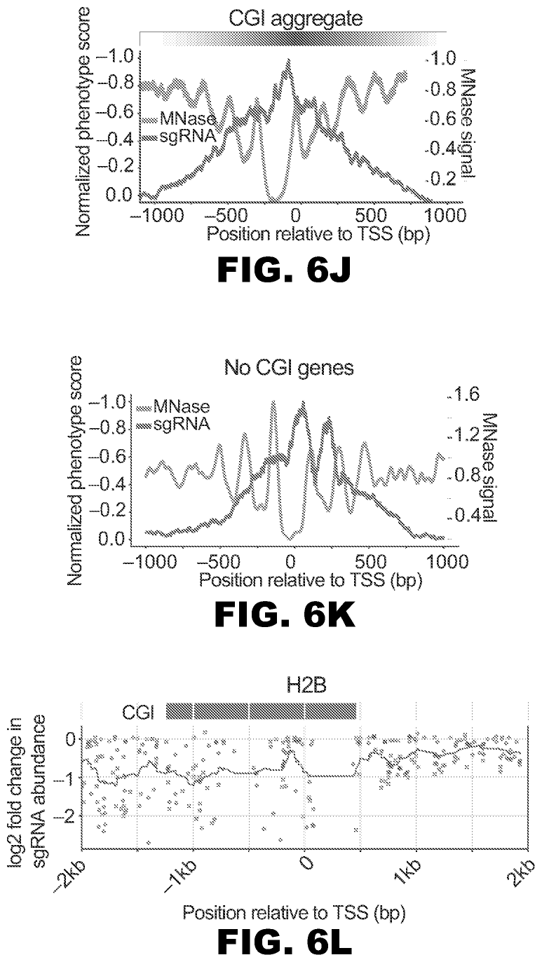

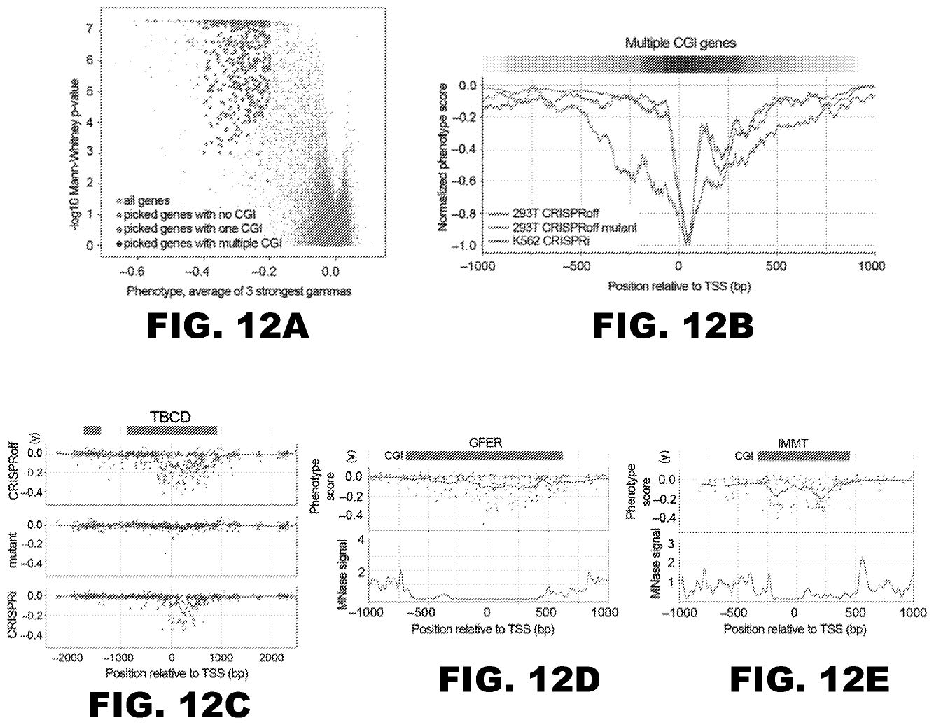

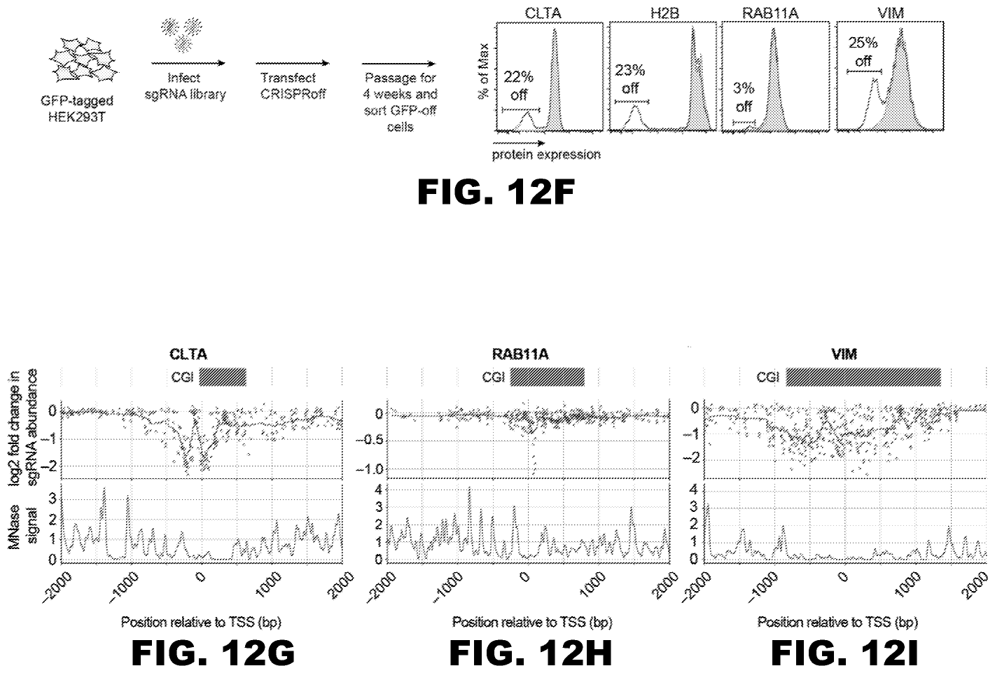

To empirically determine the targeting window of CRISPRoff, we designed a pooled sgRNA promoter tiling library against a subset of genes that are essential for cell growth based on previous CRISPRi screens in K562 cells and our genome-scale CRISPRoff screen (FIG. 12A). The library tiles PAM-containing sequences +/−1 kb from the TSS of 520 genes (425 with one annotated CGI, 56 with multiple CGIs, and 39 with no annotated CGI; defined by the presence of a CGI within 2.5 kb of the TSS), totaling ˜116,000 unique sgRNAs (FIGS. 6A-6B). We performed the CRISPRoff screens in HEK293T cells using the same experimental workflow as the genome-wide screens, in parallel with the CRISPRoff D3A^{E765A} methyltransferase mutant. We also transduced the sgRNA tiling library into K562 cells stably expressing dCas9-KRAB and performed a CRISPRi screen to compare gene silencing mediated by dCas9-KRAB alone.

To evaluate the screens, we calculated the phenotype score (γ) for the three most active sgRNAs per gene and compared phenotypes across the three screens. We first focused on the 425 genes with one annotated CGI, as these were predicted to be canonical targets for CRISPRoff-mediated silencing. The phenotypes for the CRISPRoff screen (median γ=−0.33) were more pronounced compared to the CRISPRoff mutant screen phenotypes (median γ=−0.15), establishing that strong phenotypes observed in the CRISPRoff screen are DNA methylation-dependent (FIG. 6C).

To compare the optimal sgRNAs between CRISPRoff and CRISPRi, we normalized the phenotypes across the screens and generated an aggregate plot of sgRNA activities relative to the TSS (FIG. 6D). Consistent with our previous work, highly active sgRNAs for CRISPRi were centered on a narrow window (about 75 bp) directly downstream of the TSS. Similarly, active sgRNAs for the CRISPRoff mutant mirrored CRISPRi, which we expected because the KRAB domain remains functional in this fusion protein despite the lack of DNA methylation activity. In contrast, the active CRISPRoff sgRNAs were broadly distributed across the TSS, notably within a 1 kb window centered on the TSS. Representative gene analysis of DKC1, GPN2, and ZCCHC9 shows that even within a single promoter, active sgRNAs for CRISPRoff are distributed across −500 to +500 bp from the TSS—a greatly widened targeting window for silencing compared to CRISPRi (FIGS. 6E-6G). We also observed effective sgRNAs outside the CGI that were DNA methylation-dependent (FIG. 6G), indicating that functional CpGs are not necessarily confined to canonical CGIs as observed in our WGBS data. Our aggregate plot analysis of active sgRNAs targeting the 56 genes with multiple annotated CGIs also shows a broad targeting window, similar to genes with one annotated CGI, and centered at the TSS (FIGS. 12B-12C).

Analysis of the 39 genes without promoter CGIs showed many highly active sgRNAs of comparable phenotype strength to genes with annotated CGIs (FIG. 6C, colored red) and the phenotypes were strongly diminished in the CRISPRoff mutant screen. A representative gene plot of ORC5 shows that similarly to genes with annotated CGIs, active CRISPRoff sgRNAs are spread across −500 to +500 bp from the TSS (FIG. 6H). Moreover, we observed that CRISPRoff has a broadened targeting window despite the lack of an annotated CGI for these 39 genes (FIG. 6I). Our experiments demonstrate that the optimal window for CRISPRoff gene silencing is similarly broad for genes with and without annotated CGIs, likely due to low density CpG sites that are functional for methylation-dependent gene silencing as we demonstrated for DYNC2LI1, LAMP2, MYL6, and VPS25 (FIG. 5C).

We observed that active sgRNAs are not evenly distributed but instead appear in a periodic pattern within the −500 to +500 bp window, as shown for DKC1, GPN2, and ZCCHC9 (FIGS. 6E-6G). Overlaying nucleosome occupancy with CRISPRoff sgRNA activity scores for all genes showed that the most active sgRNAs are located in nucleosome-depleted regions of gene promoters, as we and others have shown previously for Cas9 and dCas9-based tools (FIGS. 6J-6K, 12D and 12E) (Horlbeck et al., 2016b; Isaac et al., 2016).

To validate that the CRISPRoff targeting window is similar for genes that do not have a growth phenotype upon knockdown, we designed tiling sgRNA libraries spanning +/−2.5 kb from the TSS to target four endogenous genes: CLTA, H2B, RAB11A, and VIM. For each custom sgRNA library screen, we utilized the corresponding HEK293T cell line that expresses the endogenously GFP-tagged gene (Leonetti et al., 2016b). Each cell line was transduced with the respective sgRNA library, transfected with CRISPRoff, and the cells were passaged for 4 weeks to ensure that gene silencing was durable (FIG. 12F). We then sorted GFP positive and GFP negative cell populations for each screen and processed the samples as described above. We calculated sgRNA efficacy by identifying sgRNAs in the gene-off (GFP−) population compared to the gene-on (GFP+) population.

Similar to the growth screens, active sgRNAs for CLTA, H2B, and VIM spanned a large window across the TSS (FIGS. 6L and 12G-12I). Active CRISPRoff sgRNAs for CLTA were within two distinct regions, with one region upstream of the TSS outside of the annotated CGI (FIG. 12G). Unexpectedly, sgRNAs targeting ˜2 kb upstream of the H2B TSS were highly active (FIG. 6L). Similarly, for VIM, active sgRNAs spanned a 2 kb window +/−1 kb from the TSS. By contrast, active sgRNAs for RAB11A were constricted to a narrow window at the TSS. Overlaying nucleosome occupancy with sgRNA activity showed that the RAB11A promoter is nucleosome-dense (FIG. 12H). From these data, we interpret that CRISPRoff accessibility is restricted by nucleosomes; however, once bound, CRISPRoff can silence gene expression even when distal to the TSS.

### Durable Gene Silencing is Dependent on H3K9Me3 and DNA Methylation Maintenance

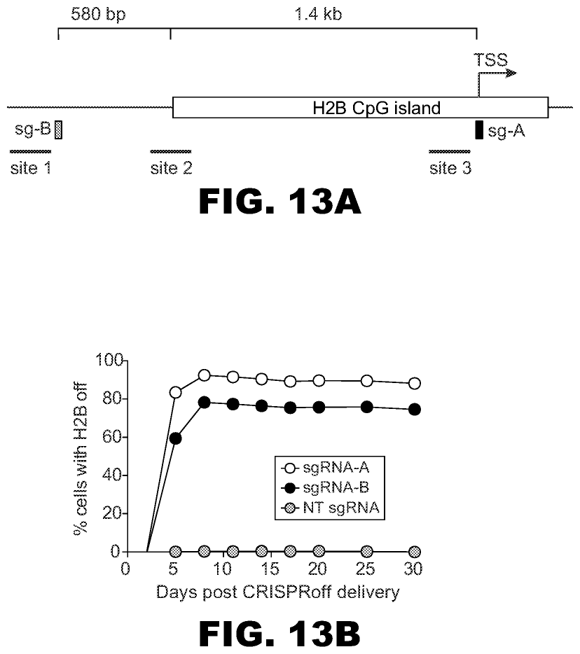

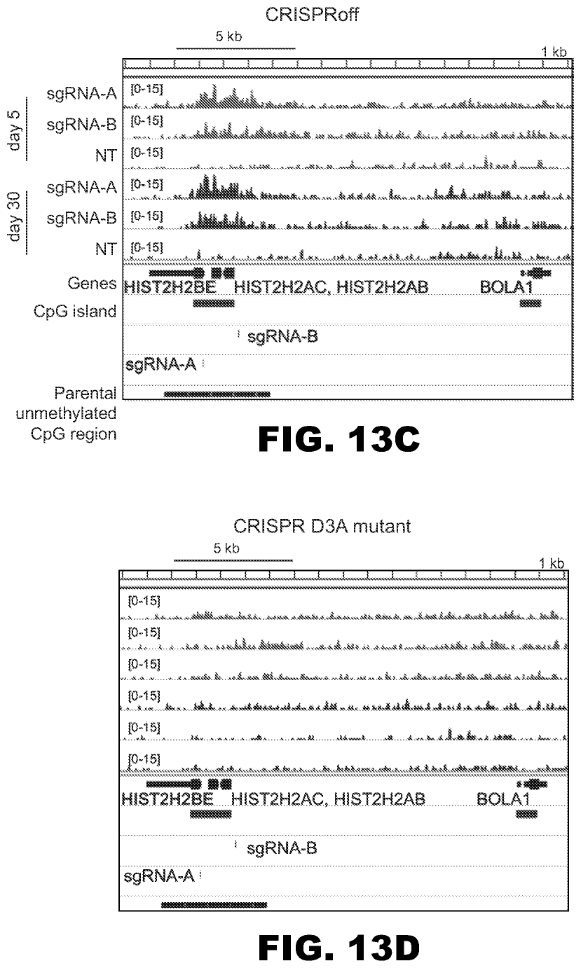

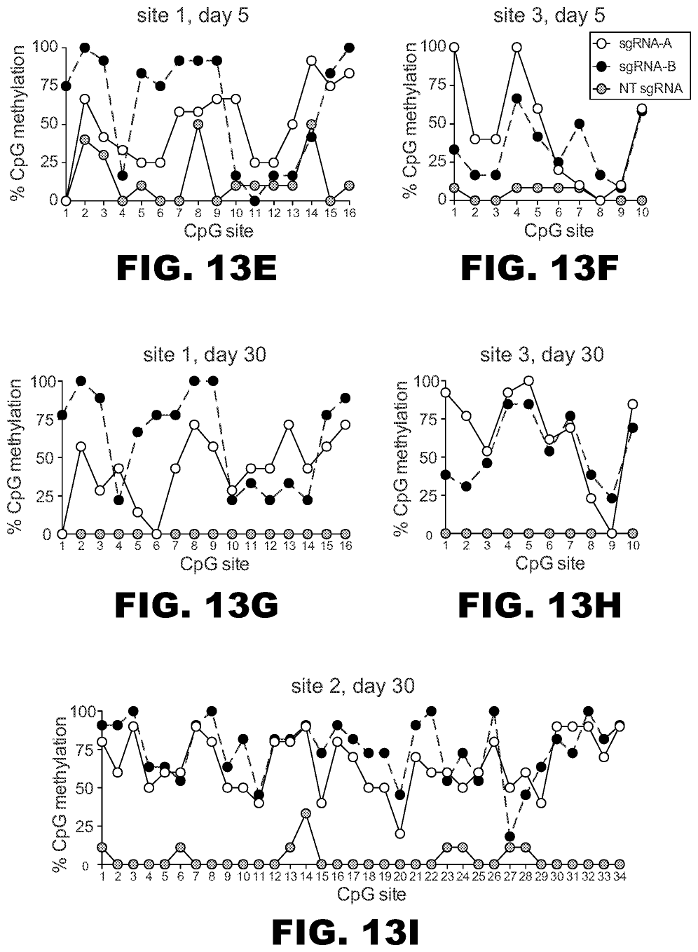

To explore the mechanism underlying CRISPRoff-mediated heritable memory, we made use of the wide targeting window across the H2B promoter to investigate the establishment, spreading, and maintenance of H3K9me3 histone modifications and CpG methylation marks. We targeted CRISPRoff to the TSS (sgRNA-A) and to a distal site ˜2 kb upstream of the TSS (sgRNA-B) (FIG. 13A). At 30 days post CRISPRoff transfection, 89% of cells maintained H2B silencing when sgRNA-A was delivered compared to 76% with sgRNA-B (FIG. 13B). Using ChIP-seq, we showed that both sgRNAs induced deposition at day 5 and maintenance at day 30 of H3K9me3 across the locus despite the ˜2 kb distance between the sgRNA binding sites (FIG. 13C). The acquired H3K9me3 modifications in CRISPRoff-treated cells overlapped with the unmethylated CpG region in untreated parental cells (bottom track in FIG. 13C). In contrast, deposition and maintenance of H3K9me3 was far weaker with CRISPRoff bearing the D3A mutation, consistent with the failure to sustain gene repression with the mutant (FIG. 13D).

We next profiled CRISPRoff-mediated CpG methylation at the targeted distal and TSS regions. At day 5, we detected establishment and spreading of CpG methylation from the site of initiation to an about 2 kb site from the sgRNA binding site (labeled site 1 and site 3, FIGS. 13E-13F). By day 30, we detected stable maintenance of DNA methylation at both sgRNA binding sites (FIGS. 13G-13H). We also detected a high degree of CpG methylation between the sgRNA binding sites, suggesting a linear movement of spreading from the site of initiation (site 2, FIG. 13I). These data, together with our WGBS data, highlight the orchestration of histone and DNA methylation deposition, spreading, and maintenance and indicate that there are underlying regulatory principles that likely depend on the genomic context.

CRISPRoff Gene Silencing in iPSCs and iPSC-Derived Neurons

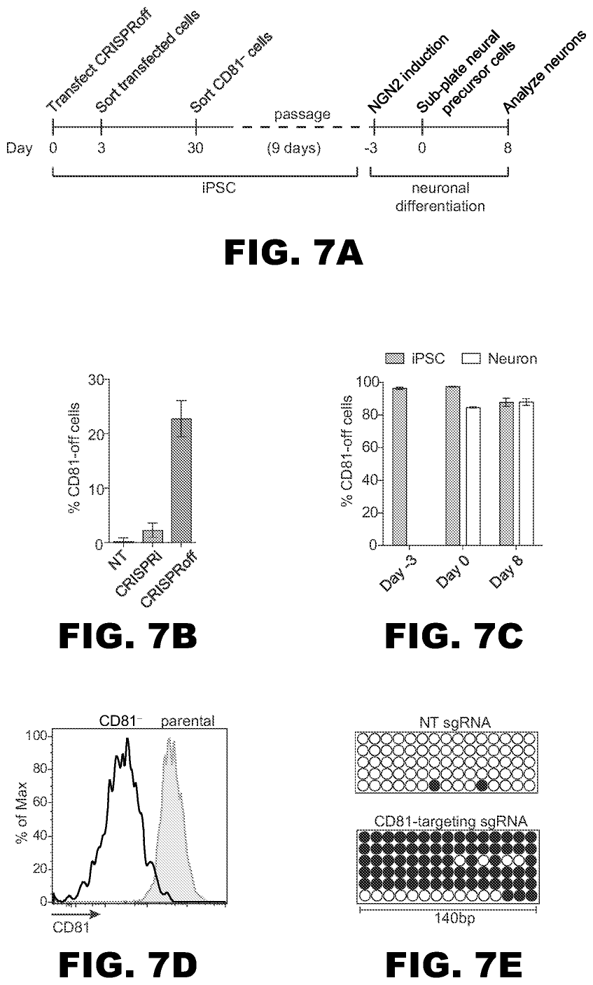

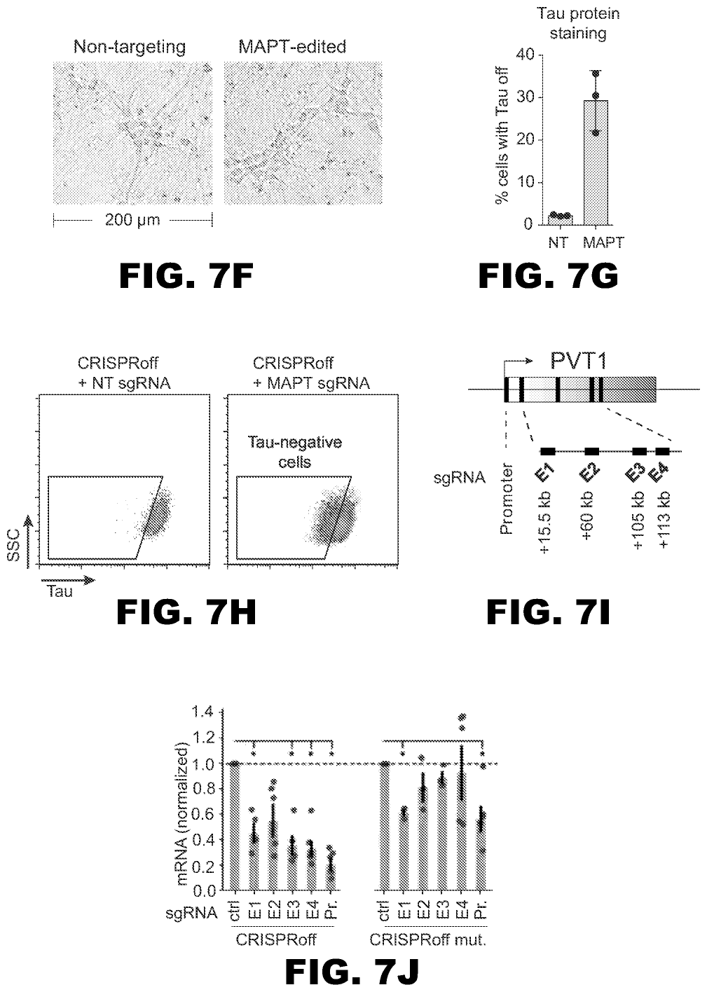

Due to the utility of stem cells for studying the development and function of specific cell types, we employed CRISPRoff in induced pluripotent stem cells. We transfected iPSCs with CRISPRoff and sgRNAs targeting CD81 or a non-targeting control and found that at 30 days post-transfection, many iPSCs had stably silenced CD81 (FIGS. 7A-7B). Thus, CRISPRoff-encoded memory of silencing is stably maintained in stem cells.

We isolated CD81-off iPSCs and differentiated the cells into neurons, as previously described (Tian et al., 2019) (FIG. 7A). We then measured CD81 protein levels at the neural precursor cell stage (day 0 of differentiation) and after cells had differentiated into neurons (8 days post-differentiation). We observed that CD81 remained silenced during and after neuronal differentiation in 90% of cells (FIGS. 7C-7D). A similar fraction of undifferentiated iPSCs maintained CD81 silencing during the same time course, suggesting that the reactivation of CD81 in about 10% of cells was not due to the differentiation process. We harvested genomic DNA from CD81-off neurons and detected heavily methylated promoter CpG dinucleotides compared to neurons treated with CRISPRoff and a non-targeting sgRNA (FIG. 7E).

We next applied CRISPRoff-mediated editing of iPSC-derived neurons to silence MAPT, a gene that encodes the Tau protein and is implicated in various neurological diseases (Iqbal et al., 2016). MAPT is transcriptionally repressed in iPSCs by H3K27me3 rather than by DNA methylation and H3K9me3 and its expression increases substantially during neuronal differentiation (Guenther et al., 2010). We hypothesized that CRISPRoff could write an epigenetic memory of silencing at the MAPT locus that would persist through neuronal differentiation to silence MAPT in neurons. We transiently transfected CRISPRoff into iPSCs along with sgRNAs targeting MAPT or a non-targeting control (FIG. 7F). At day 10 of the differentiation protocol, we measured Tau protein levels and found ˜30% of cells with reduced Tau expression compared to a non-targeting control (FIGS. 7G-7H). Together, these data support CRISPRoff-mediated epigenome editing as an applicable technology for rewriting gene expression programs in iPSC-derived cells, especially for modulating gene expression in cells where delivery of gene editing platforms remains a challenge.

### CRISPRoff Targeting of Enhancer Elements

Finally, we explored the potential utility of CRISPRoff for silencing promoter-distal elements by targeting enhancers that control the expression of the PVT1 long noncoding RNA (Cho et al., 2018; Fulco et al., 2016). We transiently expressed CRISPRoff in the MDA-MB-231 breast cancer cell line with sgRNAs targeting either the PVT1 promoter or at four previously identified enhancer elements downstream of the PVT1 promoter: E1 (+15.5 kb), E2 (+60 kb), E3 (+105 kb), and E4 (+113 kb) (FIG. 7I). We detected a significant reduction of PVT1 transcript levels with sgRNAs targeting E1, E3, and E4 (˜40-60%) compared to 80% knockdown with promoter-targeting sgRNAs (FIG. 7J, left). In contrast, parallel experiments using the CRISPRoff-Dnmt3A^{E765A} mutant resulted in less robust knockdown (FIG. 7J, right). These results highlight the potential use of CRISPRoff for mapping and dissecting the functions of enhancer elements and noncoding regulatory elements in the human genome.

### Discussion

Here, we present CRISPRoff and CRISPRon, two technologies for programmably writing and erasing epigenetic memories to control gene expression programs. Transient expression of CRISPRoff writes a robust, specific, and multiplexable gene silencing program that is memorized by cells through cell division and differentiation, which can be rapidly reversed by CRISPRon. We show that CRISPRoff can specifically and robustly silence the large majority of human genes. Our experiments demonstrate CRISPRoff can perturb enhancers, opening the potential to target genome elements that control tissue-specific gene expression (Fulco et al., 2016; Tarjan et al., 2019).

Our finding that targeted DNA methylation outside of annotated CGIs can lead to robust memorized gene silencing extends the canonical model of methylation-based gene silencing, which posits that a high density of CpG methylation is a requirement for stable propagation of silencing (Boyes and Bird, 1992). Although CRISPRoff-mediated writing of DNA methylation and histone modification are artificially programmed, our results motivate the need to define functional DNA methylation and dissect regulatory DNA elements and other host factors that mediate initiation, spreading, and maintenance of histone and DNA methylation marks. For example, targeting CRISPRoff 2 kb upstream of the H2B TSS leads to acquisition and maintenance of H3K9me3 and DNA methylation marks at the same genomic positions as targeting CRISPRoff directly proximal to the TSS, pointing to the existence of preexisting boundaries that restrict epigenetic spreading. By allowing the initiation of silencing at a defined time and genomic location, CRISPRoff provides a unique tool for addressing these and other fundamental questions regarding the mechanism and biological role of heritable gene silencing in mammalian cells (Audergon et al., 2015; Iglesias et al., 2018; Ragunathan et al., 2015; Yu et al., 2018).

CRISPRoff provides a valuable complement to existing CRISPRi and CRISPR nuclease approaches (Doench, 2018; Hanna and Doench, 2020; Shalem et al., 2015). CRISPRoff gene silencing can lead to effectively complete nulls without inducing a DNA damage response facilitating multigene targeting screens or therapeutic cell engineering (Ihry et al., 2018). The ability to target CRISPRoff to a large window upstream of the TSS allows access to promoter SNPs that can be utilized for allele-specific targeting of disease-associated mutations. This will broadly enable approaches to silence dominant negative alleles. Similarly, silencing of long noncoding RNAs and regulatory RNAs provides a new avenue for stable reprograming of gene expression. Silencing of inhibitory elements such as antisense transcripts, will result in a heritable increase in expression of some genes, enabling therapeutic efforts to mitigate haploinsufficiency or imprinting disorders (Buiting et al., 2016). More broadly, heritable epigenetic silencing provides a general tool for rewiring human gene expression programs.

### Example 2

### Blocking Flavivirus Infection by Epigenome Editing

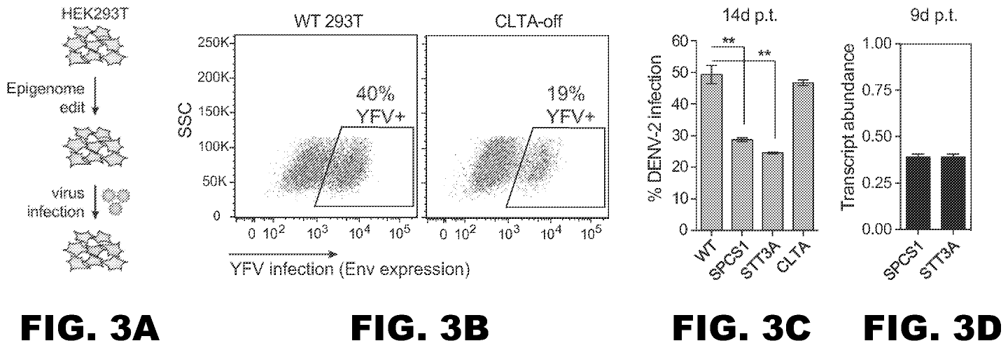

Medical interventions that promote immunity to viruses, such as vaccines, are one of the most promising classes of antiviral therapies. However, vaccines can take years to develop and deploy. We reasoned that epigenome editing is an attractive platform for blocking viral infections due to the transient pulse of editor expression, durability and reversibility of silencing, and unlike genome editing, there is no permanent change in the underlying DNA sequence (FIG. 3A).

As an initial test to assess the efficacy of epigenome editing in blocking viral infections, we challenged CRISPRoff-treated HEK293T cells that have maintained silencing of CLTA for >1 year with the live attenuated yellow fever virus (YFV) vaccine 17D. YFV is a Flavivirus that enter host cells through clathrin-mediated endocytosis. We harvested cells two days after YFV infection and detected a 50% reduction in infection of CLTA-off cells compared to WT HEK293T (FIG. 3B). CLTA is one of two clathrin light chains that can form the functional clathrin triskelion and we reasoned that the infected cells are due to the virus utilizing CLTB light chain-containing clathrin triskelion molecules for entry.

We next applied epigenome editing to block Dengue Virus (DENV-2) infection by turning off SPCS1 and STT3A, which are key factors in DENV pathogenesis. We transfected CRISPRoff into HEK293T and infected the cells with DENV-2 at 14 days post transfection. Infection of DENV-2 is reduced significantly in SPCS1 and STT3A targeted cells (FIG. 3C). Gene silencing of SPCS1 and STT3A was measured at about 60% knockdown, mirroring the DENV-2 infection in edited cells (FIG. 3D). CLTA-silenced cells were infected at the same level as unedited WT cells, suggesting that DENV-2 entry is CLTA-independent.

### Example 3

The MAPT knockdown experiments were performed by transfecting CRISPRoff with MAPT-targeting sgRNAs, as described below. To increase signal, iPSCs were co-transfected with MAPT-targeting sgRNAs along with CD81-targeting sgRNAs. As controls, iPSCs were transfected with CRISPRoff and non-targeting sgRNAs. One week post-transfection, the iPSCs were stained with CD81 antibody and cells with CD81 knocked down were FACS-sorted and passaged for 1 week prior to differentiation into neurons. The neuronal differentiations were performed as described below. At 10 days post-differentiation, neurons were harvested for qPCR analysis and antibody body staining for Tau protein and analyzed by flow cytometry. The quantification of MAPT/Tau knockdowns were normalized to WT untransfected cells. The results are shown in FIGS. 8A-8C.

### Materials and Methods

### Plasmid Design and Construction

The dCas9 and KRAB sequences were obtained from a previous CRISPRi construct (Gilbert et al., 2013). The D3A and D3L sequences, including the D3A-D3L fusion, originated from (Stepper et al., 2017) and were assembled with dCas9 and KRAB DNA sequences into a CAG-expression plasmid using NEBuilder® HiFi DNA Assembly (NEB). All CRISPRoff fusion proteins include BFP as either a direct fusion or with a P2A-cleavage sequence to measure transfection efficiency by flow cytometry. The dSaCas9 (D10A, N508A) sequence was PCR amplified from pX603 (Addgene #61594) and the dLbCas12a sequence was PCR amplified from (Tak et al., 2017). The GAPDH-Snrpn-GFP lentiviral reporter originated from Addgene #70148 (Liu et al., 2016; Stelzer et al., 2015).

The sgRNA plasmids were constructed by restriction cloning of protospacers downstream of a U6 promoter using BstXI and BlpI cut sites, as previously described. The sgRNA expression plasmids also express a T2A-mCherry marker to measure transfection efficiency. The sgRNA sequences used for CRISPRoff experiments are listed in Table 5. The sgRNA sequences were chosen based on our previous algorithm to predict active CRISPRi sgRNAs (Horlbeck et al., 2016a).

All mRNA constructs were synthesized using the mMESSAGE mMachine™ T7 Ultra Transcription Kit (Thermo Fisher Scientific). The T7 promoter sequence (SEQ ID NO:111) was first cloned upstream of the CRISPRoff sequence. The T7-CRISPRoff sequence was PCR amplified and used as template for in vitro synthesis reactions. Following the manufacturer protocol for synthesis, the reactions were cleaned by chloroform extraction and isopropanol precipitation.

### Cell Culture, DNA Transfections, and Flow Cytometry

All cell lines were cultured at 37° C. with 5% CO2 tissue culture incubators. HEK293T (female), HeLa (female), and U20S (female) cells were cultured in Dulbecco's modified eagle medium (DMEM) in 10% FBS (HyClone), 100 units/mL streptomycin, 100 μg/ml penicillin, and 2 mM glutamine. K562 (female) cells were maintained in RPMI-1640 with 25 mM HEPES and 2.0 g/L NaHCo3 in 10% FBS, 2 mM glutamine, 100 units/mL streptomycin, and 100 mg/mL penicillin. WTC Gen1c iPSCs (male) were cultured in mTESR media (STEMCELL Technologies) under feeder-free conditions on growth factor-reduced Matrigel (BD Biosciences). Cells were passaged using Accutase (STEMCELL Technologies) and seeded on Matrigel coated plates with mTESR media supplemented with p16-Rho-associated coiled-coil kinase (ROCK) inhibitor Y-27632 (10 μM; Selleckchem).

Lentiviral particles were produced by transfecting standard packaging vectors into HEK293T using TransIT-LT1 Transfection Reagent (Mirus, MIR2306). Media was changed 24 hours post-transfection with complete DMEM supplemented with 15 mM HEPES. Viral supernatants were harvested 48-60 hours after transfection and filtered through a 0.45 μm PVDF syringe filter. Lentiviral infections included polybrene (8 μg/ml).

### CRISPRoff Transfections and Analysis

Transient transfection experiments in HEK293T were performed in 24-well plates using TransIT-LT1 Transfection Reagent (Mirus) and Opti-MEM™ Reduced Serum Medium (Thermo Fisher Scientific). Cells at 70-80% confluency were transfected with 300 ng of plasmid encoding CRISPRoff, dCas9-KRAB, or dCas9-D3A-3L and 150 ng of plasmids encoding sgRNAs. CRISPRoff experiments in HeLa and U20S cells were performed by nucleofection of plasmids using the SE Cell Line 96-well Nucleofector Kit (Lonza) and a 96-well Shuttle™ Device (Lonza), per manufacturer protocol. Transfected cells were sorted 2 days after transfection using a BD FACSAria II or FACSAria Fusion and sorted cells were passaged every 2-3 days to measure durability of gene silencing. Experiments that compare the silencing activity of different CRISPRoff fusions (FIGS. 1E, 4C, 8B, and 8F) were performed in cells that stably express the targeting sgRNA to normalize sgRNA expression. To generate cell lines stably expressing sgRNAs, cells were transduced with lentiviral particles that express the sgRNAs and sorted for sgRNA-positive cells 2-3 days after transduction.

Quantification of ITGB1, CD81, and CD151 protein levels were measured by cell surface antibody staining of live cells. Cells were incubated with APC- or PE-labeled antibody (BioLegend) for ˜30 min in the dark at RT, washed twice with PBS containing 10% FBS, and protein expression was measured on a BD LSR II flow cytometer.

All flow cytometry data were analyzed using FlowJo and the raw FACS plots presented in the figures are in log 10 scale.

### PVT1 Enhancer Targeting

Quantitative RT-PCR quantification of PVT1 expression was done as described in (Cho et al., 2018). Briefly, MB-MDA-231 cells were transfected with CRISPR DNA together with a sgRNA vector using Neon (1400 volt, 10 ms, 4 pulse). Double positive cells were sorted after 2 days and continued to culture for 3 days. RNA were extracted with Zymo spin column and gene expression was quantified with SYBR qPCR mix (LightCycler 480) using 45 ng of RNA. The expression of PVT1 were normalized to GAPDH gene in ddCt method. T-test was used to calculate the statistical significance based on 3-5 biological replicates per condition. The primer sequence used are: PVT1 forward (SEQ ID NO:112), PVT1 reverse (SEQ ID NO:113), GAPDH forward (SEQ ID NO:114), GAPDH reverse (SEQ ID NO:115). The sgRNAs targeting PVT1 enhancers 1-4, promoter, and lambda (controls) were from (Cho et al., 2018) and listed on Table 5.

### RNA Sequencing

HEK293T cells that have maintained stable silencing of target genes were harvested 33 days (ITGB1, CD81, and CD151) or 28 days (CLTA, HIST2H2BE, RAB11A, and VIM) post CRISPRoff transfection. Cells were dislodged from plates with PBS, centrifuged at 500×g for 5 min and washed again with PBS. Total RNA was extracted using Direct-zol RNA MiniPrep (Zymo R2051). Library preparations were carried out using TruSeq Stranded mRNA Library Preparation Kit (Illumina RS-111-2101), starting with 1000 ng total RNA. Final libraries were assessed using a 2100 Bioanalyzer (Agilent), quantified using Qubit dsDNA HS Assay Kit (Thermo Fisher Scientific), and sequenced as single end 50 base pair reads on a HiSeq 4000 (Illumina). For processing the sequencing reads, linker sequences (SEQ ID NO:116) were removed using FASTX-clipper (FASTX-Toolkit). The reads were then aligned to the human genome (GRCh37) using the STAR (Spliced Transcripts Alignment to a Reference, version 2.5) aligner against the Gencode Gene V24lift37 transcriptome annotation. Read quantification was carried out with featureCounts (Liao et al., 2014). All downstream analyses were performed with Python (version 2.7) using a combination of Numpy (v1.12.1), Pandas (v0.17.1), and Scipy (v0.17.0) libraries. Knockdown efficiency was calculated by normalizing gene Transcripts per Million (TPM) for the experimental samples with the mean TPM of the control (non-targeting) samples. Differential expression analysis was performed using DESeq2 (Love et al., 2014). We note that non-target differentially expressed transcripts are lowly expressed genes.

### Chromatin Immunoprecipitation and Analysis

At 30 days post transfection, 10×10⁶ cells were crosslinked with 1% formaldehyde for 10 min at room temperature and quenched with 1.25 M glycine. Crosslinked cells were washed twice with cold PBS containing 1% Halt™ protease inhibitors (Thermo Fisher Scientific) and the cell pellets were flash frozen at −80° C. until sample preparation. Cells were lysed in lysis buffer (5 mM PIPES pH 8, 85 mM KCl, 1% Igepal, 1% protease inhibitors) for 10 min on ice. Nuclei were isolated after spinning the suspension at 2000 rpm at 4° C. for 5 min. Nuclei were lysed at 4° C. for 10 min in nuclei lysis buffer (50 mM Tris pH 8, 10 mM EDTA, 1% SDS, 1% protease inhibitors). Chromatin shearing was performed at 4° C. using a Diagenode Bioruptor® Pico sonication device in 1.5 ml Bioruptor® Pico Microtubes. The shearing program was optimized to obtain 200-700 bp fragments (30 seconds on, 30 seconds off for 10 cycles). The sonicated samples were centrifuged at 13,000 rpm at 4° C. for 10 min and the supernatant was collected and diluted 5-fold in IP dilution buffer (50 mM Tris pH 7.5, 150 mM NaCl, 1 mM EDTA, 1% Igepal, 0.25% deoxycholate, 1% protease inhibitors). A fraction of the input was saved and frozen (4% of total) prior to proceeding to immunoprecipitation. Immunoprecipitations were performed overnight at 4° C. using 5 μg of anti-H3K9me3 antibody (abcam ab8898). The washing steps were performed with Pierce™ Protein A/G magnetic beads (Thermo Fisher Scientific) using the following protocol: once with IP wash buffer 1 (20 mM Tris pH 8, 2 mM EDTA, 50 mM KCl, 1% Triton X-100, 0.1% SDS), twice with high salt buffer (20 mM Tris pH 8, 2 mM EDTA, 500 mM NaCl, 1% Triton X-100, 0.01% SDS), once with IP wash buffer 2 (10 mM Tris pH 8, 1 mM EDTA, 0.25 lithium chloride, 1% Igepal, 1% deoxycholate), and twice with TE buffer (10 mM Tris pH 8, 1 mM EDTA). Samples were eluted in elution buffer (50 mM sodium bicarbonate, 1% SDS) at 65° C. for 1 hour and reversed crosslinked initially in 300 mM NaCl and RNase A for 1 hour at 37° C., followed by 63° C. overnight with Proteinase K (Thermo Fisher). The DNA samples were purified using a Zymo Clean & Concentrator kit and libraries were prepared using the NEBNext® Ultra™ II DNA Library Prep kit (NEB).

Reads were aligned to the human genome (hg19) using bowtie v2.3.2 (Langmead and Salzberg, 2012). Alignments were processed using deepTools2 bamCoverage (Ramirez et al., 2016), normalizing reads to 1× average coverage. The resulting bigWig files were visualized on the Integrative Genomics Viewer (IGV). Peak analysis was performed using MACS (https://github.com/macs3-project/MACS). Downstream analysis and enrichment at promoter regions, which were defined as +/−2 kb of each TSS (with the TSSs based on previously published annotations (Horlbeck et al., 2016) were performed using Python 3.6 with the deepTools2 package. Differential H3K9me3 enrichment was analyzed using DESeq2 (Love et al., 2014), treating distinct sgRNAs against the same TSS as replicate samples. We note that BOLA1 contains two TSS annotations and our ChIP-seq data show enrichment for H3K9me3 near TSS1, the closest to the H2B promoter, and no enrichment for TSS2 located 15 kb away (FIGS. 2G-2H). We do not detect transcriptional knockdown of BOLA1 in our RNA-seq data.

### Western Blotting

Western blots of CRISPRoff constructs were performed by harvesting HEK293T cells 2 days post-transfection of CRISPRoff constructs. Cells were washed with cold PBS and lysed with RIPA buffer (Thermo Fisher Scientific) supplemented with Halt™ Protease Inhibitor Cocktail (Thermo Fisher Scientific). After 30 min of lysis at 4° C., the samples were centrifuged at 20,000×g for 20 min at 4° C. The soluble fractions were collected and protein concentrations were quantified by Pierce™ BCA Protein Assay Kit (Thermo Fisher Scientific). 40 μg of total protein was mixed and heated with SDS loading buffer, separated on Bolt™ 4-12% Bis-Tris Plus Gels (Thermo Fisher Scientific), and wet transferred into PVDF membrane in buffer containing 1×MOPS and 10% methanol. Membranes were blocked with Odyssey® Blocking Buffer (LI-COR), incubated with antibodies against S. pyogenes Cas9 (Active Motif 61577) and calnexin (Abcam ab22595) at 4° C. overnight. Membranes were washed at least 3 times with blocking buffer before incubation with IRDye® secondary antibodies against Cas9 and calnexin. Blots were imaged using Odyssey® CLx (LI-COR).

### Bisulfite Sequencing PCR

Genomic DNA was extracted from ˜1-2×10⁶ cells according to manufacturer's instructions using the PureLink Genomic DNA Mini Kit (Invitrogen). For each condition, 1 μg genomic DNA underwent bisulfite conversion and cleanup according to manufacturer's instructions using the EpiTect Bisulfite kit (Qiagen). Purified bisulfite-converted DNA was amplified using EpiMark Hot Start Taq (NEB). Amplicons were gel purified using a Gel DNA Recovery Kit (Zymo) and PCR amplified again using EpiMark Hot Start Taq. Amplicons were cloned into the pCR2.1 TOPO vector according to manufacturer's instructions using the TOPO TA Cloning Kit (Invitrogen). Cloning products were transformed into Stellar E. coli cells (Takara) and plated on carbenicillin plates with X-gal for blue-white screening. Colonies were picked per condition and sequenced by Sanger sequencing. Primer sequences for bisulfite-PCR amplification are listed in Table 6. The primer sequences for amplifying the GAPDH-Snrpn fragment was obtained from (Liu et al., 2016).

TABLE 6

| Name | Sequence (5′ to 3′) | SEQ ID NO: |
|---|---|---|
| CLTA, forward | TATTTGTTGATTGGGTAGTTTTTGAATT | SEQ ID NO: 184 |
| primer | | |
| CLTA, reverse | ACTCCCTAACCTCCTAATTCAACAAAAA | SEQ ID NO: 185 |
| primer | | |
| LAMP2, forward | ATAGGAAGGGTTGTGAATTAAAAAGTTAGG | SEQ ID NO: 186 |
| primer | | |
| LAMP2, reverse | ACAACTCACCCAAAACTAAACAAACCAAA | SEQ ID NO: 187 |
| primer | | |
| DYNC2LI1, | GAGATGTAAAAGGATTCCTGAAAATTATATTT | SEQ ID NO: 188 |
| forward primer | | |
| DYNC2LI1, | CTCTCTCCAAAAATACTCTCAATAACCTTTCC | SEQ ID NO: 189 |
| reverse primer | | |
| MYL6, forward | ATTTTAAGCGTTTGAGTGTTGCAGGTAGGG | SEQ ID NO: 190 |
| primer, fragment 1 | | |
| MYL6, reverse | TAAAACAATAAATAACCTCCTAATAACTAAAA | SEQ ID NO: 191 |
| primer, fragment 1 | | |
| MYL6, forward | TTTGATTTTTTTGCTGTTGGGAGGTGTAGAT | SEQ ID NO: 192 |
| primer, fragment 2 | | |
| MYL6, reverse | CAACTCTCCGAAACCTTTTCCTACAATAAT | SEQ ID NO: 193 |
| primer, fragment 2 | | |
| CD81, forward | AAGTTGTGGGGttAttTGTGGGtTttAGGAG | SEQ ID NO: 194 |
| primer | | |
| CD81, reverse | CCCaCaACCACCaCACCCATCACCACCACA | SEQ ID NO: 195 |
| primer | | |
| MAPT, forward | AtAAAGAtTttAAtTAtAGGAGGTGGAGAAAG | SEQ ID NO: 196 |
| primer | | |
| MAPT, reverse | aaAaCCTTCTCCTCCaaCCACTAaT | SEQ ID NO: 197 |
| primer | | |
| H2B, site 1, | tAGTGGtATGGGGGAGtAGGATAAGAAAAG | SEQ ID NO: 198 |
| forward primer | | |
| H2B, site 1, | aATCCCCTTaAaCTCTAAACTaCCAAaTC | SEQ ID NO: 199 |
| reverse primer | | |
| H2B, site 2, | TCCTTGTAAAAGAAGITGTAAtTGtAATTTTAGt | SEQ ID NO: 200 |
| forward primer | | |
| H2B, site 2, | TaaTaACACCCCCaAaTAACTTaTTaAaCTCTT | SEQ ID NO: 201 |
| reverse primer | | |
| H2B, site 3, | AttAAGAGGAAAGttTTTATAAGttTTTttTAGGGG | SEQ ID NO: 202 |
| forward primer | | |
| H2B, site 3, | CCTaATTCTTTTATAACCACCTTATaCAAATTAaa | SEQ ID NO: 203 |
| reverse primer | aCTC | |

### Whole Genome Bisulfite Sequencing and Analysis

We generated whole genome bisulfite sequencing (WGBS) libraries for 12 samples, corresponding to WT (untreated), NT (non-targeting), and T (targeting) for CLTA and DYNC2LI1 experiments and profiled in two replicates. After DNA extraction and RNAse A treatment using the PureLink Genomic DNA Mini Kit (Invitrogen), 1 μg of DNA was diluted to 7.7 ng/μL in 130 uL and sheared to 450-550 bp length using a Covaris E220 evolution with intensifier for 50s at 140V, 7° C., 10% amplitude, 200 cycles. The sonicated DNA was recovered and concentrated using Ampure XP beads and sizes of the sheared DNA were checked on an Agilent TapeStation device with a D1000 HS DNA ScreenTape. Next, the sheared DNA was bisulfite converted using the EZ DNA Lightning kit (Zymo, Cat. No. D5030) according to the manufacturer's instructions and a desulphonation step of 16 minutes. Then, 500 ng of sheared and converted DNA was subjected to library preparation using a Swift Accel®-NGS Methyl-Seq DNA Library Kit (Cat. No. 30024) and Methyl-Seq Unique Dual Indexing Primers (Cat. No. 39096). The prepared libraries were quantified using a KAPA Library Quantification kit (Roche, Cat. No. KK4873) and sequenced using paired-end 150 bp reads (300 cycles) on an Illumina NovaSeq6000 instrument with an S4 flow cell and a 35% spike-in from another non-WGBS library to diversify the sample pools. We obtained on average 707M paired-end reads (range 629-835M) across all 12 libraries.

Prior to alignment, sequencing reads were trimmed using fastp (version 0.21.0, (Chen et al., 2018)) and the following parameters: --adapter_sequence=(SEQ ID NO:117) --adapter_sequence_r2=(SEQ ID NO:118) --trim_front1=0 --trim_tail1=20 --trim_front2=20 --trim tail2=0. Trimming is required to remove bases that are added during the Adaptase reaction that could affect alignment and DNA methylation calling. Processed reads were aligned to the hg38 reference genome using methylCtools (version 1.0.0, https://github.com/hovestadt/methylCtools, (Hovestadt et al., 2014)) and bwa mem (version 0.7.17, arXiv: 1303.3997v1) using default parameters. Over 98% of reads were aligned as proper pairs across samples. After marking of PCR duplicates using sambamba (version 0.8.0, (Tarasov et al., 2015)), genome-wide CpG methylation values were called using methylCtools using the --trimPE parameter. Average CpG coverage was ˜25-fold across samples. Bisulfite conversion efficiency was estimated to be greater than 99.5% based on non-CpG methylation.

Downstream analyses were performed in R (version 4.0.2, https://www.r-project.org/) using the bsseq (version 1.24.4, (Hansen et al., 2012)) and DSS (version 2.36.0, (Park and Wu, 2016)) packages. Specifically, we applied the DMLtest function to first call differentially methylated loci (using 500 bp smoothing windows) between treatments, and then the callDMR function to define differentially methylated regions. Results were visualized by plotting log 10-transformed p-values associated with individual loci (positive values for loci that gained methylation, negative values for loci that lost methylation). Loci were colored by their difference in beta-values (−1: blue, 0: white, +1: red). Close-ups of genomic regions were generated by visualizing beta-values of individual loci in IGV (Robinson et al., 2011). Data was displayed as bar charts (min/0: blue, mid/0.5, max/1: red).

### Genome-Wide CRISPRoff Screen and Analysis

For genome-wide CRISPRoff screens, we constructed a compact library to maximize on-target knockdown while minimizing overall library size. We targeted each gene in the human genome with two unique sgRNAs expressed from tandem U6 expression cassettes in a single vector. To select the optimal sgRNAs targeting each gene, we relied on our previously published hCRISPRi v2.1 library (Horlbeck et al., 2016a). A three tiered approach was used to balance empirical data with computational predictions and select the most active sgRNA pair for each gene. First, for strong essential genes (p-value<0.001 and gamma<−0.2 in hCRISPRi v2 growth screen), sgRNAs were ranked by growth. Next, for genes that were identified as a significant hit in previous CRISPRi screens, sgRNAs were ranked by the sum of Z-scored phenotypes across screens. Finally, for all other genes, sgRNAs were ranked by the regression scores in hCRISPRi v2.1. Using this ranking scheme, we designed a genome-wide library consisting of only 21378 elements (20360 targeting elements plus 1018 non-targeting controls).

To clone our libraries, we began by generating a modified single sgRNA lentiviral expression vector, pJR104, from the parental pJR85 (Addgene 140095) by: (i) replacing the BFP fluorescent marker with a BsmBI-negative GFP, (ii) replacing the sgRNA constant region with an unmodified constant region (i.e. without a Perturb-seq capture sequence), and (iii) incorporating a UCOE element upstream of the EF1alpha promoter to prevent silencing. Dual-sgRNA oligos were synthesized as an oligonucleotide pool (Twist Biosciences) with the structure: 5′-PCR adapter-SEQ ID NO:126-protospacer A-SEQ ID NO:119-protospacer B-SEQ ID NO: 120-PCR adapter-3′. Oligo pools were PCR-amplified, digested with BstXI/BlpI, gel extracted, ligated into the sgRNA lentiviral vector pJR104, and transformed to generate an intermediate library as previously described (Replogle et al., 2020). An insert, pJR98, consisting of a sgRNA constant region variant 3 (Adamson et al., 2016) and a hU6 promoter was synthesized (IDT), BsmBI-digested, and ligated into the BsmBI-digested intermediate library. The final dual-guide library was then transformed for amplification and sequenced by next-generation sequencing to ensure library representation and uniformity.

Pooled tiling sgRNA screens in HEK293T cells were performed by first transducing cells with lentiviral particles encoding the sgRNA library. The infection efficiency was measured 2 days post-infection by flow cytometry, aiming for 20-30% sgRNA-positive cells. The screens were performed with two technical replicates and each sgRNA was represented by at least 1000 cells throughout the duration of the screens. Two days post transduction, cells were treated with puromycin until the cell population was 90% sgRNA positive, as marked by mCherry encoded in the lentiviral vector. For transient transfection of CRISPRoff, ˜8×10⁶ cells were first seeded on 15 cm² plates. About 20-24 hr later (70-80% confluency), each 15 cm² plate of cells were transfected with 20 μg of plasmids encoding CRISPRoff or CRISPRoff-Dnmt3A^{E765A} catalytic mutant. Two days post-tranSfection, cells were sorted for CRISPRoff expression (BFP) and plated on 15 cm² plates. Four days post-sorting, cells were trypsinized and an aliquot of cells (˜110×10⁶) was harvested as an initial time point T(0) and the rest of the cell population was passaged for at least 10 more cell doublings. Cells were then collected as a final time point (T10).

DNA libraries of T(0) and T(10) were prepared for deep sequencing essentially as previously described (Jost et al., 2020). Briefly, genomic DNA was isolated using a NucleoSpin Blood XL kit (Macherey-Nagel). Then, isolated gDNA was directly amplified by 23 cycles of PCR using NEBNext Ultra II Q5 PCR MasterMix (NEB), appending Illumina adaptors and unique sample indices (oJR234 forward primer: SEQ ID NO:121; index primers SEQ ID NO:122). Sequencing was performed on a NovaSeq 6000 (Illumina) using a 19 bp read 1, 19 bp read 2, and 5 bp index read 1 with custom sequencing primers oJR326 (custom read 1, SEQ ID NO:123), oJR328 (custom read 2, SEQ ID NO:124), and oJR327 (custom index read 1, SEQ ID NO:125).

Sequencing counts from CRISPR screens were processed to calculate gene phenotypes using a custom Python script, similar to as previously described (Horlbeck et al., 2016a), except now two protospacer sequences are matched instead of just one. In this case, gene phenotype is the same as sgRNA phenotype, and is defined by log 2 sgRNA enrichment/cell doublings. All additional CRISPR screen data analyses and plotting were performed in Python 3.6 using a combination of Numpy (v1.16.2), Pandas (v0.23.4), Scipy (v1.4.1), and sklearn (v0.22.2). The DepMap essential and nonessential genes were downloaded from DepMap Public 20Q2 at https://depmap.org/portal/download/ (Blomen et al., 2015; Hart et al., 2014). Gene set enrichment analysis (GSEA) was performed using GSEAPY (v0.9.19) in Python using the 2019 Human KEGG Pathway database.

### Tiling Screen Library Design, Experimental Specifications, and Analysis

For the growth-based screen, a tiling sgRNA library targeting essential genes was designed based on our previously published genome-wide CRISPRi screen in K562s. For genes with no canonical CGIs (as defined by no CGIs within 2.5 kb of TSS, with the TSSs based on previously published annotations (Horlbeck et al., 2016a) and CGI annotations from the UCSC Genome Browser), all genes with an average growth phenotype score less than −0.2 were picked. For genes with one or multiple CGIs (also defined as within 2.5 kb of TSS), genes with an average growth phenotype score between −0.2 and −0.4 were selected. In total, 39 genes with no canonical CGIs, 425 genes with one annotated CGI, and 56 genes with multiple CGIs were chosen.

For each gene, all sequences +/−2.5 kb (or +/−1 kb depending on the position and length of the CGI) of the TSS containing 19 bp followed by an NGG PAM were extracted as potential sgRNAs. All sequences were prepended with a 5′ G to enable robust transcription from the U6 promoter, whether or not this base was present in the genomic sequence. The sgRNAs were scored for off-target sites using weighted Bowtie, as previously described (Horlbeck et al., 2016a). Briefly, sgRNAs were scored by uniqueness in the genome, as determined by an empirically derived and experimentally verified scoring metric: PAM G1=40, PAM G2=19, PAM N=0, the next 7 bases from the PAM=28, the next 5 bases=19, and the last 7 bases=10. A mismatch score was then calculated by the sum of the mismatches with the scoring metric. This mismatch score was implemented using the Phred score threshold feature of Bowtie using the --nomaqround, -n 3, -115, -a, and --best flags. For the most stringent threshold, sgRNAs were required to have no more than 1 alignment (the sgRNA target site itself) in the genome with a mismatch score of 39. Control non-targeting sgRNAs were extracted from a previously tested list of control sgRNAs (Horlbeck et al., 2016a).

The tiling libraries for endogenously GFP-tagged CLTA, HIST2H2BE (H2B), RAB11A, and VIM were designed similarly, selecting for sgRNAs+/−2.5 kb from the TSS and yielding ˜600 sgRNAs per gene. The protospacer sequences for each GFP-tagged gene library are available in Table 5.

Oligonucleotide pools were designed with flanking PCR and restriction sites (BstXI and BlpI), synthesized by Agilent Technologies, and cloned into the sgRNA expression vector pCRISPRia-v2 (Addgene #84832), as described previously (Horlbeck et al., 2016a). The expression vector contains a U6 promoter driving the sgRNA expression, as well as an EF1α promoter driving puromycin T2A-mCherry.

The tiling screens in HEK293 Ts were performed in a similar workflow as the genome-wide CRISPRoff screens. To perform the tiling screen in K562s, cells stably expressing dCas9-KRAB were first transduced with lentiviral particles of the tiling sgRNA library. Two days post transduction (20-30% infection), cells were treated with puromycin until the population consisted of 90% sgRNA-expressing cells. A T(0) time point was then collected and cells were continued to passage for 10 more cell doublings to obtain the T(10) time point.

Sequencing counts from CRISPR screens were processed using the Python-based ScreenProcessing pipeline (https://github.com/mhorlbeck/ScreenProcessing), as previously described (Horlbeck et al., 2016a) to calculate sgRNA phenotypes. sgRNA phenotype score is defined by log 2 sgRNA enrichment/cell doublings. All additional CRISPR screen data analyses and plotting were performed in Python 2.7 using a combination of Numpy (v1.12.1), Pandas (v0.17.1), and Scipy (v0.17.0). K562 and GM12878 MNase-seq data was obtained from the ENCODE consortium as processed continuous signal data (BigWig file format; Michael Snyder lab, Stanford University). The average of the K562 and GM12878 MNase-seq data was used. We note that the phenotypes in K562 CRISPRi (median γ=−0.46) are more pronounced compared to the HEK293T CRISPRoff screen (median γ=−0.33). However, as we have previously demonstrated, because the genes were chosen based on essentiality in K562s, this difference likely can be attributed to cell type variability between K562 and HEK293T.

GFP-Tagged sgRNA Tiling Screen

The tiling sgRNA screens in HEK293T GFP-tagged cell lines were performed in a similar workflow as the growth-based screens described above. The previously published endogenously GFP-tagged cell lines (CLTA, HIST2H2BE, RAB11A, VIM) were further FACS sorted to yield >99% GFP-positive cells to minimize background GFP-negative cells. After generating cell lines that stably express the respective sgRNA library, plasmids expressing CRISPRoff were transfected. Two days later, the transfected cells were sorted and subsequently passaged for 4 weeks by trypsinization every 2-3 days. At the 4 weeks time point, each cell line had the following detectable GFP-silenced population: 21.8% CLTA, 22.7% HIST2H2BE, 3.05% RAB11A, and 24.7% VIM. The GFP-on and GFP-off populations were FACS sorted into separate bins, collecting ˜2×10⁶ cells per population for each cell line. The log 2 fold change in sgRNA abundance was quantified by the presence of each sgRNA in the GFP-off population compared to the total population. Analysis was performed using Python 2.7, similar to the other tiling screens described previously.

iPSC Manipulation and Neuronal Differentiation

Transient transfections of iPSCs were performed in 6-well plates using TransIT-LT1 Transfection Reagent (Mirus). First, a mixture of 0.5 ml of mTeSR1 and 2 μl of 10 mM Y-27632 ROCK inhibitor were added to each well of a Matrigel coated 6-well plate. Then, a mixture of plasmids encoding dCas9-KRAB or CRISPRoff (1 μg), 1 μg of sgRNA plasmids, and 200 ng of plasmid encoding BCL-XL (Li et al., 2018) were added to 0.4 ml of Opti-MEM™. TransIT-LT1 (12 μl) was added to the DNA-Opti-MEM™ mixture and added to each well of a 6-well plate. Cells at 70-80% confluence were lifted with Accutase, washed with DPBS, and counted using a Countess (Thermo Fisher AMQAX1000). About 1.5×10⁶ cells were resuspended in 1 ml of mTeSR1 and added to each well containing the transfection mixtures. Transfected cells were sorted 3 days post-transfection on a BD FACS Fusion and plated in mTeSR media supplemented with 10 μM Y-27632 ROCK inhibitor.

Neuronal differentiations were performed on passage number 46 iPSCs using doxycycline-inducible NGN2 (Tian et al., 2019). On day −3, cells at 70-80% confluency were lifted with Accutase and washed with DPBS. About 7.5×10⁵ cells were resuspended in 2 ml of N2 Pre-differentiation Media each well of a Matrigel coated 6-well plate. On day 0, cells were lifted with Accutase and washed with DPBS. About 5×10⁵ cells were resuspended in 2 ml classic N2/B27 Differentiation Media and plated onto Poly-D-Lysine coated plates (Corning 354413). On day 3, the media in each well were aspirated and replaced with 2 ml of fresh N2/B27 Differentiation Media. On day 7, 1 ml of media was removed from each well and replaced with 1 ml of fresh N2/B27 Differentiation Media. N2 Pre-differentiation Media was made with 1× Knockout DMEM/F12 (Thermo Fisher 11320-033), 1×NEAA (Thermo Fisher 11140-050), 1×N2 Supplement (Thermo Fisher 17502-048), 10 ng/ml NT-3 (PreproTech 450-03), 10 ng/ml BDNF (PreproTech 450-02), 1 μg/ml Mouse Laminin (Thermo Fisher 23017-015), 10 nM Y-27632 ROCK inhibitor, and 2 μg/ml doxycycline (Sigma-Aldrich D3447-500MG). Classic N2/B27 Differentiation Media was made with 0.5×DMEM/F12 (Thermo Fisher 10888-033), 0.5× Neurobasal-A (10888-022), 1×NEAA, 0.5× GlutaMAX (Thermo Fisher 35050-061), 0.5×N2 Supplement, 0.5×B27-VA Supplement (Thermo Fisher 12587010), 10 ng/ml NT-3, 10 ng/ml BDNF, 1 μg/ml Mouse Laminin, and 2 μg/ml doxycycline.

We used iNeuron RNA-Seq (https://kampmannlab.ucsf.edu/ineuron-ma-seq) to support the activation of MAPT gene expression through neuronal differentiation of iPS cells.

### Informal Sequence Listing

In the sequences listed herein, the skilled artisan will appreciate that a methionine (M) can be present on the N-terminus of protein in order to initiate translation. Thus, SEQ ID NOS: 1-15 can optionally further comprise a methionine on the N-terminus.

SEQ ID NO: 1-CRISPRoff-V1 or V1 or P76 (described in WO 2019/204766)): KRAB
(bold; from Gilbert et al., Cell, 2013, 2014), Linkers (underlined), dCas9 (italics), HA tag
(lowercase), SV40 NLS (lowercase italics), Dnmt3A (bold italics; residues 612-912; from
Siddique et al., JMB, 2013; Stepper et al., NAR, 2016), 27 amino acid linker (italics under-
lined; from Siddique et al., JMB, 2013; Stepper et al., NAR, 2016), Dnmt3L (bold underlined;
from Siddique et al., JMB, 2013; Stepper et al., NAR, 2016), P2A peptide cleavage sequence
(lowercase bold), BFP (lowercase underlined))
DAKSLTAWSRTLVTFKDVFVDFTREEWKLLDTAQQIVYRNVMLENYKNLVSLGY
QLTKPDVILRLEKGEEPGGSGGGSMDKKYSIGLAIGTNSVGWAVITDEYKVPSKKFKVLG
NTDRHSIKKNLIGALLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKADLRLIYLALAHMIKF
RGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLF
LAAKNLSDAILLSDILRVNTEITKAPLSASMIKRYDEHHQDLTLLKALVRQQLPEKYKEIFFDQS
KNGYAGYIDGGASQEEFYKFIKPILEKMDGTEELLVKLNREDLLRKQRTFDNGSIPHQIHLGE
LHAILRRQEDFYPFLKDNREKIEKILTFRIPYYVGPLARGNSRFAWMTRKSEETITPWNFEEVV
DKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFL
DNEENEDILEDIVLTLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMKRIEEGIKELGSQI
LKEHPVENTQLQNEKLYLYYLQNGRDMYVDQELDINRLSDYDVDAIVPQSFLKDDSIDNKVL
TRSDKNRGKSDNVPSEEVVKKMKNYWRQLLNAKLITQRKFDNLTKAERGGLSELDKAGFIKR
QLVETRQITKHVAQILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKATAKYFFYSNIMNF
FKTEITLANGEIRKRPLIETNGETGEIVWDKGRDFATVRKVLSMPQVNIVKKTEVQTGGFSKE
SILPKRNSDKLIARKKDWDPKKYGGFDSPTVAYSVLVVAKVEKGKSKKLKSVKELLGITIMERS
SFEKNPIDFLEAKGYKEVKKDLIIKLPKYSLFELENGRKRMLASAGELQKGNELALPSKYVNF
LYLASHYEKLKGSPEDNEQKQLFVEQHKHYLDEIIEQISEFSKRVILADANLDKVLSAYNKHR
DKPIREQAENIIHLFTLTNLGAPAAFKYFDTTIDRKRYTSTKEVLDATLIHQSITGLYETRIDLSQ
LGGDSRADypydvpdyaSGSpkkkrkvEASGSGRASPGIPGSTRNHDQEFDPPKVYPPVPAEKR
KPIRVLSLFDGIATGLLVLKDLGIQVDRYIASEVCEDSITVGMVRHQGKIMYVGDVRSVT
QKHIQEWGPFDLVIGGSPCNDLSIVNPARKGLYEGTGRLFFEFYRLLHDARPKEGDDR
PFFWLFENVVAMGVSDKRDISRFLESNPVMIDAKEVSAAHRARYFWGNLPGMNRPLA
STVNDKLELQECLEHGRIAKFSKVRTITTRSNSIKQGKDQHFPVFMNEKEDILWCTEME
RVFGFPVHYTDVSNMSRLARQRLLGRSWSVPVIRHLFAPLKEYFACVSSGNSNANSRGPS
FSSGLVPLSLRGSHMGPMEIYKTVSAWKRQPVRVLSLFRNIDKVLKSLGFLESGSGSG
GGTLKYVEDVTNVVRRDVEKWGPFDLVYGSTQPLGSSCDRCPGWYMFQFHRILQ
YALPRQESQRPFFWIFMDNLLLTEDDQETTTRFLQTEAVTLQDVRGRDYQNAMRV
WSNIPGLKSKHAPLTPKEEEYLQAQVRSRSKLDAPKVDLLVKNCLLPLREYFKYFS
QNSLPLSRADpkkkrkvGSGatnfsllkqagdveenpgpselikenmhmklymegtvdnhhfkctsegegkpyegtqt
mrikvveggplpfafdilatsflygsktfinhtqgipdffkqsfpegftwervttyedggvltatqdtslqdgcliynvkirgvnftsngpv
mqkktlgweaftetlypadgglegrndmalklvggshlianikttyrskkpaknlkmpgvyyvdyrlerikeannetyveghevav
arycdlpsklghkln*
SEQ ID NO: 2 (p90 (KRAB-dCas9-XTEN16-Dnmt3A-Dnmt3L-P2A-BFP): KRAB
(bold, from Gilbert et al., Cell, 2013, 2014); Linkers (underlined), dCas9 (italics); HA tag
(lowercase), SV40 NLS (lowercase italics), XTEN16 (uppercase, 16 amino acid sequence),
Dnmt3A (bold italics; from Siddique et al., JMB, 2013; Stepper et al., NAR, 2016), 27 amino
acid linker (italics underlined, from Siddique et al., JMB, 2013; Stepper et al., NAR, 2016),
Dnmt3L (bold underlined, from Siddique et al., JMB, 2013; Stepper et al., NAR, 2016), P2A
peptide cleavage sequence (lowercase bold), BFP (lowercase underlined))
DAKSLTAWSRTLVTFKDVFVDFTREEWKLLDTAQQIVYRNVMLENYKNLVSLGY
QLTKPDVILRLEKGEEPGGSGGGSMDKKYSIGLAIGTNSVGWAVITDEYKVPSKKFKVLG
NTDRHSIKKNLIGALLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKADLRLIYLALAHMIKF
RGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLF
LAAKNLSDAILLSDILRVNTEITKAPLSASMIKRYDEHHQDLTLLKALVRQQLPEKYKEIFFDQS
KNGYAGYIDGGASQEEFYKFIKPILEKMDGTEELLVKLNREDLLRKQRTFDNGSIPHQIHLGE
LHAILRRQEDFYPFLKDNREKIEKILTFRIPYYVGPLARGNSRFAWMTRKSEETITPWNFEEVV
DKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFL
DNEENEDILEDIVLTLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMKRIEEGIKELGSQI
LKEHPVENTQLQNEKLYLYYLQNGRDMYVDQELDINRLSDYDVDAIVPQSFLKDDSIDNKVL
TRSDKNRGKSDNVPSEEVVKKMKNYWRQLLNAKLITQRKFDNLTKAERGGLSELDKAGFIKR
QLVETRQITKHVAQILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKATAKYFFYSNIMNF
FKTEITLANGEIRKRPLIETNGETGEIVWDKGRDFATVRKVLSMPQVNIVKKTEVQTGGFSKE
SILPKRNSDKLIARKKDWDPKKYGGFDSPTVAYSVLVVAKVEKGKSKKLKSVKELLGITIMERS
SFEKNPIDFLEAKGYKEVKKDLIIKLPKYSLFELENGRKRMLASAGELQKGNELALPSKYVNF
LYLASHYEKLKGSPEDNEQKQLFVEQHKHYLDEIIEQISEFSKRVILADANLDKVLSAYNKHR
DKPIREQAENIIHLFTLTNLGAPAAFKYFDTTIDRKRYTSTKEVLDATLIHQSITGLYETRIDLSQ
LGGDSRADypydvpdyaSGSpkkkrkvSPGSGSETPGTSESATPESNHDQEFDPPKVYPPVPAE
KRKPIRVLSLFDGIATGLLVLKDLGIQVDRYIASEVCEDSITVGMVRHQGKIMYVGDVRS
VTQKHIQEWGPFDLVIGGSPCNDLSIVNPARKGLYEGTGRLFFEFYRLLHDARPKEGD
DRPFFWLFENVVAMGVSDKRDISRFLESNPVMIDAKEVSAAHRARYFWGNLPGMNRP
LASTVNDKLELQECLEHGRIAKFSKVRTITTRSNSIKQGKDQHFPVFMNEKEDILWCTE
MERVFGFPVHYTDVSNMSRLARQRLLGRSWSVPVIRHLFAPLKEYFACVSSGNSNANSR
GPSFSSGLVPLSLRGSHMGPMEIYKTVSAWKRQPVRVLSLFRNIDKVLKSLGFLESGS
GSGGGTLKYVEDVTNVVRRDVEKWGPFDLVYGSTQPLGSSCDRCPGWYMFQFHR
ILQYALPRQESQRPFFWIFMDNLLLTEDDQETTTRFLQTEAVTLQDVRGRDYQNA
MRVWSNIPGLKSKHAPLTPKEEEYLQAQVRSRSKLDAPKVDLLVKNCLLPLREYF
KYFSQNSLPLSRADpkkkrkvGSGatnfsllkqagdveenpgpselikenmhmklymegtvdnhhfkctsegegk
pyegtqtmrikvveggplpfafdilatsflygsktfinhtqgipdffkqsfpegftwervttyedggvltatqdtslqdgcliynvkirgvn
ftsngpvmqkktlgweaftetlypadgglegrndmalklvggshlianikttyrskkpaknlkmpgvyyvdyrlerikeannetyve
ghevavarycdlpsklghkln*
SEQ ID NO: 3 (p91 (KRAB-dCas9-Dnmt3A-Dnmt3L-P2A-P2A-BFP): KRAB (bold,
from Gilbert et al., Cell, 2013, 2014), Linkers (underlined), dCas9 (italics), HA tag
(lowercase), SV40 NLS (lowercase italics), Dnmt3A (bold italics, from Siddique et al., JMB,
2013; Stepper et al., NAR, 2016), 27 amino acid linker (italics underlined, from Siddique et
al., JMB, 2013; Stepper et al., NAR, 2016), Dnmt3L (bold underlined, from Siddique et al.,
JMB, 2013; Stepper et al., NAR, 2016), P2A peptide cleavage sequence (lowercase bold),
BFP (lowercase underlined))
DAKSLTAWSRTLVTFKDVFVDFTREEWKLLDTAQQIVYRNVMLENYKNLVSLGY
QLTKPDVILRLEKGEEPGGSGGGSMDKKYSIGLAIGTNSVGWAVITDEYKVPSKKFKVLG
NTDRHSIKKNLIGALLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKADLRLIYLALAHMIKF
RGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLF
LAAKNLSDAILLSDILRVNTEITKAPLSASMIKRYDEHHQDLTLLKALVRQQLPEKYKEIFFDQS
KNGYAGYIDGGASQEEFYKFIKPILEKMDGTEELLVKLNREDLLRKQRTFDNGSIPHQIHLGE
LHAILRRQEDFYPFLKDNREKIEKILTFRIPYYVGPLARGNSRFAWMTRKSEETITPWNFEEVV
DKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFL
DNEENEDILEDIVLTLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMKRIEEGIKELGSQI
LKEHPVENTQLQNEKLYLYYLQNGRDMYVDQELDINRLSDYDVDAIVPQSFLKDDSIDNKVL
TRSDKNRGKSDNVPSEEVVKKMKNYWRQLLNAKLITQRKFDNLTKAERGGLSELDKAGFIKR
QLVETRQITKHVAQILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKATAKYFFYSNIMNF
FKTEITLANGEIRKRPLIETNGETGEIVWDKGRDFATVRKVLSMPQVNIVKKTEVQTGGFSKE
SILPKRNSDKLIARKKDWDPKKYGGFDSPTVAYSVLVVAKVEKGKSKKLKSVKELLGITIMERS
SFEKNPIDFLEAKGYKEVKKDLIIKLPKYSLFELENGRKRMLASAGELQKGNELALPSKYVNF
LYLASHYEKLKGSPEDNEQKQLFVEQHKHYLDEIIEQISEFSKRVILADANLDKVLSAYNKHR
DKPIREQAENIIHLFTLTNLGAPAAFKYFDTTIDRKRYTSTKEVLDATLIHQSITGLYETRIDLSQ
LGGDSRADypydvpdyaSGSpkkkrkvEASGSGRASPGIPGSTRNHDQEFDPPKVYPPVPAEKR
KPIRVLSLFDGIATGLLVLKDLGIQVDRYIASEVCEDSITVGMVRHQGKIMYVGDVRSVT
QKHIQEWGPFDLVIGGSPCNDLSIVNPARKGLYEGTGRLFFEFYRLLHDARPKEGDDR
PFFWLFENVVAMGVSDKRDISRFLESNPVMIDAKEVSAAHRARYFWGNLPGMNRPLA
STVNDKLELQECLEHGRIAKFSKVRTITTRSNSIKQGKDQHFPVFMNEKEDILWCTEME
RVFGFPVHYTDVSNMSRLARQRLLGRSWSVPVIRHLFAPLKEYFACVSSGNSNANSRGPS
FSSGLVPLSLRGSHMGPMEIYKTVSAWKRQPVRVLSLFRNIDKVLKSLGFLESGSGSG
GGTLKYVEDVTNVVRRDVEKWGPFDLVYGSTQPLGSSCDRCPGWYMFQFHRILQ
YALPRQESQRPFFWIFMDNLLLTEDDQETTTRFLQTEAVTLQDVRGRDYQNAMRV
WSNIPGLKSKHAPLTPKEEEYLQAQVRSRSKLDAPKVDLLVKNCLLPLREYFKYFS
QNSLPLSRADpkkkrkvGSGatnfsllkqagdveenpgpGSGatnfsllkqagdveenpgpselikenmhmklym
egtvdnhhfkctsegegkpyegtqtmrikvveggplpfafdilatsflygsktfinhtqgipdffkqsfpegftwervttyedggvltatq
dtslqdgcliynvkirgvnftsngpvmqkktlgweaftetlypadgglegrndmalklvggshlianikttyrskkpaknlkmpgvy
yvdyrlerikeannetyveghevavarycdlpsklghkln*
SEQ ID NO: 4 (p92 (KRAB-dCas9-XTEN16-Dnmt3A-Dnmt3L-P2A-P2A-BFP):
KRAB (bold, from Gilbert et al., Cell, 2013, 2014), Linkers (underlined), dCas9 (italics), HA
tag (lowercase), SV40 NLS (lowercase italics), XTEN16 (16 amino acid sequence), Dnmt3A
(bold italics, from Siddique et al., JMB, 2013; Stepper et al., NAR, 2016), 27 amino acid linker
(italics underlined, from Siddique et al., JMB, 2013; Stepper et al., NAR, 2016), Dnmt3L (bold
underlined, from Siddique et al., JMB, 2013; Stepper et al., NAR, 2016), P2A peptide cleavage
sequence (lowercase bold), BFP (lowercase underlined))
DAKSLTAWSRTLVTFKDVFVDFTREEWKLLDTAQQIVYRNVMLENYKNLVSLGY
QLTKPDVILRLEKGEEPGGSGGGSMDKKYSIGLAIGTNSVGWAVITDEYKVPSKKFKVLG
NTDRHSIKKNLIGALLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKADLRLIYLALAHMIKF
RGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLF
LAAKNLSDAILLSDILRVNTEITKAPLSASMIKRYDEHHQDLTLLKALVRQQLPEKYKEIFFDQS
KNGYAGYIDGGASQEEFYKFIKPILEKMDGTEELLVKLNREDLLRKQRTFDNGSIPHQIHLGE
LHAILRRQEDFYPFLKDNREKIEKILTFRIPYYVGPLARGNSRFAWMTRKSEETITPWNFEEVV
DKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFL
DNEENEDILEDIVLTLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMKRIEEGIKELGSQI
LKEHPVENTQLQNEKLYLYYLQNGRDMYVDQELDINRLSDYDVDAIVPQSFLKDDSIDNKVL
TRSDKNRGKSDNVPSEEVVKKMKNYWRQLLNAKLITQRKFDNLTKAERGGLSELDKAGFIKR
QLVETRQITKHVAQILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKATAKYFFYSNIMNF
FKTEITLANGEIRKRPLIETNGETGEIVWDKGRDFATVRKVLSMPQVNIVKKTEVQTGGFSKE
SILPKRNSDKLIARKKDWDPKKYGGFDSPTVAYSVLVVAKVEKGKSKKLKSVKELLGITIMERS
SFEKNPIDFLEAKGYKEVKKDLIIKLPKYSLFELENGRKRMLASAGELQKGNELALPSKYVNF
LYLASHYEKLKGSPEDNEQKQLFVEQHKHYLDEIIEQISEFSKRVILADANLDKVLSAYNKHR
DKPIREQAENIIHLFTLTNLGAPAAFKYFDTTIDRKRYTSTKEVLDATLIHQSITGLYETRIDLSQ
LGGDSRADypydvpdyaSGSpkkkrkvSPGSGSETPGTSESATPESNHDQEFDPPKVYPPVPAE
KRKPIRVLSLFDGIATGLLVLKDLGIQVDRYIASEVCEDSITVGMVRHQGKIMYVGDVRS
VTQKHIQEWGPFDLVIGGSPCNDLSIVNPARKGLYEGTGRLFFEFYRLLHDARPKEGD
DRPFFWLFENVVAMGVSDKRDISRFLESNPVMIDAKEVSAAHRARYFWGNLPGMNRP
LASTVNDKLELQECLEHGRIAKFSKVRTITTRSNSIKQGKDQHFPVFMNEKEDILWCTE
MERVFGFPVHYTDVSNMSRLARQRLLGRSWSVPVIRHLFAPLKEYFACVSSGNSNANSR
GPSFSSGLVPLSLRGSHMGPMEIYKTVSAWKRQPVRVLSLFRNIDKVLKSLGFLESGS
GSGGGTLKYVEDVTNVVRRDVEKWGPFDLVYGSTQPLGSSCDRCPGWYMFQFHR
ILQYALPRQESQRPFFWIFMDNLLLTEDDQETTTRFLQTEAVTLQDVRGRDYQNA
MRVWSNIPGLKSKHAPLTPKEEEYLQAQVRSRSKLDAPKVDLLVKNCLLPLREYF
KYFSQNSLPLSRADpkkkrkvGSGatnfsllkqagdveenpgpGSGatnfsllkqagdveenpgpselikenmh
mklymegtvdnhhfkctsegegkpyegtqtmrikvveggplpfafdilatsflygsktfinhtqgipdffkqsfpegftwervttyedg
gvltatqdtslqdgcliynvkirgvnftsngpvmqkktlgweaftetlypadgglegrndmalklvggshlianikttyrskkpaknlk
mpgvyyvdyrlerikeannetyveghevavarycdlpsklghkln*
SEQ ID NO: 5 (p93 (KRAB-dCas9-XTEN80-Dnmt3A-Dnmt3L-P2A-BFP): KRAB
(bold, from Gilbert et al., Cell, 2013, 2014), Linkers (underlined), dCas9 (italics), HA tag
(lowercase), SV40 NLS (lowercase italics), XTEN80 (80 amino acid sequence), Dnmt3A (bold
italics, from Siddique et al., JMB, 2013; Stepper et al., NAR, 2016), 27 amino acid linker
(italics underlined; from Siddique et al., JMB, 2013; Stepper et al., NAR, 2016), Dnmt3L (bold
underlined, from Siddique et al., JMB, 2013; Stepper et al., NAR, 2016), P2A peptide cleavage
sequence (lowercase bold), BFP (lowercase underlined))
DAKSLTAWSRTLVTFKDVFVDFTREEWKLLDTAQQIVYRNVMLENYKNLVSLGY
QLTKPDVILRLEKGEEPGGSGGGSMDKKYSIGLAIGTNSVGWAVITDEYKVPSKKFKVLG
NTDRHSIKKNLIGALLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKADLRLIYLALAHMIKF
RGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLF
LAAKNLSDAILLSDILRVNTEITKAPLSASMIKRYDEHHQDLTLLKALVRQQLPEKYKEIFFDQS
KNGYAGYIDGGASQEEFYKFIKPILEKMDGTEELLVKLNREDLLRKQRTFDNGSIPHQIHLGE
LHAILRRQEDFYPFLKDNREKIEKILTFRIPYYVGPLARGNSRFAWMTRKSEETITPWNFEEVV
DKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFL
DNEENEDILEDIVLTLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMKRIEEGIKELGSQI
LKEHPVENTQLQNEKLYLYYLQNGRDMYVDQELDINRLSDYDVDAIVPQSFLKDDSIDNKVL
TRSDKNRGKSDNVPSEEVVKKMKNYWRQLLNAKLITQRKFDNLTKAERGGLSELDKAGFIKR
QLVETRQITKHVAQILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKATAKYFFYSNIMNF
FKTEITLANGEIRKRPLIETNGETGEIVWDKGRDFATVRKVLSMPQVNIVKKTEVQTGGFSKE
SILPKRNSDKLIARKKDWDPKKYGGFDSPTVAYSVLVVAKVEKGKSKKLKSVKELLGITIMERS
SFEKNPIDFLEAKGYKEVKKDLIIKLPKYSLFELENGRKRMLASAGELQKGNELALPSKYVNF
LYLASHYEKLKGSPEDNEQKQLFVEQHKHYLDEIIEQISEFSKRVILADANLDKVLSAYNKHR
DKPIREQAENIIHLFTLTNLGAPAAFKYFDTTIDRKRYTSTKEVLDATLIHQSITGLYETRIDLSQ
LGGDSRADypydvpdyaSGSpkkkrkvSPGGGPSSGAPPPSGGSPAGSPTSTEEGTSESATPESG
PGTSTEPSEGSAPGSPAGSPTSTEEGTSTEPSEGSAPGTSTEPSENHDQEFDPPKVYPPVP
AEKRKPIRVLSLFDGIATGLLVLKDLGIQVDRYIASEVCEDSITVGMVRHQGKIMYVGDV
RSVTQKHIQEWGPFDLVIGGSPCNDLSIVNPARKGLYEGTGRLFFEFYRLLHDARPKEG
DDRPFFWLFENVVAMGVSDKRDISRFLESNPVMIDAKEVSAAHRARYFWGNLPGMNR
PLASTVNDKLELQECLEHGRIAKFSKVRTITTRSNSIKQGKDQHFPVFMNEKEDILWCT
EMERVFGFPVHYTDVSNMSRLARQRLLGRSWSVPVIRHLFAPLKEYFACVSSGNSNANS
RGPSFSSGLVPLSLRGSHMGPMEIYKTVSAWKRQPVRVLSLFRNIDKVLKSLGFLESG
SGSGGGTLKYVEDVTNVVRRDVEKWGPFDLVYGSTQPLGSSCDRCPGWYMFQFH
RILQYALPRQESQRPFFWIFMDNLLLTEDDQETTTRFLQTEAVTLQDVRGRDYQN
AMRVWSNIPGLKSKHAPLTPKEEEYLQAQVRSRSKLDAPKVDLLVKNCLLPLREY
FKYFSQNSLPLSRADpkkkrkvGSGatnfsllkqagdveenpgpselikenmhmklymegtvdnhhfkctsegeg
kpyegtqtmrikvveggplpfafdilatsflygsktfinhtqgipdffkqsfpegftwervttyedggvltatqdtslqdgcliynvkirgv
nftsngpvmqkktlgweaftetlypadgglegrndmalklvggshlianikttyrskkpaknlkmpgvyyvdyrlerikeannetyv
eqhevavarycdlpsklghkln*
SEQ ID NO: 6 (p94 (KRAB-dCas9-XTEN80-Dnmt3A-Dnmt3L-P2A-P2A-BFP):
KRAB (bold, from Gilbert et al., Cell, 2013, 2014), Linkers (underlined), dCas9 (italics), HA
tag (lowercase), SV40 NLS (lowercase italics), XTEN80 (80 amino acid sequence), Dnmt3 A
(bold italics, from Siddique et al., JMB, 2013; Stepper et al., NAR, 2016), 27 amino acid linker
(italics underlined, from Siddique et al., JMB, 2013; Stepper et al., NAR, 2016), Dnmt3L (bold
underlined, from Siddique et al., JMB, 2013; Stepper et al., NAR, 2016), P2A peptide cleavage
sequence (lowercase bold), BFP (lowercase underlined))
DAKSLTAWSRTLVTFKDVFVDFTREEWKLLDTAQQIVYRNVMLENYKNLVSLGY
QLTKPDVILRLEKGEEPGGSGGGSMDKKYSIGLAIGTNSVGWAVITDEYKVPSKKFKVLG
NTDRHSIKKNLIGALLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKADLRLIYLALAHMIKF
RGHFLIEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQYADLF
LAAKNLSDAILLSDILRVNTEITKAPLSASMIKRYDEHHQDLTLLKALVRQQLPEKYKEIFFDQS
KNGYAGYIDGGASQEEFYKFIKPILEKMDGTEELLVKLNREDLLRKQRTFDNGSIPHQIHLGE
LHAILRRQEDFYPFLKDNREKIEKILTFRIPYYVGPLARGNSRFAWMTRKSEETITPWNFEEVV
DKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFL
DNEENEDILEDIVLTLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMKRIEEGIKELGSQI
LKEHPVENTQLQNEKLYLYYLQNGRDMYVDQELDINRLSDYDVDAIVPQSFLKDDSIDNKVL
TRSDKNRGKSDNVPSEEVVKKMKNYWRQLLNAKLITQRKFDNLTKAERGGLSELDKAGFIKR
QLVETRQITKHVAQILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKATAKYFFYSNIMNF
FKTEITLANGEIRKRPLIETNGETGEIVWDKGRDFATVRKVLSMPQVNIVKKTEVQTGGFSKE
SILPKRNSDKLIARKKDWDPKKYGGFDSPTVAYSVLVVAKVEKGKSKKLKSVKELLGITIMERS
SFEKNPIDFLEAKGYKEVKKDLIIKLPKYSLFELENGRKRMLASAGELQKGNELALPSKYVNF
LYLASHYEKLKGSPEDNEQKQLFVEQHKHYLDEIIEQISEFSKRVILADANLDKVLSAYNKHR
DKPIREQAENIIHLFTLTNLGAPAAFKYFDTTIDRKRYTSTKEVLDATLIHQSITGLYETRIDLSQ
LGGDSRADypydvpdyaSGSpkkkrkvSPGGGPSSGAPPPSGGSPAGSPTSTEEGTSESATPESG
PGTSTEPSEGSAPGSPAGSPTSTEEGTSTEPSEGSAPGTSTEPSENHDQEFDPPKVYPPVP
AEKRKPIRVLSLFDGIATGLLVLKDLGIQVDRYIASEVCEDSITVGMVRHQGKIMYVGDV
RSVTQKHIQEWGPFDLVIGGSPCNDLSIVNPARKGLYEGTGRLFFEFYRLLHDARPKEG
DDRPFFWLFENVVAMGVSDKRDISRFLESNPVMIDAKEVSAAHRARYFWGNLPGMNR
PLASTVNDKLELQECLEHGRIAKFSKVRTITTRSNSIKQGKDQHFPVFMNEKEDILWCT
EMERVFGFPVHYTDVSNMSRLARQRLLGRSWSVPVIRHLFAPLKEYFACVSSGNSNANS
RGPSFSSGLVPLSLRGSHMGPMEIYKTVSAWKRQPVRVLSLFRNIDKVLKSLGFLESG
SGSGGGTLKYVEDVTNVVRRDVEKWGPFDLVYGSTQPLGSSCDRCPGWYMFQFH
RILQYALPRQESQRPFFWIFMDNLLLTEDDQETTTRFLQTEAVTLQDVRGRDYQN
AMRVWSNIPGLKSKHAPLTPKEEEYLQAQVRSRSKLDAPKVDLLVKNCLLPLREY
FKYFSQNSLPLSRADpkkkrkvGSGatnfsllkqagdveenpgpGSGatnfsllkqagdveenpgpselikenm
hmklymegtvdnhhfkctsegegkpyegtqtmrikvveggplpfafdilatsflygsktfinhtqgipdffkqsfpegftwervttyed
ggvltatqdtslqdgcliynvkirgvnftsngpvmqkktlgweaftetlypadgglegrndmalklvggshlianikttyrskkpaknl
kmpgvyyvdyrlerikeannetyveghevavarycdlpsklghkln*
SEQ ID NO: 7 (p95 (KRAB-XTEN16-dCas9-Dnmt3A-Dnmt3L-P2A-BFP): KRAB
(bold, from Gilbert et al., Cell, 2013, 2014), Linkers (underlined), XTEN16 (16 amino acid
sequence), dCas9 (italics), HA tag (lowercase), SV40 NLS (lowercase italics), Dnmt3A (bold
italics, from Siddique et al., JMB, 2013; Stepper et al., NAR, 2016), 27 amino acid linker
(italics underlined, from Siddique et al., JMB, 2013; Stepper et al., NAR, 2016), Dnmt3L (bold
underlined, from Siddique et al., JMB, 2013; Stepper et al., NAR, 2016), P2A peptide cleavage
sequence (lowercase bold), BFP (lowercase underlined))
DAKSLTAWSRTLVTFKDVFVDFTREEWKLLDTAQQIVYRNVMLENYKNLVSLGY
QLTKPDVILRLEKGEEPSGSETPGTSESATPESMDKKYSIGLAIGTNSVGWAVITDEYKVPS
KKFKVLGNTDRHSIKKNLIGALLFDSGETAEATRLKRTARRRYTRRKNRICYLQEIFSNEMAKV
DDSFFHRLEESFLVEEDKKHERHPIFGNIVDEVAYHEKYPTIYHLRKKLVDSTDKADLRLIYLA
LAHMIKFRGHFLIEGDLNPDNSDVDKLFIQLVQTYNQLFEENPINASGVDAKAILSARLSKSRR
LENLIAQLPGEKKNGLFGNLIALSLGLTPNFKSNFDLAEDAKLQLSKDTYDDDLDNLLAQIGD
QYADLFLAAKNLSDAILLSDILRVNTEITKAPLSASMIKRYDEHHQDLTLLKALVRQQLPEKYK
EIFFDQSKNGYAGYIDGGASQEEFYKFIKPILEKMDGTEELLVKLNREDLLRKQRTFDNGSIP
HQIHLGELHAILRRQEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELTKVKYVTEGMRKP
AFLSGEQKKAIVDLLFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQLKRRRYTGWGRLS
RKLINGIRDKQSGKTILDFLKSDGFANRNFMQLIHDDSLTFKEDIQKAQVSGQGDSLHEHIA
NLAGSPAIKKGILQTVKVVDELVKVMGRHKPENIVIEMARENQTTQKGQKNSRERMKRIEEG
IKELGSQILKEHPVENTQLQNEKLYLYYLQNGRDMYVDQELDINRLSDYDVDAIVPQSFLKDD
SIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGKATAKYFF
YSNIMNFFKTEITLANGEIRKRPLIETNGETGEIVWDKGRDFATVRKVLSMPQVNIVKKTEVQT
GGFSKESILPKRNSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRMLASAGELQKGNELAL
PSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYE
TRIDLSQLGGDSRADypydvpdyaSGSpkkkrkvEASGSGRASPGIPGSTRNHDQEFDPPKVYPP
VPAEKRKPIRVLSLFDGIATGLLVLKDLGIQVDRYIASEVCEDSITVGMVRHQGKIMYVG
DVRSVTQKHIQEWGPFDLVIGGSPCNDLSIVNPARKGLYEGTGRLFFEFYRLLHDARPK
EGDDRPFFWLFENVVAMGVSDKRDISRFLESNPVMIDAKEVSAAHRARYFWGNLPGM
NRPLASTVNDKLELQECLEHGRIAKFSKVRTITTRSNSIKQGKDQHFPVFMNEKEDILW
CTEMERVFGFPVHYTDVSNMSRLARQRLLGRSWSVPVIRHLFAPLKEYFACVSSGNSNA
NSRGPSFSSGLVPLSLRGSHMGPMEIYKTVSAWKRQPVRVLSLFRNIDKVLKSLGFLE
SGSGSGGGTLKYVEDVTNVVRRDVEKWGPFDLVYGSTQPLGSSCDRCPGWYMFQ
FHRILQYALPRQESQRPFFWIFMDNLLLTEDDQETTTRFLQTEAVTLQDVRGRDY
QNAMRVWSNIPGLKSKHAPLTPKEEEYLQAQVRSRSKLDAPKVDLLVKNCLLPLR
EYFKYFSQNSLPLSRADpkkkrkvGSGatnfsllkqagdveenpgpselikenmhmklymegtvdnhhfkctse
gegkpyegtqtmrikvveggplpfafdilatsflygsktfinhtqgipdffkqsfpegftwervttyedggvltatqdtslqdgcliynyki
rgvnftsngpvmqkktlgweaftetlypadgglegrndmalklvggshlianikttyrskkpaknlkmpgvyyvdyrlerikeanne
tyveqhevavarycdlpsklghkln*
SEQ ID NO: 8 (p96 (KRAB-XTEN16-dCas9-Dnmt3A-Dnmt3L-P2A-P2A-BFP):
KRAB (bold, from Gilbert et al., Cell, 2013, 2014), Linkers (underlined), XTEN16 (16 amino
acid sequence), dCas9 (italics), HA tag (lowercase), SV40 NLS (lowercase italics), Dnmt3 A
(bold italics, from Siddique et al., JMB, 2013; Stepper et al., NAR, 2016), 27 amino acid linker
(italics underlined, from Siddique et al., JMB, 2013; Stepper et al., NAR, 2016), Dnmt3L (bold
underlined, from Siddique et al., JMB, 2013; Stepper et al., NAR, 2016), P2A peptide cleavage
sequence (lowercase bold); BFP (lowercase underlined))
DAKSLTAWSRTLVTFKDVFVDFTREEWKLLDTAQQIVYRNVMLENYKNLVSLGY
QLTKPDVILRLEKGEEPSGSETPGTSESATPESMDKKYSIGLAIGTNSVGWAVITDEYKVPS
KKFKVLGNTDRHSIKKNLIGALLFDSGETAEATRLKRTARRRYTRRKNRICYLQEIFSNEMAKV
DDSFFHRLEESFLVEEDKKHERHPIFGNIVDEVAYHEKYPTIYHLRKKLVDSTDKADLRLIYLA
LAHMIKFRGHFLIEGDLNPDNSDVDKLFIQLVQTYNQLFEENPINASGVDAKAILSARLSKSRR
LENLIAQLPGEKKNGLFGNLIALSLGLTPNFKSNFDLAEDAKLQLSKDTYDDDLDNLLAQIGD
QYADLFLAAKNLSDAILLSDILRVNTEITKAPLSASMIKRYDEHHQDLTLLKALVRQQLPEKYK
EIFFDQSKNGYAGYIDGGASQEEFYKFIKPILEKMDGTEELLVKLNREDLLRKQRTFDNGSIP
HQIHLGELHAILRRQEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELTKVKYVTEGMRKP
AFLSGEQKKAIVDLLFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQLKRRRYTGWGRLS
RKLINGIRDKQSGKTILDFLKSDGFANRNFMQLIHDDSLTFKEDIQKAQVSGQGDSLHEHIA
NLAGSPAIKKGILQTVKVVDELVKVMGRHKPENIVIEMARENQTTQKGQKNSRERMKRIEEG
IKELGSQILKEHPVENTQLQNEKLYLYYLQNGRDMYVDQELDINRLSDYDVDAIVPQSFLKDD
SIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGKATAKYFF
YSNIMNFFKTEITLANGEIRKRPLIETNGETGEIVWDKGRDFATVRKVLSMPQVNIVKKTEVQT
GGFSKESILPKRNSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRMLASAGELQKGNELAL
PSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYE
TRIDLSQLGGDSRADypydvpdyaSGSpkkkrkvEASGSGRASPGIPGSTRNHDQEFDPPKVYPP
VPAEKRKPIRVLSLFDGIATGLLVLKDLGIQVDRYIASEVCEDSITVGMVRHQGKIMYVG
DVRSVTQKHIQEWGPFDLVIGGSPCNDLSIVNPARKGLYEGTGRLFFEFYRLLHDARPK
EGDDRPFFWLFENVVAMGVSDKRDISRFLESNPVMIDAKEVSAAHRARYFWGNLPGM
NRPLASTVNDKLELQECLEHGRIAKFSKVRTITTRSNSIKQGKDQHFPVFMNEKEDILW
CTEMERVFGFPVHYTDVSNMSRLARQRLLGRSWSVPVIRHLFAPLKEYFACVSSGNSNA
NSRGPSESSGLVPLSLRGSHMGPMEIYKTVSAWKRQPVRVLSLFRNIDKVLKSLGFLE
SGSGSGGGTLKYVEDVTNVVRRDVEKWGPFDLVYGSTQPLGSSCDRCPGWYMFQ
FHRILQYALPRQESQRPFFWIFMDNLLLTEDDQETTTRFLQTEAVTLQDVRGRDY
QNAMRVWSNIPGLKSKHAPLTPKEEEYLQAQVRSRSKLDAPKVDLLVKNCLLPLR
EYFKYFSQNSLPLSRADpkkkrkvGSGatnfsllkqagdveenpgpGSGatnfsllkqagdveenpgpselike
nmhmklymegtvdnhhfkctsegegkpyegtqtmrikvveggplpfafdilatsflygsktfinhtqgipdffkqsfpegftwervtt
yedggvltatqdtslqdgcliynvkirgvnftsngpvmqkktlgweaftetlypadgglegrndmalklvggshlianikttyrskkpa
knlkmpgvyyvdyrlerikeannetyveghevavarycdlpsklghkln*
SEQ ID NO: 9 (p97 (KRAB-XTEN80-dCas9-Dnmt3A-Dnmt3L-P2A-BFP): KRAB
(bold, from Gilbert et al., Cell, 2013, 2014), Linkers (underlined), XTEN80 (80 amino acid
sequence), dCas9 (italics); HA tag (lowercase), SV40 NLS (lowercase italics), Dnmt3A (bold
italics, from Siddique et al., JMB, 2013; Stepper et al., NAR, 2016), 27 amino acid linker
(italics underlined, from Siddique et al., JMB, 2013; Stepper et al., NAR, 2016), Dnmt3L (bold
underlined, from Siddique et al., JMB, 2013; Stepper et al., NAR, 2016), P2A peptide cleavage
sequence (lowercase bold), BFP (lowercase underlined))
DAKSLTAWSRTLVTFKDVFVDFTREEWKLLDTAQQIVYRNVMLENYKNLVSLGY
QLTKPDVILRLEKGEEPGGPSSGAPPPSGGSPAGSPTSTEEGTSESATPESGPGTSTEPSE
GSAPGSPAGSPTSTEEGTSTEPSEGSAPGTSTEPSEMDKKYSIGLAIGTNSVGWAVITDEYKV
PSKKFKVLGNTDRHSIKKNLIGALLFDSGETAEATRLKRTARRRYTRRKNRICYLQEIFSNEMA
KVDDSFFHRLEESFLVEEDKKHERHPIFGNIVDEVAYHEKYPTIYHLRKKLVDSTDKADLRLI
YLALAHMIKFRGHFLIEGDLNPDNSDVDKLFIQLVQTYNQLFEENPINASGVDAKAILSARLSK
SRRLENLIAQLPGEKKNGLFGNLIALSLGLTPNFKSNFDLAEDAKLQLSKDTYDDDLDNLLAQ
IGDQYADLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQRTFDN
GSIPHQIHLGELHAILRRQEDFYPFLKDNREKIEKILTFRIPYYVGPLARGNSRFAWMTRKSEE
TITPWNFEEVVDKGASAQSFIERMTNFDKNLPNEKVLPKHSLLYEYFTVYNELTKVKYVTEGM
RKPAFLSGEQKKAIVDLLFKTNRKVTVKQLKEDYFKKIECFDSVEISGVEDRFNASLGTYHDL
LKIIKDKDFLDNEENEDILEDIVLTLTLFEDREMIEERLKTYAHLFDDKVMKQLKRRRYTGWG
RLSRKLINGIRDKQSGKTILDFLKSDGFANRNFMQLIHDDSLTFKEDIQKAQVSGQGDSLHE
HIANLAGSPAIKKGILQTVKVVDELVKVMGRHKPENIVIEMARENQTTQKGQKNSRERMKRI
EEGIKELGSQILKEHPVENTQLQNEKLYLYYLQNGRDMYVDQELDINRLSDYDVDAIVPQSFL
KDDSIDNKVLTRSDKNRGKSDNVPSEEVVKKMKNYWRQLLNAKLITQRKFDNLTKAERGGLS
ELDKAGFIKRQLVETRQITKHVAQILDSRMNTKYDENDKLIREVKVITLKSKLVSDFRKDFQF
YKVREINNYHHAHDAYLNAVVGTALIKKYPKLESEFVYGDYKVYDVRKMIAKSEQEIGKATAK
YFFYSNIMNFFKTEITLANGEIRKRPLIETNGETGEIVWDKGRDFATVRKVLSMPQVNIVKKTE
VQTGGFSKESILPKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQISEFSKRVILADANLDK
VLSAYNKHRDKPIREQAENIIHLFTLTNLGAPAAFKYFDTTIDRKRYTSTKEVLDATLIHQSITG
LYETRIDLSQLGGDSRADypydvpdyaSGSpkkkrkvEASGSGRASPGIPGSTRNHDQEFDPPKV
YPPVPAEKRKPIRVLSLFDGIATGLLVLKDLGIQVDRYIASEVCEDSITVGMVRHQGKIM
YVGDVRSVTQKHIQEWGPFDLVIGGSPCNDLSIVNPARKGLYEGTGRLFFEFYRLLHDA
RPKEGDDRPFFWLFENVVAMGVSDKRDISRFLESNPVMIDAKEVSAAHRARYFWGNL
PGMNRPLASTVNDKLELQECLEHGRIAKFSKVRTITTRSNSIKQGKDQHFPVFMNEKE
DILWCTEMERVFGFPVHYTDVSNMSRLARQRLLGRSWSVPVIRHLFAPLKEYFACVSSG
NSNANSRGPSFSSGLVPLSLRGSHMGPMEIYKTVSAWKRQPVRVLSLFRNIDKVLKSL
GFLESGSGSGGGTLKYVEDVTNVVRRDVEKWGPFDLVYGSTQPLGSSCDRCPGW
YMFQFHRILQYALPRQESQRPFFWIFMDNLLLTEDDQETTTRFLQTEAVTLQDVR
GRDYQNAMRVWSNIPGLKSKHAPLTPKEEEYLQAQVRSRSKLDAPKVDLLVKNC
LLPLREYFKYFSQNSLPLSRADpkkkrkvGSGatnfsllkqagdveenpgpselikenmhmklymegtvdn
hhfkctsegegkpyegtqtmrikvveggplpfafdilatsflygsktfinhtqgipdffkqsfpegftwervttyedggvltatqdtslqd
gcliynvkirgvnftsngpvmqkktlgweaftetlypadgglegrndmalklvggshlianikttyrskkpaknlkmpgvyyvdyrl
erikeannetyveghevavarycdlpsklghkln*
SEQ ID NO: 10 (p98 (KRAB-XTEN80-dCas9-Dnmt3A-Dnmt3L-P2A-P2A-BFP):
KRAB (bold, from Gilbert et al., Cell, 2013, 2014), Linkers (underlined), XTEN80 (80 amino
acid sequence), dCas9 (italics), HA tag (lowercase), SV40 NLS (lowercase italics), Dnmt3A
(bold italics, from Siddique et al., JMB, 2013; Stepper et al., NAR, 2016), 27 amino acid linker
(italics underlined, from Siddique et al., JMB, 2013; Stepper et al., NAR, 2016), Dnmt3L (bold
underlined, from Siddique et al., JMB, 2013; Stepper et al., NAR, 2016), P2A peptide cleavage
sequence (lowercase bold), BFP (lowercase underlined))
DAKSLTAWSRTLVTFKDVFVDFTREEWKLLDTAQQIVYRNVMLENYKNLVSLGY
QLTKPDVILRLEKGEEPGGPSSGAPPPSGGSPAGSPTSTEEGTSESATPESGPGTSTEPSE
GSAPGSPAGSPTSTEEGTSTEPSEGSAPGTSTEPSEMDKKYSIGLAIGTNSVGWAVITDEYKV
PSKKFKVLGNTDRHSIKKNLIGALLFDSGETAEATRLKRTARRRYTRRKNRICYLQEIFSNEMA
KVDDSFFHRLEESFLVEEDKKHERHPIFGNIVDEVAYHEKYPTIYHLRKKLVDSTDKADLRLI
YLALAHMIKFRGHFLIEGDLNPDNSDVDKLFIQLVQTYNQLFEENPINASGVDAKAILSARLSK
SRRLENLIAQLPGEKKNGLFGNLIALSLGLTPNFKSNFDLAEDAKLQLSKDTYDDDLDNLLAQ
IGDQYADLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQRTFDN
GSIPHQIHLGELHAILRRQEDFYPFLKDNREKIEKILTFRIPYYVGPLARGNSRFAWMTRKSEE
TITPWNFEEVVDKGASAQSFIERMTNFDKNLPNEKVLPKHSLLYEYFTVYNELTKVKYVTEGM
RKPAFLSGEQKKAIVDLLFKTNRKVTVKQLKEDYFKKIECFDSVEISGVEDRFNASLGTYHDL
LKIIKDKDFLDNEENEDILEDIVLTLTLFEDREMIEERLKTYAHLFDDKVMKQLKRRRYTGWG
RLSRKLINGIRDKQSGKTILDFLKSDGFANRNFMQLIHDDSLTFKEDIQKAQVSGQGDSLHE
HIANLAGSPAIKKGILQTVKVVDELVKVMGRHKPENIVIEMARENQTTQKGQKNSRERMKRI
EEGIKELGSQILKEHPVENTQLQNEKLYLYYLQNGRDMYVDQELDINRLSDYDVDAIVPQSFL
KDDSIDNKVLTRSDKNRGKSDNVPSEEVVKKMKNYWRQLLNAKLITQRKFDNLTKAERGGLS
ELDKAGFIKRQLVETRQITKHVAQILDSRMNTKYDENDKLIREVKVITLKSKLVSDFRKDFQF
YKVREINNYHHAHDAYLNAVVGTALIKKYPKLESEFVYGDYKVYDVRKMIAKSEQEIGKATAK
YFFYSNIMNFFKTEITLANGEIRKRPLIETNGETGEIVWDKGRDFATVRKVLSMPQVNIVKKTE
VQTGGFSKESILPKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQISEFSKRVILADANLDK
VLSAYNKHRDKPIREQAENIIHLFTLTNLGAPAAFKYFDTTIDRKRYTSTKEVLDATLIHQSITG
LYETRIDLSQLGGDSRADypydvpdyaSGSpkkkrkvEASGSGRASPGIPGSTRNHDQEFDPPKV
YPPVPAEKRKPIRVLSLFDGIATGLLVLKDLGIQVDRYIASEVCEDSITVGMVRHQGKIM
YVGDVRSVTQKHIQEWGPFDLVIGGSPCNDLSIVNPARKGLYEGTGRLFFEFYRLLHDA
RPKEGDDRPFFWLFENVVAMGVSDKRDISRFLESNPVMIDAKEVSAAHRARYFWGNL
PGMNRPLASTVNDKLELQECLEHGRIAKFSKVRTITTRSNSIKQGKDQHFPVFMNEKE
DILWCTEMERVFGFPVHYTDVSNMSRLARQRLLGRSWSVPVIRHLFAPLKEYFACVSSG
NSNANSRGPSFSSGLVPLSLRGSHMGPMEIYKTVSAWKRQPVRVLSLFRNIDKVLKSL
GFLESGSGSGGGTLKYVEDVTNVVRRDVEKWGPFDLVYGSTQPLGSSCDRCPGW
YMFQFHRILQYALPRQESQRPFFWIFMDNLLLTEDDQETTTRFLQTEAVTLQDVR
GRDYQNAMRVWSNIPGLKSKHAPLTPKEEEYLQAQVRSRSKLDAPKVDLLVKNC
LLPLREYFKYFSQNSLPLSRADpkkkrkvGSGatnfsllkqagdveenpgpGSGatnfsllkqagdveenp
gpselikenmhmklymegtvdnhhfkctsegegkpyegtqtmrikvveggplpfafdilatsflygsktfinhtqgipdffkqsfpeg
ftwervttyedggvltatqdtslqdgcliynvkirgvnftsngpvmqkktlgweaftetlypadgglegrndmalklvggshlianiktt
yrskkpaknlkmpgvyyvdyrlerikeannetyveghevavarycdlpsklghkln*
SEQ ID NO: 11 (p99 (KRAB-XTEN16-dCas9-XTEN80-Dnmt3A-Dnmt3L-P2A-BFP):
KRAB (bold, from Gilbert et al., Cell, 2013, 2014), XTEN16 (16 amino acid sequence), dCas9
(italics), HA tag (lowercase), Linkers (underlined), SV40 NLS (lowercase italics), XTEN80
(lowercase italics bold, 80 amino acid sequence), Dnmt3A (bold italics, from Siddique et al.,
JMB, 2013; Stepper et al., NAR, 2016), 27 amino acid linker (italics underlined, from Siddique
et al., JMB, 2013; Stepper et al., NAR, 2016), Dnmt3L (old underlined, from Siddique et al.,
JMB, 2013; Stepper et al., NAR, 2016), P2A peptide cleavage sequence (lowercase bold), BFP
(lowercase underlined))
DAKSLTAWSRTLVTFKDVFVDFTREEWKLLDTAQQIVYRNVMLENYKNLVSLGY
QLTKPDVILRLEKGEEPSGSETPGTSESATPESMDKKYSIGLAIGTNSVGWAVITDEYKVPS
KKFKVLGNTDRHSIKKNLIGALLFDSGETAEATRLKRTARRRYTRRKNRICYLQEIFSNEMAKV
DDSFFHRLEESFLVEEDKKHERHPIFGNIVDEVAYHEKYPTIYHLRKKLVDSTDKADLRLIYLA
LAHMIKFRGHFLIEGDLNPDNSDVDKLFIQLVQTYNQLFEENPINASGVDAKAILSARLSKSRR
LENLIAQLPGEKKNGLFGNLIALSLGLTPNFKSNFDLAEDAKLQLSKDTYDDDLDNLLAQIGD
QYADLFLAAKNLSDAILLSDILRVNTEITKAPLSASMIKRYDEHHQDLTLLKALVRQQLPEKYK
EIFFDQSKNGYAGYIDGGASQEEFYKFIKPILEKMDGTEELLVKLNREDLLRKQRTFDNGSIP
HQIHLGELHAILRRQEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELTKVKYVTEGMRKP
AFLSGEQKKAIVDLLFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQLKRRRYTGWGRLS
RKLINGIRDKQSGKTILDFLKSDGFANRNFMQLIHDDSLTFKEDIQKAQVSGQGDSLHEHIA
NLAGSPAIKKGILQTVKVVDELVKVMGRHKPENIVIEMARENQTTQKGQKNSRERMKRIEEG
IKELGSQILKEHPVENTQLQNEKLYLYYLQNGRDMYVDQELDINRLSDYDVDAIVPQSFLKDD
SIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGKATAKYFF
YSNIMNFFKTEITLANGEIRKRPLIETNGETGEIVWDKGRDFATVRKVLSMPQVNIVKKTEVQT
GGFSKESILPKRNSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRMLASAGELQKGNELAL
PSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYE
TRIDLSQLGGDSRADypydvpdyaSGSpkkkrkvSPGggpssgapppsggspagsptsteegtsesatpesgpgtste
psegsapgspagsptsteegtstepsegsapgtstepseNHDQEFDPPKVYPPVPAEKRKPIRVLSLFDGIAT
GLLVLKDLGIQVDRYIASEVCEDSITVGMVRHQGKIMYVGDVRSVTQKHIQEWGPFDLV
IGGSPCNDLSIVNPARKGLYEGTGRLFFEFYRLLHDARPKEGDDRPFFWLFENVVAMG
VSDKRDISRFLESNPVMIDAKEVSAAHRARYFWGNLPGMNRPLASTVNDKLELQECLE
HGRIAKFSKVRTITTRSNSIKQGKDQHFPVFMNEKEDILWCTEMERVFGFPVHYTDVS
NMSRLARQRLLGRSWSVPVIRHLFAPLKEYFACVSSGNSNANSRGPSFSSGLVPLSLRGSH
MGPMEIYKTVSAWKRQPVRVLSLFRNIDKVLKSLGFLESGSGSGGGTLKYVEDVT
NVVRRDVEKWGPFDLVYGSTQPLGSSCDRCPGWYMFQFHRILQYALPRQESQRPF
FWIFMDNLLLTEDDQETTTRFLQTEAVTLQDVRGRDYQNAMRVWSNIPGLKSKH
APLTPKEEEYLQAQVRSRSKLDAPKVDLLVKNCLLPLREYFKYFSQNSLPLSRADpk
kkrkvGSGatnfsllkqagdveenpgpselikenmhmklymegtvdnhhfkctsegegkpyegtqtmrikvveggplpfafdi
latsflygsktfinhtqgipdffkqsfpegftwervttyedggvltatqdtslqdgcliynvkirgvnftsngpvmqkktlgweaftetlyp
adgglegrndmalklvggshlianikttyrskkpaknlkmpgvyyvdyrlerikeannetyveghevavarycdlpsklghkln*
SEQ ID NO: 12 (p100 (KRAB-XTEN16-dCas9-XTEN80-Dnmt3A-Dnmt3L-P2A-P2A-
BFP): KRAB (bold, from Gilbert et al., Cell, 2013, 2014), XTEN16 (16 amino acid sequence),
dCas9 (italics), HA tag (lowercase), Linkers (underlined), SV40 NLS (lowercase italics),
XTEN80 (lowercase bold italics, 80 amino acid sequence), Dnmt3A (bold italics, from Siddique
et al., JMB, 2013; Stepper et al., NAR, 2016), 27 amino acid linker (italics underlined, from
Siddique et al., JMB, 2013; Stepper et al., NAR, 2016), Dnmt3L (bold underlined, from
Siddique et al., JMB, 2013; Stepper et al., NAR, 2016), P2A peptide cleavage sequence
(lowercase bold), BFP (lowercase underlined))
DAKSLTAWSRTLVTFKDVFVDFTREEWKLLDTAQQIVYRNVMLENYKNLVSLGY
QLTKPDVILRLEKGEEPSGSETPGTSESATPESMDKKYSIGLAIGTNSVGWAVITDEYKVPS
KKFKVLGNTDRHSIKKNLIGALLFDSGETAEATRLKRTARRRYTRRKNRICYLQEIFSNEMAKV
DDSFFHRLEESFLVEEDKKHERHPIFGNIVDEVAYHEKYPTIYHLRKKLVDSTDKADLRLIYLA
LAHMIKFRGHFLIEGDLNPDNSDVDKLFIQLVQTYNQLFEENPINASGVDAKAILSARLSKSRR
LENLIAQLPGEKKNGLFGNLIALSLGLTPNFKSNFDLAEDAKLQLSKDTYDDDLDNLLAQIGD
QYADLFLAAKNLSDAILLSDILRVNTEITKAPLSASMIKRYDEHHQDLTLLKALVRQQLPEKYK
EIFFDQSKNGYAGYIDGGASQEEFYKFIKPILEKMDGTEELLVKLNREDLLRKQRTFDNGSIP
HQIHLGELHAILRRQEDFYPFLKDNREKIEKILTFRIPYYVGPLARGNSRFAWMTRKSEETITP
WNFEEVVDKGASAQSFIERMTNFDKNLPNEKVLPKHSLLYEYFTVYNELTKVKYVTEGMRKP
AFLSGEQKKAIVDLLFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQLKRRRYTGWGRLS
RKLINGIRDKQSGKTILDFLKSDGFANRNFMQLIHDDSLTFKEDIQKAQVSGQGDSLHEHIA
NLAGSPAIKKGILQTVKVVDELVKVMGRHKPENIVIEMARENQTTQKGQKNSRERMKRIEEG
IKELGSQILKEHPVENTQLQNEKLYLYYLQNGRDMYVDQELDINRLSDYDVDAIVPQSFLKDD
SIDNKVLTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGKATAKYFF
YSNIMNFFKTEITLANGEIRKRPLIETNGETGEIVWDKGRDFATVRKVLSMPQVNIVKKTEVQT
GGFSKESILPKRNSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRMLASAGELQKGNELAL
PSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYE
TRIDLSQLGGDSRADypydvpdyaSGSpkkkrkvSPGggpssgapppsggspagsptsteegtsesatpesgpgtste
psegsapgspagsptsteegtstepsegsapgtstepseNHDQEFDPPKVYPPVPAEKRKPIRVLSLFDGIAT
GLLVLKDLGIQVDRYIASEVCEDSITVGMVRHQGKIMYVGDVRSVTQKHIQEWGPFDLV
IGGSPCNDLSIVNPARKGLYEGTGRLFFEFYRLLHDARPKEGDDRPFFWLFENVVAMG
VSDKRDISRFLESNPVMIDAKEVSAAHRARYFWGNLPGMNRPLASTVNDKLELQECLE
HGRIAKFSKVRTITTRSNSIKQGKDQHFPVFMNEKEDILWCTEMERVFGFPVHYTDVS
NMSRLARQRLLGRSWSVPVIRHLFAPLKEYFACVSSGNSNANSRGPSFSSGLVPLSLRGSH
MGPMEIYKTVSAWKRQPVRVLSLFRNIDKVLKSLGFLESGSGSGGGTLKYVEDVT
NVVRRDVEKWGPFDLVYGSTQPLGSSCDRCPGWYMFQFHRILQYALPRQESQRPF
FWIFMDNLLLTEDDQETTTRFLQTEAVTLQDVRGRDYQNAMRVWSNIPGLKSKH
APLTPKEEEYLQAQVRSRSKLDAPKVDLLVKNCLLPLREYFKYFSQNSLPLSRADpk
kkrkvGSGatnfsllkqagdveenpgpGSGatnfsllkqagdveenpgpselikenmhmklymegtvdnhhfkctsegeg
kpyegtqtmrikvveggplpfafdilatsflygsktfinhtqgipdffkqsfpegftwervttyedggvltatqdtslqdgcliynvkirgv
nftsngpvmqkktlgweaftetlypadgglegrndmalklvggshlianikttyrskkpaknlkmpgvyyvdyrlerikeannetyv
eghevavarycdlpsklghkln*
SEQ ID NO: 13 (p101 (KRAB-XTEN80-dCas9-XTEN16-Dnmt3A-Dnmt3L-P2A-
BFP): KRAB (bold, from Gilbert et al., Cell, 2013, 2014), Linkers (underlined). XTEN80
(lowercase bold italics, 80 amino acid sequence), dCas9 (italics), HA tag (lowercase), SV40
NLS (lowercase italics), XTEN16 (16 amino acid sequence), Dnmt3A (bold italics, from
Siddique et al., JMB, 2013; Stepper et al., NAR, 2016), 27 amino acid linker (italics under-
lined, from Siddique et al., JMB, 2013; Stepper et al., NAR, 2016), Dnmt3L (bold underlined,
from Siddique et al., JMB, 2013; Stepper et al., NAR, 2016), P2A peptide cleavage sequence
(lowercase bold), BFP (lowercase underlined))
DAKSLTAWSRTLVTFKDVFVDFTREEWKLLDTAQQIVYRNVMLENYKNLVSLGY
QLTKPDVILRLEKGEEPggpssgapppsggspagsptsteegtsesatpesgpgtstepsegsapgspagsptsteegt
stepsegsapgtstepseMDKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEGDLNPDN
SDVDKLFIQLVQTYNQLFEENPINASGVDAKAILSARLSKSRRLENLIAQLPGEKKNGLFGNLI
ALSLGLTPNFKSNFDLAEDAKLQLSKDTYDDDLDNLLAQIGDQYADLFLAAKNLSDAILLSDI
LRVNTEITKAPLSASMIKRYDEHHQDLTLLKALVRQQLPEKYKEIFFDQSKNGYAGYIDGGAS
QEEFYKFIKPILEKMDGTEELLVKLNREDLLRKQRTFDNGSIPHQIHLGELHAILRRQEDFYP
FLKDNREKIEKILTFRIPYYVGPLARGNSRFAWMTRKSEETITPWNFEEVVDKGASAQSFIERM
TNFDKNLPNEKVLPKHSLLYEYFTVYNELTKVKYVTEGMRKPAFLSGEQKKAIVDLLFKTNRK
VTVKQLKEDYFKKIECFDSVEISGVEDRFNASLGTYHDLLKIIKDKDFLDNEENEDILEDIVLT
LTLFEDREMIEERLKTYAHLFDDKVMKQLKRRRYTGWGRLSRKLINGIRDKQSGKTILDFLKS
DGFANRNFMQLIHDDSLTFKEDIQKAQVSGQGDSLHEHIANLAGSPAIKKGILQTVKVVDEL
VKVMGRHKPENIVIEMARENQTTQKGQKNSRERMKRIEEGIKELGSQILKEHPVENTQLQNE
KLYLYYLQNGRDMYVDQELDINRLSDYDVDAIVPQSFLKDDSIDNKVLTRSDKNRGKSDNVP
SEEVVKKMKNYWRQLLNAKLITQRKFDNLTKAERGGLSELDKAGFIKRQLVETRQITKHVAQ
ILDSRMNTKYDENDKLIREVKVITLKSKLVSDFRKDFQFYKVREINNYHHAHDAYLNAVVGTA
LIKKYPKLESEFVYGDYKVYDVRKMIAKSEQEIGKATAKYFFYSNIMNFFKTEITLANGEIRKR
PLIETNGETGEIVWDKGRDFATVRKVLSMPQVNIVKKTEVQTGGFSKESILPKRNSDKLIARK
KDWDPKKYGGFDSPTVAYSVLVVAKVEKGKSKKLKSVKELLGITIMERSSFEKNPIDFLEAKG
YKEVKKDLIIKLPKYSLFELENGRKRMLASAGELQKGNELALPSKYVNFLYLASHYEKLKGSP
EDNEQKQLFVEQHKHYLDEIIEQISEFSKRVILADANLDKVLSAYNKHRDKPIREQAENIIHLF
TLTNLGAPAAFKYFDTTIDRKRYTSTKEVLDATLIHQSITGLYETRIDLSQLGGDSRADypydvp
dyaSGSpkkkrkvSPGSGSETPGTSESATPESNHDQEFDPPKVYPPVPAEKRKPIRVLSLFDGI
ATGLLVLKDLGIQVDRYIASEVCEDSITVGMVRHQGKIMYVGDVRSVTQKHIQEWGPFD
LVIGGSPCNDLSIVNPARKGLYEGTGRLFFEFYRLLHDARPKEGDDRPFFWLFENVVA
MGVSDKRDISRFLESNPVMIDAKEVSAAHRARYFWGNLPGMNRPLASTVNDKLELQEC
LEHGRIAKFSKVRTITTRSNSIKQGKDQHFPVFMNEKEDILWCTEMERVFGFPVHYTD
VSNMSRLARQRLLGRSWSVPVIRHLFAPLKEYFACVSSGNSNANSRGPSFSSGLVPLSLRG
SHMGPMEIYKTVSAWKRQPVRVLSLFRNIDKVLKSLGFLESGSGSGGGTLKYVED
VTNVVRRDVEKWGPFDLVYGSTQPLGSSCDRCPGWYMFQFHRILQYALPRQESQ
RPFFWIFMDNLLLTEDDQETTTRFLQTEAVTLQDVRGRDYQNAMRVWSNIPGLKS
KHAPLTPKEEEYLQAQVRSRSKLDAPKVDLLVKNCLLPLREYFKYFSQNSLPLSRA
DpkkkrkvGSGatnfsllkqagdveenpgpselikenmhmklymegtvdnhhfkctsegegkpyegtqtmrikvveggplpf
afdilatsflygsktfinhtqgipdffkqsfpegftwervttyedggvltatqdtslqdgcliynvkirgvnftsngpvmqkktlgweafte
tlypadgglegrndmalklvggshlianikttyrskkpaknlkmpgvyyvdyrlerikeannetyveghevavarycdlpsklghkl
n*
SEQ ID NO: 14 (p102 (KRAB-XTEN80-dCas9-XTEN16-Dnmt3A-Dnmt3L-P2A-
BFP): KRAB (bold, from Gilbert et al., Cell, 2013, 2014), Linkers (underlined), XTEN80
(lowercase bold italics, 80 amino acid sequence), dCas9 (italics), HA tag (lowercase), SV40
NLS (lowercase italics), XTEN16 (16 amino acid sequence), Dnmt3A (bold italics, from
Siddique et al., JMB, 2013; Stepper et al., NAR, 2016), 27 amino acid linker (italics underlined,
from Siddique et al., JMB, 2013; Stepper et al., NAR, 2016), Dnmt3L (bold underlined, from
Siddique et al., JMB, 2013; Stepper et al., NAR, 2016), P2A peptide cleavage sequence
(lowercase bold), BFP (lowercase underlined))
DAKSLTAWSRTLVTFKDVFVDFTREEWKLLDTAQQIVYRNVMLENYKNLVSLGY
QLTKPDVILRLEKGEEPggpssgapppsggspagsptsteegtsesatpesgpgtstepsegsapgspagsptsteegt
stepsegsapgtstepseMDKKYSIGLAIGTNSVGWAVITDEYKVPSKKFKVLGNTDRHSIKKNLIGAL
LFDSGETAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEGDLNPDN
SDVDKLFIQLVQTYNQLFEENPINASGVDAKAILSARLSKSRRLENLIAQLPGEKKNGLFGNLI
ALSLGLTPNFKSNFDLAEDAKLQLSKDTYDDDLDNLLAQIGDQYADLFLAAKNLSDAILLSDI
LRVNTEITKAPLSASMIKRYDEHHQDLTLLKALVRQQLPEKYKEIFFDQSKNGYAGYIDGGAS
QEEFYKFIKPILEKMDGTEELLVKLNREDLLRKQRTFDNGSIPHQIHLGELHAILRRQEDFYP
FLKDNREKIEKILTFRIPYYVGPLARGNSRFAWMTRKSEETITPWNFEEVVDKGASAQSFIERM
TNFDKNLPNEKVLPKHSLLYEYFTVYNELTKVKYVTEGMRKPAFLSGEQKKAIVDLLFKTNRK
VTVKQLKEDYFKKIECFDSVEISGVEDRFNASLGTYHDLLKIIKDKDFLDNEENEDILEDIVLT
LTLFEDREMIEERLKTYAHLFDDKVMKQLKRRRYTGWGRLSRKLINGIRDKQSGKTILDFLKS
DGFANRNFMQLIHDDSLTFKEDIQKAQVSGQGDSLHEHIANLAGSPAIKKGILQTVKVVDEL
VKVMGRHKPENIVIEMARENQTTQKGQKNSRERMKRIEEGIKELGSQILKEHPVENTQLQNE
KLYLYYLQNGRDMYVDQELDINRLSDYDVDAIVPQSFLKDDSIDNKVLTRSDKNRGKSDNVP
SEEVVKKMKNYWRQLLNAKLITQRKFDNLTKAERGGLSELDKAGFIKRQLVETRQITKHVAQ
ILDSRMNTKYDENDKLIREVKVITLKSKLVSDFRKDFQFYKVREINNYHHAHDAYLNAVVGTA
LIKKYPKLESEFVYGDYKVYDVRKMIAKSEQEIGKATAKYFFYSNIMNFFKTEITLANGEIRKR
PLIETNGETGEIVWDKGRDFATVRKVLSMPQVNIVKKTEVQTGGFSKESILPKRNSDKLIARK
KDWDPKKYGGFDSPTVAYSVLVVAKVEKGKSKKLKSVKELLGITIMERSSFEKNPIDFLEAKG
YKEVKKDLIIKLPKYSLFELENGRKRMLASAGELQKGNELALPSKYVNFLYLASHYEKLKGSP
EDNEQKQLFVEQHKHYLDEIIEQISEFSKRVILADANLDKVLSAYNKHRDKPIREQAENIIHLF
TLTNLGAPAAFKYFDTTIDRKRYTSTKEVLDATLIHQSITGLYETRIDLSQLGGDSRADypydvp
dyaSGSpkkkrkvSPGSGSETPGTSESATPESNHDQEFDPPKVYPPVPAEKRKPIRVLSLFDGI
ATGLLVLKDLGIQVDRYIASEVCEDSITVGMVRHQGKIMYVGDVRSVTQKHIQEWGPFD
LVIGGSPCNDLSIVNPARKGLYEGTGRLFFEFYRLLHDARPKEGDDRPFFWLFENVVA
MGVSDKRDISRFLESNPVMIDAKEVSAAHRARYFWGNLPGMNRPLASTVNDKLELQEC
LEHGRIAKFSKVRTITTRSNSIKQGKDQHFPVFMNEKEDILWCTEMERVFGFPVHYTD
VSNMSRLARQRLLGRSWSVPVIRHLFAPLKEYFACVSSGNSNANSRGPSFSSGLVPLSLRG
SHMGPMEIYKTVSAWKRQPVRVLSLFRNIDKVLKSLGFLESGSGSGGGTLKYVED
VTNVVRRDVEKWGPFDLVYGSTQPLGSSCDRCPGWYMFQFHRILQYALPRQESQ
RPFFWIFMDNLLLTEDDQETTTRFLQTEAVTLQDVRGRDYQNAMRVWSNIPGLKS
KHAPLTPKEEEYLQAQVRSRSKLDAPKVDLLVKNCLLPLREYFKYFSQNSLPLSRA
DpkkkrkvGSGatnfsllkqagdveenpgpGSGatnfsllkqagdveenpgpselikenmhmklymegtvdnhhfkctse
gegkpyegtqtmrikvveggplpfafdilatsflygsktfinhtqgipdffkqsfpegftwervttyedggvltatqdtslqdgcliynvki
rgvnftsngpvmqkktlgweaftetlypadgglegrndmalklvggshlianikttyrskkpaknlkmpgvyyvdyrlerikeanne
tyveghevavarycdlpsklghkln*
SEQ ID NO: 15 (V2.1 or p112 (Dnmt3A-Dnmt3L-XTEN80-dCas9-BFP-KRAB);
KRAB (bold, from Gilbert et al., Cell, 2013, 2014), Linkers (underlined), XTEN80 (lowercase
bold italics, 80 amino acid sequence), dCas9 (italics), HA tag (lowercase), SV40 NLS
(lowercase italics), Dnmt3A (bold italics, from Siddique et al., JMB, 2013; Stepper et al.,
NAR, 2016), 27 amino acid linker (italics underlined, from Siddique et al., JMB, 2013; Stepper
et al., NAR, 2016), Dnmt3L (bold underlined, from Siddique et al., JMB, 2013; Stepper et al.,
NAR, 2016), BFP (lowercase underlined))
NHDQEFDPPKVYPPVPAEKRKPIRVLSLFDGIATGLLVLKDLGIQVDRYIASEVCEDSIT
VGMVRHQGKIMYVGDVRSVTQKHIQEWGPFDLVIGGSPCNDLSIVNPARKGLYEGTGR
LFFEFYRLLHDARPKEGDDRPFFWLFENVVAMGVSDKRDISRFLESNPVMIDAKEVSA
AHRARYFWGNLPGMNRPLASTVNDKLELQECLEHGRIAKFSKVRTITTRSNSIKQGKDQ
HFPVFMNEKEDILWCTEMERVFGFPVHYTDVSNMSRLARQRLLGRSWSVPVIRHLFA
PLKEYFACVSSGNSNANSRGPSFSSGLVPLSLRGSHMGPMEIYKTVSAWKRQPVRVLSL
FRNIDKVLKSLGFLESGSGSGGGTLKYVEDVTNVVRRDVEKWGPFDLVYGSTQPL
GSSCDRCPGWYMFQFHRILQYALPRQESQRPFFWIFMDNLLLTEDDQETTTRFLQ
TEAVTLQDVRGRDYQNAMRVWSNIPGLKSKHAPLTPKEEEYLQAQVRSRSKLDAP
KVDLLVKNCLLPLREYFKYFSQNSLPLggpssgapppsggspagsptsteegtsesatpesgpgtstepsegs
apgspagsptsteegtstepsegsapgtstepseMDKKYSIGLAIGTNSVGWAVITDEYKVPSKKFKVLGNTD
RHSIKKNLIGALLFDSGETAEATRLKRTARRRYTRRKNRICYLQEIFSNEMAKVDDSFFHRLEE
SELVEEDKKHERHPIFGNIVDEVAYHEKYPTIYHLRKKLVDSTDKADLRLIYLALAHMIKFRG
HFLIEGDLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYADLFLA
AKNLSDAILLSDILRVNTEITKAPLSASMIKRYDEHHQDLTLLKALVRQQLPEKYKEIFFDQSK
NGYAGYIDGGASQEEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKSEETITPWNFEEVVD
KGASAQSFIERMTNFDKNLPNEKVLPKHSLLYEYFTVYNELTKVKYVTEGMRKPAFLSGEQK
KAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIR
DKQSGKTILDFLKSDGFANRNFMQLIHDDSLTFKEDIQKAQVSGQGDSLHEHIANLAGSPAI
KKGILQTVKVVDELVKVMGRHKPENIVIEMARENQTTQKGQKNSRERMKRIEEGIKELGSQI
LKEHPVENTQLQNEKLYLYYLQNGRDMYVDQELDINRLSDYDVDAIVPQSFLKDDSIDNKVL
TRSDKNRGKSDNVPSEEVVKKMKNYWRQLLNAKLITQRKFDNLTKAERGGLSELDKAGFIKR
QLVETRQITKHVAQILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKATAKYFFYSNIMNF
FKTEITLANGEIRKRPLIETNGETGEIVWDKGRDFATVRKVLSMPQVNIVKKTEVQTGGFSKE
SILPKRNSDKLIARKKDWDPKKYGGFDSPTVAYSVLVVAKVEKGKSKKLKSVKELLGITIMERS
SFEKNPIDFLEAKGYKEVKKDLIIKLPKYSLFELENGRKRMLASAGELQKGNELALPSKYVNF
LYLASHYEKLKGSPEDNEQKQLFVEQHKHYLDEIIEQISEFSKRVILADANLDKVLSAYNKHR
DKPIREQAENIIHLFTLTNLGAPAAFKYFDTTIDRKRYTSTKEVLDATLIHQSITGLYETRIDLSQ
LGGDAypydvpdyaSLGSGSpkkkrkvEDpkkkrkvDGIGSGSNGSSGSselikenmhmklymegtvdnhhf
kctsegegkpyegtqtmrikvveggplpfafdilatsflygsktfinhtqgipdffkqsfpegftwervttyedggvltatqdtslqdgcli
ynvkirgvnftsngpvmqkktlgweaftetlypadgglegrndmalklvggshlianikttyrskkpaknlkmpgvyyvdyrlerik
eannetyveghevavarycdlpsklghklnGGGGGMDAKSLTAWSRTLVTFKDVFVDFTREEWKL
LDTAQQIVYRNVMLENYKNLVSLGYQLTKPDVILRLEKGEEP*
SEQ ID NO: 16 (KRAB; from Gilbert et al., Cell, 2013, 2014)
DAKSLTAWSRTLVTFKDVFVDFTREEWKLLDTAQQIVYRNVMLENYKNLVSLGYQLT
KPDVILRLEKGEEP
SEQ ID NO: 17 (Linker)
GGSGGGS
SEQ ID NO: 18 (Linker)
SGS
SEQ ID NO: 19 (Linker)
EASGSGRASPGIPGSTR
SEQ ID NO: 20 (Linker)
SRAD
SEQ ID NO: 21 (Linker)
GSG
SEQ ID NO: 22 (Linker)
SPG
SEQ ID NO: 23 (dCas9)
MDKKYSIGLAIGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDN
SDVDKLFIQLVQTYNQLFEENPINASGVDAKAILSARLSKSRRLENLIAQLPGEKKNGLF
GNLIALSLGLTPNFKSNFDLAEDAKLQLSKDTYDDDLDNLLAQIGDQYADLFLAAKNLS
DAILLSDILRVNTEITKAPLSASMIKRYDEHHQDLTLLKALVRQQLPEKYKEIFFDQSKN
GYAGYIDGGASQEEFYKFIKPILEKMDGTEELLVKLNREDLLRKQRTFDNGSIPHQIHLG
ELHAILRRQEDFYPFLKDNREKIEKILTFRIPYYVGPLARGNSRFAWMTRKSEETITPWN
FEEVVDKGASAQSFIERMTNFDKNLPNEKVLPKHSLLYEYFTVYNELTKVKYVTEGMR
KPAFLSGEQKKAIVDLLFKTNRKVTVKQLKEDYFKKIECFDSVEISGVEDRFNASLGTY
HDLLKIIKDKDFLDNEENEDILEDIVLTLTLFEDREMIEERLKTYAHLFDDKVMKQLKRR
RYTGWGRLSRKLINGIRDKQSGKTILDFLKSDGFANRNFMQLIHDDSLTFKEDIQKAQV
SGQGDSLHEHIANLAGSPAIKKGILQTVKVVDELVKVMGRHKPENIVIEMARENQTTQK
GQKNSRERMKRIEEGIKELGSQILKEHPVENTQLQNEKLYLYYLQNGRDMYVDQELDI
NRLSDYDVDAIVPQSFLKDDSIDNKVLTRSDKNRGKSDNVPSEEVVKKMKNYWRQLL
NAKLITQRKFDNLTKAERGGLSELDKAGFIKRQLVETRQITKHVAQILDSRMNTKYDEN
DKLIREVKVITLKSKLVSDFRKDFQFYKVREINNYHHAHDAYLNAVVGTALIKKYPKLE
SEFVYGDYKVYDVRKMIAKSEQEIGKATAKYFFYSNIMNFFKTEITLANGEIRKRPLIET
NGETGEIVWDKGRDFATVRKVLSMPQVNIVKKTEVQTGGFSKESILPKRNSDKLIARKK
DWDPKKYGGFDSPTVAYSVLVVAKVEKGKSKKLKSVKELLGITIMERSSFEKNPIDFLE
AKGYKEVKKDLIIKLPKYSLFELENGRKRMLASAGELQKGNELALPSKYVNFLYLASH
YEKLKGSPEDNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQSITGLYETRIDL
SQLGGD
SEQ ID NO: 24 (HA tag)
YPYDVPDYA
SEQ ID NO: 25 (SV40 NLS)
PKKKRKV
SEQ ID NO: 26 (Dnmt3A; residues 612-912; from Siddique et al., JMB, 2013; Stepper
et al., NAR, 2016)
NHDQEFDPPKVYPPVPAEKRKPIRVLSLFDGIATGLLVLKDLGIQVDRYIASEVCEDSITV
GMVRHQGKIMYVGDVRSVTQKHIQEWGPFDLVIGGSPCNDLSIVNPARKGLYEGTGRL
FFEFYRLLHDARPKEGDDRPFFWLFENVVAMGVSDKRDISRFLESNPVMIDAKEVSAAH
RARYFWGNLPGMNRPLASTVNDKLELQECLEHGRIAKFSKVRTITTRSNSIKQGKDQHF
PVFMNEKEDILWCTEMERVFGFPVHYTDVSNMSRLARQRLLGRSWSVPVIRHLFAPLK
EYFACV
SEQ ID NO: 27 (27 amino acid linker; from Siddique et al., JMB, 2013; Stepper et al.,
NAR, 2016)
SSGNSNANSRGPSFSSGLVPLSLRGSH
SEQ ID NO: 28 (Dnmt3L; from Siddique et al., JMB, 2013; Stepper et al., NAR, 2016)
MGPMEIYKTVSAWKRQPVRVLSLFRNIDKVLKSLGFLESGSGSGGGTLKYVEDVTNVV
RRDVEKWGPFDLVYGSTQPLGSSCDRCPGWYMFQFHRILQYALPRQESQRPFFWIFMD
NLLLTEDDQETTTRFLQTEAVTLQDVRGRDYQNAMRVWSNIPGLKSKHAPLTPKEEEY
LQAQVRSRSKLDAPKVDLLVKNCLLPLREYFKYFSQNSLPL
SEQ ID NO: 29 (P2A peptide cleave sequence)
ATNFSLLKQAGDVEENPGP
SEQ ID NO: 30 (BFP)
SELIKENMHMKLYMEGTVDNHHFKCTSEGEGKPYEGTQTMRIKVVEGGPLPFAFDILA
TSFLYGSKTFINHTQGIPDFFKQSFPEGFTWERVTTYEDGGVLTATQDTSLQDGCLIYNV
KIRGVNFTSNGPVMQKKTLGWEAFTETLYPADGGLEGRNDMALKLVGGSHLIANIKTT
YRSKKPAKNLKMPGVYYVDYRLERIKEANNETYVEQHEVAVARYCDLPSKLGHKLN*
SEQ ID NO: 31 (XTEN16 (16 amino acid sequence))
SGSETPGTSESATPES
SEQ ID NO: 32 (XTEN80 (80 amino acid sequence))
GGPSSGAPPPSGGSPAGSPTSTEEGTSESATPESGPGTSTEPSEGSAPGSPAGSPTSTEEGT
STEPSEGSAPGTSTEPSE
SEQ ID NO: 33 (Dnmt3A-Dnmt3L domain)
NHDQEFDPPKVYPPVPAEKRKPIRVLSLFDGIATGLLVLKDLGIQVDRYIASEVCEDSITV
GMVRHQGKIMYVGDVRSVTQKHIQEWGPFDLVIGGSPCNDLSIVNPARKGLYEGTGRL
FFEFYRLLHDARPKEGDDRPFFWLFENVVAMGVSDKRDISRFLESNPVMIDAKEVSAAH
RARYFWGNLPGMNRPLASTVNDKLELQECLEHGRIAKFSKVRTITTRSNSIKQGKDQHF
PVFMNEKEDILWCTEMERVFGFPVHYTDVSNMSRLARQRLLGRSWSVPVIRHLFAPLK
EYFACVSSGNSNANSRGPSFSSGLVPLSLRGSHMGPMEIYKTVSAWKRQPVRVLSLFRN
IDKVLKSLGFLESGSGSGGGTLKYVEDVTNVVRRDVEKWGPFDLVYGSTQPLGSSCDR
CPGWYMFQFHRILQYALPRQESQRPFFWIFMDNLLLTEDDQETTTRFLQTEAVTLQDVR
GRDYQNAMRVWSNIPGLKSKHAPLTPKEEEYLQAQVRSRSKLDAPKVDLLVKNCLLPL
REYFKYFSQNSLPL
SEQ ID NO: 34 (ddAsCfp1)
MTQFEGFTNLYQVSKTLRFELIPQGKTLKHIQEQGFIEEDKARNDHYKELKPIIDRIYKTY
ADQCLQLVQLDWENLSAAIDSYRKEKTEETRNALIEEQATYRNAIHDYFIGRTDNLTDA
INKRHAEIYKGLFKAELFNGKVLKQLGTVTTTEHENALLRSFDKFTTYFSGFYENRKNV
FSAEDISTAIPHRIVQDNFPKFKENCHIFTRLITAVPSLREHFENVKKAIGIFVSTSIEEVFSF
PFYNQLLTQTQIDLYNQLLGGISREAGTEKIKGLNEVLNLAIQKNDETAHIIASLPHRFIPL
FKQILSDRNTLSFILEEFKSDEEVIQSFCKYKTLLRNENVLETAEALFNELNSIDLTHIFISH
KKLETISSALCDHWDTLRNALYERRISELTGKITKSAKEKVQRSLKHEDINLQEIISAAGK
ELSEAFKQKTSEILSHAHAALDQPLPTTLKKQEEKEILKSQLDSLLGLYHLLDWFAVDES
NEVDPEFSARLTGIKLEMEPSLSFYNKARNYATKKPYSVEKFKLNFQMPTLASGWDVN
KEKNNGAILFVKNGLYYLGIMPKQKGRYKALSFEPTEKTSEGFDKMYYDYFPDAAKMI
PKCSTQLKAVTAHFQTHTTPILLSNNFIEPLEITKEIYDLNNPEKEPKKFQTAYAKKTGDQ
KGYREALCKWIDFTRDFLSKYTKTTSIDLSSLRPSSQYKDLGEYYAELNPLLYHISFQRIA
EKEIMDAVETGKLYLFQIYNKDFAKGHHGKPNLHTLYWTGLFSPENLAKTSIKLNGQA
ELFYRPKSRMKRMAHRLGEKMLNKKLKDQKTPIPDTLYQELYDYVNHRLSHDLSDEA
RALLPNVITKEVSHEIIKDRRFTSDKFFFHVPITLNYQAANSPSKFNQRVNAYLKEHPETP
IIGIDRGERNLIYITVIDSTGKILEQRSLNTIQQFDYQKKLDNREKERVAARQAWSVVGTI
KDLKQGYLSQVIHEIVDLMIHYQAVVVLANLNFGFKSKRTGIAEKAVYQQFEKMLIDK
LNCLVLKDYPAEKVGGVLNPYQLTDQFTSFAKMGTQSGFLFYVPAPYTSKIDPLTGFVD
PFVWKTIKNHESRKHFLEGFDFLHYDVKTGDFILHFKMNRNLSFQRGLPGFMPAWDIVF
EKNETQFDAKGTPFIAGKRIVPVIENHRFTGRYRDLYPANELIALLEEKGIVFRDGSNILP
KLLENDDSHAIDTMVALIRSVLQMRNSNAATGEDYINSPVRDLNGVCFDSRFQNPEWP
MDADANGAYHIALKGQLLLNHLKESKDLKLQNGISNQDWLAYIQELRN
SEQ ID NO: 35 (ddLbCfp1)
MSKLEKFTNCYSLSKTLRFKAIPVGKTQENIDNKRLLVEDEKRAEDYKGVKKLLDRYY
LSFINDVLHSIKLKNLNNYISLFRKKTRTEKENKELENLEINLRKEIAKAFKGNEGYKSLF
KKDIIETILPEFLDDKDEIALVNSFNGFTTAFTGFFDNRENMFSEEAKSTSIAFRCINENLT
RYISNMDIFEKVDAIFDKHEVQEIKEKILNSDYDVEDFFEGEFFNFVLTQEGIDVYNAIIG
GFVTESGEKIKGLNEYINLYNQKTKQKLPKFKPLYKQVLSDRESLSFYGEGYTSDEEVL
EVFRNTLNKNSEIFSSIKKLEKLFKNFDEYSSAGIFVKNGPAISTISKDIFGEWNVIRDKW
NAEYDDIHLKKKAVVTEKYEDDRRKSFKKIGSFSLEQLQEYADADLSVVEKLKEIIIQK
VDEIYKVYGSSEKLFDADFVLEKSLKKNDAVVAIMKDLLDSVKSFENYIKAFFGEGKET
NRDESFYGDFVLAYDILLKVDHIYDAIRNYVTQKPYSKDKFKLYFQNPQFMGGWDKD
KETDYRATILRYGSKYYLAIMDKKYAKCLQKIDKDDVNGNYEKINYKLLPGPNKMLPK
VFFSKKWMAYYNPSEDIQKIYKNGTFKKGDMFNLNDCHKLIDFFKDSISRYPKWSNAY
DFNFSETEKYKDIAGFYREVEEQGYKVSFESASKKEVDKLVEEGKLYMFQIYNKDFSDK
SHGTPNLHTMYFKLLFDENNHGQIRLSGGAELFMRRASLKKEELVVHPANSPIANKNPD
NPKKTTTLSYDVYKDKRFSEDQYELHIPIAINKCPKNIFKINTEVRVLLKHDDNPYVIGIA
RGERNLLYIVVVDGKGNIVEQYSLNEIINNFNGIRIKTDYHSLLDKKEKERFEARQNWTS
IENIKELKAGYISQVVHKICELVEKYDAVIALADLNSGFKNSRVKVEKQVYQKFEKMLI
DKLNYMVDKKSNPCATGGALKGYQITNKFESFKSMSTQNGFIFYIPAWLTSKIDPSTGF
VNLLKTKYTSIADSKKFISSFDRIMYVPEEDLFEFALDYKNFSRTDADYIKKWKLYSYG
NRIRIFRNPKKNNVFDWEEVCLTSAYKELFNKYGINYQQGDIRALLCEQSDKAFYSSFM
ALMSLMLQMRNSITGRTDVDFLISPVKNSDGIFYDSRNYEAQENAILPKNADANGAYNI
ARKVLWAIGQFKKAEDEKLDKVKIAISNKEWLEYAQTSVKH
SEQ ID NO: 36 (ddFnCfp1)
MYPYDVPDYASGSGMSIYQEFVNKYSLSKTLRFELIPQGKTLENIKARGLILDDEKRAK
DYKKAKQIIDKYHQFFIEEILSSVCISEDLLQNYSDVYFKLKKSDDDNLQKDFKSAKDTI
KKQISEYIKDSEKFKNLFNQNLIDAKKGQESDLILWLKQSKDNGIELFKANSDITDIDEAL
EIIKSFKGWTTYFKGFHENRKNVYSSNDIPTSIIYRIVDDNLPKFLENKAKYESLKDKAPE
AINYEQIKKDLAEELTFDIDYKTSEVNQRVFSLDEVFEIANFNNYLNQSGITKFNTIIGGK
FVNGENTKRKGINEYINLYSQQINDKTLKKYKMSVLFKQILSDTESKSFVIDKLEDDSDV
VTTMQSFYEQIAAFKTVEEKSIKETLSLLFDDLKAQKLDLSKIYFKNDKSLTDLSQQVFD
DYSVIGTAVLEYITQQIAPKNLDNPSKKEQELIAKKTEKAKYLSLETIKLALEEFNKHRDI
DKQCRFEEILANFAAIPMIFDEIAQNKDNLAQISIKYQNQGKKDLLQASAEDDVKAIKDL
LDQTNNLLHKLKIFHISQSEDKANILDKDEHFYLVFEECYFELANIVPLYNKIRNYITQKP
YSDEKFKLNFENSTLANGWDKNKEPDNTAILFIKDDKYYLGVMNKKNNKIFDDKAIKE
NKGEGYKKIVYKLLPGANKMLPKVFFSAKSIKFYNPSEDILRIRNHSTHTKNGSPQKGY
EKFEFNIEDCRKFIDFYKQSISKHPEWKDFGFRFSDTQRYNSIDEFYREVENQGYKLTFE
NISESYIDSVVNQGKLYLFQIYNKDFSAYSKGRPNLHTLYWKALFDERNLQDVVYKLN
GEAELFYRKQSIPKKITHPAKEAIANKNKDNPKKESVFEYDLIKDKRFTEDKFFFHCPITI
NFKSSGANKFNDEINLLLKEKANDVHILSIARGERHLAYYTLVDGKGNIIKQDTFNIIGN
DRMKTNYHDKLAAIEKDRDSARKDWKKINNIKEMKEGYLSQVVHEIAKLVIEYNAIVV
FEDLNFGFKRGRFKVEKQVYQKLEKMLIEKLNYLVFKDNEFDKTGGVLRAYQLTAPFE
TFKKMGKQTGIIYYVPAGFTSKICPVTGFVNQLYPKYESVSKSQEFFSKFDKICYNLDKG
YFEFSFDYKNFGDKAAKGKWTIASFGSRLINFRNSDKNHNWDTREVYPTKELEKLLKD
YSIEYGHGECIKAAICGESDKKFFAKLTSVLNTILQMRNSKTGTELDYLISPVADVNGNF
FDSRQAPKNMPQDADANGAYHIGLKGLMLLGRIKNNQEGKKLNLVIKNEEYFEFVQNR
NN
SEQ ID NO: 97 = V2.2 (DNMT3A-DNMT3L-XTEN80-dCas9-HA-NLS-NLS-XTEN16-
KRAB-P2A-BFP)
KRAB (bold, from Gilbert et al., Cell, 2013, 2014), Linkers (underlined), XTEN80 (lowercase
bold italics, 80 amino acid sequence), dCas9 (italics), HA tag (lowercase), SV40 NLS
(lowercase italics), DNMT3A (bold italics, from Siddique et al., JMB, 2013; Stepper et al.,
NAR, 2016), 27 amino acid linker (italics underlined, from Siddique et al., JMB, 2013; Stepper
et al., NAR, 2016), DNMT3L (bold underlined, from Siddique et al., JMB, 2013; Stepper et al.,
NAR, 2016), BFP (lowercase underlined)); XTEN16 linker is identified as:
(SGSETPGTSESATPES); P2A peptide cleavage sequence is identified as
((ATNFSLLKQAGDVEENPGP)). The use of parentheses in the sequence is merely to
identify the XTEN16 linker and the PA2 peptide cleavage sequence.
MNHDQEFDPPKVYPPVPAEKRKPIRVLSLFDGIATGLLVLKDLGIQVDRYIASEVCEDSI
TVGMVRHQGKIMYVGDVRSVTQKHIQEWGPFDLVIGGSPCNDLSIVNPARKGLYEGTG
RLFFEFYRLLHDARPKEGDDRPFFWLFENVVAMGVSDKRDISRFLESNPVMIDAKEVS
AAHRARYFWGNLPGMNRPLASTVNDKLELQECLEHGRIAKFSKVRTITTRSNSIKQGKD
QHFPVFMNEKEDILWCTEMERVFGFPVHYTDVSNMSRLARQRLLGRSWSVPVIRHLF
APLKEYFACVSSGNSNANSRGPSFSSGLVPLSLRGSHMGPMEIYKTVSAWKRQPVRVLSLF
RNIDKVLKSLGFLESGSGSGGGTLKYVEDVTNVVRRDVEKWGPFDLVYGSTQPLGSSCD
RCPGWYMFQFHRILQYALPRQESQRPFFWIFMDNLLLTEDDQETTTRFLQTEAVTLQD
VRGRDYQNAMRVWSNIPGLKSKHAPLTPKEEEYLQAQVRSRSKLDAPKVDLLVKNCLL
PLREYFKYFSQNSLPLggpssgapppsggspagsptsteegtsesatpesgpgtstepsegsapgspagsptsteegtstep
segsapgtstepseMDKKYSIGLAIGTNSVGWAVITDEYKVPSKKFKVLGNTDRHSIKKNLIGALLFD
SGETAEATRLKRTARRRYTRRKNRICYLQEIFSNEMAKVDDSFFHRLEESFLVEEDKKHERHP
IFGNIVDEVAYHEKYPTIYHLRKKLVDSTDKADLRLIYLALAHMIKFRGHFLIEGDLNPDNSDV
DKLFIQLVQTYNQLFEENPINASGVDAKAILSARLSKSRRLENLIAQLPGEKKNGLFGNLIALSL
GLTPNFKSNFDLAEDAKLQLSKDTYDDDLDNLLAQIGDQYADLFLAAKNLSDAILLSDILRVN
TEITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLKD
NREKIEKILTFRIPYYVGPLARGNSRFAWMTRKSEETITPWNFEEVVDKGASAQSFIERMTNFD
KNLPNEKVLPKHSLLYEYFTVYNELTKVKYVTEGMRKPAFLSGEQKKAIVDLLFKTNRKVTVK
QLKEDYFKKIECFDSVEISGVEDRFNASLGTYHDLLKIIKDKDFLDNEENEDILEDIVLTLTLF
EDREMIEERLKTYAHLFDDKVMKQLKRRRYTGWGRLSRKLINGIRDKQSGKTILDFLKSDGF
ANRNFMQLIHDDSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVENTQLQNEKLY
LYYLQNGRDMYVDQELDINRLSDYDVDAIVPQSFLKDDSIDNKVLTRSDKNRGKSDNVPSEE
VVKKMKNYWRQLLNAKLITQRKFDNLTKAERGGLSELDKAGFIKRQLVETRQITKHVAQILD
SRMNTKYDENDKLIREVKVITLKSKLVSDFRKDFQFYKVREINNYHHAHDAYLNAVVGTALIK
KYPKLESEFVYGDYKVYDVRKMIAKSEQEIGKATAKYFFYSNIMNFFKTEITLANGEIRKRPLI
ETNGETGEIVWDKGRDFATVRKVLSMPQVNIVKKTEVQTGGFSKESILPKRNSDKLIARKKD
WDPKKYGGFDSPTVAYSVLVVAKVEKGKSKKLKSVKELLGITIMERSSFEKNPIDFLEAKGYK
EVKKDLIIKLPKYSLFELENGRKRMLASAGELQKGNELALPSKYVNFLYLASHYEKLKGSPED
NEQKQLFVEQHKHYLDEIIEQISEFSKRVILADANLDKVLSAYNKHRDKPIREQAENIIHLFTL
TNLGAPAAFKYFDTTIDRKRYTSTKEVLDATLIHQSITGLYETRIDLSQLGGDAypydvpdyaSLG
SGSpkkkrkvEDpkkkrkvDGIGSGSNGSSGS(SGSETPGTSESATPES)GGGGGMDAKSLTA
WSRTLVTFKDVFVDFTREEWKLLDTAQQIVYRNVMLENYKNLVSLGYQLTKPDVI
LRLEKGEEP((ATNFSLLKQAGDVEENPGP))selikenmhmklymegtvdnhhfkctsegegkpyegtqt
mrikvveggplpfafdilatsflygsktfinhtqgipdffkqsfpegftwervttyedggvltatqdtslqdgcliynvkirgvnftsngpv
mqkktlgweaftetlypadgglegrndmalklvggshlianikttyrskkpaknlkmpgvyyvdyrlerikeannetyveghevav
arycdlpsklghkln*
SEQ ID NO: 98 = V2.3 (DNMT3A-DNMT3L-XTEN80-NLS-dCas9-NLS-XTEN16-
KRAB-P2A-BFP)
KRAB (bold, from Gilbert et al., Cell, 2013, 2014), Linkers (underlined), XTEN80 (lowercase
bold italics, 80 amino acid sequence), dCas9 (italics), HA tag (lowercase), SV40 NLS
(lowercase italics), DNMT3A (bold italics, from Siddique et al., JMB, 2013; Stepper et al.,
NAR, 2016), 27 amino acid linker (italics underlined, from Siddique et al., JMB, 2013; Stepper
et al., NAR, 2016), DNMT3L (bold underlined, from Siddique et al., JMB, 2013; Stepper et al.,
NAR, 2016), BFP (lowercase underlined)); XTEN16 linker is identified as
(SGSETPGTSESATPES); P2A peptide cleavage sequence is identified as
((ATNFSLLKQAGDVEENPGP)). The use of parentheses in the sequence is merely to
identify the XTEN16 linker and the PA2 peptide cleavage sequence.
MNHDQEFDPPKVYPPVPAEKRKPIRVLSLFDGIATGLLVLKDLGIQVDRYIASEVCEDSI
TVGMVRHQGKIMYVGDVRSVTQKHIQEWGPFDLVIGGSPCNDLSIVNPARKGLYEGTG
RLFFEFYRLLHDARPKEGDDRPFFWLFENVVAMGVSDKRDISRFLESNPVMIDAKEVS
AAHRARYFWGNLPGMNRPLASTVNDKLELQECLEHGRIAKFSKVRTITTRSNSIKQGKD
QHFPVFMNEKEDILWCTEMERVFGFPVHYTDVSNMSRLARQRLLGRSWSVPVIRHLF
APLKEYFACVSSGNSNANSRGPSFSSGLVPLSLRGSHMGPMEIYKTVSAWKRQPVRVLSLF
RNIDKVLKSLGFLESGSGSGGGTLKYVEDVTNVVRRDVEKWGPFDLVYGSTQPLGSSCD
RCPGWYMFQFHRILQYALPRQESQRPFFWIFMDNLLLTEDDQETTTRFLQTEAVTLQD
VRGRDYQNAMRVWSNIPGLKSKHAPLTPKEEEYLQAQVRSRSKLDAPKVDLLVKNCLL
PLREYFKYFSQNSLPLggpssgapppsggspagsptsteegtsesatpesgpgtstepsegsapgspagsptsteegtstep
segsapgtstepsepkkkrkvMDKKYSIGLAIGTNSVGWAVITDEYKVPSKKFKVLGNTDRHSIKKNLIG
ALLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDLNP
DNSDVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSDAILL
SDILRVNTEITKAPLSASMIKRYDEHHQDLTLLKALVRQQLPEKYKEIFFDQSKNGYAGYIDG
GASQEEFYKFIKPILEKMDGTEELLVKLNREDLLRKQRTFDNGSIPHQIHLGELHAILRRQED
FYPFLKDNREKIEKILTFRIPYYVGPLARGNSRFAWMTRKSEETITPWNFEEVVDKGASAQSFI
ERMTNFDKNLPNEKVLPKHSLLYEYFTVYNELTKVKYVTEGMRKPAFLSGEQKKAIVDLLFK
TNRKVTVKQLKEDYFKKIECFDSVEISGVEDRFNASLGTYHDLLKIIKDKDFLDNEENEDILE
DIVLTLTLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVK
VVDELVKVMGRHKPENIVIEMARENQTTQKGQKNSRERMKRIEEGIKELGSQILKEHPVENT
QLQNEKLYLYYLQNGRDMYVDQELDINRLSDYDVDAIVPQSFLKDDSIDNKVLTRSDKNRGK
SDNVPSEEVVKKMKNYWRQLLNAKLITQRKFDNLTKAERGGLSELDKAGFIKRQLVETRQIT
KHVAQILDSRMNTKYDENDKLIREVKVITLKSKLVSDFRKDFQFYKVREINNYHHAHDAYLNA
VVGTALIKKYPKLESEFVYGDYKVYDVRKMIAKSEQEIGKATAKYFFYSNIMNFFKTEITLANG
EIRKRPLIETNGETGEIVWDKGRDFATVRKVLSMPQVNIVKKTEVQTGGFSKESILPKRNSDK
LIARKKDWDPKKYGGFDSPTVAYSVLVVAKVEKGKSKKLKSVKELLGITIMERSSFEKNPIDF
LEAKGYKEVKKDLIIKLPKYSLFELENGRKRMLASAGELQKGNELALPSKYVNFLYLASHYEK
LKGSPEDNEQKQLFVEQHKHYLDEIIEQISEFSKRVILADANLDKVLSAYNKHRDKPIREQAE
NIIHLFTLTNLGAPAAFKYFDTTIDRKRYTSTKEVLDATLIHQSITGLYETRIDLSQLGGDpkkkrk
v(SGSETPGTSESATPES)RTLVTFKDVFVDFTREEWKLLDTAQQIVYRNVMLENYK
NLVSLGYQLTKPDVILRLEKGEEP((ATNFSLLKQAGDVEENPGP))selikenmhmklymeg
tvdnhhfkctsegegkpyegtqtmrikvveggplpfafdilatsflygsktfinhtqgipdffkqsfpegftwervttyedggvltatqdt
slqdgcliynvkirgvnftsngpvmqkktlgweaftetlypadgglegrndmalklvggshlianikttyrskkpaknlkmpgvyyv
dyrlerikeannetyveghevavarycdlpsklghkln*
SEQ ID NO: 99 = V2.4 (DNMT3A-DNMT3L-XTEN80-NLS-dCas9-NLS-XTEN16-
BFP-KRAB)
KRAB (bold, from Gilbert et al., Cell, 2013, 2014), Linkers (underlined), XTEN80 (lowercase
bold italics, 80 amino acid sequence), dCas9 (italics), HA tag (lowercase), SV40 NLS
(lowercase italics), DNMT3A (bold italics, from Siddique et al., JMB, 2013; Stepper et al.,
NAR, 2016), 27 amino acid linker (italics underlined, from Siddique et al., JMB, 2013; Stepper
et al., NAR, 2016), DNMT3L (bold underlined, from Siddique et al., JMB, 2013; Stepper et al.,
NAR, 2016), BFP (lowercase underlined)); XTEN16 linker is identified as
(SGSETPGTSESATPES). The use of parentheses in the sequence is merely to identify the
XTEN16 linker sequence.
MNHDQEFDPPKVYPPVPAEKRKPIRVLSLFDGIATGLLVLKDLGIQVDRYIASEVCEDSI
TVGMVRHQGKIMYVGDVRSVTQKHIQEWGPFDLVIGGSPCNDLSIVNPARKGLYEGTG
RLFFEFYRLLHDARPKEGDDRPFFWLFENVVAMGVSDKRDISRFLESNPVMIDAKEVS
AAHRARYFWGNLPGMNRPLASTVNDKLELQECLEHGRIAKFSKVRTITTRSNSIKQGKD
QHFPVFMNEKEDILWCTEMERVFGFPVHYTDVSNMSRLARQRLLGRSWSVPVIRHLF
APLKEYFACVSSGNSNANSRGPSFSSGLVPLSLRGSHMGPMEIYKTVSAWKRQPVRVLSLF
RNIDKVLKSLGFLESGSGSGGGTLKYVEDVTNVVRRDVEKWGPFDLVYGSTQPLGSSCD
RCPGWYMFQFHRILQYALPRQESQRPFFWIFMDNLLLTEDDQETTTRFLQTEAVTLQD
VRGRDYQNAMRVWSNIPGLKSKHAPLTPKEEEYLQAQVRSRSKLDAPKVDLLVKNCLL
PLREYFKYFSQNSLPLggpssgapppsggspagsptsteegtsesatpesgpgtstepsegsapgspagsptsteegtstep
segsapgtstepsepkkkrkvMDKKYSIGLAIGTNSVGWAVITDEYKVPSKKFKVLGNTDRHSIKKNLIG
ALLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDLNP
DNSDVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSDAILL
SDILRVNTEITKAPLSASMIKRYDEHHQDLTLLKALVRQQLPEKYKEIFFDQSKNGYAGYIDG
GASQEEFYKFIKPILEKMDGTEELLVKLNREDLLRKQRTFDNGSIPHQIHLGELHAILRRQED
FYPFLKDNREKIEKILTFRIPYYVGPLARGNSRFAWMTRKSEETITPWNFEEVVDKGASAQSFI
ERMTNFDKNLPNEKVLPKHSLLYEYFTVYNELTKVKYVTEGMRKPAFLSGEQKKAIVDLLFK
TNRKVTVKQLKEDYFKKIECFDSVEISGVEDRFNASLGTYHDLLKIIKDKDFLDNEENEDILE
DIVLTLTLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVK
VVDELVKVMGRHKPENIVIEMARENQTTQKGQKNSRERMKRIEEGIKELGSQILKEHPVENT
QLQNEKLYLYYLQNGRDMYVDQELDINRLSDYDVDAIVPQSFLKDDSIDNKVLTRSDKNRGK
SDNVPSEEVVKKMKNYWRQLLNAKLITQRKFDNLTKAERGGLSELDKAGFIKRQLVETRQIT
KHVAQILDSRMNTKYDENDKLIREVKVITLKSKLVSDFRKDFQFYKVREINNYHHAHDAYLNA
VVGTALIKKYPKLESEFVYGDYKVYDVRKMIAKSEQEIGKATAKYFFYSNIMNFFKTEITLANG
EIRKRPLIETNGETGEIVWDKGRDFATVRKVLSMPQVNIVKKTEVQTGGFSKESILPKRNSDK
LIARKKDWDPKKYGGFDSPTVAYSVLVVAKVEKGKSKKLKSVKELLGITIMERSSFEKNPIDF
LEAKGYKEVKKDLIIKLPKYSLFELENGRKRMLASAGELQKGNELALPSKYVNFLYLASHYEK
LKGSPEDNEQKQLFVEQHKHYLDEIIEQISEFSKRVILADANLDKVLSAYNKHRDKPIREQAE
NIIHLFTLTNLGAPAAFKYFDTTIDRKRYTSTKEVLDATLIHQSITGLYETRIDLSQLGGDpkkkrk
v(SGSETPGTSESATPES)selikenmhmklymegtvdnhhfkctsegegkpyegtqtmrikvveggplpfafdilatsf
lygsktfinhtqgipdffkqsfpegftwervttyedggvltatqdtslqdgcliynvkirgvnftsngpvmqkktlgweaftetlypadg
glegrndmalklvggshlianikttyrskkpaknlkmpgvyyvdyrlerikeannetyveghevavarycdlpsklghklnGGGG
GMDAKSLTAWSRTLVTFKDVFVDFTREEWKLLDTAQQIVYRNVMLENYKNLVSL
GYQLTKPDVILRLEKGEEP*
SEQ ID NO: 100
GACGCTCAAATTTCCGCAGTGTTTAAGAGCTAAGCTGGAAACAGCATAGCAAGTTT
AAATAAGGCTAGTCCGTTATCAACTTGAAAAAGTGGCACCGAGTCGGTGCTTTTTTT
SEQ ID NO: 101
GTTTAAGAGCTAAGCTGGAAACAGCATAGCAAGTTTAAATAAGGCTAGTCCGTTAT
CAACTTGAAAAAGTGGCACCGAGTCGGTGCTTTTTTT
SEQ ID NO: 102
GACGCTCAAATTTCCGCAGT
SEQ ID NO: 103 (KRAB with initiating methionine)
MDAKSLTAWSRTLVTFKDVFVDFTREEWKLLDTAQQIVYRNVMLENYKNLVSLGYQL
TKPDVILRLEKGEEP
SEQ ID NO: 104 (KRAB)
RTLVTFKDVFVDFTREEWKLLDTAQQIVYRNVMLENYKNLVSLGYQLTKPDVILRLEK
GEEP
SEQ ID NO: 105 (KRAB)
MRTLVTFKDVFVDFTREEWKLLDTAQQIVYRNVMLENYKNLVSLGYQLTKPDVILRLE
KGEEP
SEQ ID NO: 106 (Dnmt3A with methionine on N-terminus)
MNHDQEFDPPKVYPPVPAEKRKPIRVLSLFDGIATGLLVLKDLGIQVDRYIASEVCEDSI
TVGMVRHQGKIMYVGDVRSVTQKHIQEWGPFDLVIGGSPCNDLSIVNPARKGLYEGTG
RLFFEFYRLLHDARPKEGDDRPFFWLFENVVAMGVSDKRDISRFLESNPVMIDAKEVSA
AHRARYFWGNLPGMNRPLASTVNDKLELQECLEHGRIAKFSKVRTITTRSNSIKQGKDQ
HFPVFMNEKEDILWCTEMERVFGFPVHYTDVSNMSRLARQRLLGRSWSVPVIRHLFAP
LKEYFACV
SEQ ID NO: 107 (V2.1 or p112 (Dnmt3A-Dnmt3L-XTEN80-dCas9-BFP-KRAB);
KRAB (bold, from Gilbert et al., Cell, 2013, 2014), Linkers (underlined), XTEN80 (lowercase
bold italics, 80 amino acid sequence), dCas9 (italics), SV40 NLS (lowercase italics), Dnmt3A
(bold italics, from Siddique et al., JMB, 2013; Stepper et al., NAR, 2016), 27 amino acid linker
(italics underlined, from Siddique et al., JMB, 2013; Stepper et al., NAR, 2016), Dnmt3L (bold
underlined, from Siddique et al., JMB, 2013; Stepper et al., NAR, 2016),
NHDQEFDPPKVYPPVPAEKRKPIRVLSLFDGIATGLLVLKDLGIQVDRYIASEVCEDSIT
VGMVRHQGKIMYVGDVRSVTQKHIQEWGPFDLVIGGSPCNDLSIVNPARKGLYEGTGR
LFFEFYRLLHDARPKEGDDRPFFWLFENVVAMGVSDKRDISRFLESNPVMIDAKEVSA
AHRARYFWGNLPGMNRPLASTVNDKLELQECLEHGRIAKFSKVRTITTRSNSIKQGKDQ
HFPVFMNEKEDILWCTEMERVFGFPVHYTDVSNMSRLARQRLLGRSWSVPVIRHLFA
PLKEYFACVSSGNSNANSRGPSFSSGLVPLSLRGSHMGPMEIYKTVSAWKRQPVRVLSL
FRNIDKVLKSLGFLESGSGSGGGTLKYVEDVTNVVRRDVEKWGPFDLVYGSTQPL
GSSCDRCPGWYMFQFHRILQYALPRQESQRPFFWIFMDNLLLTEDDQETTTRELQ
TEAVTLQDVRGRDYQNAMRVWSNIPGLKSKHAPLTPKEEEYLQAQVRSRSKLDAP
KVDLLVKNCLLPLREYFKYFSQNSLPLggpssgapppsggspagsptsteegtsesatpesgpgtstepsegs
apgspagsptsteegtstepsegsapgtstepseMDKKYSIGLAIGTNSVGWAVITDEYKVPSKKFKVLGNTD
RHSIKKNLIGALLFDSGETAEATRLKRTARRRYTRRKNRICYLQEIFSNEMAKVDDSFFHRLEE
SFLVEEDKKHERHPIFGNIVDEVAYHEKYPTIYHLRKKLVDSTDKADLRLIYLALAHMIKFRG
HFLIEGDLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYADLFLA
AKNLSDAILLSDILRVNTEITKAPLSASMIKRYDEHHQDLTLLKALVRQQLPEKYKEIFFDQSK
NGYAGYIDGGASQEEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKSEETITPWNFEEVVD
KGASAQSFIERMTNFDKNLPNEKVLPKHSLLYEYFTVYNELTKVKYVTEGMRKPAFLSGEQK
KAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIR
DKQSGKTILDFLKSDGFANRNFMQLIHDDSLTFKEDIQKAQVSGQGDSLHEHIANLAGSPAI
KKGILQTVKVVDELVKVMGRHKPENIVIEMARENQTTQKGQKNSRERMKRIEEGIKELGSQI
LKEHPVENTQLQNEKLYLYYLQNGRDMYVDQELDINRLSDYDVDAIVPQSFLKDDSIDNKVL
TRSDKNRGKSDNVPSEEVVKKMKNYWRQLLNAKLITQRKFDNLTKAERGGLSELDKAGFIKR
QLVETRQITKHVAQILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKATAKYFFYSNIMNF
FKTEITLANGEIRKRPLIETNGETGEIVWDKGRDFATVRKVLSMPQVNIVKKTEVQTGGFSKE
SILPKRNSDKLIARKKDWDPKKYGGFDSPTVAYSVLVVAKVEKGKSKKLKSVKELLGITIMERS
SFEKNPIDFLEAKGYKEVKKDLIIKLPKYSLFELENGRKRMLASAGELQKGNELALPSKYVNF
LYLASHYEKLKGSPEDNEQKQLFVEQHKHYLDEIIEQISEFSKRVILADANLDKVLSAYNKHR
DKPIREQAENIIHLFTLTNLGAPAAFKYFDTTIDRKRYTSTKEVLDATLIHQSITGLYETRIDLSQ
LGGDASLGSGSpkkkrkvEDpkkkrkvDGIGSGSNGSSGSGGGGGMDAKSLTAWSRTLVTFK
DVFVDFTREEWKLLDTAQQIVYRNVMLENYKNLVSLGYQLTKPDVILRLEKGEEP*
SEQ ID NO: 108 = V2.2 (DNMT3A-DNMT3L-XTEN80-dCas9-HA-NLS-NLS-
XTEN16-KRAB-P2A-BFP)
KRAB (bold, from Gilbert et al., Cell, 2013, 2014), Linkers (underlined), XTEN80 (lowercase
bold italics, 80 amino acid sequence), dCas9 (italics), SV40 NLS (lowercase italics), DNMT3A
(bold italics, from Siddique et al., JMB, 2013; Stepper et al., NAR, 2016), 27 amino acid linker
(italics underlined, from Siddique et al., JMB, 2013; Stepper et al., NAR, 2016), DNMT3L (bold
underlined, from Siddique et al., JMB, 2013; Stepper et al., NAR, 2016), BFP (lowercase
underlined)); XTEN16 linker is identified as: (SGSETPGTSESATPES). The use of
parentheses in the sequence is merely to identify the XTEN16 linker.
MNHDQEFDPPKVYPPVPAEKRKPIRVLSLFDGIATGLLVLKDLGIQVDRYIASEVCEDSI
TVGMVRHQGKIMYVGDVRSVTQKHIQEWGPFDLVIGGSPCNDLSIVNPARKGLYEGTG
RLFFEFYRLLHDARPKEGDDRPFFWLFENVVAMGVSDKRDISRFLESNPVMIDAKEVS
AAHRARYFWGNLPGMNRPLASTVNDKLELQECLEHGRIAKFSKVRTITTRSNSIKQGKD
QHFPVFMNEKEDILWCTEMERVFGFPVHYTDVSNMSRLARQRLLGRSWSVPVIRHLF
APLKEYFACVSSGNSNANSRGPSFSSGLVPLSLRGSHMGPMEIYKTVSAWKRQPVRVLSLF
RNIDKVLKSLGFLESGSGSGGGTLKYVEDVTNVVRRDVEKWGPFDLVYGSTQPLGSSCD
RCPGWYMFQFHRILQYALPRQESQRPFFWIFMDNLLLTEDDQETTTRFLQTEAVTLQD
VRGRDYQNAMRVWSNIPGLKSKHAPLTPKEEEYLQAQVRSRSKLDAPKVDLLVKNCLL
PLREYFKYFSQNSLPLggpssgapppsggspagsptsteegtsesatpesgpgtstepsegsapgspagsptsteegtstep
segsapgtstepseMDKKYSIGLAIGTNSVGWAVITDEYKVPSKKFKVLGNTDRHSIKKNLIGALLFD
SGETAEATRLKRTARRRYTRRKNRICYLQEIFSNEMAKVDDSFFHRLEESFLVEEDKKHERHP
IFGNIVDEVAYHEKYPTIYHLRKKLVDSTDKADLRLIYLALAHMIKFRGHFLIEGDLNPDNSDV
DKLFIQLVQTYNQLFEENPINASGVDAKAILSARLSKSRRLENLIAQLPGEKKNGLFGNLIALSL
GLTPNFKSNFDLAEDAKLQLSKDTYDDDLDNLLAQIGDQYADLFLAAKNLSDAILLSDILRVN
TEITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLKD
NREKIEKILTFRIPYYVGPLARGNSRFAWMTRKSEETITPWNFEEVVDKGASAQSFIERMTNED
KNLPNEKVLPKHSLLYEYFTVYNELTKVKYVTEGMRKPAFLSGEQKKAIVDLLFKTNRKVTVK
QLKEDYFKKIECFDSVEISGVEDRFNASLGTYHDLLKIIKDKDFLDNEENEDILEDIVLTLTLF
EDREMIEERLKTYAHLFDDKVMKQLKRRRYTGWGRLSRKLINGIRDKQSGKTILDFLKSDGF
ANRNFMQLIHDDSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVENTQLQNEKLY
LYYLQNGRDMYVDQELDINRLSDYDVDAIVPQSFLKDDSIDNKVLTRSDKNRGKSDNVPSEE
VVKKMKNYWRQLLNAKLITQRKFDNLTKAERGGLSELDKAGFIKRQLVETRQITKHVAQILD
SRMNTKYDENDKLIREVKVITLKSKLVSDFRKDFQFYKVREINNYHHAHDAYLNAVVGTALIK
KYPKLESEFVYGDYKVYDVRKMIAKSEQEIGKATAKYFFYSNIMNFFKTEITLANGEIRKRPLI
ETNGETGEIVWDKGRDFATVRKVLSMPQVNIVKKTEVQTGGFSKESILPKRNSDKLIARKKD
WDPKKYGGFDSPTVAYSVLVVAKVEKGKSKKLKSVKELLGITIMERSSFEKNPIDFLEAKGYK
EVKKDLIIKLPKYSLFELENGRKRMLASAGELQKGNELALPSKYVNFLYLASHYEKLKGSPED
NEQKQLFVEQHKHYLDEIIEQISEFSKRVILADANLDKVLSAYNKHRDKPIREQAENIIHLFTL
TNLGAPAAFKYFDTTIDRKRYTSTKEVLDATLIHQSITGLYETRIDLSQLGGDASLGSGSpkkkrk
vEDpkkkrkvDGIGSGSNGSSGS(SGSETPGTSESATPES)GGGGGMDAKSLTAWSRTLVT
FKDVFVDFTREEWKLLDTAQQIVYRNVMLENYKNLVSLGYQLTKPDVILRLEKGE
EP
SEQ ID NO: 109 = V2.3 (DNMT3A-DNMT3L-XTEN80-NLS-dCas9-NLS-XTEN16-
KRAB-P2A-BFP)
KRAB (bold, from Gilbert et al., Cell, 2013, 2014), Linkers (underlined), XTEN80 (lowercase
bold italics, 80 amino acid sequence), dCas9 (italics), SV40 NLS (lowercase italics), DNMT3A
(bold italics, from Siddique et al., JMB, 2013; Stepper et al., NAR, 2016), 27 amino acid
linker (italics underlined, from Siddique et al., JMB, 2013; Stepper et al., NAR, 2016), DNMT3L
(bold underlined, from Siddique et al., JMB, 2013; Stepper et al., NAR, 2016), XTEN16 linker is
identified as (SGSETPGTSESATPES). The use of parentheses in the sequence is merely to
identify the XTEN16 linker.
MNHDQEFDPPKVYPPVPAEKRKPIRVLSLFDGIATGLLVLKDLGIQVDRYIASEVCEDSI
TVGMVRHQGKIMYVGDVRSVTQKHIQEWGPFDLVIGGSPCNDLSIVNPARKGLYEGTG
RLFFEFYRLLHDARPKEGDDRPFFWLFENVVAMGVSDKRDISRFLESNPVMIDAKEVS
AAHRARYFWGNLPGMNRPLASTVNDKLELQECLEHGRIAKFSKVRTITTRSNSIKQGKD
QHFPVFMNEKEDILWCTEMERVFGFPVHYTDVSNMSRLARQRLLGRSWSVPVIRHLF
APLKEYFACVSSGNSNANSRGPSFSSGLVPLSLRGSHMGPMEIYKTVSAWKRQPVRVLSLF
RNIDKVLKSLGFLESGSGSGGGTLKYVEDVTNVVRRDVEKWGPFDLVYGSTQPLGSSCD
RCPGWYMFQFHRILQYALPRQESQRPFFWIFMDNLLLTEDDQETTTRFLQTEAVTLQD
VRGRDYQNAMRVWSNIPGLKSKHAPLTPKEEEYLQAQVRSRSKLDAPKVDLLVKNCLL
PLREYFKYFSQNSLPLggpssgapppsggspagsptsteegtsesatpesgpgtstepsegsapgspagsptsteegtstep
segsapgtstepsepkkkrkvMDKKYSIGLAIGTNSVGWAVITDEYKVPSKKFKVLGNTDRHSIKKNLIG
ALLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDLNP
DNSDVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSDAILL
SDILRVNTEITKAPLSASMIKRYDEHHQDLTLLKALVRQQLPEKYKEIFFDQSKNGYAGYIDG
GASQEEFYKFIKPILEKMDGTEELLVKLNREDLLRKQRTFDNGSIPHQIHLGELHAILRRQED
FYPFLKDNREKIEKILTFRIPYYVGPLARGNSRFAWMTRKSEETITPWNFEEVVDKGASAQSFI
ERMTNFDKNLPNEKVLPKHSLLYEYFTVYNELTKVKYVTEGMRKPAFLSGEQKKAIVDLLFK
TNRKVTVKQLKEDYFKKIECFDSVEISGVEDRFNASLGTYHDLLKIIKDKDFLDNEENEDILE
DIVLTLTLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVK
VVDELVKVMGRHKPENIVIEMARENQTTQKGQKNSRERMKRIEEGIKELGSQILKEHPVENT
QLQNEKLYLYYLQNGRDMYVDQELDINRLSDYDVDAIVPQSFLKDDSIDNKVLTRSDKNRGK
SDNVPSEEVVKKMKNYWRQLLNAKLITQRKFDNLTKAERGGLSELDKAGFIKRQLVETRQIT
KHVAQILDSRMNTKYDENDKLIREVKVITLKSKLVSDFRKDFQFYKVREINNYHHAHDAYLNA
VVGTALIKKYPKLESEFVYGDYKVYDVRKMIAKSEQEIGKATAKYFFYSNIMNFFKTEITLANG
EIRKRPLIETNGETGEIVWDKGRDFATVRKVLSMPQVNIVKKTEVQTGGFSKESILPKRNSDK
LIARKKDWDPKKYGGFDSPTVAYSVLVVAKVEKGKSKKLKSVKELLGITIMERSSFEKNPIDF
LEAKGYKEVKKDLIIKLPKYSLFELENGRKRMLASAGELQKGNELALPSKYVNFLYLASHYEK
LKGSPEDNEQKQLFVEQHKHYLDEIIEQISEFSKRVILADANLDKVLSAYNKHRDKPIREQAE
NIIHLFTLTNLGAPAAFKYFDTTIDRKRYTSTKEVLDATLIHQSITGLYETRIDLSQLGGDpkkkrk
v(SGSETPGTSESATPES)RTLVTFKDVFVDFTREEWKLLDTAQQIVYRNVMLENYK
NLVSLGYQLTKPDVILRLEKGEEP
SEQ ID NO: 110 = V2.4 (DNMT3A-DNMT3L-XTEN80-NLS-dCas9-NLS-XTEN16-
BFP-KRAB)
KRAB (bold, from Gilbert et al., Cell, 2013, 2014), Linkers (underlined), XTEN80 (lowercase
bold italics, 80 amino acid sequence), dCas9 (italics), SV40 NLS (lowercase italics), DNMT3A
(bold italics, from Siddique et al., JMB, 2013; Stepper et al., NAR, 2016), 27 amino acid
linker (italics underlined, from Siddique et al., JMB, 2013; Stepper et al., NAR, 2016), DNMT3L
(bold underlined, from Siddique et al., JMB, 2013; Stepper et al., NAR, 2016), XTEN16 linker is
identified as (SGSETPGTSESATPES). The use of parentheses in the sequence is merely to
identify the XTEN16 linker sequence.
MNHDQEFDPPKVYPPVPAEKRKPIRVLSLFDGIATGLLVLKDLGIQVDRYIASEVCEDSI
TVGMVRHQGKIMYVGDVRSVTQKHIQEWGPFDLVIGGSPCNDLSIVNPARKGLYEGTG
RLFFEFYRLLHDARPKEGDDRPFFWLFENVVAMGVSDKRDISRFLESNPVMIDAKEVS
AAHRARYFWGNLPGMNRPLASTVNDKLELQECLEHGRIAKFSKVRTITTRSNSIKQGKD
QHFPVFMNEKEDILWCTEMERVFGFPVHYTDVSNMSRLARQRLLGRSWSVPVIRHLF
APLKEYFACVSSGNSNANSRGPSFSSGLVPLSLRGSHMGPMEIYKTVSAWKRQPVRVLSLF
RNIDKVLKSLGFLESGSGSGGGTLKYVEDVTNVVRRDVEKWGPFDLVYGSTQPLGSSCD
RCPGWYMFQFHRILQYALPRQESQRPFFWIFMDNLLLTEDDQETTTRFLQTEAVTLQD
VRGRDYQNAMRVWSNIPGLKSKHAPLTPKEEEYLQAQVRSRSKLDAPKVDLLVKNCLL
PLREYFKYFSQNSLPLggpssgapppsggspagsptsteegtsesatpesgpgtstepsegsapgspagsptsteegtstep
segsapgtstepsepkkkrkvMDKKYSIGLAIGTNSVGWAVITDEYKVPSKKFKVLGNTDRHSIKKNLIG
ALLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDLNP
DNSDVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSDAILL
SDILRVNTEITKAPLSASMIKRYDEHHQDLTLLKALVRQQLPEKYKEIFFDQSKNGYAGYIDG
GASQEEFYKFIKPILEKMDGTEELLVKLNREDLLRKQRTFDNGSIPHQIHLGELHAILRRQED
FYPFLKDNREKIEKILTFRIPYYVGPLARGNSRFAWMTRKSEETITPWNFEEVVDKGASAQSFI
ERMTNFDKNLPNEKVLPKHSLLYEYFTVYNELTKVKYVTEGMRKPAFLSGEQKKAIVDLLFK
TNRKVTVKQLKEDYFKKIECFDSVEISGVEDRFNASLGTYHDLLKIIKDKDFLDNEENEDILE
DIVLTLTLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVK
VVDELVKVMGRHKPENIVIEMARENQTTQKGQKNSRERMKRIEEGIKELGSQILKEHPVENT
QLQNEKLYLYYLQNGRDMYVDQELDINRLSDYDVDAIVPQSFLKDDSIDNKVLTRSDKNRGK
SDNVPSEEVVKKMKNYWRQLLNAKLITQRKFDNLTKAERGGLSELDKAGFIKRQLVETRQIT
KHVAQILDSRMNTKYDENDKLIREVKVITLKSKLVSDFRKDFQFYKVREINNYHHAHDAYLNA
VVGTALIKKYPKLESEFVYGDYKVYDVRKMIAKSEQEIGKATAKYFFYSNIMNFFKTEITLANG
EIRKRPLIETNGETGEIVWDKGRDFATVRKVLSMPQVNIVKKTEVQTGGFSKESILPKRNSDK
LIARKKDWDPKKYGGFDSPTVAYSVLVVAKVEKGKSKKLKSVKELLGITIMERSSFEKNPIDF
LEAKGYKEVKKDLIIKLPKYSLFELENGRKRMLASAGELQKGNELALPSKYVNFLYLASHYEK
LKGSPEDNEQKQLFVEQHKHYLDEIIEQISEFSKRVILADANLDKVLSAYNKHRDKPIREQAE
NIIHLFTLTNLGAPAAFKYFDTTIDRKRYTSTKEVLDATLIHQSITGLYETRIDLSQLGGDpkkkrk
v(SGSETPGTSESATPES)GGGGGMDAKSLTAWSRTLVTFKDVFVDFTREEWKLLDT
AQQIVYRNVMLENYKNLVSLGYQLTKPDVILRLEKGEEP*
SEQ ID NO: 111
5′-TAATACGACTCACTATAGG-3′
SEQ ID NO: 112
CGAGCTGCGAGCAAAGAT
SEQ ID NO: 113
CGTGTCTCCACAGGTCACAG
SEQ ID NO: 114
GGGCTCTCCAGAACATCATC
SEQ ID NO: 115
CCTGCTTCACCACCTTCTTG
SEQ ID NO: 116
AGATCGGAAGAGCACACGTCTGAACTC
SEQ ID NO: 117
AGATCGGAAGAGCACACGTCTGAACTCCAGTCA
SEQ ID NO: 118
AGATCGGAAGAGCGTCGTGTAGGGAAAGAGTGTCCACCTTGTTG
SEQ ID NO: 119
gtttcagagcgagacgtgcctgcaggatacgtctcagaaacatg
SEQ ID NO: 120
GTTTAAGAGCTAAGCTG
SEQ ID NO: 121
AATGATACGGCGACCACCGAGATCTACACCGCGGTCTGTATCCCTTGGAGAACCAC
CT
SEQ ID NO: 122
CAAGCAGAAGACGGCATACGAGATXXXXXGCGGCCGGCTGTTTCCAGCTTAGCTCT
TAAA
SEQ ID NO: 123
CGCGGTCTGTATCCCTTGGAGAACCACCTTGTTGG
SEQ ID NO: 124
GCGGCCGGCTGTTTCCAGCTTAGCTCTTAAAC
SEQ ID NO: 125
GTTTAAGAGCTAAGCTGGAAACAGCCGGCCGC
SEQ ID NO: 126
CCACCTTGTTG

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

### REFERENCES

Adamson, B., Norman, T. M., Jost, M., Cho, M. Y., Nuñez, J. K., Chen, Y., Villalta, J. E., Gilbert, L. A., Horlbeck, M. A., Hein, M. Y., et al. (2016). A Multiplexed Single-Cell CRISPR Screening Platform Enables Systematic Dissection of the Unfolded Protein Response. Cell 167, 1867-1882.e21. Amabile, A., Migliara, A., Capasso, P., Biffi, M., Cittaro, D., Naldini, L., and Lombardo, A. (2016). Inheritable Silencing of Endogenous Genes by Hit-and-Run Targeted Epigenetic Editing. Cell 167, 219-232.e14. Anzalone, A. V., Koblan, L. W., and Liu, D. R. (2020). Genome editing with CRISPR-Cas nucleases, base editors, transposases and prime editors. Nat. Biotechnol. 38, 824-844. Audergon, P. N. C. B., Catania, S., Kagansky, A., Tong, P., Shukla, M., Pidoux, A. L., and Allshire, R. C. (2015). Restricted epigenetic inheritance of H3K9 methylation. Science 348, 132-135. Bintu, L., Yong, J., Antebi, Y. E., McCue, K., Kazuki, Y., Uno, N., Oshimura, M., and Elowitz, M. B. (2016). Dynamics of epigenetic regulation at the single-cell level. Science 351, 720-724. Blomen, V. A., Májek, P., Jae, L. T., Bigenzahn, J. W., Nieuwenhuis, J., Staring, J., Sacco, R., van Diemen, F. R., Olk, N., Stukalov, A., et al. (2015). Gene essentiality and synthetic lethality in haploid human cells. Science 350, 1092-1096. Boyes, J., and Bird, A. (1992). Repression of genes by DNA methylation depends on CpG density and promoter strength: evidence for involvement of a methyl-CpG binding protein. EMBO J. 11, 327-333. Buiting, K., Williams, C., and Horsthemke, B. (2016). Angelman syndrome—insights into a rare neurogenetic disorder. Nat. Rev. Neurol. 12, 584-593. Chavez, A., Scheiman, J., Vora, S., Pruitt, B. W., Tuttle, M., Iyer, E., Lin, S., Kiani, S., Guzman, C. D., Wiegand, D. J., et al. (2015). Highly-efficient Cas9-mediated transcriptional programming. Nat. Methods 12, 326-328. Chen, S., Zhou, Y., Chen, Y., and Gu, J. (2018). fastp: an ultra-fast all-in-one FASTQ preprocessor. Bioinforma. Oxf. Engl. 34, i884-i890. Cho, S. W., Xu, J., Sun, R., Mumbach, M. R., Carter, A. C., Chen, Y. G., Yost, K. E., Kim, J., He, J., Nevins, S. A., et al. (2018). Promoter of lncRNA Gene PVT1 Is a Tumor-Suppressor DNA Boundary Element. Cell 173, 1398-1412.e22. Deaton, A. M., and Bird, A. (2011). CpG islands and the regulation of transcription. Genes Dev. 25, 1010-1022. Doench, J. G. (2018). Am I ready for CRISPR? A user's guide to genetic screens. Nat. Rev. Genet. 19, 67-80. Fulco, C. P., Munschauer, M., Anyoha, R., Munson, G., Grossman, S. R., Perez, E. M., Kane, M., Cleary, B., Lander, E. S., and Engreitz, J. M. (2016). Systematic mapping of functional enhancer-promoter connections with CRISPR interference. Science 354, 769-773. Galonska, C., Charlton, J., Mattei, A. L., Donaghey, J., Clement, K., Gu, H., Mohammad, A. W., Stamenova, E. K., Cacchiarelli, D., Klages, S., et al. (2018). Genome-wide tracking of dCas9-methyltransferase footprints. Nat. Commun. 9. Gilbert, L. A., Larson, M. H., Morsut, L., Liu, Z., Brar, G. A., Torres, S. E., Stern-Ginossar, N., Brandman, O., Whitehead, E. H., Doudna, J. A., et al. (2013). CRISPR-Mediated Modular RNA-Guided Regulation of Transcription in Eukaryotes. Cell 154, 442-451. Gilbert, L. A., Horlbeck, M. A., Adamson, B., Villalta, J. E., Chen, Y., Whitehead, E. H., Guimaraes, C., Panning, B., Ploegh, H. L., Bassik, M. C., et al. (2014). Genome-Scale CRISPR-Mediated Control of Gene Repression and Activation. Cell 159, 647-661. Guenther, M. G., Frampton, G. M., Soldner, F., Hockemeyer, D., Mitalipova, M., Jaenisch, R., and Young, R. A. (2010). Chromatin Structure and Gene Expression Programs of Human Embryonic and Induced Pluripotent Stem Cells. Cell Stem Cell 7, 249-257. Hanna, R. E., and Doench, J. G. (2020). Design and analysis of CRISPR-Cas experiments. Nat. Biotechnol. 38, 813-823. Hansen, K. D., Langmead, B., and Irizarry, R. A. (2012). BSmooth: from whole genome bisulfite sequencing reads to differentially methylated regions. Genome Biol. 13, R83. Hart, T., Brown, K. R., Sircoulomb, F., Rottapel, R., and Moffat, J. (2014). Measuring error rates in genomic perturbation screens: gold standards for human functional genomics. Mol. Syst. Biol. 10, 733. Hathaway, N. A., Bell, O., Hodges, C., Miller, E. L., Neel, D. S., and Crabtree, G. R. (2012). Dynamics and memory of heterochromatin in living cells. Cell 149, 1447-1460. Hofacker, D., Broche, J., Laistner, L., Adam, S., Bashtrykov, P., and Jeltsch, A. (2020). Engineering of Effector Domains for Targeted DNA Methylation with Reduced Off-Target Effects. Int. J. Mol. Sci. 21. Holtzman, L., and Gersbach, C. A. (2018). Editing the Epigenome: Reshaping the Genomic Landscape. Annu. Rev. Genomics Hum. Genet. 19, 43-71. Horlbeck, M. A., Gilbert, L. A., Villalta, J. E., Adamson, B., Pak, R. A., Chen, Y., Fields, A. P., Park, C. Y., Corn, J. E., Kampmann, M., et al. (2016a). Compact and highly active next-generation libraries for CRISPR-mediated gene repression and activation. ELife 5, e19760. Horlbeck, M. A., Witkowsky, L. B., Guglielmi, B., Replogle, J. M., Gilbert, L. A., Villalta, J. E., Torigoe, S. E., Tjian, R., and Weissman, J. S. (2016b). Nucleosomes impede Cas9 access to DNA in vivo and in vitro. ELife 5. Hovestadt, V., Jones, D. T. W., Picelli, S., Wang, W., Kool, M., Northcott, P. A., Sultan, M., Stachurski, K., Ryzhova, M., Warnatz, H.-J., et al. (2014). Decoding the regulatory landscape of medulloblastoma using DNA methylation sequencing. Nature 510, 537-541. Iglesias, N., Currie, M. A., Jih, G., Paulo, J. A., Siuti, N., Kalocsay, M., Gygi, S. P., and Moazed, D. (2018). Automethylation-induced conformational switch in Clr4 (Suv39h) maintains epigenetic stability. Nature 560, 504-508. Ihry, R. J., Worringer, K. A., Salick, M. R., Frias, E., Ho, D., Theriault, K., Kommineni, S., Chen, J., Sondey, M., Ye, C., et al. (2018). p53 inhibits CRISPR-Cas9 engineering in human pluripotent stem cells. Nat. Med. 24, 939-946. Iqbal, K., Liu, F., and Gong, C.-X. (2016). Tau and neurodegenerative disease: the story so far. Nat. Rev. Neurol. 12, 15-27. Isaac, R. S., Jiang, F., Doudna, J. A., Lim, W. A., Narlikar, G. J., and Almeida, R. (2016). Nucleosome breathing and remodeling constrain CRISPR-Cas9 function. ELife 5, e13450. Jost, M., Santos, D. A., Saunders, R. A., Horlbeck, M. A., Hawkins, J. S., Scaria, S. M., Norman, T. M., Hussmann, J. A., Liem, C. R., Gross, C. A., et al. (2020). Titrating gene expression using libraries of systematically attenuated CRISPR guide RNAs. Nat. Biotechnol. 38, 355-364. Knott, G. J., and Doudna, J. A. (2018). CRISPR-Cas guides the future of genetic engineering. Science 361, 866-869. Konermann, S., Brigham, M. D., Trevino, A. E., Joung, J., Abudayyeh, O. O., Barcena, C., Hsu, P. D., Habib, N., Gootenberg, J. S., Nishimasu, H., et al. (2015). Genome-scale transcriptional activation by an engineered CRISPR-Cas9 complex. Nature 517, 583-588. Langmead, B., and Salzberg, S. L. (2012). Fast gapped-read alignment with Bowtie 2. Nat. Methods 9, 357-359. Leonetti, M. D., Sekine, S., Kamiyama, D., Weissman, J. S., and Huang, B. (2016a). A scalable strategy for high-throughput GFP tagging of endogenous human proteins. Proc. Natl. Acad. Sci. U.S.A 113, E3501-3508. Leonetti, M. D., Sekine, S., Kamiyama, D., Weissman, J. S., and Huang, B. (2016b). A scalable strategy for high-throughput GFP tagging of endogenous human proteins. Proc. Natl. Acad. Sci. 113, E3501-E3508. Li, X.-L., Li, G.-H., Fu, J., Fu, Y.-W., Zhang, L., Chen, W., Arakaki, C., Zhang, J.-P., Wen, W., Zhao, M., et al. (2018). Highly efficient genome editing via CRISPR-Cas9 in human pluripotent stem cells is achieved by transient BCL-XL overexpression. Nucleic Acids Res. 46, 10195-10215. Liao, Y., Smyth, G. K., and Shi, W. (2014). featureCounts: an efficient general purpose program for assigning sequence reads to genomic features. Bioinformatics 30, 923-930. Liu, X. S., Wu, H., Ji, X., Stelzer, Y., Wu, X., Czaudema, S., Shu, J., Dadon, D., Young, R. A., and Jaenisch, R. (2016). Editing DNA Methylation in the Mammalian Genome. Cell 167, 233-247.e17. Love, M. I., Huber, W., and Anders, S. (2014). Moderated estimation of fold change and dispersion for RNA-seq data with DESeq2. Genome Biol. 15, 550. Maeder, M. L., Angstman, J. F., Richardson, M. E., Linder, S. J., Cascio, V. M., Tsai, S. Q., Ho, Q. H., Sander, J. D., Reyon, D., Bernstein, B. E., et al. (2013). Targeted DNA demethylation and activation of endogenous genes using programmable TALE-TET1 fusion proteins. Nat. Biotechnol. 31, 1137-1142. Meyers, R. M., Bryan, J. G., McFarland, J. M., Weir, B. A., Sizemore, A. E., Xu, H., Dharia, N. V., Montgomery, P. G., Cowley, G. S., Pantel, S., et al. (2017). Computational correction of copy number effect improves specificity of CRISPR-Cas9 essentiality screens in cancer cells. Nat. Genet. 49, 1779-1784. Mlambo, T., Nitsch, S., Hildenbeutel, M., Romito, M., Müller, M., Bossen, C., Diederichs, S., Cornu, T. I., Cathomen, T., and Mussolino, C. (2018). Designer epigenome modifiers enable robust and sustained gene silencing in clinically relevant human cells. Nucleic Acids Res. 46, 4456-4468. O'Geen, H., Bates, S. L., Carter, S. S., Nisson, K. A., Halmai, J., Fink, K. D., Rhie, S. K., Famham, P. J., and Segal, D. J. (2019). Ezh2-dCas9 and KRAB-dCas9 enable engineering of epigenetic memory in a context-dependent manner. Epigenetics Chromatin 12, 26. Park, Y., and Wu, H. (2016). Differential methylation analysis for BS-seq data under general experimental design. Bioinforma. Oxf. Engl. 32, 1446-1453. Park, M., Patel, N., Keung, A. J., and Khalil, A. S. (2019). Engineering Epigenetic Regulation Using Synthetic Read-Write Modules. Cell 176, 227-238.e20. Ragunathan, K., Jih, G., and Moazed, D. (2015). Epigenetics. Epigenetic inheritance uncoupled from sequence-specific recruitment. Science 348, 1258699. Ramirez, F., Ryan, D. P., Grüning, B., Bhardwaj, V., Kilpert, F., Richter, A. S., Heyne, S., Dündar, F., and Manke, T. (2016). deepTools2: a next generation web server for deep-sequencing data analysis. Nucleic Acids Res. 44, W160-165. Replogle, J. M., Norman, T. M., Xu, A., Hussmann, J. A., Chen, J., Cogan, J. Z., Meer, E. J., Terry, J. M., Riordan, D. P., Srinivas, N., et al. (2020). Combinatorial single-cell CRISPR screens by direct guide RNA capture and targeted sequencing. Nat. Biotechnol. 38, 954-961. Robinson, J. T., Thorvaldsdóttir, H., Winckler, W., Guttman, M., Lander, E. S., Getz, G., and Mesirov, J. P. (2011). Integrative genomics viewer. Nat. Biotechnol. 29, 24-26. Schellenberger, V., Wang, C.-W., Geething, N. C., Spink, B. J., Campbell, A., To, W., Scholle, M. D., Yin, Y., Yao, Y., Bogin, O., et al. (2009). A recombinant polypeptide extends the in vivo half-life of peptides and proteins in a tunable manner. Nat. Biotechnol. 27, 1186-1190. Shalem, O., Sanjana, N. E., and Zhang, F. (2015). High-throughput functional genomics using CRISPR-Cas9. Nat. Rev. Genet. 16, 299-311. Stelzer, Y., Shivalila, C. S., Soldner, F., Markoulaki, S., and Jaenisch, R. (2015). Tracing Dynamic Changes of DNA Methylation at Single-Cell Resolution. Cell 163, 218-229. Stepper, P., Kungulovski, G., Jurkowska, R. Z., Chandra, T., Krueger, F., Reinhardt, R., Reik, W., Jeltsch, A., and Jurkowski, T. P. (2017). Efficient targeted DNA methylation with chimeric dCas9-Dnmt3a-Dnmt3L methyltransferase. Nucleic Acids Res. 45, 1703-1713. Tak, Y. E., Kleinstiver, B. P., Nuñez, J. K., Hsu, J. Y., Horng, J. E., Gong, J., Weissman, J. S., and Joung, J. K. (2017). Inducible and multiplex gene regulation using CRISPR-Cpf1-based transcription factors. Nat. Methods 14, 1163-1166. Tarasov, A., Vilella, A. J., Cuppen, E., Nijman, I. J., and Prins, P. (2015). Sambamba: fast processing of NGS alignment formats. Bioinforma. Oxf. Engl. 31, 2032-2034. Tarjan, D. R., Flavahan, W. A., and Bernstein, B. E. (2019). Epigenome editing strategies for the functional annotation of CTCF insulators. Nat. Commun. 10, 4258. Tian, R., Gachechiladze, M. A., Ludwig, C. H., Laurie, M. T., Hong, J. Y., Nathaniel, D., Prabhu, A. V., Fernandopulle, M. S., Patel, R., Abshari, M., et al. (2019). CRISPR Interference-Based Platform for Multimodal Genetic Screens in Human iPSC-Derived Neurons. Neuron 104, 239-255.e12. Van, M. V., Fujimori, T., and Bintu, L. (2021). Nanobody-mediated control of gene expression and epigenetic memory. Nat. Commun. 12, 537. Xu, X., and Qi, L. S. (2019). A CRISPR-dCas Toolbox for Genetic Engineering and Synthetic Biology. J. Mol. Biol. 431, 34-47. Xu, X., Tao, Y., Gao, X., Zhang, L., Li, X., Zou, W., Ruan, K., Wang, F., Xu, G., and Hu, R. (2016). A CRISPR-based approach for targeted DNA demethylation. Cell Discov. 2, 1-12. Yeh, C. D., Richardson, C. D., and Corn, J. E. (2019). Advances in genome editing through control of DNA repair pathways. Nat. Cell Biol. 21, 1468-1478. Yu, R., Wang, X., and Moazed, D. (2018). Epigenetic inheritance mediated by coupling of RNAi and histone H3K9 methylation. Nature 558, 615-619. Zhang, Z.-M., Lu, R., Wang, P., Yu, Y., Chen, D., Gao, L., Liu, S., Ji, D., Rothbart, S. B., Wang, Y., et al. (2018). Structural basis for DNMT3A-mediated de novo DNA methylation. Nature 554, 387-391.

What is claimed is:

1. A fusion protein comprising, from N-terminus to C-terminus, a DNA methyltransferase domain, a first XTEN linker, a nuclease-deficient RNA-guided endonuclease enzyme, a second XTEN linker, and a Krüppel-associated box domain.

2. The fusion protein of claim 1, wherein the first XTEN linker comprises from about 5 to about 864 amino acid residues, and the second XTEN linker comprises from about 5 to about 864 amino acid residues, and wherein the nuclease-deficient RNA-guided DNA endonuclease enzyme is a CRISPR-associated protein, a leucine zipper domain, a winged helix domain, a helix-turn-helix motif, a helix-loop-helix domain, an HMB-box domain, a Wor3 domain, an OB-fold domain, an immunoglobulin domain, or a B3 domain.

3. (canceled)

4. (canceled)

5. (canceled)

6. (canceled)

7. The fusion protein of claim 2, wherein the nuclease-deficient RNA-guided DNA endonuclease enzyme is dCas9, dCpf1, or ddCpf1, the DNA methyltransferase domain comprises a Dnmt3A domain, and the Dnmt3A domain is linked to a Dnmt3L domain (Dnmt3A-3L domain).

8. (canceled)

9. (canceled)

10. (canceled)

11. (canceled)

12. The fusion protein of claim 1, comprising, from N-terminus to C-terminus, the DNA methyltransferase domain, the first XTEN linker, the nuclease-deficient RNA-guided endonuclease enzyme, a nuclear localization signal peptide, and the Krüppel-associated box domain, or comprising, from N-terminus to C-terminus, the DNA methyltransferase domain, the first XTEN linker, the nuclease-deficient RNA-guided endonuclease enzyme, a nuclear localization signal peptide, the second XTEN linker, and the Krüppel-associated box domain, or comprising, from N-terminus to C-terminus, the DNA methyltransferase domain, the first XTEN linker, the nuclease-deficient RNA-guided endonuclease enzyme, an epitope tag, a nuclear localization signal peptide, the second XTEN linker, the Krüppel-associated box domain, a 2A cleavable peptide, and a fluorescent protein tag.

13. (canceled)

14. (canceled)

15. A fusion protein having at least 85% sequence identity to the amino acid sequence of Formula (A); where the amino acid sequence of Formula (A) is, from N-terminus to C-terminus:
C₁-R³-C₂-R²-A-R¹-R⁴-B (A),
wherein:
C₁ comprises SEQ ID NO: 26 or SEQ ID NO: 106;
R³ is absent or R³ comprises SEQ ID NO: 27;
C₂ comprises SEQ ID NO: 28;
R² is absent or R² comprises SEQ ID NO: 32;
A comprises SEQ ID NO: 23;
R¹ is absent or R¹ comprises SEQ ID NO: 25;
R⁴ is absent or R⁴ comprises SEQ ID NO: 31; and
B comprises SEQ ID NO: 16, SEQ ID NO: 103, SEQ ID NO: 104, or SEQ ID NO: 105.

16. (canceled)

17. (canceled)

18. A fusion protein having at least 85% sequence identity to SEQ ID NO:97, 98, 99, 107, 108, 109, or 110.

19. (canceled)

20. A cell comprising the fusion protein of claim 1.

21. (canceled)

22. A method of silencing a target nucleic acid sequence in a cell, the method comprising:
(i) delivering a first polynucleotide encoding a fusion protein of claim 1 to a cell containing the target nucleic acid; and
(ii) delivering to the cell a second polynucleotide comprising sgRNA or cr:tracrRNA; thereby silencing the target nucleic acid sequence.

23. (canceled)

24. (canceled)

25. (canceled)

26. A method of silencing a target nucleic acid sequence in a cell, the method comprising:
(i) delivering a first polynucleotide encoding a fusion protein to a cell containing the target nucleic acid, wherein the target nucleic acid does not comprise a CpG island; wherein the fusion protein comprises a nuclease-deficient RNA-guided DNA endonuclease enzyme, a Krüppel associated box domain, and a DNA methyltransferase domain; and
(ii) delivering to the cell a second polynucleotide comprising sgRNA or cr:tracrRNA; thereby silencing the target nucleic acid sequence in the cell.

27. A method of treating Angelman syndrome, an infectious disease, a tau pathology, or a neurodegenerative disease in a subject in need thereof, the method comprising:
(i) delivering to the subject an effective amount of a first polynucleotide encoding a fusion protein comprising a nuclease-deficient RNA-guided DNA endonuclease enzyme, a Krüppel associated box domain, and a DNA methyltransferase domain; and
(ii) delivering to the subject an effective amount of a second polynucleotide comprising sgRNA or cr:tracrRNA; thereby treating Angelman syndrome, the infectious disease, the tau pathology, or the neurodegenerative disease.

28. (canceled)

29. The method of claim 26, wherein the fusion protein comprises, from N-terminus to C-terminus, the DNA methyltransferase domain, the nuclease-deficient RNA-guided DNA endonuclease enzyme, and the Krüppel associated box domain; and the nuclease-deficient RNA-guided DNA endonuclease enzyme is a CRISPR-associated protein, a leucine zipper domain, a winged helix domain, a helix-turn-helix motif, a helix-loop-helix domain, an HMB-box domain, a Wor3 domain, an OB-fold domain, an immunoglobulin domain, or a B3 domain; and the DNA methyltransferase domain comprises a Dnmt3A domain linked to a Dnmt3L domain (Dnmt3A-3L domain).

30. (canceled)

31. (canceled)

32. (canceled)

33. (canceled)

34. (canceled)

35. (canceled)

36. (canceled)

37. The method of claim 26, wherein the fusion protein comprises, from N-terminus to C-terminus, the Krüppel associated box, the nuclease-deficient RNA-guided DNA endonuclease enzyme, and the DNA methyltransferase domain; the nuclease-deficient RNA-guided DNA endonuclease enzyme is a CRISPR-associated protein, a leucine zipper domain, a winged helix domain, a helix-turn-helix motif, a helix-loop-helix domain, an HMB-box domain, a Wor3 domain, an OB-fold domain, an immunoglobulin domain, or a B3 domain; and the DNA methyltransferase domain comprises a Dnmt3A domain linked to a Dnmt3L domain (Dnmt3A-3L domain).

38. (canceled)

39. (canceled)

40. (canceled)

41. (canceled)

42. (canceled)

43. The method of claim 39, wherein the dCas9 is covalently linked to the Dnmt3A domain via a peptide linker and wherein the Krüppel associated box domain is covalently linked to the dCas9 via a peptide linker, and the peptide linker is a XTEN linker.

44. (canceled)

45. The method of claim 37, wherein the nuclease-deficient RNA-guided DNA endonuclease enzyme is covalently linked to the Krüppel associated box domain via a peptide linker, or wherein the nuclease-deficient RNA-guided DNA endonuclease enzyme is covalently linked to the DNA methyltransferase domain via a peptide linker, or wherein the Krüppel associated box domain is covalently linked to the DNA methyltransferase domain via a peptide linker.

46. (canceled)

47. (canceled)

48. (canceled)

49. (canceled)

50. The method of claim 26, wherein the fusion protein has at least 85% sequence identity to the amino acid sequence of Formula (A); where the amino acid sequence of Formula (A) is, from N-terminus to C-terminus:
C₁-R³-C₂-R²-A-R¹-R⁴-B (A),
wherein:
C₁ comprises SEQ ID NO:26 or SEQ ID NO:106;
R³ is absent or R³ comprises SEQ ID NO:27;
C₂ comprises SEQ ID NO:28;
R² is absent or R² comprises SEQ ID NO:32;
A comprises SEQ ID NO:23;
R¹ is absent or R¹ comprises SEQ ID NO:25;
R⁴ is absent or R⁴ comprises SEQ ID NO:31; and
B comprises SEQ ID NO:16, SEQ ID NO: 103, SEQ ID NO:104, or SEQ ID NO:105.

51. (canceled)

52. (canceled)

53. A method of treating Angelman syndrome, an infectious disease, a tau pathology, or a neurodegenerative disease in a subject in need thereof, the method comprising:
(i) delivering to the subject an effective amount of a first polynucleotide encoding a fusion protein of claim 1; and
(ii) delivering to the subject an effective amount of second polynucleotide comprising sgRNA or cr:tracrRNA; thereby treating Angelman syndrome, the infectious disease, the tau pathology, or the neurodegenerative disease in the subject.

54. (canceled)

55. (canceled)

56. A fusion protein comprising, from N-terminus to C-terminus, a DNA methyltransferase domain, a first XTEN linker, a nuclease-deficient endonuclease enzyme, a second XTEN linker, and a Krüppel-associated box domain, wherein the first XTEN linker comprises from about 5 to about 864 amino acid residues, the second XTEN linker comprises from about 5 to about 864 amino acid residues, and the nuclease-deficient DNA endonuclease enzyme is a zinc finger domain or a transcription activator-like effector.

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. The fusion protein of claim 56, comprising, from N-terminus to C-terminus, the DNA methyltransferase domain, the first XTEN linker, the nuclease-deficient endonuclease enzyme, an epitope tag, a nuclear localization signal peptide, the second XTEN linker, the Krüppel-associated box domain, a 2A cleavable peptide, and a fluorescent protein tag.

69. A cell comprising the fusion protein of claim 56.

70. (canceled)

71. A method of silencing a target nucleic acid sequence in a cell, the method comprising delivering a first polynucleotide encoding a fusion protein of claim 56 to a cell containing the target nucleic acid; thereby silencing the target nucleic acid sequence.

72. (canceled)

73. (canceled)

74. A method of treating Angelman syndrome, an infectious disease, a tau pathology, or a neurodegenerative disease in a subject in need thereof, the method comprising delivering to the subject an effective amount of a first polynucleotide encoding a fusion protein of claim 56; thereby treating Angelman syndrome, the infectious disease, the tau pathology, or the neurodegenerative disease.

75. (canceled)

76. (canceled)


SEQUENCE LISTING (203 sequences)

SEQ ID NO: 1 - protein, 2294 aa, Artificial Sequence
  Synthetic polypeptide
DAKSLTAWSRTLVTFKDVFVDFTREEWKLLDTAQQIVYRNVMLENYKNLVSLGYQLTKPDVILRLEKGEEPGGSGGGSMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADYPYDVPDYASGSPKKKRKVEASGSGRASPGIPGSTRNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLSRADPKKKRKVGSGATNFSLLKQAGDVEENPGPSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 2 - protein, 2296 aa, Artificial Sequence
  Synthetic polypeptide
DAKSLTAWSRTLVTFKDVFVDFTREEWKLLDTAQQIVYRNVMLENYKNLVSLGYQLTKPDVILRLEKGEEPGGSGGGSMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADYPYDVPDYASGSPKKKRKVSPGSGSETPGTSESATPESNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLSRADPKKKRKVGSGATNFSLLKQAGDVEENPGPSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 3 - protein, 2316 aa, Artificial Sequence
  Synthetic polypeptide
DAKSLTAWSRTLVTFKDVFVDFTREEWKLLDTAQQIVYRNVMLENYKNLVSLGYQLTKPDVILRLEKGEEPGGSGGGSMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADYPYDVPDYASGSPKKKRKVEASGSGRASPGIPGSTRNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLSRADPKKKRKVGSGATNFSLLKQAGDVEENPGPGSGATNFSLLKQAGDVEENPGPSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 4 - protein, 2318 aa, Artificial Sequence
  Synthetic polypeptide
DAKSLTAWSRTLVTFKDVFVDFTREEWKLLDTAQQIVYRNVMLENYKNLVSLGYQLTKPDVILRLEKGEEPGGSGGGSMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADYPYDVPDYASGSPKKKRKVSPGSGSETPGTSESATPESNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLSRADPKKKRKVGSGATNFSLLKQAGDVEENPGPGSGATNFSLLKQAGDVEENPGPSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 5 - protein, 2360 aa, Artificial Sequence
  Synthetic polypeptide
DAKSLTAWSRTLVTFKDVFVDFTREEWKLLDTAQQIVYRNVMLENYKNLVSLGYQLTKPDVILRLEKGEEPGGSGGGSMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADYPYDVPDYASGSPKKKRKVSPGGGPSSGAPPPSGGSPAGSPTSTEEGTSESATPESGPGTSTEPSEGSAPGSPAGSPTSTEEGTSTEPSEGSAPGTSTEPSENHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLSRADPKKKRKVGSGATNFSLLKQAGDVEENPGPSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 6 - protein, 2382 aa, Artificial Sequence
  Synthetic polypeptide
DAKSLTAWSRTLVTFKDVFVDFTREEWKLLDTAQQIVYRNVMLENYKNLVSLGYQLTKPDVILRLEKGEEPGGSGGGSMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADYPYDVPDYASGSPKKKRKVSPGGGPSSGAPPPSGGSPAGSPTSTEEGTSESATPESGPGTSTEPSEGSAPGSPAGSPTSTEEGTSTEPSEGSAPGTSTEPSENHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLSRADPKKKRKVGSGATNFSLLKQAGDVEENPGPGSGATNFSLLKQAGDVEENPGPSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 7 - protein, 2303 aa, Artificial Sequence
  Synthetic polypeptide
DAKSLTAWSRTLVTFKDVFVDFTREEWKLLDTAQQIVYRNVMLENYKNLVSLGYQLTKPDVILRLEKGEEPSGSETPGTSESATPESMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADYPYDVPDYASGSPKKKRKVEASGSGRASPGIPGSTRNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLSRADPKKKRKVGSGATNFSLLKQAGDVEENPGPSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 8 - protein, 2325 aa, Artificial Sequence
  Synthetic polypeptide
DAKSLTAWSRTLVTFKDVFVDFTREEWKLLDTAQQIVYRNVMLENYKNLVSLGYQLTKPDVILRLEKGEEPSGSETPGTSESATPESMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADYPYDVPDYASGSPKKKRKVEASGSGRASPGIPGSTRNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLSRADPKKKRKVGSGATNFSLLKQAGDVEENPGPGSGATNFSLLKQAGDVEENPGPSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 9 - protein, 2367 aa, Artificial Sequence
  Synthetic polypeptide
DAKSLTAWSRTLVTFKDVFVDFTREEWKLLDTAQQIVYRNVMLENYKNLVSLGYQLTKPDVILRLEKGEEPGGPSSGAPPPSGGSPAGSPTSTEEGTSESATPESGPGTSTEPSEGSAPGSPAGSPTSTEEGTSTEPSEGSAPGTSTEPSEMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADYPYDVPDYASGSPKKKRKVEASGSGRASPGIPGSTRNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLSRADPKKKRKVGSGATNFSLLKQAGDVEENPGPSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 10 - protein, 2389 aa, Artificial Sequence
  Synthetic polypeptide
DAKSLTAWSRTLVTFKDVFVDFTREEWKLLDTAQQIVYRNVMLENYKNLVSLGYQLTKPDVILRLEKGEEPGGPSSGAPPPSGGSPAGSPTSTEEGTSESATPESGPGTSTEPSEGSAPGSPAGSPTSTEEGTSTEPSEGSAPGTSTEPSEMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADYPYDVPDYASGSPKKKRKVEASGSGRASPGIPGSTRNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLSRADPKKKRKVGSGATNFSLLKQAGDVEENPGPGSGATNFSLLKQAGDVEENPGPSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 11 - protein, 2369 aa, Artificial Sequence
  Synthetic polypeptide
DAKSLTAWSRTLVTFKDVFVDFTREEWKLLDTAQQIVYRNVMLENYKNLVSLGYQLTKPDVILRLEKGEEPSGSETPGTSESATPESMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADYPYDVPDYASGSPKKKRKVSPGGGPSSGAPPPSGGSPAGSPTSTEEGTSESATPESGPGTSTEPSEGSAPGSPAGSPTSTEEGTSTEPSEGSAPGTSTEPSENHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLSRADPKKKRKVGSGATNFSLLKQAGDVEENPGPSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 12 - protein, 2391 aa, Artificial Sequence
  Synthetic polypeptide
DAKSLTAWSRTLVTFKDVFVDFTREEWKLLDTAQQIVYRNVMLENYKNLVSLGYQLTKPDVILRLEKGEEPSGSETPGTSESATPESMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADYPYDVPDYASGSPKKKRKVSPGGGPSSGAPPPSGGSPAGSPTSTEEGTSESATPESGPGTSTEPSEGSAPGSPAGSPTSTEEGTSTEPSEGSAPGTSTEPSENHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLSRADPKKKRKVGSGATNFSLLKQAGDVEENPGPGSGATNFSLLKQAGDVEENPGPSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 13 - protein, 2369 aa, Artificial Sequence
  Synthetic polypeptide
DAKSLTAWSRTLVTFKDVFVDFTREEWKLLDTAQQIVYRNVMLENYKNLVSLGYQLTKPDVILRLEKGEEPGGPSSGAPPPSGGSPAGSPTSTEEGTSESATPESGPGTSTEPSEGSAPGSPAGSPTSTEEGTSTEPSEGSAPGTSTEPSEMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADYPYDVPDYASGSPKKKRKVSPGSGSETPGTSESATPESNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLSRADPKKKRKVGSGATNFSLLKQAGDVEENPGPSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 14 - protein, 2391 aa, Artificial Sequence
  Synthetic polypeptide
DAKSLTAWSRTLVTFKDVFVDFTREEWKLLDTAQQIVYRNVMLENYKNLVSLGYQLTKPDVILRLEKGEEPGGPSSGAPPPSGGSPAGSPTSTEEGTSESATPESGPGTSTEPSEGSAPGSPAGSPTSTEEGTSTEPSEGSAPGTSTEPSEMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADYPYDVPDYASGSPKKKRKVSPGSGSETPGTSESATPESNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLSRADPKKKRKVGSGATNFSLLKQAGDVEENPGPGSGATNFSLLKQAGDVEENPGPSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 15 - protein, 2345 aa, Artificial Sequence
  Synthetic polypeptide
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGIGSGSNGSSGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLNGGGGGMDAKSLTAWSRTLVTFKDVFVDFTREEWKLLDTAQQIVYRNVMLENYKNLVSLGYQLTKPDVILRLEKGEEP

SEQ ID NO: 16 - protein, 71 aa, Homo sapiens
DAKSLTAWSRTLVTFKDVFVDFTREEWKLLDTAQQIVYRNVMLENYKNLVSLGYQLTKPDVILRLEKGEEP

SEQ ID NO: 17 - protein, 7 aa, Artificial Sequence
  Synthetic polypeptide
GGSGGGS

SEQ ID NO: 18 - protein, 3 aa, Artificial Sequence
  Synthetic polypeptide
SGS

SEQ ID NO: 19 - protein, 17 aa, Artificial Sequence
  Synthetic polypeptide
EASGSGRASPGIPGSTR

SEQ ID NO: 20 - protein, 4 aa, Artificial Sequence
  Synthetic polypeptide
SRAD

SEQ ID NO: 21 - protein, 3 aa, Artificial Sequence
  Synthetic polypeptide
GSG

SEQ ID NO: 22 - protein, 3 aa, Artificial Sequence
  Synthetic polypeptide
SPG

SEQ ID NO: 23 - protein, 1368 aa, Artificial Sequence
  Synthetic polypeptide
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 24 - protein, 9 aa, Artificial Sequence
  Synthetic polypeptide
YPYDVPDYA

SEQ ID NO: 25 - protein, 7 aa, Artificial Sequence
  Synthetic polypeptide
PKKKRKV

SEQ ID NO: 26 - protein, 301 aa, Homo sapiens
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 27 - protein, 27 aa, Artificial Sequence
  Synthetic polypeptide
SSGNSNANSRGPSFSSGLVPLSLRGSH

SEQ ID NO: 28 - protein, 215 aa, Artificial Sequence
  Synthetic polypeptide
MGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPL

SEQ ID NO: 29 - protein, 19 aa, Artificial Sequence
  Synthetic polypeptide
ATNFSLLKQAGDVEENPGP

SEQ ID NO: 30 - protein, 232 aa, Artificial Sequence
  Synthetic polypeptide
SELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 31 - protein, 16 aa, Artificial Sequence
  Synthetic polypeptide
SGSETPGTSESATPES

SEQ ID NO: 32 - protein, 80 aa, Artificial Sequence
  Synthetic polypeptide
GGPSSGAPPPSGGSPAGSPTSTEEGTSESATPESGPGTSTEPSEGSAPGSPAGSPTSTEEGTSTEPSEGSAPGTSTEPSE

SEQ ID NO: 33 - protein, 543 aa, Artificial Sequence
  Synthetic polypeptide
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPL

SEQ ID NO: 34 - protein, 1307 aa, Artificial Sequence
  Synthetic polypeptide
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLANLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 35 - protein, 1228 aa, Artificial Sequence
  Synthetic polypeptide
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALADLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 36 - protein, 1314 aa, Artificial Sequence
  Synthetic polypeptide
MYPYDVPDYASGSGMSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 37 - DNA, 19 bp, Artificial Sequence
  Synthetic polynucleotide
actgcggaaatttgagcgt

SEQ ID NO: 38 - RNA, 19 nt, Artificial Sequence
  Synthetic polynucleotide
acgcucaaauuuccgcagu

SEQ ID NO: 39 - DNA, 19 bp, Artificial Sequence
  Synthetic polynucleotide
aggcaatggctgcacatgc

SEQ ID NO: 40 - RNA, 19 nt, Artificial Sequence
  Synthetic polynucleotide
gcaugugcagccauugccu

SEQ ID NO: 41 - DNA, 19 bp, Artificial Sequence
  Synthetic polynucleotide
gacgcttggttctgaggag

SEQ ID NO: 42 - RNA, 19 nt, Artificial Sequence
  Synthetic polynucleotide
cuccucagaaccaagcguc

SEQ ID NO: 43 - DNA, 19 bp, Artificial Sequence
  Synthetic polynucleotide
tccggaaacgcattcctct

SEQ ID NO: 44 - RNA, 19 nt, Artificial Sequence
  Synthetic polynucleotide
agaggaaugcguuuccgga

SEQ ID NO: 45 - DNA, 19 bp, Artificial Sequence
  Synthetic polynucleotide
ccgcgtcagcccggcccgg

SEQ ID NO: 46 - RNA, 19 nt, Artificial Sequence
  Synthetic polynucleotide
ccgggccgggcugacgcgg

SEQ ID NO: 47 - DNA, 19 bp, Artificial Sequence
  Synthetic polynucleotide
cgactcccgctgggcctct

SEQ ID NO: 48 - RNA, 19 nt, Artificial Sequence
  Synthetic polynucleotide
agaggcccagcgggagucg

SEQ ID NO: 49 - DNA, 19 bp, Artificial Sequence
  Synthetic polynucleotide
ccgttgcgcgctcgctctc

SEQ ID NO: 50 - RNA, 19 nt, Artificial Sequence
  Synthetic polynucleotide
gagagcgagcgcgcaacgg

SEQ ID NO: 51 - DNA, 19 bp, Artificial Sequence
  Synthetic polynucleotide
ccgcgcatcctgccaggcc

SEQ ID NO: 52 - RNA, 19 nt, Artificial Sequence
  Synthetic polynucleotide
ggccuggcaggaugcgcgg

SEQ ID NO: 53 - DNA, 19 bp, Artificial Sequence
  Synthetic polynucleotide
ccaacttggcgcgtttcgg

SEQ ID NO: 54 - RNA, 19 nt, Artificial Sequence
  Synthetic polynucleotide
ccgaaacgcgccaaguugg

SEQ ID NO: 55 - DNA, 19 bp, Artificial Sequence
  Synthetic polynucleotide
accacgcgtccgagtccgg

SEQ ID NO: 56 - RNA, 19 nt, Artificial Sequence
  Synthetic polynucleotide
ccggacucggacgcguggu

SEQ ID NO: 57 - DNA, 19 bp, Artificial Sequence
  Synthetic polynucleotide
tgctcattgtccctggaca

SEQ ID NO: 58 - RNA, 19 nt, Artificial Sequence
  Synthetic polynucleotide
uguccagggacaaugagca

SEQ ID NO: 59 - DNA, 19 bp, Artificial Sequence
  Synthetic polynucleotide
ggacaccctgctcattgtc

SEQ ID NO: 60 - RNA, 19 nt, Artificial Sequence
  Synthetic polynucleotide
gacaaugagcagggugucc

SEQ ID NO: 61 - DNA, 19 bp, Artificial Sequence
  Synthetic polynucleotide
accggcagcctgcgcgtcc

SEQ ID NO: 62 - RNA, 19 nt, Artificial Sequence
  Synthetic polynucleotide
ggacgcgcaggcugccggu

SEQ ID NO: 63 - DNA, 19 bp, Artificial Sequence
  Synthetic polynucleotide
cgatgggcacccactgctc

SEQ ID NO: 64 - RNA, 19 nt, Artificial Sequence
  Synthetic polynucleotide
gagcagugggugcccaucg

SEQ ID NO: 65 - DNA, 19 bp, Artificial Sequence
  Synthetic polynucleotide
ccttcacgtggacgcgcag

SEQ ID NO: 66 - RNA, 19 nt, Artificial Sequence
  Synthetic polynucleotide
cugcgcguccacgugaagg

SEQ ID NO: 67 - DNA, 19 bp, Artificial Sequence
  Synthetic polynucleotide
cgtgaaggtggaagccttc

SEQ ID NO: 68 - RNA, 19 nt, Artificial Sequence
  Synthetic polynucleotide
gaaggcuuccaccuucacg

SEQ ID NO: 69 - DNA, 19 bp, Artificial Sequence
  Synthetic polynucleotide
ctccttggtcaggcgccgg

SEQ ID NO: 70 - RNA, 19 nt, Artificial Sequence
  Synthetic polynucleotide
ccggcgccugaccaaggag

SEQ ID NO: 71 - DNA, 19 bp, Artificial Sequence
  Synthetic polynucleotide
tggtcaggcgccggttccg

SEQ ID NO: 72 - RNA, 19 nt, Artificial Sequence
  Synthetic polynucleotide
cggaaccggcgccugacca

SEQ ID NO: 73 - DNA, 19 bp, Artificial Sequence
  Synthetic polynucleotide
tagaggtcgccttctcctc

SEQ ID NO: 74 - RNA, 19 nt, Artificial Sequence
  Synthetic polynucleotide
gaggagaaggcgaccucua

SEQ ID NO: 75 - DNA, 19 bp, Artificial Sequence
  Synthetic polynucleotide
cgacgctcgggtcgcggtg

SEQ ID NO: 76 - RNA, 19 nt, Artificial Sequence
  Synthetic polynucleotide
caccgcgacccgagcgucg

SEQ ID NO: 77 - DNA, 19 bp, Artificial Sequence
  Synthetic polynucleotide
atgctgtcgccgcgcgggg

SEQ ID NO: 78 - RNA, 19 nt, Artificial Sequence
  Synthetic polynucleotide
ccccgcgcggcgacagcau

SEQ ID NO: 79 - DNA, 19 bp, Artificial Sequence
  Synthetic polynucleotide
ctcaccctcaccggagcca

SEQ ID NO: 80 - RNA, 19 nt, Artificial Sequence
  Synthetic polynucleotide
uggcuccggugagggugag

SEQ ID NO: 81 - DNA, 19 bp, Artificial Sequence
  Synthetic polynucleotide
ccgcaaactttactcctta

SEQ ID NO: 82 - RNA, 19 nt, Artificial Sequence
  Synthetic polynucleotide
uaaggaguaaaguuugcgg

SEQ ID NO: 83 - DNA, 19 bp, Artificial Sequence
  Synthetic polynucleotide
ctcctaagattggcttcac

SEQ ID NO: 84 - RNA, 19 nt, Artificial Sequence
  Synthetic polynucleotide
gugaagccaaucuuaggag

SEQ ID NO: 85 - DNA, 19 bp, Artificial Sequence
  Synthetic polynucleotide
ccggagccactcctaagat

SEQ ID NO: 86 - RNA, 19 nt, Artificial Sequence
  Synthetic polynucleotide
aucuuaggaguggcuccgg

SEQ ID NO: 87 - DNA, 19 bp, Artificial Sequence
  Synthetic polynucleotide
ttctctaccctacgtctca

SEQ ID NO: 88 - RNA, 19 nt, Artificial Sequence
  Synthetic polynucleotide
ugagacguaggguagagaa

SEQ ID NO: 89 - DNA, 19 bp, Artificial Sequence
  Synthetic polynucleotide
tacgtctcattctccgcaa

SEQ ID NO: 90 - RNA, 19 nt, Artificial Sequence
  Synthetic polynucleotide
uugcggagaaugagacgua

SEQ ID NO: 91 - DNA, 19 bp, Artificial Sequence
  Synthetic polynucleotide
gctaggcctccagcccttc

SEQ ID NO: 92 - RNA, 19 nt, Artificial Sequence
  Synthetic polynucleotide
gaagggcuggaggccuagc

SEQ ID NO: 93 - DNA, 19 bp, Artificial Sequence
  Synthetic polynucleotide
acaggtggcgccgcaactt

SEQ ID NO: 94 - RNA, 19 nt, Artificial Sequence
  Synthetic polynucleotide
aaguugcggcgccaccugu

SEQ ID NO: 95 - DNA, 19 bp, Artificial Sequence
  Synthetic polynucleotide
agccggaggcgcgagagtc

SEQ ID NO: 96 - RNA, 19 nt, Artificial Sequence
  Synthetic polynucleotide
gacucucgcgccuccggcu

SEQ ID NO: 97 - protein, 2381 aa, Artificial Sequence
  Synthetic polypeptide
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGIGSGSNGSSGSSGSETPGTSESATPESGGGGGMDAKSLTAWSRTLVTFKDVFVDFTREEWKLLDTAQQIVYRNVMLENYKNLVSLGYQLTKPDVILRLEKGEEPATNFSLLKQAGDVEENPGPSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 98 - protein, 2335 aa, Artificial Sequence
  Synthetic polypeptide
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPKKKRKVSGSETPGTSESATPESRTLVTFKDVFVDFTREEWKLLDTAQQIVYRNVMLENYKNLVSLGYQLTKPDVILRLEKGEEPATNFSLLKQAGDVEENPGPSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 99 - protein, 2331 aa, Artificial Sequence
  Synthetic polypeptide
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPKKKRKVSGSETPGTSESATPESSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLNGGGGGMDAKSLTAWSRTLVTFKDVFVDFTREEWKLLDTAQQIVYRNVMLENYKNLVSLGYQLTKPDVILRLEKGEEP

SEQ ID NO: 100 - DNA, 113 bp, Artificial Sequence
  Synthetic polynucleotide
gacgctcaaatttccgcagtgtttaagagctaagctggaaacagcatagcaagtttaaataaggctagtccgttatcaacttgaaaaagtggcaccgagtcggtgcttttttt

SEQ ID NO: 101 - DNA, 93 bp, Artificial Sequence
  Synthetic polynucleotide
gtttaagagctaagctggaaacagcatagcaagtttaaataaggctagtccgttatcaacttgaaaaagtggcaccgagtcggtgcttttttt

SEQ ID NO: 102 - DNA, 20 bp, Artificial Sequence
  Synthetic polynucleotide
gacgctcaaatttccgcagt

SEQ ID NO: 103 - protein, 72 aa, Homo sapiens
MDAKSLTAWSRTLVTFKDVFVDFTREEWKLLDTAQQIVYRNVMLENYKNLVSLGYQLTKPDVILRLEKGEEP

SEQ ID NO: 104 - protein, 62 aa, Homo sapiens
RTLVTFKDVFVDFTREEWKLLDTAQQIVYRNVMLENYKNLVSLGYQLTKPDVILRLEKGEEP

SEQ ID NO: 105 - protein, 63 aa, Artificial Sequence
  Synthetic polypeptide
MRTLVTFKDVFVDFTREEWKLLDTAQQIVYRNVMLENYKNLVSLGYQLTKPDVILRLEKGEEP

SEQ ID NO: 106 - protein, 302 aa, Artificial Sequence
  Synthetic polypeptide
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 107 - protein, 2104 aa, Artificial Sequence
  Synthetic polypeptide
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDASLGSGSPKKKRKVEDPKKKRKVDGIGSGSNGSSGSGGGGGMDAKSLTAWSRTLVTFKDVFVDFTREEWKLLDTAQQIVYRNVMLENYKNLVSLGYQLTKPDVILRLEKGEEP

SEQ ID NO: 108 - protein, 2121 aa, Artificial Sequence
  Synthetic polypeptide
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDASLGSGSPKKKRKVEDPKKKRKVDGIGSGSNGSSGSSGSETPGTSESATPESGGGGGMDAKSLTAWSRTLVTFKDVFVDFTREEWKLLDTAQQIVYRNVMLENYKNLVSLGYQLTKPDVILRLEKGEEP

SEQ ID NO: 109 - protein, 2084 aa, Artificial Sequence
  Synthetic polypeptide
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPKKKRKVSGSETPGTSESATPESRTLVTFKDVFVDFTREEWKLLDTAQQIVYRNVMLENYKNLVSLGYQLTKPDVILRLEKGEEP

SEQ ID NO: 110 - protein, 2099 aa, Artificial Sequence
  Synthetic polypeptide
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPKKKRKVSGSETPGTSESATPESGGGGGMDAKSLTAWSRTLVTFKDVFVDFTREEWKLLDTAQQIVYRNVMLENYKNLVSLGYQLTKPDVILRLEKGEEP

SEQ ID NO: 111 - DNA, 19 bp, Artificial Sequence
  Synthetic polynucleotide
taatacgactcactatagg

SEQ ID NO: 112 - DNA, 18 bp, Artificial Sequence
  Synthetic polynucleotide
cgagctgcgagcaaagat

SEQ ID NO: 113 - DNA, 20 bp, Artificial Sequence
  Synthetic polynucleotide
cgtgtctccacaggtcacag

SEQ ID NO: 114 - DNA, 20 bp, Artificial Sequence
  Synthetic polynucleotide
gggctctccagaacatcatc

SEQ ID NO: 115 - DNA, 20 bp, Artificial Sequence
  Synthetic polynucleotide
cctgcttcaccaccttcttg

SEQ ID NO: 116 - DNA, 27 bp, Artificial Sequence
  Synthetic polynucleotide
agatcggaagagcacacgtctgaactc

SEQ ID NO: 117 - DNA, 33 bp, Artificial Sequence
  Synthetic polynucleotide
agatcggaagagcacacgtctgaactccagtca

SEQ ID NO: 118 - DNA, 44 bp, Artificial Sequence
  Synthetic polynucleotide
agatcggaagagcgtcgtgtagggaaagagtgtccaccttgttg

SEQ ID NO: 119 - DNA, 44 bp, Artificial Sequence
  Synthetic polynucleotide
gtttcagagcgagacgtgcctgcaggatacgtctcagaaacatg

SEQ ID NO: 120 - DNA, 17 bp, Artificial Sequence
  Synthetic polynucleotide
gtttaagagctaagctg

SEQ ID NO: 121 - DNA, 58 bp, Artificial Sequence
  Synthetic polynucleotide
aatgatacggcgaccaccgagatctacaccgcggtctgtatcccttggagaaccacct

SEQ ID NO: 122 - DNA, 60 bp, Artificial Sequence
  Synthetic polynucleotide
  misc_feature (25)..(25): A, C, G or T
  misc_feature (26)..(26): A, C, G or T
  misc_feature (27)..(27): A, C, G or T
  misc_feature (28)..(28): A, C, G or T
  misc_feature (29)..(29): A, C, G or T
caagcagaagacggcatacgagatnnnnngcggccggctgtttccagcttagctcttaaa

SEQ ID NO: 123 - DNA, 35 bp, Artificial Sequence
  Synthetic polynucleotide
cgcggtctgtatcccttggagaaccaccttgttgg

SEQ ID NO: 124 - DNA, 32 bp, Artificial Sequence
  Synthetic polynucleotide
gcggccggctgtttccagcttagctcttaaac

SEQ ID NO: 125 - DNA, 32 bp, Artificial Sequence
  Synthetic polynucleotide
gtttaagagctaagctggaaacagccggccgc

SEQ ID NO: 126 - DNA, 11 bp, Artificial Sequence
  Synthetic polynucleotide
ccaccttgttg

SEQ ID NO: 127 - DNA, 19 bp, Artificial Sequence
  Synthetic polynucleotide
ctcctcagaaccaagcgtc

SEQ ID NO: 128 - DNA, 19 bp, Artificial Sequence
  Synthetic polynucleotide
gtaagacacagtacaaacg

SEQ ID NO: 129 - DNA, 19 bp, Artificial Sequence
  Synthetic polynucleotide
gaaccggcaaaatccgctc

SEQ ID NO: 130 - DNA, 19 bp, Artificial Sequence
  Synthetic polynucleotide
gggccggagcggattttgc

SEQ ID NO: 131 - DNA, 19 bp, Artificial Sequence
  Synthetic polynucleotide
gaagccctacccgtgtatc

SEQ ID NO: 132 - DNA, 19 bp, Artificial Sequence
  Synthetic polynucleotide
cttgtcctcctctcccagt

SEQ ID NO: 133 - DNA, 19 bp, Artificial Sequence
  Synthetic polynucleotide
ctcccagtcggcaccacag

SEQ ID NO: 134 - DNA, 19 bp, Artificial Sequence
  Synthetic polynucleotide
agaggaatgcgtttccgga

SEQ ID NO: 135 - DNA, 19 bp, Artificial Sequence
  Synthetic polynucleotide
ccgggccgggctgacgcgg

SEQ ID NO: 136 - DNA, 19 bp, Artificial Sequence
  Synthetic polynucleotide
agaggcccagcgggagtcg

SEQ ID NO: 137 - DNA, 19 bp, Artificial Sequence
  Synthetic polynucleotide
gagagcgagcgcgcaacgg

SEQ ID NO: 138 - DNA, 19 bp, Artificial Sequence
  Synthetic polynucleotide
ggcctggcaggatgcgcgg

SEQ ID NO: 139 - DNA, 19 bp, Artificial Sequence
  Synthetic polynucleotide
ccgaaacgcgccaagttgg

SEQ ID NO: 140 - DNA, 19 bp, Artificial Sequence
  Synthetic polynucleotide
ccggactcggacgcgtggt

SEQ ID NO: 141 - DNA, 19 bp, Artificial Sequence
  Synthetic polynucleotide
tgtccagggacaatgagca

SEQ ID NO: 142 - DNA, 19 bp, Artificial Sequence
  Synthetic polynucleotide
gacaatgagcagggtgtcc

SEQ ID NO: 143 - DNA, 19 bp, Artificial Sequence
  Synthetic polynucleotide
ttggctggggtgtcgtcat

SEQ ID NO: 144 - DNA, 19 bp, Artificial Sequence
  Synthetic polynucleotide
tctgggaggggccaaggaa

SEQ ID NO: 145 - DNA, 19 bp, Artificial Sequence
  Synthetic polynucleotide
gagatgcctgtcattccct

SEQ ID NO: 146 - DNA, 19 bp, Artificial Sequence
  Synthetic polynucleotide
cctcccgactgtaaacata

SEQ ID NO: 147 - DNA, 19 bp, Artificial Sequence
  Synthetic polynucleotide
aggcaggctgcatccacct

SEQ ID NO: 148 - DNA, 19 bp, Artificial Sequence
  Synthetic polynucleotide
gccgagaatgagatgtaaa

SEQ ID NO: 149 - DNA, 19 bp, Artificial Sequence
  Synthetic polynucleotide
cgcggttgcgtggggagac

SEQ ID NO: 150 - DNA, 19 bp, Artificial Sequence
  Synthetic polynucleotide
gtggggagacgggcatcat

SEQ ID NO: 151 - DNA, 19 bp, Artificial Sequence
  Synthetic polynucleotide
gtcgcggccgcggttgcgt

SEQ ID NO: 152 - DNA, 19 bp, Artificial Sequence
  Synthetic polynucleotide
gggagcgacgtcgcggccg

SEQ ID NO: 153 - DNA, 19 bp, Artificial Sequence
  Synthetic polynucleotide
tctctcaggagcataggaa

SEQ ID NO: 154 - DNA, 19 bp, Artificial Sequence
  Synthetic polynucleotide
gagaggggtggtgatgtag

SEQ ID NO: 155 - DNA, 19 bp, Artificial Sequence
  Synthetic polynucleotide
ccgtccgcgcatatctctc

SEQ ID NO: 156 - DNA, 19 bp, Artificial Sequence
  Synthetic polynucleotide
cgaccacgccctggctttt

SEQ ID NO: 157 - DNA, 19 bp, Artificial Sequence
  Synthetic polynucleotide
cgcttgaaagcggcgagag

SEQ ID NO: 158 - DNA, 19 bp, Artificial Sequence
  Synthetic polynucleotide
cggaggtggggggggtcgt

SEQ ID NO: 159 - DNA, 19 bp, Artificial Sequence
  Synthetic polynucleotide
gggcacagaacccgctcgg

SEQ ID NO: 160 - DNA, 19 bp, Artificial Sequence
  Synthetic polynucleotide
accccccccacctccgagc

SEQ ID NO: 161 - DNA, 19 bp, Artificial Sequence
  Synthetic polynucleotide
aacccgctcggaggtgggg

SEQ ID NO: 162 - DNA, 19 bp, Artificial Sequence
  Synthetic polynucleotide
cacagaacccgctcggagg

SEQ ID NO: 163 - DNA, 19 bp, Artificial Sequence
  Synthetic polynucleotide
taggataacagcgagggac

SEQ ID NO: 164 - DNA, 19 bp, Artificial Sequence
  Synthetic polynucleotide
tggaaagagagagaccggc

SEQ ID NO: 165 - DNA, 19 bp, Artificial Sequence
  Synthetic polynucleotide
aaggccggaagtccttagc

SEQ ID NO: 166 - DNA, 19 bp, Artificial Sequence
  Synthetic polynucleotide
ccaaaagatagggcttttt

SEQ ID NO: 167 - DNA, 19 bp, Artificial Sequence
  Synthetic polynucleotide
aggcaatgcagaaaggttc

SEQ ID NO: 168 - DNA, 20 bp, Artificial Sequence
  Synthetic polynucleotide
gaggtcatgattcatcccac

SEQ ID NO: 169 - DNA, 20 bp, Artificial Sequence
  Synthetic polynucleotide
ggctactggtctccaccggc

SEQ ID NO: 170 - DNA, 20 bp, Artificial Sequence
  Synthetic polynucleotide
gaagctcgcttacaggaggg

SEQ ID NO: 171 - DNA, 20 bp, Artificial Sequence
  Synthetic polynucleotide
gctgctgagcgatgactcag

SEQ ID NO: 172 - DNA, 20 bp, Artificial Sequence
  Synthetic polynucleotide
gtccctgcgccagggtaaac

SEQ ID NO: 173 - DNA, 20 bp, Artificial Sequence
  Synthetic polynucleotide
gcaatctcggtccgccttac

SEQ ID NO: 174 - DNA, 20 bp, Artificial Sequence
  Synthetic polynucleotide
gccggctgttgagtcagctc

SEQ ID NO: 175 - DNA, 20 bp, Artificial Sequence
  Synthetic polynucleotide
gctggatgacaccactctgt

SEQ ID NO: 176 - DNA, 20 bp, Artificial Sequence
  Synthetic polynucleotide
gcggtggagacctcatgatc

SEQ ID NO: 177 - DNA, 20 bp, Artificial Sequence
  Synthetic polynucleotide
gcagcctcttgttcgctgct

SEQ ID NO: 178 - DNA, 20 bp, Artificial Sequence
  Synthetic polynucleotide
gttaagatagagaatgccat

SEQ ID NO: 179 - DNA, 20 bp, Artificial Sequence
  Synthetic polynucleotide
gttccgtacacagtcctttc

SEQ ID NO: 180 - DNA, 20 bp, Artificial Sequence
  Synthetic polynucleotide
gcctccgggcagagcgcgtg

SEQ ID NO: 181 - DNA, 21 bp, Artificial Sequence
  Synthetic polynucleotide
gatcgtcggtcgggtcatacg

SEQ ID NO: 182 - DNA, 21 bp, Artificial Sequence
  Synthetic polynucleotide
gttcgtcgcgatagatgatcg

SEQ ID NO: 183 - DNA, 21 bp, Artificial Sequence
  Synthetic polynucleotide
gcgcgtatcgtttcatcggcg

SEQ ID NO: 184 - DNA, 28 bp, Artificial Sequence
  Synthetic polynucleotide
tatttgttgattgggtagtttttgaatt

SEQ ID NO: 185 - DNA, 28 bp, Artificial Sequence
  Synthetic polynucleotide
actccctaacctcctaattcaacaaaaa

SEQ ID NO: 186 - DNA, 30 bp, Artificial Sequence
  Synthetic polynucleotide
ataggaagggttgtgaattaaaaagttagg

SEQ ID NO: 187 - DNA, 29 bp, Artificial Sequence
  Synthetic polynucleotide
acaactcacccaaaactaaacaaaccaaa

SEQ ID NO: 188 - DNA, 32 bp, Artificial Sequence
  Synthetic polynucleotide
gagatgtaaaaggattcctgaaaattatattt

SEQ ID NO: 189 - DNA, 32 bp, Artificial Sequence
  Synthetic polynucleotide
ctctctccaaaaatactctcaataacctttcc

SEQ ID NO: 190 - DNA, 30 bp, Artificial Sequence
  Synthetic polynucleotide
attttaagcgtttgagtgttgcaggtaggg

SEQ ID NO: 191 - DNA, 32 bp, Artificial Sequence
  Synthetic polynucleotide
taaaacaataaataacctcctaataactaaaa

SEQ ID NO: 192 - DNA, 31 bp, Artificial Sequence
  Synthetic polynucleotide
tttgatttttttgctgttgggaggtgtagat

SEQ ID NO: 193 - DNA, 30 bp, Artificial Sequence
  Synthetic polynucleotide
caactctccgaaaccttttcctacaataat

SEQ ID NO: 194 - DNA, 31 bp, Artificial Sequence
  Synthetic polynucleotide
aagttgtggggttatttgtgggttttaggag

SEQ ID NO: 195 - DNA, 30 bp, Artificial Sequence
  Synthetic polynucleotide
cccacaaccaccacacccatcaccaccaca

SEQ ID NO: 196 - DNA, 32 bp, Artificial Sequence
  Synthetic polynucleotide
ataaagattttaattataggaggtggagaaag

SEQ ID NO: 197 - DNA, 25 bp, Artificial Sequence
  Synthetic polynucleotide
aaaaccttctcctccaaccactaat

SEQ ID NO: 198 - DNA, 30 bp, Artificial Sequence
  Synthetic polynucleotide
tagtggtatgggggagtaggataagaaaag

SEQ ID NO: 199 - DNA, 29 bp, Artificial Sequence
  Synthetic polynucleotide
aatccccttaaactctaaactaccaaatc

SEQ ID NO: 200 - DNA, 34 bp, Artificial Sequence
  Synthetic polynucleotide
tccttgtaaaagaagttgtaattgtaattttagt

SEQ ID NO: 201 - DNA, 33 bp, Artificial Sequence
  Synthetic polynucleotide
taataacacccccaaataacttattaaactctt

SEQ ID NO: 202 - DNA, 36 bp, Artificial Sequence
  Synthetic polynucleotide
attaagaggaaagtttttataagttttttttagggg

SEQ ID NO: 203 - DNA, 39 bp, Artificial Sequence
  Synthetic polynucleotide
cctaattcttttataaccaccttatacaaattaaaactc